(19) United States
(12) Patent Application Publication
(10) Pub. No.: US 2021/0046177 A1
(43) Pub. Date: Feb. 18, 2021

(54) COMPOSITIONS AND METHODS FOR COMBINATION CANCER VACCINE AND IMMUNOLOGIC ADJUVANT THERAPY

(71) Applicants: NANTCELL, INC., Culver City, CA (US); NANTBIO, INC., Culver City, CA (US); NANT HOLDINGS IP, LLC, Culver City, CA (US)

(72) Inventors: Adrian E. RICE, Culver City, CA (US); Frank R. JONES, Culver City, CA (US); Kayvan NIAZI, Culver City, CA (US); Shahrooz RABIZADEH, Culver City, CA (US); Patrick SOON-SHIONG, Culver City, CA (US)

(21) Appl. No.: 16/964,348

(22) PCT Filed: Jan. 25, 2019
(86) PCT No.: PCT/US2019/015130

§ 371 Date: Jul. 23, 2020

Related U.S. Application Data
(60) Provisional application No. 62/622,773, filed on Jan. 26, 2018.

(51) Int. Cl. A61K 39/235; A61P 35/00; C12N 15/86; C12N 15/11; C12N 15/10

(52) U.S. Cl. CPC A61K 39/235; A61P 35/00; C07K 16/3007; C12N 15/11; C12N 15/102; C12N 15/86

(57) ABSTRACT

Methods and compositions for generating enhanced immune responses using adenovirus vectors that encode for an antigen and calreticulin, which serves as an immunologic adjuvant.

### CROSS REFERENCE

This application claims the benefit of U.S. Provisional Patent Application No. 62/622,773, filed Jan. 26, 2018, the entire contents of which are incorporated by reference herein.

### REFERENCE TO SEQUENCE LISTING

This application contains a Sequence Listing submitted as an electronic text file named “8774ETU-29_Sequence_Listing_ST25.txt”, having a size in bytes of 278000 bytes, and created on Jan. 25, 2019. The information contained in this electronic file is hereby incorporated by reference in its entirety pursuant to 37 CFR § 1.52(e)(5).

### BACKGROUND

Vaccines help the body fight disease by training the immune system to recognize and destroy harmful substances and diseased cells. Viral vaccines are currently being developed to help fight infectious diseases and cancers. These viral vaccines work by inducing expression of a small fraction of genes associated with a disease within the host's cells, which in turn, enhance the host's immune system to identify and destroy diseased cells. Cancer immunotherapy achieved by delivering viral vaccines encoding tumor-associated antigens (TAA) may have survival benefits; however, limitations to these strategies exist and more immunologically potent vaccines are needed. The present invention addresses this limitation by combining the administration of a vaccine encoding for an fusion protein of antigen of interest with calreticulin, to boost the resulting immune response, thereby, enhancing the efficacy and effectiveness of the vaccine in a subject.

### SUMMARY

In various aspects, the present disclosure provides a composition comprising: a recombinant replication defective viral vector comprising a nucleic acid sequence encoding an antigen and an E2b deletion; and a nucleic acid sequence encoding calreticulin. In some aspects, the antigen and calreticulin are expressed together as a fusion protein in a cell. In some aspects, the fusion protein induces apoptosis of the cell. In some aspects, the fusion protein induces phagocytosis of the cell by a second cell. In further aspects, the second cell is an antigen presenting cell. In some aspects, the antigen presenting cell cross-presents the antigen.

In some aspects, calreticulin boosts a host immune response to the composition. In some aspects, the host immune response is cytokine secretion, T cell proliferation, or a combination thereof. In further aspects, the nucleic acid sequence encoding calreticulin has at least 70%, at least 75%, at least 80%, at least 85%, at least 87%, at least 90%, at least 92%, at least 95%, at least 97%, or at least 99% sequence identity to SEQ ID NO: 107.

In some aspects, the antigen is a CEA antigen, a MUC1-C antigen, or a Brachyury antigen. In some aspects, the antigen is a tumor neo-antigen or a tumor-neo-epitope. In some aspects, the composition further comprises a second replication defective virus vector comprising a nucleic acid sequence encoding one or more additional target antigens or immunological epitopes thereof and a nucleic acid sequence encoding an additional calreticulin. In some aspects, the composition further comprises a third replication defective virus vector comprising a nucleic acid sequence encoding one or more additional target antigens or immunological epitopes thereof and a nucleic acid sequence encoding an additional calreticulin.

In some aspects, the replication defective virus vector further comprises a nucleic acid sequence encoding one or more additional target antigens or immunological epitopes thereof and a nucleic acid sequence encoding an additional calreticulin.

In further aspects, the one or more additional target antigens or immunological epitopes thereof is a tumor-specific antigen, a tumor-associated antigen, a bacterial antigen, a viral antigen, a yeast antigen, a fungal antigen, a protozoan antigen, a parasite antigen, a mitogen, or a combination thereof. In some aspects, the one or more additional target antigens or immunological epitopes thereof is human epidermal growth factor receptor 1 (HER1), human epidermal growth factor receptor 2 (HER2/neu), human epidermal growth factor receptor 3 (HER3), human epidermal growth factor receptor 4 (HER4), prostate-specific antigen (PSA), PSMA, folate receptor alpha, WT1, p53, MAGE-A1, MAGE-A2, MAGE-A3, MAGE-A4, MAGE-A6, MAGE-A10, MAGE-A12, BAGE, DAM-6, DAM-10, GAGE-1, GAGE-2, GAGE-8, GAGE-3, GAGE-4, GAGE-5, GAGE-6, GAGE-7B, NA88-A, NY-ESO-1, MART-1, MC1R, Gp100, PSA, PSM, Tyrosinase, TRP-1, TRP-2, ART-4, CAMEL, CEA, Cyp-B, BRCA1, Brachyury, Brachyury (TIVS7-2, polymorphism), Brachyury (IVS7 T/C polymorphism), T Brachyury, T, hTERT, hTRT, iCE, MUC1, MUC1 (VNTR polymorphism), MUC1c, MUCin, MUC2, PRAME, P15, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, HPV E6, HPV E7, and TEL/AM1.

In some aspects, the nucleic acid sequence encoding the antigen or the one or more additional antigens has at least 70%, at least 75%, at least 80%, at least 85%, at least 87%, at least 90%, at least 92%, at least 95%, at least 97%, or at least 99% sequence identity to SEQ ID NO: 1, SEQ ID NO: 3, SEQ ID NO: 4, SEQ ID NO: 100, or positions 1057 to 3165 of SEQ ID NO: 2.

In some aspects, the nucleic acid sequence encoding the antigen or the one or more additional antigens has at least 70%, at least 75%, at least 80%, at least 85%, at least 87%, at least 90%, at least 92%, at least 95%, at least 97%, or at least 99% sequence identity to SEQ ID NO: 5, SEQ ID NO: 6, SEQ ID NO: 7, SEQ ID NO: 101, or positions 93, 141-142, 149-151, 392, 404, 406, 422, 430-431, 444-445, or 460 of SEQ ID NO: 7.

In some aspects, the nucleic acid sequence encoding the antigen or the one or more additional antigens has at least 70%, at least 75%, at least 80%, at least 85%, at least 87%, at least 90%, at least 92%, at least 95%, at least 97%, or at least 99% sequence identity to SEQ ID NO: 9, SEQ ID NO: 10, SEQ ID NO: 14, SEQ ID NO: 102, or positions 1033 to 2283 of SEQ ID NO: 13.

In some aspects, the replication defective viral vector is an adenovirus vector. In some aspects, the adenovirus vector is an adenovirus subtype 5 (Ad5)-based vector. In further aspects, the replication defective viral vector comprises a deletion in an E1 region, an E2 region, an E3 region, an E4 region, or any combination thereof. In some aspects, the replication defective viral vector comprises a deletion in an E1 region. In some aspects, the replication defective viral vector comprises a deletion in an E1 region and E2 region.

In some aspects, the composition comprises at least 1×10⁹ viral particles, at least 1×10¹ viral particles, at least 1×10¹¹ viral particles, at least 5×10¹¹ viral particles, at least 1×10² viral particles, or at least 5×10¹² viral particles in a single dose.

In further aspects, the composition comprises 1×10⁹-5×10¹² viral particles in a single dose. In some aspects, the MUC1 antigen is a modified antigen having one or more mutations at positions 93, 141-142, 149-151, 392, 404, 406, 422, 430-431, 444-445, or 460 of SEQ ID NO: 7. In some aspects, the MUC1 antigen binds to HLA-A2, HLA-A3, HLA-A24, or a combination thereof. In some aspects, the Brachyury antigen is a modified Brachyury antigen comprising an amino acid sequence set forth in WLLPGTSTV (SEQ ID NO: 15). In some aspects, the Brachyury antigen binds to HLA-A2.

In some aspects, the composition or the replication-defective virus vector further comprises a nucleic acid sequences encoding a costimulatory molecule. In further aspects, the costimulatory molecule comprises B7, ICAM-1, LFA-3, or a combination thereof. In some aspects, the costimulatory molecule comprises a combination of B7, ICAM-1, and LFA-3. In some aspects, the composition further comprises a plurality of nucleic acid sequences encoding a plurality of costimulatory molecules positioned in the same replication-defective virus vector. In some aspects, the composition further comprises a plurality of nucleic acid sequences encoding a plurality of costimulatory molecules positioned in separate replication-defective virus vectors.

In further aspects, the composition further comprises an immune pathway checkpoint modulator. In some aspects, the immune pathway checkpoint modulator activates or potentiates an immune response. In some aspects, the immune pathway checkpoint inhibits an immune response. In some aspects, the immune pathway checkpoint modulator targets an endogenous immune pathway checkpoint protein or fragment thereof selected from the group consisting of: PD1, PDL1, PDL2, CD28, CD80, CD86, CTLA4, B7RP1, ICOS, B7RPI, B7-H3, B7-H4, BTLA, HVEM, KIR, TCR, LAG3, CD137, CD137L, OX40, OX40L, CD27, CD70, CD40, CD40L, TIM3, GAL9, ADORA, CD276, VTCN1, IDO1, KIR3DL1, HAVCR2, VISTA, and CD244. In some aspects, the immune pathway checkpoint modulator targets a PD1 protein. In some aspects, the immune pathway checkpoint modulator comprises siRNAs, antisense, small molecules, mimic, a recombinant form of a ligand, a recombinant form of a receptor, antibodies, or a combination thereof.

In some aspects, the immune pathway checkpoint inhibitor is an anti-PD-1 antibody or an anti-PD-L1 antibody. In further aspects, the immune pathway checkpoint inhibitor is Avelumab. In some aspects, the immune response is increased at least 2-, at least 3-, at least 4-, at least 5-, at least 6-, at least 7-, at least 8-, at least 9-, at least 10-, at least 15-, at least 20-, or at least 25-fold.

In further aspects, the composition further comprises an anti-CEA antibody. In some aspects, the anti-CEA antibody is NEO-201, COL1, COL2, COL3, COL4, COL5, COL6, COL7, COL8, COL9, COL10, COL11, COL12, COL3, COL14, COL15, arcitumomab, besilesomab, labetuzumab, or altumomab. In some aspects, the anti-CEA antibody is NEO-201.

In some aspects, the composition further comprises a chemotherapeutic agent. In some aspects, the chemotherapeutic agent is 5-FU, leucovorin, or oxaliplatin, or any combination thereof. In some aspects, the composition further comprises a population of engineered natural killer (NK) cells. In some aspects, the engineered NK cells comprise one or more NK cells that have been modified as essentially lacking the expression of KIR (killer inhibitory receptors), one or more NK cells that have been modified to express a high affinity CD16 variant, and one or more NK cells that have been modified to express one or more CARs (chimeric antigen receptors), or any combinations thereof.

In some aspects, the engineered NK cells comprise one or more NK cells that have been modified as essentially lacking the expression KIR. In other aspects, the engineered NK cells comprise one or more NK cells that have been modified to express a high affinity CD16 variant. In some aspects, the engineered NK cells comprise one or more NK cells that have been modified to express one or more CARs.

In further aspects, the CAR is a CAR for a tumor neo-antigen, tumor neo-epitope, WT1, p53, MAGE-A1, MAGE-A2, MAGE-A3, MAGE-A4, MAGE-A6, MAGE-A10, MAGE-A12, BAGE, DAM-6, DAM-10, Folate receptor alpha, GAGE-1, GAGE-2, GAGE-8, GAGE-3, GAGE-4, GAGE-5, GAGE-6, GAGE-7B, NA88-A, NY-ESO-1, MART-1, MC1R, Gp100, Tyrosinase, TRP-1, TRP-2, ART-4, CAMEL, CEA, Cyp-B, Her2/neu, Her3, BRCA1, Brachyury, Brachyury (TIVS7-2, polymorphism), Brachyury (IVS7 T/C polymorphism), T Brachyury, T, hTERT, hTRT, iCE, MUC1, MUC1 (VNTR polymorphism), MUC1c, MUC1n, MUC2, PRAME, P15, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDCl27/m, TPl/mbcr-abl, ETV6/AML, LDLR/FUT, Pml/RARα, TEL/AML, or any combination thereof.

In some aspects, the composition further comprises an IL-15 superagonist complex. In some aspects, the replication defective viral vector further comprises a nucleic acid sequence encoding for the IL-15 superagonist complex. In some aspects, the IL-15 super agonist complex is ALT-803. In further aspects, ALT-803 comprises two IL-15N72D domains and a dimeric IL-15 RαSu/Fc domain, wherein the IL-15N72D domain comprises at least 80%, at least 85%, at least 87%, at least 90%, at least 92%, at least 95%, at least 97%, or at least 99% sequence identity to SEQ ID NO: 84 and wherein the IL-15RαSu/Fc domain comprises at least 80%, at least 85%, at least 87%, at least 90%, at least 92%, at least 95%, at least 97%, or at least 99% sequence identity to SEQ ID NO: 85.

In various aspects, the present disclosure provides a method of treating a subject in need thereof, the method comprising administering to the subject any of the above compositions.

In various aspects, the present disclosure provides a method of treating a subject in need thereof, the method comprising administering to the subject: a recombinant replication defective viral vector comprising a nucleic acid sequence encoding an antigen; and a nucleic acid sequence encoding calreticulin.

In some aspects, the antigen and calreticulin are expressed together as a fusion protein in a cell. In some aspects, the fusion protein induces apoptosis of the cell. In some aspects, the fusion protein induces phagocytosis of the cell by a second cell. In some aspects, the second cell is an antigen presenting cell. In further aspects, the antigen presenting cell cross-presents the antigen. In some aspects, calreticulin boosts a host immune response to the antigen.

In some aspects, the host immune response is cytokine secretion, T cell proliferation, or a combination thereof. In some aspects, the nucleic acid sequence encoding calreticulin has at least 70%, at least 75%, at least 80%, at least 85%, at least 87%, at least 90%, at least 92%, at least 95%, at least 97%, or at least 99% sequence identity to SEQ ID NO: 107. In some aspects, the antigen is a CEA antigen, a MUC1-C antigen, or a Brachyury antigen. In some aspects, the antigen is a tumor neo-antigen or a tumor-neo-epitope.

In further aspects, the method further comprises a second replication defective virus vector comprising a nucleic acid sequence encoding one or more additional target antigens or immunological epitopes thereof and a nucleic acid sequence encoding an additional calreticulin. In still further aspects, the method further comprises a third replication defective virus vector comprising a nucleic acid sequence encoding one or more additional target antigens or immunological epitopes thereof and a nucleic acid sequence encoding an additional calreticulin.

In some aspects, the replication defective virus vector further comprises a nucleic acid sequence encoding one or more additional target antigens or immunological epitopes thereof and a nucleic acid sequence encoding an additional calreticulin. In some aspects, the one or more additional target antigens or immunological epitopes thereof is a tumor-specific antigen, a tumor-associated antigen, a bacterial antigen, a viral antigen, a yeast antigen, a fungal antigen, a protozoan antigen, a parasite antigen, a mitogen, or a combination thereof.

In some aspects, the one or more additional target antigens or immunological epitopes thereof is human epidermal growth factor receptor 1 (HER1), human epidermal growth factor receptor 2 (HER2/neu), human epidermal growth factor receptor 3 (HER3), human epidermal growth factor receptor 4 (HER4), prostate-specific antigen (PSA), PSMA, folate receptor alpha, WT1, p53, MAGE-A1, MAGE-A2, MAGE-A3, MAGE-A4, MAGE-A6, MAGE-A10, MAGE-A12, BAGE, DAM-6, DAM-10, GAGE-1, GAGE-2, GAGE-8, GAGE-3, GAGE-4, GAGE-5, GAGE-6, GAGE-7B, NA88-A, NY-ESO-1, MART-1, MC1R, Gp100, PSA, PSM, Tyrosinase, TRP-1, TRP-2, ART-4, CAMEL, CEA, Cyp-B, BRCA1, Brachyury, Brachyury (TIVS7-2, polymorphism), Brachyury (IVS7 T/C polymorphism), T Brachyury, T, hTERT, hTRT, iCE, MUC1, MUC1 (VNTR polymorphism), MUC1c, MUC1n, MUC2, PRAME, P15, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDCl27/m, TPI/mbcr-abl, ETV6/AML, LDLR/FUT, Pml/RARα, HPV E6, HPV E7, and TEL/AM1.

In some aspects, the nucleic acid sequence encoding the antigen or the one or more additional antigens has at least 70%, at least 75%, at least 80%, at least 85%, at least 87%, at least 90%, at least 92%, at least 95%, at least 97%, or at least 99% sequence identity to SEQ ID NO: 1, SEQ ID NO: 3, SEQ ID NO: 4, SEQ ID NO: 100, or positions 1057 to 3165 of SEQ ID NO: 2.

In other aspects, the nucleic acid sequence encoding the antigen or the one or more additional antigens has at least 70%, at least 75%, at least 80%, at least 85%, at least 87%, at least 90%, at least 92%, at least 95%, at least 97%, or at least 99% sequence identity to SEQ ID NO: 5, SEQ ID NO: 6, SEQ ID NO: 7, SEQ ID NO: 101, or positions 93, 141-142, 149-151, 392, 404, 406, 422, 430-431, 444-445, or 460 of SEQ ID NO: 7.

In still other aspects, the nucleic acid sequence encoding the antigen or the one or more additional antigens has at least 70%, at least 75%, at least 80%, at least 85%, at least 87%, at least 90%, at least 92%, at least 95%, at least 97%, or at least 99% sequence identity to SEQ ID NO: 9, SEQ ID NO: 10, SEQ ID NO: 14, SEQ ID NO: 102, or positions 1033 to 2283 of SEQ ID NO: 13.

In some aspects, the replication defective viral vector is an adenovirus vector. In some aspects, the adenovirus vector is an adenovirus subtype 5 (Ad5)-based vector. In some aspects, the replication defective viral vector comprises a deletion in an E1 region, an E2 region, an E3 region, an E4 region, or any combination thereof. In some aspects, the replication defective viral vector comprises a deletion in an E1 region. In some aspects, the replication defective viral vector comprises a deletion in an E1 region and E2 region.

In some aspects, the method comprises administering at least 1×10⁹ viral particles, at least 1×10¹⁰ viral particles, at least 1×10¹¹ viral particles, at least 5×10¹¹ viral particles, at least 1×10¹² viral particles, or at least 5×10¹² viral particles in a single dose. In some aspects, the method comprises administering 1×10⁹-5×10¹² viral particles in a single dose.

In some aspects, the MUC1 antigen is a modified antigen having one or more mutations at positions 94, 141-142, 149-151, 392, 404, 406, 422, 430-431, 444-445, or 460 of SEQ ID NO: 7. In some aspects, the MUC1 antigen binds to HLA-A2, HLA-A3, HLA-A24, or a combination thereof.

In other aspects, the Brachyury antigen is a modified Brachyury antigen comprising an amino acid sequence set forth in WLLPGTSTV (SEQ ID NO: 15). In some aspects, the Brachyury antigen binds to HLA-A2. In some aspects, the method further comprises administering the replication-defective virus vector, wherein the replication-defective virus vector further comprises a nucleic acid sequences encoding a costimulatory molecule.

In further aspects, the costimulatory molecule comprises B7, ICAM-1, LFA-3, or a combination thereof. In some aspects, the costimulatory molecule comprises a combination of B7, ICAM-1, and LFA-3. In some aspects, the method further comprises administering to the subject a plurality of nucleic acid sequences encoding a plurality of costimulatory molecules positioned in the same replication-defective virus vector.

In some aspects, the method further comprises administering to the subject a plurality of nucleic acid sequences encoding a plurality of costimulatory molecules positioned in separate replication-defective virus vectors. In some aspects, the method further comprises administering to the subject an immune pathway checkpoint modulator.

In some aspects, the immune pathway checkpoint modulator activates or potentiates an immune response. In some aspects, the immune pathway checkpoint inhibits an immune response. In some aspects, the immune pathway checkpoint modulator targets an endogenous immune pathway checkpoint protein or fragment thereof selected from the group consisting of: PD1, PDL1, PDL2, CD28, CD80, CD86, CTLA4, B7RP1, ICOS, B7RPI, B7-H3, B7-H4, BTLA, HVEM, KIR, TCR, LAG3, CD137, CD137L, OX40, OX40L, CD27, CD70, CD40, CD40L, TIM3, GAL9, ADORA, CD276, VTCN1, IDO1, KIR3DL1, HAVCR2, VISTA, and CD244.

In some aspects, the immune pathway checkpoint modulator targets a PD1 protein. In some aspects, the immune pathway checkpoint modulator comprises siRNAs, antisense, small molecules, mimic, a recombinant form of a ligand, a recombinant form of a receptor, antibodies, or a combination thereof. In some aspects, the immune pathway checkpoint inhibitor is an anti-PD-1 antibody or an anti-PD-L1 antibody. In further aspects, the immune pathway checkpoint inhibitor is Avelumab.

In some aspects, an immune response is increased at least 2, at least 3, at least 4, at least 5, at least 6, at least 7, at least 8, at least 9, at least 10, at least 15, at least 20, or at least 25 fold. In some aspects, the method further comprises administering to the subject an anti-CEA antibody.

In further aspects, the anti-CEA antibody is NEO-201, COL1, COL2, COL3, COL4, COL5, COL6, COL7, COL8, COL9, COL10, COL11, COL12, COL3, COL14, COL15, arcitumomab, besilesomab, labetuzumab, or altumomab. In still further aspects, the anti-CEA antibody is NEO-201.

In some aspects, the method further comprises administering to the subject a chemotherapeutic agent. In some aspects, the chemotherapeutic agent is 5-FU, leucovorin, or oxaliplatin, or any combination thereof.

In further aspects, the method further comprises administering to the subject a population of engineered natural killer (NK) cells. In some aspects, the engineered NK cells comprise one or more NK cells that have been modified as essentially lacking the expression of KIR (killer inhibitory receptors), one or more NK cells that have been modified to express a high affinity CD16 variant, and one or more NK cells that have been modified to express one or more CARs (chimeric antigen receptors), or any combinations thereof. In some aspects, the engineered NK cells comprise one or more NK cells that have been modified as essentially lacking the expression KIR. In some aspects, the engineered NK cells comprise one or more NK cells that have been modified to express a high affinity CD16 variant.

In some aspects, the engineered NK cells comprise one or more NK cells that have been modified to express one or more CARs. In some aspects, the CAR is a CAR for a tumor neo-antigen, tumor neo-epitope, WT1, p53, MAGE-A1, MAGE-A2, MAGE-A3, MAGE-A4, MAGE-A6, MAGE-A10, MAGE-A12, BAGE, DAM-6, DAM-10, Folate receptor alpha, GAGE-1, GAGE-2, GAGE-8, GAGE-3, GAGE-4, GAGE-5, GAGE-6, GAGE-7B, NA88-A, NY-ESO-1, MART-1, MC1R, Gp100, Tyrosinase, TRP-1, TRP-2, ART-4, CAMEL, CEA, Cyp-B, Her2/neu, Her3, BRCA1, Brachyury, Brachyury (TIVS7-2, polymorphism), Brachyury (IVS7 T/C polymorphism), T Brachyury, T, hTERT, hTRT, iCE, MUC1, MUC1 (VNTR polymorphism), MUC1c, MUC1n, MUC2, PRAME, P15, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDCl27/m, TPl/mbcr-abl, ETV6/AML, LDLR/FUT, Pml/RARα, TEL/AML1, or any combination thereof.

In some aspects, the administering is of a single dose of the recombinant replication defective viral vector comprising a nucleic acid sequence encoding an antigen is administered more than once over a 21 day period. In some aspects, the administering is of a single dose of the recombinant replication defective viral vector comprising a nucleic acid sequence encoding an antigen at a dose of 5×10¹¹ viral particles (VPs) three times at three week intervals or three times at four week intervals.

In some aspects, the administering is of a single dose of the recombinant replication defective viral vector comprises subcutaneous administration. In some aspects, monthly booster immunizations are given at one to two month intervals. In some aspects, the administering is of the recombinant replication defective viral vector comprising a nucleic acid sequence encoding an antigen is administered at least once, at least twice, at least three times, at least four times, or at least five times in a dosing regimen.

In some aspects, the antigen induces an immune response. In further aspects, the immune response is measured as antigen specific antibody response. In further aspects, the immune response is measured as antigen specific cell-mediated immunity (CMI). In still further aspects, the immune response is measured as antigen specific IFN-γ secretion. In some aspects, the immune response is measured as antigen specific IL-2 secretion. In some aspects, the immune response against the antigen is measured by ELISpot assay. In some aspects, the immune response is measured by T-cell lysis of CAP-1 pulsed antigen-presenting cells, allogeneic antigen expressing cells from a tumor cell line or from an autologous tumor.

In some aspects, the replication defective adenovirus infects dendritic cells in the subject and wherein the infected dendritic cells present the antigen, thereby inducing the immune response. In some aspects, the administering comprises subcutaneous, parenteral, intravenous, intramuscular, or intraperitoneal administration.

In some aspects, the subject has or does not have a proliferative disease cancer. In some aspects, the subject has colorectal adenocarcinoma, metastatic colorectal cancer, advanced CEA expressing colorectal cancer, breast cancer, lung cancer, bladder cancer, or pancreas cancer.

In some aspects, the subject has at least 1, 2, or 3 sites of metastatic disease. In some aspects, the subject comprises cells overexpressing CEA. In further aspects, the cells overexpressing CEA, overexpress CEA by at least 2, 3, 4, 5, 6, 7, 8, 9, or 10 times over a baseline CEA expression in a non-cancer cell.

In further aspects, cells overexpressing CEA comprise cancer cells. In some aspects, the subject has a diagnosed disease predisposition. In some aspects, the subject has a stable disease. In some aspects, the subject has a genetic predisposition for a disease. In some aspects, the disease is a cancer. In some aspects, the cancer is selected from the group consisting of prostate cancer, colon cancer, breast cancer, or gastric cancer.

In further aspects, the cancer is prostate cancer. In other aspects, the cancer is colon cancer. In some aspects, the subject is a human. In some aspects, the replication defective viral vector further comprises a nucleic acid sequence encoding for the IL-15 superagonist complex. In some aspects, the composition further comprises an IL-15 superagonist complex. In some aspects, the IL-15 superagonist complex is ALT-803.

In further aspects, ALT-803 comprises two IL-15N72D domains and a dimeric IL-15 RαSu/Fc domain, wherein the IL-15N72D domain comprises at least 80%, at least 85%, at least 87%, at least 90%, at least 92%, at least 95%, at least 97%, or at least 99% sequence identity to SEQ ID NO: 84 and wherein the IL-15RαSu/Fc domain comprises at least 80%, at least 85%, at least 87%, at least 90%, at least 92%, at least 95%, at least 97%, or at least 99% sequence identity to SEQ ID NO: 85.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a schematic showing each step in the process of manufacturing personalized neo-antigen vaccines. These steps include patient-specific identification of neo-antigens and/or neo-epitopes, design of a vector encoding for the neo-antigens and/or neo-epitope, cloning, vector construction, purification of the vector, release assays, and therapy with the resulting products in patients in need thereof.

### DETAILED DESCRIPTION

The following passages describe different aspects of certain embodiments in greater detail. Each aspect may be combined with any other aspect or aspects unless clearly indicated to the contrary. In particular, any feature indicated as being preferred or advantageous may be combined with any other feature of features indicated as being preferred or advantageous.

Unless otherwise indicated, any embodiment can be combined with any other embodiment. A variety of aspects can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges include the range endpoints.

To address the low immunogenicity of tumor associated antigens (TAA), a variety of advanced, multi-component vaccination strategies including combination therapy a calreticulin (CRT)-TAA fusion are disclosed herein. Some embodiments relate to recombinant viral vectors that provide innate pro-inflammatory signals, while simultaneously engineered to express the antigen of interest, such as CEA. Of particular interest are adenovirus serotype-5 (Ad5)-based immunotherapeutics that can be used in humans to induce robust T-cell-mediated immune (CMI) responses, all while maintaining an extensive safety profile.

Compared to first generation adenovirus vectors, certain embodiments of the Second Generation E2b deleted adenovirus vectors contain additional deletions in the DNA polymerase gene (pol) and deletions of the pre-terminal protein (pTP). E2b deleted vectors have up to a 13 kb gene-carrying capacity as compared to the 5 to 6 kb capacity of First Generation adenovirus vectors, easily providing space for nucleic acid sequences encoding any of a variety of target antigens. The E2b deleted adenovirus vectors also have reduced adverse reactions as compared to first generation adenovirus vectors.

It has been discovered that Ad5 [E1−, E2b−] vectors are not only are safer than, but appear to be superior to Ad5 [E1−] vectors in regard to induction of antigen specific immune responses, making them much better suitable as a platform to deliver CEA vaccines that can result in a clinical response. In other cases, immune induction may take months. Ad5 [E1−, E2b−] vectors not only are safer than, but appear to be superior to Ad5 [E1−] vectors in regard to induction of antigen specific immune responses, making them much better suitable as a platform to deliver CEA vaccines that can result in a clinical response.

Certain embodiments use the new Ad5 [E1−, E2b−] vector system to deliver a long sought-after need for a develop a therapeutic vaccine against CEA, overcome barriers found with other Ad5 systems and permit the immunization of people who have previously been exposed to Ad5.

The innate immune response to wild type Ad can be complex, and it appears that Ad proteins expressed from adenovirus vectors play an important role. Specifically, the deletions of pre-terminal protein and DNA polymerase in the E2b deleted vectors appear to reduce inflammation during the first 24 to 72 h following injection, whereas First Generation adenovirus vectors stimulate inflammation during this period. In addition, it has been reported that the additional replication block created by E2b deletion also leads to a 10,000-fold reduction in expression of Ad late genes, well beyond that afforded by E1, E3 deletions alone. The decreased levels of Ad proteins produced by E2b deleted adenovirus vectors effectively reduce the potential for competitive, undesired, immune responses to Ad antigens, responses that prevent repeated use of the platform in Ad immunized or exposed individuals. The reduced induction of inflammatory response by second generation E2b deleted vectors results in increased potential for the vectors to express desired vaccine antigens during the infection of antigen presenting cells (i.e., dendritic cells), decreasing the potential for antigenic competition, resulting in greater immunization of the vaccine to the desired antigen relative to identical attempts with First Generation adenovirus vectors. E2b deleted adenovirus vectors provide an improved Ad-based vaccine candidate that is safer, more effective, and more versatile than previously described vaccine candidates using First Generation adenovirus vectors. Thus, first generation, E1-deleted Adenovirus subtype 5 (Ad5)-based vectors, although promising platforms for use as cancer vaccines, are impeded in activity by naturally occurring or induced Ad-specific neutralizing antibodies. Without being bound by theory, Ad5-based vectors with deletions of the E1 and the E2b regions (Ad5 [E1−, E2b−]), the latter encoding the DNA polymerase and the pre-terminal protein, for example by virtue of diminished late phase viral protein expression, may avoid immunological clearance and induce more potent immune responses against the encoded tumor antigen transgene in Ad-immune hosts.

Some embodiments relate to methods and compositions (e.g., viral vectors) for generating immune responses against target antigens, in particular, those associated or related to infectious disease or proliferative cell disease such as cancer. Some embodiments relate to methods and compositions for generating immune responses in an individual against target antigens, in particular, those related to cell proliferation diseases such as cancer. In some embodiments, compositions and methods described herein relate to generating an immune response in an individual against cells expressing and/or presenting a target antigen or a target antigen signature comprising at least one target antigen.

The compositions and methods can be used to generate an immune response against a target antigen expressed and/or presented by a cell. For example, the compositions and methods can be used to generate immune responses against a carcinoembryonic antigen (CEA), such as CEA expressed or presented by a cell. For example, the compositions and methods can be used to generate an immune response against CEA(6D) expressed or presented by a cell. For example, the compositions and methods can be used to generate an immune response against Mucin 1 (MUC1) expressed and/or presented by a cell. For example, the compositions and methods can be used to generate an immune response against MUC1c expressed and/or presented by a cell. For example, the compositions and methods can be used to generate an immune response against Brachyury (T protein (T)) expressed and/or presented by a cell.

The compositions and methods can be used to generate an immune response against multiple target antigens expressed and/or presented by a cell. For example, the compositions and methods can be used to generate an immune response against CEA.

A modified form of CEA can be used in a vaccine directed to raising an immune response against CEA or cells expressing and/or presenting CEA. In particular, some embodiments provide an improved Ad-based vaccine such that multiple vaccinations against one or more antigenic target entity can be achieved. In some embodiments, the improved Ad-based vaccine comprises a replication defective adenovirus carrying a target antigen, a fragment, a variant or a variant fragment thereof, such as Ad5 [E1−, E2b−]-CEA(6D). Variants or fragments of target antigens, such as CEA, can be selected based on a variety of factors, including immunogenic potential. A mutant CEA, CEA(6D) can utilized for its increased capability to raise an immune response relative to the CEA(WT). Importantly, vaccination can be performed in the presence of preexisting immunity to the Ad or administered to subjects previously immunized multiple times with the Ad vector as described herein or other Ad vectors. The Ad vectors can be administered to subjects multiple times to induce an immune response against an antigen of interest, such as CEA, including but not limited to, the production of antibodies and CMI responses against one or more target antigens.

As used herein, unless otherwise indicated, the article “a” means one or more unless explicitly otherwise provided for. As used herein, unless otherwise indicated, terms such as “contain,” “containing,” “include,” “including,” and the like mean “comprising.” As used herein, unless otherwise indicated, the term “or” can be conjunctive or disjunctive. As used herein, unless otherwise indicated, any embodiment can be combined with any other embodiment.

An “adenovirus” (Ad) refers to non-enveloped DNA viruses from the family Adenoviridae. These viruses can be found in, but are not limited to, human, avian, bovine, porcine and canine species. Some embodiments contemplate the use of any Ad from any of the four genera of the family Adenoviridae (e.g., Aviadenovirus, Mastadenovirus, Atadenovirus and Siadenovirus) as the basis of an E2b deleted virus vector, or vector containing other deletions as described herein. In addition, several serotypes are found in each species. Ad also pertains to genetic derivatives of any of these viral serotypes, including but not limited to, genetic mutations, deletions or transpositions.

A “helper adenovirus” or “helper virus” refers to an Ad that can supply viral functions that a particular host cell cannot (the host may provide Ad gene products such as E1 proteins). This virus is used to supply, in trans, functions (e.g., proteins) that are lacking in a second virus, or helper dependent virus (e.g., a gutted or gutless virus, or a virus deleted for a particular region such as E2b or other region as described herein); the first replication-incompetent virus is said to “help” the second, helper dependent virus thereby permitting the production of the second viral genome in a cell.

An “adenovirus 5 null (Ad5-null)” refers to a non-replicating Ad that does not contain any heterologous nucleic acid sequences for expression.

A “first generation adenovirus” refers to an Ad that has the early region 1 (E1) deleted. In additional cases, the early region 3 (E3) may also be deleted.

“Gutted” or “gutless” refers to an Ad vector that has been deleted of all viral coding regions.

“Transfection” refers to the introduction of foreign nucleic acid into eukaryotic cells. Exemplary means of transfection include calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

“Stable transfection” or “stably transfected” refers to the introduction and integration of foreign nucleic acid, DNA or RNA, into the genome of the transfected cell. The term “stable transfectant” refers to a cell which has stably integrated foreign DNA into the genomic DNA.

A “reporter gene” indicates a nucleotide sequence that encodes a reporter molecule (e.g., an enzyme). A “reporter molecule” is detectable in any of a variety of detection systems, including, but not limited to, enzyme-based detection assays (e.g., ELISA, histochemical assays), fluorescent, radioactive, and luminescent systems. The E. coli-galactosidase gene, green fluorescent protein (GFP), the human placental alkaline phosphatase gene, the chloramphenicol acetyltransferase (CAT) gene; and other reporter genes may be employed.

A “heterologous sequence” refers to a nucleotide sequence that is ligated to, or is manipulated to become ligated to, a nucleic acid sequence to which it is not ligated in nature, or to which it is ligated at a different location in nature. Heterologous nucleic acid may include a naturally occurring nucleotide sequence or some modification relative to the naturally occurring sequence.

A “transgene” refers to any gene coding region, either natural or heterologous nucleic acid sequences or fused homologous or heterologous nucleic acid sequences, introduced into cells or a genome of subject. Transgenes may be carried on any viral vector used to introduce transgenes to the cells of the subject.

A “second generation adenovirus” refers to an Ad that has all or parts of the E1, E2, E3, and, in certain embodiments, E4 DNA gene sequences deleted (removed) from the virus.

A “subject” refers to any animal, including, but not limited to, humans, non-human primates (e.g., rhesus or other types of macaques), mice, pigs, horses, donkeys, cows, sheep, rats and fowls.

An “immunogenic fragment” refers to a fragment of a polypeptide that is specifically recognized (i.e., specifically bound) by a B-cell and/or T-cell surface antigen receptor resulting in a generation of an immune response specifically against a fragment.

A “target antigen” or “target protein” refers to a molecule, such as a protein, against which an immune response is to be directed.

“E2b deleted” refers to a DNA sequence mutated in such a way so as to prevent expression and/or function of at least one E2b gene product. Thus, in certain embodiments, “E2b deleted” is used in relation to a specific DNA sequence that is deleted (removed) from an Ad genome. E2b deleted or “containing a deletion within an E2b region” refers to a deletion of at least one base pair within an E2b region of an Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, a deletion is of more than 150, 160, 170, 180, 190, 200, 250, or 300 base pairs within an E2b region of an Ad genome. An E2b deletion may be a deletion that prevents expression and/or function of at least one E2b gene product and therefore, encompasses deletions within exons of encoding portions of E2b-specific proteins as well as deletions within promoter and leader sequences. In certain embodiments, an E2b deletion is a deletion that prevents expression and/or function of one or both a DNA polymerase and a preterminal protein of an E2b region. In a further embodiment, “E2b deleted” refers to one or more point mutations in a DNA sequence of this region of an Ad genome such that one or more encoded proteins is non-functional. Such mutations include residues that are replaced with a different residue leading to a change in an amino acid sequence that result in a nonfunctional protein.

“E1-deleted” refers to a DNA sequence that is mutated in such a way so as to prevent expression and/or function of at least one E1 gene product. Thus, in certain embodiments, “E1 deleted” is used in relation to a specific DNA sequence that is deleted (removed) from the Ad genome. E1 deleted or “containing a deletion within the E1 region” refers to a deletion of at least one base pair within the E1 region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E1 region of the Ad genome. An E1 deletion may be a deletion that prevents expression and/or function of at least one E1 gene product and therefore, encompasses deletions within exons of encoding portions of E1-specific proteins as well as deletions within promoter and leader sequences. In certain embodiments, an E1 deletion is a deletion that prevents expression and/or function of one or both of a trans-acting transcriptional regulatory factor of the E1 region. In a further embodiment, “E1 deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

“Generating an immune response” or “inducing an immune response” refers to a statistically significant change, e.g., increase or decrease, in the number of one or more immune cells (T-cells, B-cells, antigen-presenting cells, dendritic cells, neutrophils, and the like) or in the activity of one or more of these immune cells (CTL activity, HTL activity, cytokine secretion, change in profile of cytokine secretion, etc.).

The terms “nucleic acid” and “polynucleotide” are used essentially interchangeably herein. Polynucleotides may be single-stranded (coding or antisense) or double-stranded, and may be DNA (e.g., genomic, cDNA, or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide as described herein, and a polynucleotide may, but need not, be linked to other molecules and/or support materials. An isolated polynucleotide, as used herein, means that a polynucleotide is substantially away from other coding sequences. For example, an isolated DNA molecule as used herein does not contain large portions of unrelated coding DNA, such as large chromosomal fragments or other functional genes or polypeptide coding regions. This refers to the DNA molecule as originally isolated, and does not exclude genes or coding regions later added to the segment recombinantly in the laboratory.

As will be understood by those skilled in the art, the polynucleotides can include genomic sequences, extra-genomic and plasmid-encoded sequences and smaller engineered gene segments that express, or may be adapted to express target antigens as described herein, fragments of antigens, peptides and the like. Such segments may be naturally isolated, or modified synthetically by the hand of man.

Typically, polynucleotide variants will contain one or more substitutions, additions, deletions and/or insertions, preferably such that the immunogenicity of the epitope of the polypeptide encoded by the variant polynucleotide or such that the immunogenicity of the heterologous target protein is not substantially diminished relative to a polypeptide encoded by the native polynucleotide sequence. In some cases, the one or more substitutions, additions, deletions and/or insertions may result in an increased immunogenicity of the epitope of the polypeptide encoded by the variant polynucleotide. As described elsewhere herein, the polynucleotide variants can encode a variant of the target antigen, or a fragment (e.g., an epitope) thereof wherein the propensity of the variant polypeptide or fragment (e.g., epitope) thereof to react with antigen-specific antisera and/or T-cell lines or clones is not substantially diminished relative to the native polypeptide. The polynucleotide variants can encode a variant of the target antigen, or a fragment thereof wherein the propensity of the variant polypeptide or fragment thereof to react with antigen-specific antisera and/or T-cell lines or clones is substantially increased relative to the native polypeptide.

The term “variants” should also be understood to encompass homologous genes of xenogenic origin. In particular embodiments, variants or fragments of target antigens are modified such that they have one or more reduced biological activities. For example, an oncogenic protein target antigen may be modified to reduce or eliminate the oncogenic activity of the protein, or a viral protein may be modified to reduce or eliminate one or more activities or the viral protein. An example of a modified CEA protein is a CEA having a N610D mutation, resulting in a variant protein with increased immunogenicity.

When comparing polynucleotide sequences, two sequences are “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned. Optimal alignment of sequences for comparison may be conducted using the Megalign program in the Lasergene suite of bioinformatics software using default parameters. Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA), or by inspection. One example of algorithms that are suitable for determining percent sequence identity and sequence similarity are the BLAST and BLAST 2.0 algorithms. BLAST and BLAST 2.0 can be used, for example with the parameters described herein, to determine percent sequence identity for the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program uses as defaults a word length (W) of 11, and expectation (E) of 10, and the BLOSUM62 scoring matrix alignments, (B) of 50, expectation (E) of 10, M=5, N=−4 and a comparison of both strands.

The “percentage of sequence identity” can be determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence and multiplying the results by 100 to yield the percentage of sequence identity.

It will be appreciated by those of ordinary skill in the art that, as a result of the degeneracy of the genetic code, there are many nucleotide sequences that encode a particular antigen of interest, or fragment thereof, as described herein. Some of these polynucleotides bear minimal homology to the nucleotide sequence of any native gene. Nonetheless, polynucleotides that vary due to differences in codon usage are specifically contemplated. Further, alleles of the genes comprising the polynucleotide sequences provided herein are within the scope of some embodiments. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

### Viral Vectors for Immunotherapies and Vaccines

Recombinant viral vectors can be used to express protein coding genes or antigens (e.g., TAAs (tumor-associated antigens) and/or IDAAs (infectious-disease associated antigens)). The advantages of recombinant viral vector based vaccines and immunotherapy include high efficiency gene transduction, highly specific delivery of genes to target cells, induction of robust immune responses, and increased cellular immunity. Certain embodiments provide for recombinant adenovirus vectors comprising deletions or insertions of crucial regions of the viral genome. The viral vectors of provided herein can comprise heterologous nucleic acid sequences that encode one or more target antigens of interest, or variants, fragments or fusions thereof, against which it is desired to generate an immune response.

Suitable viral vectors that can be used with the methods and compositions as provided herein, include but are not limited to retroviruses, lentiviruses, provirus, Vaccinia virus, adenoviruses, adeno-associated viruses, self-complementary adeno-associated virus, Cytomegalovirus, Sendai virus, HPV virus, or adenovirus. In some embodiments, the viral vector can be replication-competent. In some embodiments, the viral vector can be replication-defective. For replication-defective viral vectors, the viruses' genome can have the coding regions necessary for additional rounds of replication and packaging replaced with other genes, or deleted. These viruses are capable of infecting their target cells and delivering their viral payload, but then fail to continue the typical lytic pathway that leads to cell lysis and death. Depending on the viral vector, the typical maximum length of an allowable DNA or cDNA insert in a replication-defective viral vector is can be about 8-10 kilobases (kB).

Retroviruses have been used to express antigens, such as an enveloped, single-stranded RNA virus that contains reverse transcriptase. Retrovirus vectors can be replication-defective. Retrovirus vectors can be of murine or avian origin. Retrovirus vectors can be from Moloney murine leukemia virus (MoMLV). Retrovirus vectors can be used that require genome integration for gene expression. Retrovirus vectors can be used to provide long-term gene expression. For example, retrovirus vectors can have a genome size of approximately 7-11 kb and the vector can harbor 7-8 kb long foreign DNA inserts. Retrovirus vectors can be used to display low immunogenicity and most patients do not show pre-existing immunity to retroviral vectors. Retrovirus vectors can be used to infect dividing cells. Retrovirus vectors can be used to not infect non-dividing cells.

Lentivirus vectors have been used to express antigens. Lentiviruses constitute a subclass of retroviruses. Lentivirus vectors can be used to infect non-dividing cells. Lentivirus vectors can be used to infect dividing cells. Lentivirus vectors can be used to infect both non-dividing and dividing cells. Lentiviruses generally exhibit broader tropism than retroviruses. Several proteins such as tat and rev regulate the replication of lentiviruses. These regulatory proteins are typically absent in retroviruses. HIV is an exemplary lentivirus that can been engineered into a transgene delivery vector. The advantages of lentivirus vectors are similar to those of retroviral vectors. Although lentiviruses can potentially trigger tumorigenesis, the risk is lower than that of retroviral vectors, as the integration sites of lentiviruses are away from the sites harboring cellular promoters. HIV-based vectors can be generated, for example, by deleting the HIV viral envelope and some of the regulatory genes not required during vector production. Instead of parental envelope, several chimeric or modified envelope vectors are generated because it determines the cell and tissue specificity.

Cytomegalovirus (CMV) vectors have been used to express antigens and is a member of the herpesviruses. Species-specific CMVs can be used (e.g., human CMV (HCMV), e.g., human herpesvirus type 5. HCMV contains a 235-kb double-stranded linear DNA genome surrounded by a capsid. The envelope contains glycoproteins gB and gH, which bind to cellular receptors.

Sendai virus (SeV) vectors have been used to express antigens. SeV is an enveloped, single-stranded RNA virus of the family Paramyxovirus. The SeV genome encodes six protein and two envelope glycoproteins, HN and F proteins, that mediate cell entry and determine its tropism. SeV vectors that lack F protein can be used as a replication-defective virus to improve the safety of the vector. SeV vector produced in a packaging cell can be used to expresses the F protein. An F gene-deleted and transgene-inserted genome can be transfected into a packaging cell. SeV contains RNA dependent RNA polymerase and viral genome localizes to the cytoplasm. This ensures that fast gene expression occurs soon after infection and the genotoxic advantage of SeV. SeV vectors can be used to exhibit highly efficient gene transfer. SeV vectors can be used to transduce both dividing and non-dividing cells. SeV vectors can be used to transduce non-dividing cells. SeV vectors can be used to transduce dividing cells. SeV vectors can be used, for example, to efficiently transduce human airway epithelial cells. SeV vectors can be, for example, administered by a mucosal (e.g., oral and nasal) route. Intranasal administration can be used to potentially reduce the influence of a pre-existing immunity to SeV, as compared to intramuscular administration. Compared to other viral vectors, its transgene capacity (3.4 kb) is low. SeV is highly homologous to the human parainfluenza type 1 (hPIV-1) virus; thus, a pre-existing immunity against hPIV-1 can work against the use of SeV.

### Adenovirus Vectors

In general, adenoviruses are attractive for clinical because they can have a broad tropism, they can infect a variety of dividing and non-dividing cell types, and they can be used systemically as well as through more selective mucosal surfaces in a mammalian body. In addition, their relative thermostability further facilitates their clinical use. Adenoviruses (Ads) are a family of DNA viruses characterized by an icosahedral, non-enveloped capsid containing a linear double-stranded genome. Generally, adenoviruses are found as non-enveloped viruses comprising double-stranded DNA genome approximated ˜30-35 kilobases in size. Of the human Ads, none are currently associated with any neoplastic disease, and only cause relatively mild, self-limiting illness in immunocompetent individuals. The first genes expressed by the virus are the E1 genes, which act to initiate high-level gene expression from the other Ad5 gene promoters present in the wild type genome. Viral DNA replication and assembly of progeny virions occur within the nucleus of infected cells, and the entire life cycle takes about 36 hr with an output of approximately 10⁴ virions per cell. The wild type Ad5 genome is approximately 36 kb, and encodes genes that are divided into early and late viral functions, depending on whether they are expressed before or after DNA replication. The early/late delineation is nearly absolute, since it has been demonstrated that super-infection of cells previously infected with an Ad5 results in lack of late gene expression from the super-infecting virus until after it has replicated its own genome. Without bound by theory, this is likely due to a replication dependent cis-activation of the Ad5 major late promoter (MLP), preventing late gene expression (primarily the Ad5 capsid proteins) until replicated genomes are present to be encapsulated. The composition and methods as described herein, in some embodiments, take advantage of feature in the development of advanced generation Ad vectors/vaccines. The linear genome of the adenovirus is generally flanked by two origins for DNA replication (ITRs) and has eight units for RNA polymerase II-mediated transcription. The genome carries five early units E1A, E1B, E2, E3, E4, and E5, two units that are expressed with a delay after initiation of viral replication (IX and IVa2), and one late unit (L) that is subdivided into L1-L5. Some adenoviruses can further encode one or two species of RNA called virus-associated (VA) RNA.

Adenoviruses that induce innate and adaptive immune responses in human patient are provided. By deletion or insertion of crucial regions of the viral genome, recombinant vectors are provided that have been engineered to increase their predictability and reduce unwanted side effects. In some aspects, there is provided an adenovirus vector comprising the genome deletion or insertion selected from the group consisting of: E1A, E1B, E2, E3, E4, E5, IX, IVa2, L1, L2, L3, L4, and L5, and any combination thereof.

Certain embodiments provide recombinant adenovirus vectors comprising an altered capsid. Generally, the capsid of an adenovirus primarily comprises 20 triangular facets of an icosahedron, each icosahedron containing 12 copies of hexon trimers. In addition, there are also other several additional minor capsid proteins, IIIa, VI, VIII, and IX.

Certain embodiments provide recombinant adenovirus vectors comprising one or more altered fiber proteins. In general, the fiber proteins, which also form trimers, are inserted at the 12 vertices into the pentameric penton bases. The fiber can comprise of a thin N-terminal tail, a shaft, and a knob domain. The shaft can comprise a variable number of β-strand repeats. The knob can comprise one or more loops of A, B, C, D, E, F, G, H, I, and/or J. The fiber knob loops can bind to cellular receptors. Certain embodiments provide adenovirus vectors to be used in vaccine systems for the treatment of cancers and infectious diseases.

Suitable adenoviruses that can be used with the present methods and compositions of the disclosure include but are not limited to species-specific adenovirus including human subgroups A, B1, B2, C, D, E and F or their crucial genomic regions as provided herein, which subgroups can further be classified into immunologically distinct serotypes. Further, suitable adenoviruses that can be used with the present methods and compositions of the disclosure include, but are not limited to, species-specific adenovirus or their crucial genomic regions identified from primates, bovines, fowls, reptiles, or frogs.

Some adenoviruses serotypes preferentially target distinct organs. Serotypes such as AdHu1, AdHu2, and AdHu5 (subgenus C), generally effect the infect upper respiratory, while subgenera A and F effect gastrointestinal organs. Certain embodiments provide recombinant adenovirus vectors to be used in preferentially target distinct organs for the treatment of organ-specific cancers or organ-specific infectious diseases. In some applications, the recombinant adenovirus vector is altered to reduce tropism to a specific organ in a mammal. In some applications, the recombinant adenovirus vector is altered to increase tropism to a specific organ in a mammal.

The tropism of an adenovirus can be determined by their ability to attach to host cell receptors. In some instances, the process of host cell attachment can involve the initial binding of the distal knob domain of the fiber to a host cell surface molecule followed by binding of the RGD motif within the penton base with αV integrins. Certain embodiments provide recombinant adenovirus vectors with altered tropism such that they can be genetic engineered to infect specific cell types of a host. Certain embodiments provide recombinant adenovirus vectors with altered tropism for the treatment of cell-specific cancers or cell-specific infectious diseases. Certain embodiments provide recombinant adenovirus vectors with altered fiber knob from one or more adenoviruses of subgroups A, B, C, D, or F, or a combination thereof or the insertion of RGD sequences. In some applications, the recombinant adenovirus vectors comprising an altered fiber knob results in a vector with reduced tropism for one or more particular cell types. In some applications, the recombinant adenovirus vectors comprising an altered fiber knob results in a vector with enhanced tropism for one or more particular cell types. In some applications, the recombinant adenovirus vectors comprising an altered fiber knob results in a vector with reduced product-specific B or T-cell responses. In some applications, the recombinant adenovirus vectors comprising an altered fiber knob results in a vector with enhanced product-specific B or T-cell responses.

Certain embodiments provide recombinant adenovirus vectors that are coated with other molecules to circumvent the effects of virus-neutralizing antibodies or improve transduction in to a host cell. Certain embodiments provide recombinant adenovirus vectors that are coated with an adaptor molecule that aids in the attachment of the vector to a host cell receptor. By way of example an adenovirus vector can be coated with adaptor molecule that connects coxsackie Ad receptor (CAR) with CD40L resulting in increased transduction of dendritic cells (DCs), thereby enhancing immune responses in a subject. Other adenovirus vectors similarly engineered for enhancing the attachment to other target cell types are also contemplated.

### Ad5 Vectors

Studies in humans and animals have demonstrated that pre-existing immunity against Ad5 can be an inhibitory factor to commercial use of Ad-based vaccines. The preponderance of humans have antibody against Ad5, the most widely used subtype for human vaccines, with two-thirds of humans studied having lympho-proliferative responses against Ad5. This pre-existing immunity can inhibit immunization or re-immunization using typical Ad5 vaccines and can preclude the immunization of a vaccine against a second antigen, using an Ad5 vector, at a later time. Overcoming the problem of pre-existing anti-vector immunity has been a subject of intense investigation. Investigations using alternative human (non-Ad5 based) Ad5 subtypes or even non-human forms of Ad5 have been examined. Even if these approaches succeed in an initial immunization, subsequent vaccinations can be problematic due to immune responses to the novel Ad5 subtype. To avoid the Ad5 immunization barrier, and improve upon the limited efficacy of first generation Ad5 [E1−] vectors to induce optimal immune responses, some embodiments relate to a next generation Ad5 vector based vaccine platform.

First generation, or E1-deleted adenovirus vectors Ad5 [E1−] are constructed such that a transgene replaces only the E1 region of genes. Typically, about 90% of the wild-type Ad5 genome is retained in the vector. Ad5 [E1−] vectors have a decreased ability to replicate and cannot produce infectious virus after infection of cells that do not express the Ad5 E1 genes. The recombinant Ad5 [E1−] vectors are propagated in human cells (e.g., 293 cells) allowing for Ad5 [E1−] vector replication and packaging. Ad5 [E1−] vectors have a number of positive attributes; one of the most important is their relative ease for scale up and cGMP production. Currently, well over 220 human clinical trials utilize Ad5 [E1−] vectors, with more than two thousand subjects given the virus subcutaneously, intra muscularly, or intravenously. Additionally, Ad5 vectors do not integrate; their genomes remain episomal. Generally, for vectors that do not integrate into the host genome, the risk for insertional mutagenesis and/or germ-line transmission is extremely low if at all. Conventional Ad5 [E1−] vectors have a carrying capacity that approaches 7 kb.

Ad5-based vectors with deletions of the E1 and the E2b regions (Ad5 [E1−, E2b−]), the latter encoding the DNA polymerase and the pre-terminal protein, by virtue of diminished late phase viral protein expression, provide an opportunity to avoid immunological clearance and induce more potent immune responses against the encoded tumor antigen transgene in Ad-immune hosts. The new Ad5 platform has additional deletions in the E2b region, removing the DNA polymerase and the preterminal protein genes. The Ad5 [E1−, E2b−] platform has an expanded cloning capacity that is sufficient to allow inclusion of many possible genes. Ad5 [E1−, E2b−] vectors have up to about 12 kb gene-carrying capacity as compared to the 7 kb capacity of Ad5 [E1−] vectors, providing space for multiple genes if needed. In some embodiments, an insert of more than 1, 2, 3, 4, 5, 6, 7, 8, 9, 10, or 11 kb is introduced into an Ad5 vector, such as the Ad5 [E1−, E2b−] vector. Deletion of the E2b region confers advantageous immune properties on the Ad5 vectors, often eliciting potent immune responses to target transgene antigens while minimizing the immune responses to Ad viral proteins.

In various embodiments, Ad5 [E1−, E2b−] vectors induce potent cell-mediated immunity (CMI), as well as antibodies against the vector expressed vaccine antigens even in the presence of Ad immunity. Ad5 [E1−, E2b−] vectors also have reduced adverse reactions as compared to Ad5 [E1−] vectors, in particular the appearance of hepatotoxicity and tissue damage. A key aspect of these Ad5 vectors is that expression of Ad late genes is greatly reduced. For example, production of the capsid fiber proteins could be detected in vivo for Ad5 [E1−] vectors, while fiber expression was ablated from Ad5 [E1−, E2b−] vector vaccines. The innate immune response to wild type Ad is complex. Proteins deleted from the Ad5 [E1−, E2b−] vectors generally play an important role. Specifically, Ad5 [E1−, E2b−] vectors with deletions of preterminal protein or DNA polymerase display reduced inflammation during the first 24 to 72 h following injection compared to Ad5 [E1−] vectors. In various embodiments, the lack of Ad5 gene expression renders infected cells invisible to anti-Ad activity and permits infected cells to express the transgene for extended periods of time, which develops immunity to the target.

Some embodiments contemplate increasing the capability for the Ad5 [E1−, E2b−] vectors to transduce dendritic cells, improving antigen specific immune responses in the vaccine by taking advantage of the reduced inflammatory response against Ad5 [E1−, E2b−] vector viral proteins and the resulting evasion of pre-existing Ad immunity.

### Replication Defective Ad5 Vectors

Attempts to overcome anti-Ad immunity have included use of alternative Ad serotypes and/or alternations in the Ad5 viral capsid protein each with limited success and the potential for significantly altering biodistribution of the resultant vaccines. Therefore, a completely novel approach was attempted by further reducing the expression of viral proteins from the E1 deleted Ad5 vectors, proteins known to be targets of pre-existing Ad immunity. Specifically, a novel recombinant Ad5 platform has been described with deletions in the early 1 (E1) gene region and additional deletions in the early 2b (E2b) gene region (Ad5 [E1−, E2b−]). Deletion of the E2b region (that encodes DNA polymerase and the pre-terminal protein) results in decreased viral DNA replication and late phase viral protein expression. This vector platform can be used to induce CMI responses in animal models of cancer and infectious disease and more importantly, this recombinant Ad5 gene delivery platform overcomes the barrier of Ad5 immunity and can be used in the setting of pre-existing and/or vector-induced Ad immunity thus enabling multiple homologous administrations of the vaccine. In particular embodiments, some embodiments relate to a replication defective adenovirus vector of serotype 5 comprising a sequence encoding an immunogenic polypeptide. The immunogenic polypeptide can be a mutant, natural variant, or a fragment thereof.

In some embodiments, the replication defective adenovirus vector comprises a modified sequence encoding a polypeptide with at least 50%, 60%, 65%, 70%, 75%, 80%, 85%, 90%, 95%, 98%, 99%, 99.5%, 99.9%, or 100% identity to a wild-type immunogenic polypeptide or a fragment thereof. In some embodiments, the replication defective adenovirus vector comprises a modified sequence encoding a subunit of a wild-type polypeptide. The compositions and methods, in some embodiments, relate to an adenovirus-derived vector comprising at least 60% sequence identity to SEQ ID NO: 3 or SEQ ID NO: 100.

In some embodiments, an adenovirus-derived vector, optionally relating to a replication defective adenovirus, comprises a sequence with at least 75%, 80%, 85%, 90%, 95%, 98%, 99%, 99.5%, 99.8%, or 99.9% identity to SEQ ID NO: 3 or SEQ ID NO: 100 or a sequence generated from SEQ ID NO: 3 or SEQ ID NO: 100 by alternative codon replacements. In various embodiments, the adenovirus-derived vectors described herein have a deletion in the E2b region, and optionally, in the E1 region, the deletion conferring a variety of advantages to the use of the vectors in immunotherapy as described herein.

Certain regions within the adenovirus genome serve essential functions and may need to be substantially conserved when constructing the replication defective adenovirus vectors. These regions are further described in Lauer et al., J. Gen. Virol., 85, 2615-25 (2004), Leza et al., J. Virol., p. 3003-13 (1988), and Miralles et al., J. Bio Chem., Vol. 264, No. 18, p. 10763-72 (1983), which are incorporated by reference in their entirety. Recombinant nucleic acid vectors comprising a sequence with identity values of at least 50%, 60%, 65%, 70%, 75%, 80%, 85%, 90%, 95%, 98%, 99%, 99.5%, 99.8%, 99.9%, or 100% to a portion of SEQ ID NO: 3 or SEQ ID NO: 100, such as a portion comprising at least about 100, 250, 500, 1000 or more bases of SEQ ID NO: 3 or SEQ ID NO: 100 are used in some embodiments.

Certain embodiments contemplate the use of E2b deleted adenovirus vectors, such as those described in U.S. Pat. Nos. 6,063,622; 6,451,596; 6,057,158; 6,083,750; and 8,298,549, which are each incorporated herein by reference in their entirety. The vectors with deletions in the E2b regions in many cases cripple viral protein expression and/or decrease the frequency of generating replication competent Ad (RCA). Propagation of these E2b deleted adenovirus vectors can be done utilizing cell lines that express the deleted E2b gene products. Such packaging cell lines are provided herein; e.g., E.C7 (formally called C-7), derived from the HEK-2p3 cell line.

Further, the E2b gene products, DNA polymerase and preterminal protein, can be constitutively expressed in E.C7, or similar cells along with the E1 gene products. Transfer of gene segments from the Ad genome to the production cell line has immediate benefits: (1) increased carrying capacity; and, (2) a decreased potential of RCA generation, typically requiring two or more independent recombination events to generate RCA. The E1, Ad DNA polymerase and/or preterminal protein expressing cell lines used in some embodiments can enable the propagation of adenovirus vectors with a carrying capacity approaching 13 kb, without the need for a contaminating helper virus. In addition, when genes critical to the viral life cycle are deleted (e.g., the E2b genes), a further crippling of Ad to replicate or express other viral gene proteins occurs. This can decrease immune recognition of infected cells, and extend durations of foreign transgene expression.

E1, DNA polymerase, and preterminal protein deleted vectors are typically unable to express the respective proteins from the E1 and E2b regions. Further, they can show a lack of expression of most of the viral structural proteins. For example, the major late promoter (MLP) of Ad is responsible for transcription of the late structural proteins L1 through L5. Though the MLP is minimally active prior to Ad genome replication, the highly toxic Ad late genes are primarily transcribed and translated from the mLP only after viral genome replication has occurred. This cis-dependent activation of late gene transcription is a feature of DNA viruses in general, such as in the growth of polyoma and SV-40. The DNA polymerase and preterminal proteins are important for Ad replication (unlike the E4 or protein IX proteins). Their deletion can be extremely detrimental to adenovirus vector late gene expression, and the toxic effects of that expression in cells such as APCs.

The adenovirus vectors can include a deletion in the E2b region of the Ad genome and, optionally, the E1 region. In some cases, such vectors do not have any other regions of the Ad genome deleted. The adenovirus vectors can include a deletion in the E2b region of the Ad genome and deletions in the E1 and E3 regions. In some cases, such vectors have no other regions deleted. The adenovirus vectors can include a deletion in the E2b region of the Ad genome and deletions in the E1, E3 and partial or complete removal of the E4 regions. In some cases, such vectors have no other deletions. The adenovirus vectors can include a deletion in the E2b region of the Ad genome and deletions in the E1 and/or E4 regions. In some cases, such vectors contain no other deletions. The adenovirus vectors can include a deletion in the E2a, E2b and/or E4 regions of the Ad genome. In some cases, such vectors have no other deletions. The adenovirus vectors can have the E1 and/or DNA polymerase functions of the E2b region deleted. In some cases, such vectors have no other deletions. The adenovirus vectors can have the E1 and/or the preterminal protein functions of the E2b region deleted. In some cases, such vectors have no other deletions. The adenovirus vectors can have the E1, DNA polymerase and/or the preterminal protein functions deleted. In some cases, such vectors have no other deletions. The adenovirus vectors can have at least a portion of the E2b region and/or the E1 region. In some cases, such vectors are not gutted adenovirus vectors. In this regard, the vectors can be deleted for both the DNA polymerase and the preterminal protein functions of the E2b region. The adenovirus vectors can have a deletion in the E1, E2b and/or 100K regions of the adenovirus genome. The adenovirus vectors can comprise vectors having the E1, E2b and/or protease functions deleted. In some cases, such vectors have no other deletions. The adenovirus vectors can have the E1 and/or the E2b regions deleted, while the fiber genes have been modified by mutation or other alterations (for example to alter Ad tropism). Removal of genes from the E3 or E4 regions can be added to any of the adenovirus vectors mentioned. In certain embodiments, adenovirus vectors can have a deletion in the E1 region, the E2b region, the E3 region, the E4 region, or any combination thereof. In certain embodiments, the adenovirus vector can be a gutted adenovirus vector.

Other regions of the Ad genome can be deleted. A “deletion” in a particular region of the Ad genome refers to a specific DNA sequence that is mutated or removed in such a way so as to prevent expression and/or function of at least one gene product encoded by that region (e.g., E2b functions of DNA polymerase or preterminal protein function). Deletions encompass deletions within exons encoding portions of proteins as well as deletions within promoter and leader sequences. A deletion within a particular region refers to a deletion of at least one base pair within that region of the Ad genome. More than one base pair can be deleted. For example, at least 20, 30, 40, 50, 60, 70, 80, 90, 100, 110, 120, 130, 140, or 150 base pairs can be deleted from a particular region. The deletion can be more than 150, 160, 170, 180, 190, 200, 250, or 300 base pairs within a particular region of the Ad genome. These deletions can prevent expression and/or function of the gene product encoded by the region. For example, a particular region of the Ad genome can include one or more point mutations such that one or more encoded proteins is non-functional. Such mutations include residues that are replaced with a different residue leading to a change in the amino acid sequence that result in a nonfunctional protein. Exemplary deletions or mutations in the Ad genome include one or more of E1a, E1b, E2a, E2b, E3, E4, L1, L2, L3, L4, L5, TP, POL, IV, and VA regions. Deleted adenovirus vectors can be made, for example, using recombinant techniques.

Ad vectors in certain embodiments can be successfully grown to high titers using an appropriate packaging cell line that constitutively expresses E2b gene products and products of any of the necessary genes that can be deleted. HEK-293-derived cells that not only constitutively express the E1 and DNA polymerase proteins, but also the Ad-preterminal protein, can be used. E.C7 cells can be used, for example, to grow high titer stocks of the adenovirus vectors.

To delete critical genes from self-propagating adenovirus vectors, proteins encoded by the targeted genes can first be coexpressed in HEK-293 cells, or similar, along with E1 proteins. For example, those proteins which are non-toxic when coexpressed constitutively (or toxic proteins inducibly-expressed) can be selectively utilized. Coexpression in HEK-293 cells of the E1 and E4 genes is possible (for example utilizing inducible, not constitutive, promoters). The E1 and protein IX genes, a virion structural protein, can be coexpressed. Further coexpression of the E1, E4, and protein IX genes is also possible. E1 and 100K genes can be expressed in trans-complementing cell lines, as can E1 and protease genes.

Cell lines co-expressing E1 and E2b gene products for use in growing high titers of E2b deleted Ad particles can be used. Useful cell lines constitutively express the approximately 140 kDa Ad-DNA polymerase and/or the approximately 90 kDa preterminal protein. Cell lines that have high-level, constitutive co-expression of the E1, DNA polymerase, and preterminal proteins, without toxicity (e.g., E.C7), are desirable for use in propagating Ad for use in multiple vaccinations. These cell lines permit the propagation of adenovirus vectors deleted for the E1, DNA polymerase, and preterminal proteins.

The recombinant Ad can be propagated using, for example, tissue culture plates containing E.C7 cells infected with Ad vector virus stocks at an appropriate multiplicity of infection (MOI) (e.g., 5) and incubated at 37° C. for 40-96 h.

In some embodiments, the successful production of infectious Ad5 virions can be confirmed using a hexon assay, which is an antibody based cellular assay in which hexon positive cells are manually counted by microscopy. For example, a small sample of E.C7 cells propagating the Ad5 vector can be analyzed for hexon expression using an antibody-based detection assay to quantify the infectious units (IFUs)/mL of Ad5 virions. Cells infected with virions can be capable of driving expression of hexon and hexon expression can be indicative of completion of the replication cycle of the virus. In some embodiments, hexon expression can occur if fully formed virions are present. In some embodiments, the hexon assay can be carried out via an anti-hexon antibody mediated immunostaining method. In some embodiments, after incubation of cells with the anti-hexon antibody, cells can be further incubated with a secondary antibody conjugated to horse radish peroxidase (HRP) enzyme. Cells can then be incubated with a DAB substrate. In some embodiments, the hexon assay can be carried out by manually counting dark cells by eye using a microscope. Cells that are darkened indicate accumulation of insoluble DAB peroxidase reaction products. However, the hexon assay can be an expensive assay due to costly reagents and can be labor intensive.

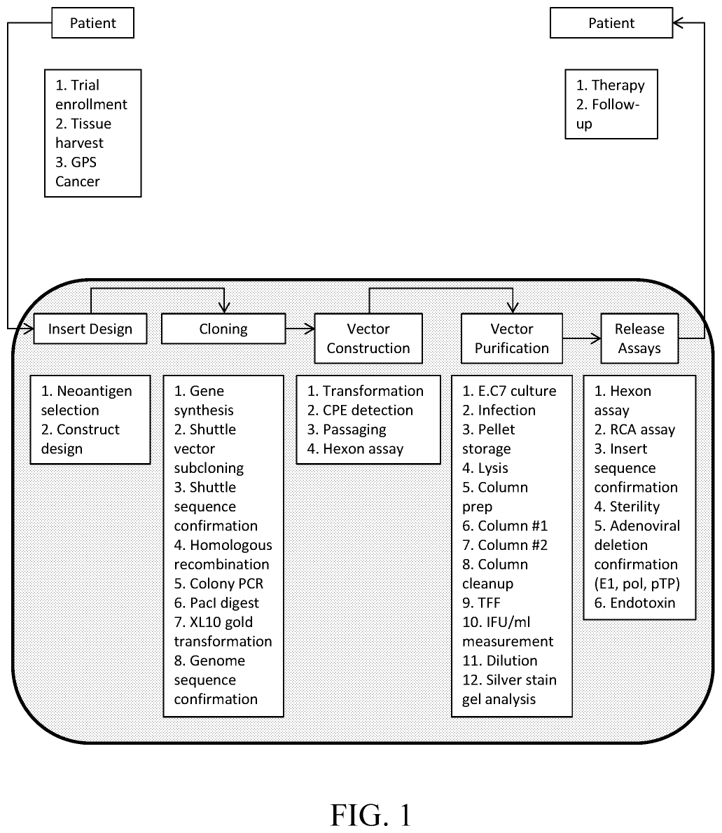

Thus, in some embodiments, the present disclosure provides a hexon assay alternative (see step 4 of vector construction in FIG. 1). In some embodiments, the hexon assay alternative is an antibody-mediated flow cytometry assay for detection of hexon expression in suspension E.C7 cells. For example, a small sample of E.C7 cells propagating the Ad5 vector can be sampled, lysed by freezing and thawing with a cryoprotectant, and concentrated by centrifugation. A small sample of the supernatant, comprising the Ad5 virions, can be serially diluted and incubated at various concentrations with a separate culture of suspension E.C7 cells in serum-free media. Suspension E.C7 cells can be incubated with Ad5 virions for 48 hours and can be further analyzed with a live/dead stain and with anti-hexon, fluorophore-labeled monoclonal antibody. Flow cytometry analysis can reveal the percentage of cells that are hexon positive, thereby indicating the infectivity of the Ad5 virions. In some embodiments, flow cytometry detection of hexon expression in suspension E.C7 cells can take up to 2-2.5 days.

In other embodiments, the hexon assay alternative can be an antibody-mediated flow cytometry assay for detection of hexon expression in suspension cells including, but not limited to, bone marrow-derived cells (e.g., K-562 cells), T-lymphoblast-derived cells (e.g., MOLT-4 cells), or T cell lymphoma (e.g., Jurkat E6-1 cells). Suspension cells (e.g., K-562 cells, MOLT-4 cells, or Jurkat E6-1 cells) can be transfected with plasmids and can, thus, express adenovirus 5 pol, pTP, E1a, and E1b, allowing for replication of Ad5 [E1−, E2b−] virions. Suspension cells (e.g., K-562 cells, MOLT-4 cells, or Jurkat E6-1 cells) can then be incubated with Ad5 virions obtained from E.C7 cells propagating the Ad5 vector by lysing and freeze/thaw techniques, as described above. Suspension cells (e.g., K-562 cells, MOLT-4 cells, or Jurkat E6-1 cells) can be incubated with Ad5 virions for 48 hours and can be further analyzed with a live/dead stain and with anti-hexon, fluorophore-labeled monoclonal antibody. Flow cytometry analysis can reveal the percentage of cells that are hexon positive, thereby indicating the infectivity of the Ad5 virions. In some embodiments, flow cytometry detection of hexon expression in suspension cells (e.g., K-562 cells, MOLT-4 cells, or Jurkat E6-1 cells) can take up to 2-2.5 days.

In still other embodiments, the hexon assay alternative can be hexon quantitation and correlation with infectivity via bio-layer interferometry (BLI) with the BLItz® System or Octet® System from Pall ForteBio. In some embodiments, optical glass biosensors can be coated with an anti-hexon monoclonal antibody and a sample of clarified cell lysate from the E.C7 cells propagating the Ad5 vectors can be loaded onto the glass biosensor. Mass accumulation on the tip of the optical glass biosensor can be measured by the BLItz® System or Octet® System, thereby allowing for quantification of hexon-positive cells. In some embodiments, hexon quantification via bio-layer interferometry can be carried out in 5-30 minutes, 5-10 minutes, 10-15 minutes, 15-20 minutes, 20-25 minutes, or 25-30 minutes.

In some embodiments, any one of the above described hexon assay alternatives can be used to quantitate infectivity after E.C7 cells are transfected with any Ad5 vector of the present disclosure and have been propagated and passaged for 10 days.

The infected cells can be harvested, resuspended in 10 mM Tris-Cl (pH 8.0), and sonicated, and the virus can be purified by two rounds of cesium chloride density centrifugation. The virus containing band can be desalted over a column, sucrose or glycerol can be added, and aliquots can be stored at −80° C. However, the use of cesium chloride columns for density based purification of adenovirus can require long processing times and can be inefficient at purifying small-scale and large scale sample volumes. Moreover dialysis can be required to remove cesium chloride, which can be cytotoxic.

Thus, in other embodiments, the virus can be purified through an ion exchange based separation mechanism followed by a Source 30Q column (a Q sepharose column), which is a column purifier also based on an ion exchange mechanism. For example, in some embodiments, the ion exchange based separation mechanism can be a Q sepharose column. A Q sepharose column can contain a resin slurry with charged residues that bind the virus, while allowing undesired cellular components to pass. In some embodiments, the resin slurry is comprised of 30 m polystyrene beads displaying quaternary cations. In some embodiments, the charged residues on the resin slurry are of an opposite charge to the virus in a first buffer. For example, in a first buffer with a particular ionic strength, the virus can be negatively charged and the charged residues on the resin slurry confer a positive charge, which can allow for the virus to bind the slurry. Subsequently, the virus can be eluted off the Q sepharose column by flowing through a second buffer with a different ionic strength that competes with the virus for binding to the Q sepharose column resin, causing the virus to elute. Finally, post-Q sepharose column purification, the virus can be passed through a Source 30Q column for a second round of purification, which can remove additional cellular proteins. In general, the Q sepharose column can be a polishing column, which removes residual cellular proteins not removed by a previous purification membrane or column.

In still other embodiments, in place of the Q sepharose column described above, virus vectors can be purified from infected E.C7 cells using a membrane (e.g., SARTOBIND® Q Membrane or MUSTANG® Q Membrane) that provides an ion exchange separation mechanism to bind undesirable components and purify intact viral vectors, including the adenovirus vectors of the present disclosure. For example, the SARTOBIND® Q Membrane or MUSTANG® Q Membrane can be used to purify the adenovirus vectors of the present disclosure. The SARTOBIND® Q Membrane or MUSTANG® Q Membrane adsorbs adenovirus due to its macro-porous structure which displays a positive ionic charge and has pore sizes of greater than 800 nm or greater than 3000 nm. Adenovirus, which is negatively charged at physiological pH can, thus, have a high binding capacity for the SARTOBIND® Q Membrane or MUSTANG® Q Membrane, while undesired cell lysates and proteins are filtered through. For example, the cell lysate containing the adenovirus can be loaded onto the SARTOBIND® Q Membrane or MUSTANG® Q Membrane in a salt buffer, also referred to herein as a “loading salt buffer.” In some embodiments, the loading salt buffer, such as an NaCl salt buffer, can have an ionic strength of 300 mM-310 mM, 310 mM-320 mM, 320 mM-330 mM, 330 mM-340 mM, 340 mM-350 mM or 300 mM-350 mM. In some embodiments, the loading salt buffer, such as an NaCl salt buffer, can have an ionic strength of 325 mM NaCl. Upon completion of membrane purifying a cell lysate preparation, the adenovirus can be eluted off the SARTOBIND® Q Membrane or MUSTANG® Q Membrane by washing the membrane with a salt buffer, also referred to herein as a “elution salt buffer,” at an ionic strength in which adenovirus becomes positively charged. For example, in some embodiments, the elution salt buffer, such as an NaCl salt buffer, can have an ionic strength of 450 mM-540 mM, 450 mM-460 mM, 460 mM-470 mM, 470 mM-480 mM, 480 mM-490 mM, 490 mM-500 mM, 500 mM-510 mM, 510 mM-520 mM, 520 mM-530 mM, 530 mM-540 mM, 540 mM-550 mM, 550 mM-560 mM, 560 mM-570 mM, 570 mM-580 mM, 580 mM-590 mM, 590 mM-600 mM, 600 mM-610 mM, 610 mM-620 mM, 620 mM-630 mM, 630 mM-640 mM, 640 mM-650 mM, or 550 mM-650 mM. In some embodiments, the elution salt buffer, such as an NaCl salt buffer, can have an ionic strength of 450-540 mM NaCl. In some embodiments, the adenovirus can elute with an elution salt buffer of 450-540 mM NaCl. The loading or elution salt buffers can be a sodium chloride (NaC)-based buffer. In some embodiments, use of the SARTOBIND® Q membrane or MUSTANG® Q Membrane can accelerate the purification process as compared to use of the Q Sepharose column. For example, the SARTOBIND® Q membrane or MUSTANG® Q Membrane can provide greater scalability and speed in purification of adenovirus from the cell lysate. Thus, in some embodiments, the SARTOBIND® Q membrane or MUSTANG® Q Membrane replaces the Q Sepharose column and a subsequent round of purification is performed using a Source 30Q column. In other embodiments, the SARTOBIND® Q membrane or MUSTANG® Q Membrane replaces the Q Sepharose column and the Source 30Q column and, thus, the adenovirus is purified in a single step. Vector purification steps of the present disclosure can include purification of cell lysate containing Ad5 vectors through a Q membrane (e.g., the SARTOBIND® Q membrane or MUSTANG® Q Membrane).

In some embodiments, the membrane purification step with the SARTOBIND® Q membrane or MUSTANG® Q Membrane is conducted using a fast protein liquid chromatography (FPLC) system, in which all aspects of the purification are computer controlled. For example, but adapting the SARTOBIND® Q membrane or MUSTANG® Q Membrane to an FPLC, the pump, buffer systems, and fraction collectors are all computer controlled.

In some embodiments, the membrane used is any ion exchange membrane. In some embodiments, the membrane has positively charged moieties (e.g., quarternary ammonium ligands) covalently conjugated to its inner surface. For example, the SARTOBIND® Q Membrane or MUSTANG® Q Membrane is a membrane with positively charged quarternary ammonium ligands covalently conjugated to its inner surface. These types of membranes can be used to purify negatively charged compositions of interest (e.g., Ad5). In other embodiments, the membrane has negatively charged moieties (e.g., sulfonic acid ligands) covalently conjugated to its inner surface. For example, the SARTOBIND® S Membrane or the MUSTANG® S Membrane is a membrane with negatively charged sulfonic acid ligands covalently conjugated to its inner surface. In some embodiments, the membrane used is a SARTOBIND® Q Membrane or MUSTANG® Q Membrane.

In some embodiments, the membrane purification involves lysing infected E.C7 cells to retrieve the Ad5 viral vectors of interest. For example, Ad5-expressing E.C7 cells can be lysed with an appropriate lysis buffer and then loaded onto a SARTOBIND® Q Membrane or MUSTANG® Q Membrane that has been equilibrated. After loading the cell lysate onto the SARTOBIND® Q Membrane or MUSTANG® Q Membrane and washing the membrane, Ad5 can be eluted with an appropriate buffer, for example, a solution of 650 mM NaCl. In some embodiments, the SARTOBIND® Q Membrane or MUSTANG® Q Membrane purification step takes 30 minutes to 2 hours, 30 minutes to 45 minutes, 30 minutes to 1 hour, 45 minutes to 1 hour, 1 hour to 1.5 hours, 1.5 hours to 2 hours, or 1 hour to 2 hours. In some embodiments, 50-200 mL of the cell lysate is filtered through the membrane purification system in any of the above described times. In some embodiments 1E13-1E14 virus particles (VPs)/mL of the neo-antigen vector is purified from the membrane purification system. In some embodiments, the SARTOBIND® Q Membrane or MUSTANG® Q Membrane purification step can process 1E8 to 4E9 cells/mL of membrane, wherein mL of membrane corresponds to the bed volume of the membrane, in 0.2-4 L of cell culture and retrieve 1E12 to 4.9E13 virus particles (VPs)/mL membrane.

Membrane purified adenovirus vectors can be further filtered through a Source 30Q column that has been equilibrated and Ad5 vectors can be eluted with an appropriate buffer, for example, a linear gradient of 0.15-1M NaCl. Subsequently, column purified adenovirus vectors can be subject to tangential flow filtration with a hollow-fiber (HF) membrane module using a KrosFlo instrument. Tangential flow filtration allows for concentration and buffer exchange of the purified, but diluted, adenovirus, by running the purified adenovirus under pressure against a buffer of choice. By passing the purified adenovirus through HF membranes, solutes are pushed out and exchanged. Adenovirus vectors can be stored in an appropriate storage buffer, for example, 2% 1M Tris at pH 8.0, 0.834% 3M NaCl, 5% glycerol and 92.166% H₂O.

In some embodiments, ion-exchange membranes of the present disclosure and purification columns of the present disclosure are disposed after a single use. In some embodiments, columns of the present disclosure are cleaned for further use. For example, cleanup of Q sepharose columns adapted to an FPLC instrument can be performed as follows. The sample pump inlet tubing can be cleaned with 0.5M NaOH by wetting a paper towel and cleaning the outside of the tubing, which was exposed to virus during sample load. The sample pump inlet can be placed in 0.5M NaOH. Columns can be cleaned with an all column cleaning run at 2 mL/min in upflow mode. For the Q sepharose column, 2-3 column volumes (CVs), for example 50 ml, of 0.5 M NaOH can be run from the sample pump, the run can be paused for 1 hour and the sample pump inlet can be placed into 2M NaCl, and 2-3 CVs, for example 50 mL, of 2 M NaOH can be run through the column without pausing. The sample pump inlet can be placed in H₂O and 3-5 CVs, for example 150 mL, of H₂O can be run through the column (Q sepharose or Source 30Q) until a conductivity detector is stable at less than 1 mS/cm. Source30Q columns can be cleaned by running the following solutions through the column from the sample pump, as described above, 30 mL of 0.5M NaOH, 30 mL of 2M NaCl, and 50 mL of H₂O. If the FPLC columns are not used for a period of greater than 10 days, they can be stored in 20% EtOH, which can be run through the columns and pumps at no more than 2 mL/min.

Virus can be placed in a solution designed to enhance its stability, such as A195, which can comprise 20 mM Tris, pH8.0, 25 mM NaCl, 2.5% glycerol. The titer of the stock can be measured (e.g., by measurement of the optical density at 260 nm of an aliquot of the virus after lysis). Plasmid DNA, either linear or circular, encompassing the entire recombinant E2b deleted adenovirus vector can be transfected into E.C7, or similar cells, and incubated at 37° C. until evidence of viral production is present (e.g., cytopathic effect). Conditioned media from cells can be used to infect more cells to expand the amount of virus produced before purification. Purification can be accomplished, for example, by two rounds of cesium chloride density centrifugation or selective filtration. Virus may be purified by chromatography using commercially available products or custom chromatographic columns.

The compositions as described herein can comprise enough virus to ensure that cells to be infected are confronted with a certain number of viruses. Thus, some embodiments provide a stock of recombinant Ad, such as an RCA-free stock of recombinant Ad. Viral stocks can vary considerably in titer, depending largely on viral genotype and the protocol and cell lines used to prepare them. Viral stocks can have a titer of at least about 10⁶, 10⁷, or 10⁸ infectious units (IFU)/mL, or higher, such as at least about 10⁹, 10¹⁰, 10¹¹, or 10¹² IFU/mL. Depending on the nature of the recombinant virus and the packaging cell line, a viral stock can have a titer of even about 10¹³ particles/ml or higher.

A replication defective adenovirus vector (e.g., SEQ ID NO: 2) can comprise a sequence encoding a target antigen, a fragment thereof, or a variant thereof, at a suitable position. In some embodiments, a replication defective adenovirus vector (e.g., SEQ ID NO: 2) can comprise a sequence encoding a target antigen described herein, or a fragment, a variant, or a variant fragment thereof, at a position replacing the nucleic acid sequence encoding a CEA or a variant CEA (e.g., SEQ ID NO: 1 or SEQ ID NO: 100). In some embodiments, a replication defective adenovirus vector (e.g., SEQ ID NO: 2) can comprise a sequence encoding a target antigen described herein, or a fragment, a variant, or a variant fragment thereof, at a position replacing the nucleic acid sequence encoding a CEA or a variant CEA (e.g., SEQ ID NO: 1, SEQ ID NO: 3, SEQ ID NO: 4, or SEQ ID NO: 100).

### Polynucleotides and Variants Encoding Antigen Targets

Certain embodiments provide nucleic acid sequences, also referred to herein as polynucleotides that encode one or more target antigens of interest, or fragments or variants thereof. As such, some embodiments provide polynucleotides that encode target antigens from any source as described further herein and vectors comprising such polynucleotides and host cells transformed or transfected with such expression vectors. In order to express a desired target antigen polypeptide, nucleotide sequences encoding the polypeptide, or functional equivalents, can be inserted into an appropriate Ad vector (e.g., using recombinant techniques). The appropriate adenovirus vector can contain the necessary elements for the transcription and translation of the inserted coding sequence and any desired linkers. Standard methods can be used to construct these adenovirus vectors containing sequences encoding a polypeptide of interest and appropriate transcriptional and translational control elements. These methods can include in vitro recombinant DNA techniques, synthetic techniques, and in vivo genetic recombination, or any combination thereof.

Polynucleotides can comprise a native sequence (i.e., an endogenous sequence that encodes a target antigen polypeptide/protein/epitope or a portion thereof) or can comprise a sequence that encodes a variant, fragment, or derivative of such a sequence. Polynucleotide sequences can encode target antigen proteins. In some embodiments, polynucleotides represent a novel gene sequence optimized for expression in specific cell types that can substantially vary from the native nucleotide sequence or variant but encode a similar protein antigen.

In other related embodiments, polynucleotide variants have substantial identity to native sequences encoding proteins (e.g., target antigens of interest), for example those comprising at least 70% sequence identity, preferably at least 75%, 80%, 85%, 90%, 95%, 96%, 97%, 98%, or 99% or higher, sequence identity compared to a native polynucleotide sequence encoding the polypeptides (e.g., BLAST analysis using standard parameters). These values can be appropriately adjusted to determine corresponding identity of proteins encoded by two nucleotide sequences by taking into account codon degeneracy, amino acid similarity, reading frame positioning and the like. Polynucleotides can encode a protein comprising for example at least 70% sequence identity, preferably at least 75%, 80%, 85%, 90%, 95%, 96%, 97%, 98%, or 99% or higher, sequence identity compared to a protein sequence encoded by a native polynucleotide sequence.

Polynucleotides can comprise at least about 5, 10, 15, 20, 25, 30, 35, 40, 45, 50, 55, 60, 65, 70, 75, 80, 85, 90, 95, 100, 11, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 or more contiguous nucleotides encoding a polypeptide (e.g., target protein antigens), and all intermediate lengths there between. “Intermediate lengths”, in this context, refers to any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence can be extended at one or both ends by additional nucleotides not found in the native sequence encoding a polypeptide, such as an epitope or heterologous target protein. This additional sequence can consist of 1, 2, 3, 4, 5, 6, 7, 8, 9, 10, 11, 12, 13, 14, 15, 16, 17, 18, 19, or 20 nucleotides or more, at either end of the disclosed sequence or at both ends of the disclosed sequence.

The polynucleotides, regardless of the length of the coding sequence itself, can be combined with other DNA sequences, such as promoters, expression control sequences, polyadenylation signals, additional restriction enzyme sites, multiple cloning sites, other coding segments, and the like, such that their overall length can vary considerably. It is therefore contemplated that a nucleic acid fragment of almost any length can be employed, with the total length preferably being limited by the ease of preparation and use in the intended recombinant DNA protocol. Illustrative polynucleotide segments with total lengths of about 1000, 2000, 3000, 4000, 5000, 6000, 7000, 8000, 9000, 10,000, about 500, about 200, about 100, about 50 base pairs in length, and the like, (including all intermediate lengths) are contemplated to be useful in many embodiments.

A mutagenesis approach, such as site-specific mutagenesis, can be employed to prepare target antigen sequences. Specific modifications in a polypeptide sequence can be made through mutagenesis of the underlying polynucleotides that encode them. Site-specific mutagenesis can be used to make mutants through the use of oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. For example, a primer comprising from about 14 to about 25 nucleotides or so in length can be employed, with from about 5 to about 10 residues on both sides of the junction of the sequence being altered. Mutations can be made in a selected polynucleotide sequence to improve, alter, decrease, modify, or otherwise change the properties of the polynucleotide, and/or alter the properties, activity, composition, stability, or primary sequence of the encoded polypeptide.

Mutagenesis of polynucleotide sequences can be used to alter one or more properties of the encoded polypeptide, such as the immunogenicity of an epitope comprised in a polypeptide or the oncogenicity of a target antigen. Assays to test the immunogenicity of a polypeptide include, but are not limited to, T-cell cytotoxicity assays (CTL/chromium release assays), T-cell proliferation assays, intracellular cytokine staining, ELISA, ELISpot, etc. Other ways to obtain sequence variants of peptides and the DNA sequences encoding them can be employed. For example, recombinant vectors encoding the desired peptide sequence can be treated with mutagenic agents, such as hydroxylamine, to obtain sequence variants.

Polynucleotide segments or fragments encoding the polypeptides as described herein can be readily prepared by, for example, directly synthesizing the fragment by chemical means. Fragments can be obtained by application of nucleic acid reproduction technology, such as PCR, by introducing selected sequences into recombinant vectors for recombinant production.

A variety of vector/host systems can be utilized to contain and produce polynucleotide sequences. Exemplary systems include microorganisms such as bacteria transformed with recombinant bacteriophage, plasmid, or cosmid DNA vectors; yeast transformed with yeast vectors; insect cell systems infected with virus vectors (e.g., baculovirus); plant cell systems transformed with virus vectors (e.g., cauliflower mosaic virus, CaMV; tobacco mosaic virus, TMV) or with bacterial vectors (e.g., Ti or pBR322 plasmids); or animal cell systems.

Control elements or regulatory sequences present in an Ad vector can include those non-translated regions of the vector-enhancers, promoters, and 5′ and 3′ untranslated regions. Such elements can vary in their strength and specificity. Depending on the vector system and host utilized, any number of suitable transcription and translation elements, including constitutive and inducible promoters, can be used. For example, sequences encoding a polypeptide of interest can be ligated into an Ad transcription/translation complex consisting of the late promoter and tripartite leader sequence. Insertion in a non-essential E1 or E3 region of the viral genome can be used to obtain a viable virus which is capable of expressing the polypeptide in infected host cells. In addition, transcription enhancers, such as the Rous sarcoma virus (RSV) enhancer, can be used to increase expression in mammalian host cells.

Specific initiation signals can also be used to achieve more efficient translation of sequences encoding a polypeptide of interest (e.g., ATG initiation codon and adjacent sequences). Exogenous translational elements and initiation codons can be of various origins, both natural and synthetic. The efficiency of expression can be enhanced by the inclusion of enhancers which are appropriate for the particular cell system which is used. Specific termination sequences, either for transcription or translation, can also be incorporated in order to achieve efficient translation of the sequence encoding the polypeptide of choice.

A variety of protocols for detecting and measuring the expression of polynucleotide-encoded products (e.g., target antigens), can be used (e.g., using polyclonal or monoclonal antibodies specific for the product). Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide can be preferred for some applications, but a competitive binding assay can also be employed.

The Ad vectors can comprise a product that can be detected or selected for, such as a reporter gene whose product can be detected, such as by fluorescence, enzyme activity on a chromogenic or fluorescent substrate, and the like, or selected for by growth conditions. Exemplary reporter genes include green fluorescent protein (GFP), β-galactosidase, chloramphenicol acetyltransferase (CAT), luciferase, neomycin phosphotransferase, secreted alkaline phosphatase (SEAP), and human growth hormone (HGH). Exemplary selectable markers include drug resistances, such as neomycin (G418), hygromycin, and the like.

The Ad vectors can also comprise a promoter or expression control sequence. The choice of the promoter will depend in part upon the targeted cell type and the degree or type of control desired. Promoters that are suitable include, without limitation, constitutive, inducible, tissue specific, cell type specific, temporal specific, or event-specific. Examples of constitutive or nonspecific promoters include the SV40 early promoter, the SV40 late promoter, CMV early gene promoter, bovine papilloma virus promoter, and adenovirus promoter. In addition to viral promoters, cellular promoters are also amenable and useful in some embodiments. In particular, cellular promoters for the so-called housekeeping genes are useful (e.g., β-actin). Viral promoters are generally stronger promoters than cellular promoters. Inducible promoters can also be used. These promoters include MMTV LTR, inducible by dexamethasone, metallothionein, inducible by heavy metals, and promoters with cAMP response elements, inducible by cAMP, heat shock promoter. By using an inducible promoter, the nucleic acid can be delivered to a cell and will remain quiescent until the addition of the inducer. This allows further control on the timing of production of the protein of interest. Event-type specific promoters (e.g., HIV LTR) can be used, which are active or upregulated only upon the occurrence of an event, such as tumorigenicity or viral infection, for example. The HIV LTR promoter is inactive unless the tat gene product is present, which occurs upon viral infection. Some event-type promoters are also tissue-specific. Preferred event-type specific promoters include promoters activated upon viral infection.

Examples of promoters include promoters for α-fetoprotein, α-actin, myo D, carcinoembryonic antigen, VEGF-receptor; FGF receptor; TEK or tie 2; tie; urokinase receptor; E- and P-selectins; VCAM-1; endoglin; endosialin; αV-β3 integrin; endothelin-1; ICAM-3; E9 antigen; von Willebrand factor; CD44; CD40; vascular-endothelial cadherin; notch 4, high molecular weight melanoma-associated antigen; prostate specific antigen-1, probasin, FGF receptor, VEGF receptor, erb B2; erb B3; erb B4; MUC-1; HSP-27; int-1; int-2, CEA, HBEGF receptor; EGF receptor; tyrosinase, MAGE, IL-2 receptor; prostatic acid phosphatase, probasin, prostate specific membrane antigen, α-crystallin, PDGF receptor, integrin receptor, α-actin, SM1 and SM2 myosin heavy chains, calponin-hl, SM22 α-angiotensin receptor, IL-1, IL-2, IL-3, IL-4, IL-5, IL-6, IL-7, IL-8, IL-9, IL-10, IL-11, IL-12, IL-13, IL-14, immunoglobulin heavy chain, immunoglobulin light chain, and CD4.

Repressor sequences, negative regulators, or tissue-specific silencers can be inserted to reduce non-specific expression of the polynucleotide. Multiple repressor elements can be inserted in the promoter region. Repression of transcription is independent of the orientation of repressor elements or distance from the promoter. One type of repressor sequence is an insulator sequence. Such sequences inhibit transcription and can silence background transcription. Negative regulatory elements can be located in the promoter regions of a number of different genes. The repressor element can function as a repressor of transcription in the absence of factors, such as steroids, as does the NSE in the promoter region of the ovalbumin gene. These negative regulatory elements can bind specific protein complexes from oviduct, none of which are sensitive to steroids. Three different elements are located in the promoter of the ovalbumin gene. In some embodiments, oligonucleotides corresponding to portions of these elements can repress viral transcription of the TK reporter. For example, one such silencer element is TCTCTCCNA (SEQ ID NO: 11), which has a similar sequence identity as silencers that are present in other genes.

Elements that increase the expression of the desired target antigen can be incorporated into the nucleic acid sequence of the Ad vectors described herein. Exemplary elements include internal ribosome binding sites (RESs). RESs can increase translation efficiency. As well, other sequences can enhance expression. For some genes, sequences especially at the 5′ end can inhibit transcription and/or translation. These sequences are usually palindromes that can form hairpin structures. In some cases, such sequences in the nucleic acid to be delivered are deleted. Expression levels of the transcript or translated product can be assayed to confirm or ascertain which sequences affect expression. Transcript levels can be assayed by any known method, including Northern blot hybridization, RNase probe protection and the like. Protein levels can be assayed by any known method, including ELISA.

### Antigen-Specific Immunotherapies and Vaccines

Certain embodiments provide single antigen immunization against CEA utilizing such vectors and other vectors as provided herein. Certain embodiments provide prophylactic vaccines against CEA. Further, in various embodiments, the composition and methods provide herein can lead to clinical responses, such as altered disease progression or life expectancy.

Ad5 [E1−] vectors encoding a variety of antigens can be used to efficiently transduce 95% of ex vivo exposed DC's to high titers of the vector. In certain embodiments, increasing levels of foreign gene expression in the DC was found to correlate with increasing multiplicities of infection (MOI) with the vector. DCs infected with Ad5 [E1−] vectors can encode a variety of antigens (including the tumor antigens MART-1, MAGE-A4, DF3/MUC1, p53, hugp100 melanoma antigen, polyoma virus middle-T antigen) that have the propensity to induce antigen specific CTL responses, have an enhanced antigen presentation capacity, and/or have an improved ability to initiate T-cell proliferation in mixed lymphocyte reactions. Immunization of animals with dendritic cells (DCs) previously transduced by Ad5 vectors encoding tumor specific antigens can be used to induce significant levels of protection for the animals when challenged with tumor cells expressing the respective antigen. Interestingly, intra-tumoral injection of Ads encoding IL-7 is less effective than injection of DCs transduced with IL-7 encoding Ad5 vectors at inducing antitumor immunity. Ex vivo transduction of DCs by Ad5 vectors is contemplated in certain embodiments. Ex vivo DC transduction strategies can been used to induce recipient host tolerance. For example, Ad5 mediated delivery of the CTLA4Ig into DCs can block interactions of the DCs CD80 with CD28 molecules present on T-cells.

Ad5 vector capsid interactions with DCs can trigger several beneficial responses, which can enhance the propensity of DCs to present antigens encoded by Ad5 vectors. For example, immature DCs, though specialized in antigen uptake, are relatively inefficient effectors of T-cell activation. DC maturation coincides with the enhanced ability of DCs to drive T-cell immunity. In some instances, the compositions and methods take advantage of an Ad5 infection resulting in direct induction of DC maturation Ad vector infection of immature bone marrow derived DCs from mice can upregulate cell surface markers normally associated with DC maturation (MHC I and II, CD40, CD80, CD86, and ICAM-1) as well as down-regulation of CD11c, an integrin down regulated upon myeloid DC maturation. In some instances, Ad vector infection triggers IL-12 production by DCs, a marker of DC maturation. Without being bound by theory, these events can possibly be due to Ad5 triggered activation of NF-κB pathways. Mature DCs can be efficiently transduced by Ad vectors, and do not lose their functional potential to stimulate the proliferation of naive T-cells at lower MOI, as demonstrated by mature CD83+ human DC (derived from peripheral blood monocytes). However, mature DCs can also be less vulnerable to infection than immature ones. Modification of capsid proteins can be used as a strategy to optimize infection of DC by Ad vectors, as well as enhancing functional maturation, for example using the CD40L receptor as a viral vector receptor, rather than using the normal CAR receptor infection mechanisms.

In some embodiments, the compositions and methods comprising an Ad5 [E1−, E2b−] vector(s) CEA vaccine have effects of increased overall survival (OS) within the bounds of technical safety. In some embodiments, the compositions and methods comprising an Ad5 [E1−, E2b−] vector(s) CEA vaccine have effects of increased overall survival (OS) within the bounds of technical safety. In certain embodiments, the compositions and methods comprising an Ad5 [E1−, E2b−] vector(s) CEA vaccine have effects of increased overall survival (OS) within the bounds of technical safety.

In some embodiments, the antigen targets are associated with benign tumors. In some embodiments, the antigens targeted are associated with pre-cancerous tumors.

In some embodiments, the antigens targeted are associated with carcinomas, in situ carcinomas, metastatic tumors, neuroblastoma, sarcomas, myosarcoma, leiomyosarcoma, retinoblastoma, hepatoma, rhabdomyosarcoma, plasmocytomas, adenomas, gliomas, thymomas, or osteosarcoma. In some embodiments, the antigens targeted are associated with a specific type of cancer such as neurologic cancers, brain cancer, thyroid cancer, head and neck cancer, melanoma, leukemia, acute lymphoblastic leukemia (ALL), acute myelogenous leukemia (AML), chronic myelogenous leukemia (CML), and chronic lymphocytic leukemia (CLL), non-Hodgkin's lymphoma, multiple myeloma, Hodgkin's disease, breast cancer, bladder cancer, prostate cancer, colorectal cancer, colon cancer, kidney cancer, renal cell carcinoma, pancreatic cancer, esophageal cancer, lung cancer, mesothelioma, ovarian cancer, cervical cancer, endometrial cancer, uterine cancer, germ cell tumors, testicular cancer, gastric cancer, or other cancers, or any clinical (e.g., TNM, Histopathological, Staging or Grading systems or a combination thereof) or molecular subtype thereof. In some embodiments, the antigens targeted are associated with a specific clinical or molecular subtype of cancer. By way of example, breast cancer can be divided into at least four molecular subtypes including Luminal A, Luminal B, Triple negative/basal-like, and HER2 type. By way of example, prostate cancer can be subdivided TNM, Gleason score, or molecular expression of the PSA protein.

As noted above, an adenovirus vector can comprise a nucleic acid sequence that encodes one or more target proteins or antigens of interest. In this regard, the vectors can contain nucleic acid encoding 1, 2, 3, 4, 5, 6, 7, 8, 9, 10, 11, 12, 13, 14, 15, 16, 17, 18, 19, 20 or more different target antigens of interest. The target antigens can be a full-length protein or can be a fragment (e.g., an epitope) thereof. The adenovirus vectors can contain nucleic acid sequences encoding multiple fragments or epitopes from one target protein of interest or can contain one or more fragments or epitopes from numerous different target proteins of interest. A target antigen can comprise any substance against which it is desirable to generate an immune response but generally, the target antigen is a protein. A target antigen can comprise a full-length protein, a subunit of a protein, an isoform of a protein, or a fragment thereof that induces an immune response (i.e., an immunogenic fragment). A target antigen or fragment thereof can be modified, e.g., to reduce one or more biological activities of the target antigen or to enhance its immunogenicity. The target antigen or target protein can be CEA.

In certain embodiments, immunogenic fragments bind to an MHC class I or class II molecule. An immunogenic fragment can “bind to” an MHC class I or class II molecule if such binding is detectable using any assay known in the art. For example, the ability of a polypeptide to bind to MHC class I can be evaluated indirectly by monitoring the ability to promote incorporation of ¹²⁵I labeled β-2-microglobulin (β-2m) into MHC class I/β2m/peptide heterotrimeric complexes. Alternatively, functional peptide competition assays that are known in the art can be employed. Immunogenic fragments of polypeptides can generally be identified using well known techniques. Representative techniques for identifying immunogenic fragments include screening polypeptides for the ability to react with antigen-specific antisera and/or T-cell lines or clones. An immunogenic fragment of a particular target polypeptide is a fragment that reacts with such antisera and/or T-cells at a level that is not substantially less than the reactivity of the full-length target polypeptide (e.g., in an ELISA and/or T-cell reactivity assay). In other words, an immunogenic fragment can react within such assays at a level that is similar to or greater than the reactivity of the full-length polypeptide. Such screens can be performed using methods known in the art.

In some embodiments, the viral vectors comprise heterologous nucleic acid sequences that encode one or more proteins, variants thereof, fusions thereof, or fragments thereof, that can modulate the immune response. In some embodiments, the viral vector encodes one or more antibodies against specific antigens, such as anthrax protective antigen, permitting passive immunotherapy. In some embodiments, the viral vectors comprise heterologous nucleic acid sequences encoding one or more proteins having therapeutic effect (e.g., anti-viral, anti-bacterial, anti-parasitic, or anti-tumor function). In some embodiments, the Second Generation E2b deleted adenovirus vectors comprise a heterologous nucleic acid sequence. In some embodiments, the heterologous nucleic acid sequence is CEA, a variant, a portion, or any combination thereof.

Target antigens include, but are not limited to, antigens derived from a variety of tumor proteins. In some embodiments, parts or variants of tumor proteins are employed as target antigens. In some embodiments, parts or variants of tumor proteins being employed as target antigens have a modified, for example, increased ability to effect and immune response against the tumor protein or cells containing the same. A vaccine can vaccinate against an antigen. A vaccine can also target an epitope. An antigen can be a tumor cell antigen. An epitope can be a tumor cell epitope. Such a tumor cell epitope can be derived from a wide variety of tumor antigens, such as antigens from tumors resulting from mutations, shared tumor specific antigens, differentiation antigens, and antigens overexpressed in tumors. Tumor-associated antigens (TAAs) can be antigens not normally expressed by the host; they can be mutated, truncated, misfolded, or otherwise abnormal manifestations of molecules normally expressed by the host; they can be identical to molecules normally expressed but expressed at abnormally high levels; or they can be expressed in a context or environment that is abnormal. Tumor-associated antigens can be, for example, proteins or protein fragments, complex carbohydrates, gangliosides, haptens, nucleic acids, other biological molecules or any combinations thereof.

Illustrative useful tumor proteins include, but are not limited to any one or more of, CEA, human epidermal growth factor receptor 1 (HER1), human epidermal growth factor receptor 2 (HER2/neu), human epidermal growth factor receptor 3 (HER3), human epidermal growth factor receptor 4 (HER4), MUC1, Prostate-specific antigen (PSA), PSMA, WT1, p53, MAGE-A1, MAGE-A2, MAGE-A3, MAGE-A4, MAGE-A6, MAGE-A10, MAGE-A12, BAGE, DAM-6, DAM-10, GAGE-1, GAGE-2, GAGE-8, GAGE-3, GAGE-4, GAGE-5, GAGE-6, GAGE-7B, NA88-A, NY-ESO-1, MART-1, MC1R, Gp100, PSA, PSM, Tyrosinase, TRP-1, TRP-2, ART-4, CAMEL, CEA, Cyp-B, BRCA1, Brachyury, Brachyury (TIVS7-2, polymorphism), Brachyury (IVS7 T/C polymorphism), T Brachyury, T, hTERT, hTRT, iCE, MUC1, MUC1 (VNTR polymorphism), MUC1c, MUC1n, MUC2, PRAME, P15, RU1, RU2, SART-1, SART-3, AFP, 0-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDCl27/m, TPI/mbcr-abl, ETV6/AML, LDLR/FUT, Pml/RARα, HPV E6, HPV E7, and TEL/AML1.

In some embodiments, the viral vector comprises a target antigen sequence encoding a modified polypeptide selected from CEA, human epidermal growth factor receptor 1 (HER1), human epidermal growth factor receptor 2 (HER2/neu), human epidermal growth factor receptor 3 (HER3), human epidermal growth factor receptor 4 (HER4), MUC1, Prostate-specific antigen (PSA), PSMA (i.e., PSM), WT1, p53, MAGE-A1, MAGE-A2, MAGE-A3, MAGE-A4, MAGE-A6, MAGE-A10, MAGE-A12, BAGE, DAM-6, DAM-10, GAGE-1, GAGE-2, GAGE-8, GAGE-3, GAGE-4, GAGE-5, GAGE-6, GAGE-7B, NA88-A, NY-ESO-1, MART-1, MC1R, Gp100, Tyrosinase, TRP-1, TRP-2, ART-4, CAMEL, Cyp-B, BRCA1, Brachyury, Brachyury (TIVS7-2, polymorphism), Brachyury (IVS7 T/C polymorphism), T Brachyury, T, hTERT, hTRT, iCE, MUC1 (VNTR polymorphism), MUC1c, MUC1n, MUC2, PRAME, P15, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDCl27/m, TPI/mbcr-abl, ETV6/AML, LDLR/FUT, Pml/RARα, HPV E6, HPV E7, and TEL/AML1, wherein the polypeptide or a fragment thereof has at least 60%, 65%, 70%, 75%, 80%, 85%, 90%, 95%, 98%, 99%, 99.5%, or 99.9% identity to the corresponding native sequence.

Additional illustrative useful tumor proteins useful include, but are not limited to any one or more of alpha-actinin-4, ARTC1, CAR-ABL fusion protein (b3a2), B-RAF, CASP-5, CASP-8, beta-catenin, Cdc27, CDK4, CDKN2A, COA-1, dek-can fusion protein, EFTUD2, Elongation factor 2, ETV6-AML1 fusion protein, FLT3-ITD, FN1, GPNMB, LDLR-fucosyltransferase fusion protein, HLA-A2d, HLA-A1 ld, hsp70-2, KIAAO205, MART2, ME1, MUM-lf, MUM-2, MUM-3, neo-PAP, Myosin class I, NFYC, OGT, OS-9, p53, pml-RARalpha fusion protein, PRDX5, PTPRK, K-ras, N-ras, RBAF600, SIRT2, SNRPD1, SYT-SSX1- or -SSX2 fusion protein, TGF-betaRII, triosephosphate isomerase, BAGE-1, GnTVf, HERV-K-MEL, KK-LC-1, KM-HN-1, LAGE-1, MAGE-A9, MAGE-C2, mucink, NA-88, NY-ESO-1/LAGE-2, SAGE, Sp17, SSX-2, SSX-4, TAG-1, TAG-2, TRAG-3, TRP2-INT2g, XAGE-1b, gp100/Pmel17, Kallikrein 4, mammaglobin-A, Melan-A/MART-1, NY-BR-1, OA1, PSA, RAB38/NY-MEL-1, TRP-1/gp75, TRP-2, tyrosinase, adipophilin, AIM-2, ALDH1A1, BCLX (L), BCMA, BING-4, CPSF, cyclin D1, DKK1, ENAH (hMena), EP-CAM, EphA3, EZH2, FGF5, G250/MN/CAIX, IL13Ralpha2, intestinal carboxyl esterase, alpha fetoprotein, M-CSFT, MCSP, mdm-2, MMP-2, PBF, PRAME, RAGE-1, RGS5, RNF43, RU2AS, secernin 1, SOX10, STEAP1, survivin, Telomerase, and/or VEGF.

Tumor-associated antigens can be antigens from infectious agents associated with human malignancies. Examples of infectious agents associated with human malignancies include Epstein-Barr virus, Helicobacter pylori, Hepatitis B virus, Hepatitis C virus, Human heresvirus-8, Human immunodeficiency virus, Human papillomavirus, Human T-cell leukemia virus, liver flukes, and Schistosoma haematobium.

### CEA Antigen Targets

CEA represents an attractive target antigen for immunotherapy since it is over-expressed in nearly all colorectal cancers and pancreatic cancers, and is also expressed by some lung and breast cancers, and uncommon tumors such as medullary thyroid cancer, but is not expressed in other cells of the body except for low-level expression in gastrointestinal epithelium. CEA contains epitopes that may be recognized in an MHC restricted fashion by T-cells.

It was discovered that multiple homologous immunizations with Ad5 [E1−, E2b−]-CEA(6D), encoding the tumor antigen CEA, induced CEA-specific cell-mediated immune (CMI) responses with antitumor activity in mice despite the presence of pre-existing or induced Ad5-neutralizing antibody. In the present phase I/II study, cohorts of patients with advanced colorectal cancer were immunized with escalating doses of Ad5 [E1−, E2b−]-CEA(6D). CEA-specific CMI responses were observed despite the presence of pre-existing Ad5 immunity in a majority (61.3%) of patients. Importantly, there was minimal toxicity, and overall patient survival (48% at 12 months) was similar regardless of pre-existing Ad5 neutralizing antibody titers. The results demonstrate that, in cancer patients, the novel Ad5 [E1−, E2b−] gene delivery platform generates significant CMI responses to the tumor antigen CEA in the setting of both naturally acquired and immunization-induced Ad5 specific immunity.

CEA antigen specific CMI can be, for example, greater than 10, 20, 30, 40, 50, 100, 200, 300, 400, 500, 600, 700, 800, 900, 1000, 5000, 10000, or more IFN-γ spot forming cells (SFC) per 10⁶ peripheral blood mononuclear cells (PBMC). In some embodiments, the immune response is raised in a human subject with a preexisting inverse Ad5 neutralizing antibody titer of greater than 50, 100, 150, 200, 300, 400, 500, 600, 700, 800, 900, 1000, 1500, 2000, 2500, 3000, 3500, 4000, 4500, 5000, 6000, 7000, 8000, 9000, 1000, 12000, 15000 or higher. The immune response may comprise a cell-mediated immunity and/or a humoral immunity as described herein. The immune response may be measured by one or more of intracellular cytokine staining (ICS), ELISpot, proliferation assays, cytotoxic T-cell assays including chromium release or equivalent assays, and gene expression analysis using any number of polymerase chain reaction (PCR) or RT-PCR based assays, as described herein and to the extent they are available to a person skilled in the art, as well as any other suitable assays known in the art for measuring immune response.

In some embodiments, the replication defective adenovirus vector comprises a modified sequence encoding a subunit with at least 75%, 80%, 85%, 90%, 95%, 98%, 99%, 99.5%, or 99.9% identity to a wild-type subunit of the polypeptide.

The immunogenic polypeptide may be a mutant CEA or a fragment thereof. In some embodiments, the immunogenic polypeptide comprises a mutant CEA with an Asn->Asp substitution at position 610. In some embodiments, the replication defective adenovirus vector comprises a sequence encoding a polypeptide with at least 75%, 80%, 85%, 90%, 95%, 98%, 99%, 99.5%, or 99.9% identity to the immunogenic polypeptide. In some embodiments, the sequence encoding the immunogenic polypeptide comprises the sequence of SEQ ID NO: 1 or SEQ ID NO: 100.

In some embodiments, the sequence encoding the immunogenic polypeptide comprises a sequence with at least 70% 75%, 80%, 85%, 90%, 95%, 98%, 99%, 99.5%, or 99.9% identity to SEQ ID NO: 1 or SEQ ID NO: 100 or a sequence generated from SEQ ID NO: 1 or SEQ ID NO: 100 by alternative codon replacements. In some embodiments, the immunogenic polypeptide encoded by the adenovirus vectors comprise up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human CEA sequence.

In some embodiments, the immunogenic polypeptide comprises a sequence from SEQ ID NO: 2 or a modified version, e.g., comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, of SEQ ID NO: 1 or SEQ ID NO: 100.

Members of the CEA gene family are subdivided into three subgroups based on sequence similarity, developmental expression patterns and their biological functions: the CEA-related Cell Adhesion Molecule (CEACAM) subgroup containing twelve genes (CEACAM, CEACAM3-CEACAM8, CEACAM16 and CEACAM18-CEACAM21), the Pregnancy Specific Glycoprotein (PSG) subgroup containing eleven closely related genes (PSG1-PSG11) and a subgroup of eleven pseudogenes (CEACAMP1-CEACAMP11). Most members of the CEACAM subgroup have similar structures that consist of an extracellular Ig-like domains composed of a single N-terminal V-set domain, with structural homology to the immunoglobulin variable domains, followed by varying numbers of C2-set domains of A or B subtypes, a transmembrane domain and a cytoplasmic domain. There are two members of CEACAM subgroup (CEACAM16 and CEACAM20) that show a few exceptions in the organization of their structures. CEACAM16 contains two Ig-like V-type domains at its N and C termini and CEACAM20 contains a truncated Ig-like V-type 1 domain. The CEACAM molecules can be anchored to the cell surface via their transmembrane domains (CEACAM5 thought CEACAM8) or directly linked to glycophosphatidylinositol (GPI) lipid moiety (CEACAM5, CEACAM18 thought CEACAM21).

CEA family members are expressed in different cell types and have a wide range of biological functions. CEACAMs are found prominently on most epithelial cells and are present on different leucocytes. In humans, CEACAM1, the ancestor member of CEA family, is expressed on the apical side of epithelial and endothelial cells as well as on lymphoid and myeloid cells. CEACAM1 mediates cell-cell adhesion through hemophilic (CEACAM to CEACAM) as well as heterothallic (e.g., CEACAM1 to CEACAM5) interactions. In addition, CEACAM1 is involved in many other biological processes, such as angiogenesis, cell migration, and immune functions. CEACAM3 and CEACAM4 expression is largely restricted to granulocytes, and they are able to convey uptake and destruction of several bacterial pathogens including Neisseria, Moraxella, and Haemophilus species.

Thus, in various embodiments, compositions and methods relate to raising an immune response against a CEA, selected from the group consisting of CEACAM1, CEACAM3, CEACAM4, CEACAM5, CEACAM6, CEACAM7, CEACAM8, CEACAM16, CEACAM18, CEACAM19, CEACAM20, CEACAM21, PSG1, PSG2, PSG3, PSG4, PSG5, PSG6, PSG7, PSG8, PSG9, and PSG11. An immune response may be raised against cells, e.g., cancer cells, expressing or overexpressing one or more of the CEAs, using the methods and compositions. In some embodiments, the overexpression of the one or more CEAs in such cancer cells is over 5, 10, 20, 30, 40, 50, 60, 70, 80, 90, 100 fold or more compared to non-cancer cells.

In certain embodiments, the CEA antigen used herein is a wild-type CEA antigen or a modified CEA antigen having a least a mutation in YLSGANLNL (SEQ ID NO: 3), a CAP1 epitope of CEA. The mutation can be conservative or non-conservative, substitution, addition, or deletion. In certain embodiments, the CEA antigen used herein has an amino acid sequence set forth in YLSGADLNL (SEQ ID NO: 4), a mutated CAP1 epitope. In further embodiments, the first replication-defective vector or a replication-defective vectors that express CEA has a nucleotide sequence at least 50%, 60%, 65%, 70%, 75%, 80%, 85%, 90%, 95%, 98%, 99%, 99.5%, 99.9%, or 100% identical to any portion of SEQ ID NO: 2 (the predicted sequence of an adenovirus vector expressing a modified CEA antigen), such as positions 1057 to 3165 of SEQ ID NO: 2 or full-length SEQ ID NO: 2.

### Mucin Family Antigen Targets

The human mucin family (MUC1 to MUC21) includes secreted and transmembrane mucins that play a role in forming protective mucous barriers on epithelial surfaces in the body. These proteins function in to protecting the epithelia lining the respiratory, gastrointestinal tracts, and lining ducts in important organs such as, for example the mammary gland, liver, stomach, pancreas, and kidneys.

MUC1 (CD227) is a TAA that is over-expressed on a majority of human carcinomas and several hematologic malignancies. MUC1 (GenBank: X80761.1, NCBI: NM_001204285.1) and activates many important cellular pathways known to be involved in human disease. MUC1 is a heterodimeric protein formed by two subunits that is commonly overexpressed in several human cancers. MUC1 undergoes autoproteolysis to generate two subunits MUC1n and MUC1c that, in turn, form a stable noncovalent heterodimer.

The MUC1 C-terminal subunit (MUC1c) can comprise a 58 amino acid extracellular domain (ED), a 28 amino acid transmembrane domain (TM), and a 72 amino acid cytoplasmic domain (CD). The MUC1c also can contains a “CQC” motif that can allow for dimerization of MUC1 and it can also impart oncogenic function to a cell. In some cases, MUC1 can in part oncogenic function through inducing cellular signaling via MUC1c. MUC1c can interact with EGFR, ErbB2 and other receptor tyrosine kinases and contributing to the activation of the PI3K→AKT and MEK→ERK cellular pathways. In the nucleus, MUC1c activates the Wnt/β-catenin, STAT and NF-κB RelA cellular pathways. In some cases, MUC1 can impart oncogenic function through inducing cellular signaling via MUC1n. The MUC1 N-terminal subunit (MUC1n) can comprise variable numbers of 20 amino acid tandem repeats that can be glycosylated. MUC1 is normally expressed at the surface of glandular epithelial cells and is over-expressed and aberrantly glycosylated in carcinomas. MUC1 is a TAA that can be utilized as a target for tumor immunotherapy. Several clinical trials have been and are being performed to evaluate the use of MUC1 in immunotherapeutic vaccines. Importantly, these trials indicate that immunotherapy with MUC1 targeting is safe and may provide survival benefit.

However, clinical trials have also shown that MUC1 is a relatively poor immunogen. To overcome this, the present invention describes identifying a T lymphocyte immune enhancer peptide sequence in the C terminus region of the MUC1 oncoprotein (MUC1-C or MUC1c). Compared with the native peptide sequence, the agonist in their modified MUC1-C (a) bound HLA-A2 at lower peptide concentrations, (b) demonstrated a higher avidity for HLA-A2, (c) when used with antigen-presenting cells, induced the production of more IFN-γ by T-cells than with the use of the native peptide, and (d) was capable of more efficiently generating MUC1-specific human T-cell lines from cancer patients. Importantly, T-cell lines generated using the agonist epitope were more efficient than those generated with the native epitope for the lysis of targets pulsed with the native epitope and in the lysis of HLA-A2 human tumor cells expressing MUC1. Additionally, the the present disclosure describes identification additional CD8+ cytotoxic T lymphocyte immune enhancer agonist sequence epitopes of MUC1-C.

Certain embodiments provide a potent MUC1-C modified for immune enhancer capability (mMUC1-C or MUC1-C or MUC1c). Certain embodiments provide a potent MUC1-C modified for immune enhancer capability incorporated it into a recombinant Ad5 [E1−, E2b−] platform to produce a new and more potent immunotherapeutic vaccine. For example, the immunotherapeutic vaccine can be Ad5 [E1−, E2b−]-mMUC1-C for treating MUC1 expressing cancers or infectious diseases.

Post-translational modifications play an important role in controlling protein function in the body and in human disease. For example, in addition to proteolytic cleavage discussed above, MUC1 can have several post-translational modifications such as glycosylation, sialylation, palmitoylation, or a combination thereof at specific amino acid residues. Provided herein are immunotherapies targeting glycosylation, sialylation, phosphorylation, or palmitoylation modifications of MUC1.

MUC1 can be highly glycosylated (N- and O-linked carbohydrates and sialic acid at varying degrees on serine and threonine residues within each tandem repeat, ranging from mono- to penta-glycosylation). Differentially O-glycosylated in breast carcinomas with 3,4-linked GlcNAc. N-glycosylation consists of high-mannose, acidic complex-type and hybrid glycans in the secreted form MUC1/SEC, and neutral complex-type in the transmembrane form, MUC1/TM.4. Certain embodiments provide immunotherapies targeting differentially 0-glycosylated forms of MUC1.

Further, MUC1 can be sialylated. Membrane-shed glycoproteins from kidney and breast cancer cells have preferentially sialyated core 1 structures, while secreted forms from the same tissues display mainly core 2 structures. The O-glycosylated content is overlapping in both these tissues with terminal fucose and galactose, 2- and 3-linked galactose, 3- and 3,6-linked GaNAc-ol and 4-linked GlcNAc predominating. Certain embodiments provide immunotherapies targeting various sialylation forms of MUC1. Dual palmitoylation on cysteine residues in the CQC motif is required for recycling from endosomes back to the plasma membrane. Certain embodiments provide for immunotherapies targeting various palmitoylation forms of MUC1.

Phosphorylation can affect MUC1's ability to induces specific cell signaling responses that are important for human health. Certain embodiments provide for immunotherapies targeting various phosphorylated forms of MUC1. For example, MUC1 can be phosphorylated on tyrosine and serine residues in the C-terminal domain. Phosphorylation on tyrosines in the C-terminal domain can increase nuclear location of MUC1 and β-catenin. Phosphorylation by PKC delta can induce binding of MUC1 to β-catenin/CTNNB1 and decrease formation of β-catenin/E-cadherin complexes. Src-mediated phosphorylation of MUC1 can inhibits interaction with GSK3B. Src- and EGFR-mediated phosphorylation of MUC1 on Tyr-1229 can increase binding to β-catenin/CTNNB1. GSK3B-mediated phosphorylation of MUC1 on Ser-1227 can decrease this interaction but restores the formation of the β-cadherin/E-cadherin complex. PDGFR-mediated phosphorylation of MUC1 can increase nuclear colocalization of MUC1CT and CTNNB1. Certain embodiments provide immunotherapies targeting different phosphorylated forms of MUC1, MUC1c and MUC1n known to regulate its cell signaling abilities.

The disclosure provides for immunotherapies that modulate MUC1c cytoplasmic domain and its functions in the cell. The disclosure provides for immunotherapies that comprise modulating a CQC motif in MUC1c. The disclosure provides for immunotherapies that comprise modulating the extracellular domain (ED), the transmembrane domain (TM), the cytoplasmic domain (CD) of MUC1c, or a combination thereof. The disclosure provides for immunotherapies that comprise modulating MUC1c's ability to induce cellular signaling through EGFR, ErbB2 or other receptor tyrosine kinases. The disclosure provides for immunotherapies that comprise modulating MUC1c's ability to induce PI3K→AKT, MEK→ERK, Wnt/β-catenin, STAT, NF-κB RelA cellular pathways, or combination thereof. In some embodiments, the MUC1c immunotherapy can further comprise CEA.

The disclosure also provides for immunotherapies that modulate MUC1n and its cellular functions. The disclosure also provides for immunotherapies comprising tandem repeats of MUC1n, the glycosylation sites on the tandem repeats of MUC1n, or a combination thereof. In some embodiments, the MUC1n immunotherapy further comprises CEA.

The disclosure also provides vaccines comprising MUC1n, MUC1c, CEA, or a combination thereof. The disclosure provides vaccines comprising MUC1c and CEA. The disclosure also provides vaccines targeting MUC1n and CEA. In some embodiments, the antigen combination is contained in one vector as provided herein. In some embodiments, the antigen combination is contained in a separate vector as provided herein.

Some embodiments relate to a replication defective adenovirus vector of serotype 5 comprising a sequence encoding an immunogenic polypeptide. The immunogenic polypeptide may be an isoform of MUC1 or a subunit or a fragment thereof. In some embodiments, the replication defective adenovirus vector comprises a sequence encoding a polypeptide with at least 75%, 80%, 85%, 90%, 95%, 98%, 99%, 99.5%, or 99.9% identity to the immunogenic polypeptide. In some embodiments, the sequence encoding the immunogenic polypeptide comprises the sequence of SEQ ID NO: 102. In some embodiments, the sequence encoding the immunogenic polypeptide comprises the sequence of SEQ ID NO: 5. In some embodiments, the sequence encoding the immunogenic polypeptide comprises the following sequence identified by SEQ ID NO: 6. In some embodiments, the sequence encoding the immunogenic polypeptide comprises the following sequence identified by SEQ ID NO: 9. In some embodiments, the sequence encoding the immunogenic polypeptide comprises the sequence of SEQ ID NO: 102. In some embodiments, the sequence encoding the immunogenic polypeptide comprises a sequence with at least 70%, 75%, 80%, 85%, 90%, 95%, 98%, 99%, 99.5%, 99.9% identity to SEQ ID NO: 5, SEQ ID NO: 6, SEQ ID NO: 101, SEQ ID NO: 9, SEQ ID NO: 102 or a sequence generated from SEQ ID NO: 5, SEQ ID NO: 6, SEQ ID NO: 101, SEQ ID NO: 9 or SEQ ID NO: 102 by alternative codon replacements. In some embodiments, the immunogenic polypeptide encoded by the adenovirus vectors described herein comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human MUC1 sequence.

In certain embodiments, the MUC1 antigen used herein is a wild-type MUC1 antigen or a modified MUC1 antigen. In certain embodiments, the modified MUC1 antigen has at least 50%, 60%, 65%, 70%, 75%, 80%, 85%, 90%, 95%, 98%, 99%, 99.5%, 99.9%, 100% identity to SEQ ID NO: 7 (a mutated MUC1 protein sequence) or SEQ ID NO: 101 (a modified MUC1 nucleotide sequence). In certain embodiments, the MUC-1 antigen is a modified antigen having one or more mutations at positions 93, 141-142, 149-151, 392, 404, 406, 422, 430-431, 444-445, or 460 of SEQ ID NO: 7. The mutation can be conservative or non-conservative, substitution, addition, or deletion. In further embodiments, the MUC-1 antigen binds to HLA-A2, HLA-A3, HLA-A24, or a combination thereof. In certain embodiments, the third replication-defective vector or a replication-defective vector that express MUC1 has a nucleotide sequence at least 50%, 60%, 65%, 70%, 75%, 80%, 85%, 90%, 95%, 98%, 99%, 99.5%, 99.9%, or 100% identical to SEQ ID NO: 5 (MUC_1 wild-type nucleotide sequence). In further embodiments, the third replication-defective vector or a replication-defective vector that express MUC1 has a nucleotide sequence at least 50%, 60%, 65%, 70%, 75%, 80%, 85%, 90%, 95%, 98%, 99%, 99.5%, 99.9%, or 100% identical to SEQ ID NO: 6 (a mutated MUC1 nucleotide sequence). In further embodiments, the third replication-defective vector or a replication-defective vector that express MUC1 has a nucleotide sequence at least 50%, 60%, 65%, 70%, 75%, 80%, 85%, 90%, 95%, 98%, 99%, 99.5%, 99.9%, or 100% identical to SEQ ID NO: 101 (a modified MUC1 nucleotide sequence, also referred to herein as MUC1-c). In certain embodiments, the third replication-defective vector or a replication-defective vector that express MUC1 has a nucleotide sequence at least 50%, 60%, 65%, 70%, 75%, 80%, 85%, 90%, 95%, 98%, 99%, 99.5%, 99.9%, or 100% identical to any portion of or full-length SEQ ID NO: 8 (the predicted sequence of an adenovirus vector expressing a modified CEA antigen), such as positions 1033-2858 of SEQ ID NO: 8.

### Brachyury Antigen Targets

Certain embodiments provide immunotherapies that comprise one or more antigens to Brachyury. Brachyury (also known as the “T” protein in humans) is a member of the T-box family of transcription factors that play key roles during early development, mostly in the formation and differentiation of normal mesoderm and is characterized by a highly conserved DNA-binding domain designated as T-domain. The epithelial to mesenchymal transition (EMT) is a key step during the progression of primary tumors into a metastatic state in which Brachyury plays a crucial role. The expression of Brachyury in human carcinoma cells induces changes characteristic of EMT, including up-regulation of mesenchymal markers, down-regulation of epithelial markers, and an increase in cell migration and invasion. Conversely, inhibition of Brachyury resulted in down-regulation of mesenchymal markers and loss of cell migration and invasion and diminished the ability of human tumor cells to form metastases. Brachyury can function to mediate epithelial-mesenchymal transition and promotes invasion.

The disclosure also provides for immunotherapies that modulate Brachyury effect on epithelial-mesenchymal transition function in cell proliferation diseases, such as cancer. The disclosure also provides for immunotherapies that modulate Brachyury's ability to promote invasion in cell proliferation diseases, such as cancer. The disclosure also provides for immunotherapies that modulate the DNA binding function of T-box domain of Brachyury. In some embodiments, the Brachyury immunotherapy can further comprise one or more antigens to CEA or MUC1, MUC1c, or MUC1n.

Brachyury expression is nearly undetectable in most normal human tissues and is highly restricted to human tumors and often overexpressed making it an attractive target antigen for immunotherapy. In human, Brachyury is encoded by the T gene (GenBank: AJ001699.1, NCBI: NM_003181.3). There are at least two different isoforms produced by alternative splicing found in humans. Each isoform has a number of natural variants.

Brachyury is immunogenic and Brachyury-specific CD8+ T-cells expanded in vitro can lyse Brachyury expressing tumor cells. These features of Brachyury make it an attractive TAA for immunotherapy. The Brachyury protein is a T-box transcription factor. It can bind to a specific DNA element, a near palindromic sequence “TCACACCT” (SEQ ID NO:108) through a region in its N-terminus, called the T-box to activate gene transcription when bound to such a site.

The disclosure also provides vaccines comprising Brachyury, CEA, or a combination thereof. In some embodiments, the antigen combination is contained in one vector as provided herein. In some embodiments, the antigen combination is contained in a separate vector as provided herein.

In particular embodiments, there is provided a replication defective adenovirus vector of serotype 5 comprising a sequence encoding an immunogenic polypeptide. The immunogenic polypeptide may be an isoform of Brachyury or a subunit or a fragment thereof. In some embodiments, the replication defective adenovirus vector comprises a sequence encoding a polypeptide with at least 70%, 75%, 80%, 85%, 90%, 95%, 98%, 99%, 99.5%, or 99.9% identity to the immunogenic polypeptide. In some embodiments, the sequence encoding the immunogenic polypeptide comprises the following sequence identified by SEQ ID NO: 101. In some embodiments, the sequence encoding the immunogenic polypeptide comprises the following sequence identified by SEQ ID NO: 7. In some embodiments, the replication defective adenovirus vector comprises a sequence encoding a polypeptide with at least 70%, 75%, 80%, 85%, 90%, 95%, 98%, 99%, 99.5%, or 99.9% identity to the immunogenic polypeptide. In some embodiments, the sequence encoding the immunogenic polypeptide comprises the following sequence identified by SEQ ID NO: 102. In some embodiments, the sequence encoding the immunogenic polypeptide comprises the sequence of SEQ ID NO: 8. In some embodiments, the sequence encoding the immunogenic polypeptide comprises a sequence with at least 70%, 75%, 80%, 85%, 90%, 95%, 98%, 99%, 99.5%, or 99.9% identity to SEQ ID NO: 7, SEQ ID NO: 101, SEQ ID NO: 8 or a sequence generated from SEQ ID NO: 7, SEQ ID NO: 101, or SEQ ID NO: 8 by alternative codon replacements. In some embodiments, the immunogenic polypeptide encoded by the adenovirus vectors described herein comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human Brachyury sequence.

In certain embodiments, the Brachyury antigen used herein is a wild-type antigen or a modified antigen. In certain embodiments, the Brachyury antigen binds to HLA-A2. In further embodiments, the Brachyury antigen is a modified Brachyury antigen comprising an amino acid sequence set forth in WLLPGTSTV (SEQ ID NO: 15), a HLA-A2 epitope of Brachyury. In further embodiments, the Brachyury antigen is a modified Brachyury antigen having an amino acid sequence at least 50%, 60%, 65%, 70%, 75%, 80%, 85%, 90%, 95%, 98%, 99%, 99.5%, 99.9%, or 100% identity to SEQ ID NO: 14, a modified Brachyury protein sequence. In certain embodiments, the replication-defective vector has a nucleotide sequence at least 80% identical SEQ ID NO: 10 or positions 1033 to 2283 of SEQ ID NO: 13. In further embodiments, the second replication-defective vector has a nucleotide sequence at least 50%, 60%, 65%, 70%, 75%, 80%, 85%, 90%, 95%, 98%, 99%, 99.5%, 99.9%, or 100% identical to any portion or full-length of SEQ ID NO: 13 (the predicted sequence of an adenovirus vector express a modified Brachyury antigen), such as positions 1033 to 2283 of SEQ ID NO: 13. In some embodiments, the Brachyury antigen is a modified Brachyury antigen having an amino acid sequence at least 80% identical to SEQ ID NO: 12 (another mutated Brachyury protein sequence). In certain embodiments, the second replication-defective vector or a replication-defective vector that express Brachyury has a nucleotide sequence at least 50%, 60%, 65%, 70%, 75%, 80%, 85%, 90%, 95%, 98%, 99%, 99.5%, 99.9%, or 100% identical to positions 520-1824 of SEQ ID NO: 9 (wild-type Brachyury), SEQ ID NO: 9, SEQ ID NO: 10, or SEQ ID NO: 102. In certain embodiments, the second replication-defective vector or a replication-defective vector that express Brachyury has a nucleotide sequence at least 50%, 60%, 65%, 70%, 75%, 80%, 85%, 90%, 95%, 98%, 99%, 99.5%, 99.9%, or 100% identical to SEQ ID NO: 102.

### Infectious Disease-Associated Antigen Targets

Target antigens include, but are not limited to, antigens derived from any of a variety of infectious agents such as parasites, bacteria, virus, prions, and the like. An infectious agent may refer to any living organism capable of infecting a host. Infectious agents include, for example, bacteria, any variety of viruses, such as, single stranded RNA viruses, single stranded DNA viruses, fungi, parasites, and protozoa.

Examples of infectious disease associated target antigens that can be used with the compositions and the methods can be derived from the following: Actinobacillus spp., Actinomyces spp., Adenovirus (types 1, 2, 3, 4, 5, 6, and 7), Adenovirus (types 40 and 41), Aerococcus spp., Aeromonas hydrophila, Ancylostoma duodenale, Angiostrongylus cantonensis, Ascaris lumbricoides, Ascaris spp., Aspergillus spp., Babesia spp, B. microti, Bacillus anthracis, Bacillus cereus, Bacteroides spp., Balantidium coli, Bartonella bacilliformis, Blastomyces dermatitidis, Bluetongue virus, Bordetella bronchiseptica, Bordetella pertussis, Borrelia afzelii, Borrelia burgdorferi, Borrelia garinii, Branhamella catarrhalis, Brucella spp. (B. abortus, B. canis, B. melitensis, B. suis), Brugia spp., Burkholderia, (Pseudomonas) mallei, Burkholderia (Pseudomonas) pseudomallei, California serogroup, Campylobacter fetus subsp. Fetus, Campylobacter jejuni, C. coli, C. fetus subsp. Jejuni, Candida albicans, Capnocytophaga spp., Chikungunya virus, Chlamydia psittaci, Chlamydia trachomatis, Citrobacter spp., Clonorchis sinensis, Clostridium botulinum, Clostridium difficile, Clostridium perfringens, Clostridium tetani, Clostridium spp. (with the exception of those species listed above), Coccidioides immitis, Colorado tick fever virus, Corynebacterium diphtheriae, Coxiella burnetii, Coxsackievirus, Creutzfeldt-Jakob agent, Kuru agent, Crimean-Congo hemorrhagic fever virus, Cryptococcus neoformans, Cryptosporidium parvum, Cytomegalovirus, Cyclospora cayatanesis, Dengue virus (1, 2, 3, 4), Diphtheroids, Eastern (Western) equine encephalitis virus, Ebola virus, Echinococcus granulosus, Echinococcus multilocularis, Echovirus, Edwardsiella tarda, Entamoeba histolytica, Enterobacter spp., Enterovirus 70, Epidermophyton floccosum, Ehrlichia spp, Ehrlichia sennetsu, Microsporum spp. Trichophyton spp., Epstein-Barr virus, Escherichia coli, enterohemorrhagic, Escherichia coli, enteroinvasive, Escherichia coli, enteropathogenic, Escherichia coli, enterotoxigenic, Fasciola hepatica, Francisella tularensis, Fusobacterium spp., Gemella haemolysans, Giardia lamblia, Guanarito virus, Haemophilus ducreyi, Haemophilus influenzae (group b), Hantavirus, Hepatitis A virus, Hepatitis B virus, Hepatitis C virus, Hepatitis D virus, Hepatitis E virus, Herpes simplex virus, Herpesvirus simiae, Histoplasma capsulatum, Human coronavirus, Human immunodeficiency virus, Human papillomavirus, Human rotavirus, Human T-lymphotrophic virus, Influenza virus including H5N1, Junin virus/Machupo virus, Klebsiella spp., Kyasanur Forest disease virus, Lactobacillus spp., Lassa virus, Legionella pneumophila, Leishmania major, Leishmania infantum, Leishmania spp., Leptospira interrogans, Listeria monocytogenes, Lymphocytic choriomeningitis virus, Machupo virus, Marburg virus, Measles virus, Micrococcus spp., Moraxella spp., Mycobacterium spp. (other than M. bovis, M. tuberculosis, M. avium, M. leprae), Mycobacterium tuberculosis, M. bovis, Mycoplasma hominis, M. orale, M. salivarium, M. fermentans, Mycoplasma pneumoniae, Naegleria fowleri, Necator americanus, Neisseria gonorrhoeae, Neisseria meningitides, Neisseria spp. (other than N. gonorrhoeae and N. meningitidis), Nocardia spp., Norwalk virus, Omsk hemorrhagic fever virus, Onchocerca volvulus, Opisthorchis spp., Parvovirus B19, Pasteurella spp., Peptococcus spp., Peptostreptococcus spp., Plasmodium falciparum, Plasmodium vivax, Plasmodium spp., Plesiomonas shigelloides, Powassan encephalitis virus, Proteus spp., Pseudomonas spp. (other than P. mallei, P. pseudomallei), Rabies virus, Respiratory syncytial virus, Rhinovirus, Rickettsia akari, Rickettsia prowazekii, R. canada, Rickettsia rickettsii, Rift Valley virus, Ross river virus/O'Nyong-Nyong virus, Rubella virus, Salmonella choleraesuis, Salmonella paratyphi, Salmonella typhi, Salmonella spp. (with the exception of those species listed above), Schistosoma spp., Scrapie agent, Serratia spp., Shigella spp., Sindbis virus, Sporothrix schenckii, St. Louis encephalitis virus, Murray Valley encephalitis virus, Staphylococcus aureus, Streptobacillus moniliformis, Streptococcus agalactiae, Streptococcus faecalis, Streptococcus pneumoniae, Streptococcus pyogenes, Streptococcus salivarius, Taenia saginata, Taenia solium, Toxocara canis, T. cati, T. cruzi, Toxoplasma gondii, Treponema pallidum, Trichinella spp., Trichomonas vaginalis, Trichuris trichiura, Trypanosoma brucei, Trypanosoma cruzi, Ureaplasma urealyticum, Vaccinia virus, Varicella-zoster virus, eastern equine encephalitis virus (EEEV), severe acute respiratory virus (SARS), Venezuelan equine encephalitis virus (VEEV), Vesicular stomatitis virus, Vibrio cholerae, serovar 01, Vibrio parahaemolyticus, West Nile virus, Wuchereria bancrofti, Yellow fever virus, Yersinia enterocolitica, Yersinia pseudotuberculosis, and Yersinia pestis. Target antigens can include proteins, or variants or fragments thereof, produced by any of the infectious organisms.

A number of viruses are associated with viral hemorrhagic fever, including filoviruses (e.g., Ebola, Marburg, and Reston), arenaviruses (e.g., Lassa, Junin, and Machupo), and bunyaviruses. In addition, phleboviruses, including, for example, Rift Valley fever virus, have been identified as etiologic agents of viral hemorrhagic fever. Etiological agents of hemorrhagic fever and associated inflammation can also include paramyxoviruses, particularly respiratory syncytial virus. In addition, other viruses causing hemorrhagic fevers in man have been identified as belonging to the following virus groups: togavirus (Chikungunya), flavivirus (dengue, yellow fever, Kyasanur Forest disease, Omsk hemorrhagic fever), nairovirus (Crimian-Congo hemorrhagic fever) and hantavirus (hemorrhagic fever with renal syndrome, nephropathic epidemia). Furthermore, Sin Nombre virus was identified as the etiologic agent of the 1993 outbreak of hantavirus pulmonary syndrome in the American Southwest.

Target antigens can include viral coat proteins, i.e., influenza neuraminidase and hemagglutinin, HIV gp160 or derivatives thereof, HIV Gag, HIV Nef, HIV Pol, SARS coat proteins, herpes virion proteins, WNV proteins, etc. Target antigens can also include bacterial surface proteins including pneumococcal PsaA, PspA, LytA, surface or virulence associated proteins of bacterial pathogens such as Nisseria gonnorhea, outer membrane proteins or surface proteases.

### Personalized Tumor-Associated Antigens

In certain embodiments tumor-associated antigens used with the compositions and methods as described herein can be identified directly from an individual with a proliferative disease or cancer. In certain embodiments, cancers can include benign tumors, metastatic tumors, carcinomas, or sarcomas and the like. In some embodiments, a personalized tumor antigen comprises CEA characterized from a patient and further utilized as the target antigen as a whole, in part or as a variant.

In this regard, screens can be carried out using a variety of known technologies to identify tumor target antigens from an individual. For example, in one embodiment, a tumor biopsy is taken from a patient, RNA is isolated from the tumor cells and screened using a gene chip (for example, from AFFYMETRIX®, Santa Clara, Calif.) and a tumor antigen is identified. Once the tumor target antigen is identified, it can then be cloned, expressed, and purified using techniques known in the art.

This target antigen can then linked to one or more epitopes or incorporated or linked to cassettes or viral vectors described herein and administered to the patient in order to alter the immune response to the target molecule isolated from the tumor. In this manner, “personalized” immunotherapy and vaccines are contemplated in certain embodiments. Where cancer is genetic (i.e., inherited), for example, the patient has been identified to have a BRAC1 or BRAC2 mutation, the vaccine can be used prophylactically. When the cancer is sporadic this immunotherapy can be used to reduce the size of the tumor, enhance overall survival and reduce reoccurrence of the cancer in a subject.

### Tumor Neo-Antigens

In some embodiments, the present disclosure provides identification of tumor neo-antigens to be used in a personalized vaccine to a subject in need thereof using any adenovirus vector described herein, such as the Ad5 [E1−, E2b−] virus vectors. Neo-antigens can also be referred to herein as “neo-epitopes.” Tumor neo-antigens can result from various mutations, for example any category of DNA mutation, which can occur during tumorigenesis.

In some embodiments, neo-antigens can be more advantageous as a vaccine target as compared to other tumor antigens as described by Martin et al. (Ann Oncol. 2015 December; 26(12): 2367-2374.). For example, T cells that are capable of targeting neo-antigens do not face tolerance and, thus, can be more cytotoxic against target neo-antigen bearing cancer cells and can be less affected by mechanisms of immune suppression. Because, neo-antigens result from mutations during tumorigenesis, neo-antigens can be wholly unique to cancer cells and can be absent from occurring in host cells. Incorporation of said neo-antigens in an effective adenovirus vector such as the Ad5 [E1−, E2b−] vectors described herein can, thus, be a powerful way of selectively vaccinating against tumors while minimizing off target cytotoxic effects on non-tumor host cells. Finally, multiple neo-antigens can be presented at the cell surface of tumor cells.

Mutations that can give rise to tumor neo-antigens, also referred to as somatic mutations, can be present at any residue in the neo-antigen. However, because neo-antigens must be (1) presented on an MHC molecule, such as MHC class I or MHC class II and (2) recognized as a complex with an MHC molecule by a T cell receptor (TCR), mutations that result in especially immunogenic neo-antigens can be located in residues that interact with an MHC molecule or interact with a TCR. Examples of mutations that can result in neo-antigens include non-synonymous mutations, read-through mutations, splice site mutations, chromosomal rearrangements, and frameshift mutations as described in detail in US Patent Application No. 20160331822. Sequencing techniques described in further detail below, can be used to identify said mutations in order to differentiate between tumor cells and host cell. Neo-antigens of the present application can also include mutations that are known to be drivers of tumor genesis, for example any of those described in the Catalogue of Somatic Mutations in Cancer (COSMIC) database (http://cancer.sanger.ac.uk/cosmic). Neo-antigens can be derived from driver and passenger genes as described by Martin et al. (Ann Oncol. 2015 December; 26(12): 2367-2374.) and can be present in several different types of tumors.

### Sequencing Methods

In some embodiments, methods and assays for identifying the neo-antigens described herein are provided. In some embodiments, the present disclosure provides sequencing techniques, such as next-generation sequencing techniques, to identify tumor neo-epitopes associated with cancer cells. Processed tissue samples are DNA or RNA sequencing to identify mutations that are unique to tumor neo-antigens, which are distinct from host cells. Sequencing can be performed on patient-derived samples to identify possible neo-epitopes to target utilizing an adenovirus vector-based vaccine. For example, in some embodiments, tissue from a subject in need thereof is obtained and processed for sequencing analysis. Sequencing analysis can be combined with genomics, bioinformatics, and immunological approaches to identify mutant tumor associated antigens and epitopes.

In some embodiments, sequencing methods and assays for obtaining a sequence-verified neo-antigen vector are described herein. For example, any sequencing method described herein can be used to analyze the sequence of a replication-defective vector of the present disclosure with or without a desired neo-antigen construct inserted into the vector. Said sequencing of the replication-defective vector can confirm that the desired construct was designed and produced. Said sequencing can be performed at any step of producing a sequence-verified neo-antigen vector. For example, in some embodiments, sequencing of a neo-antigen vector comprising a neo-antigen sequence and a sequence for an Ad5 [E1−, E2b−] vector of the present disclosure, to obtain a sequence-verified neo-antigen vector, can be performed following homologous recombination of the neo-antigen into the vector, following membrane purification of the vector, or any combination thereof. The goal of obtaining a sequence-verified neo-antigen vector can be to confirm that a polynucleotide sequence of a final packaged virion is 100% identical to a polynucleotide sequence of a shuttle plasmid, to confirm that a polynucleotide sequence of a final packaged virion is 100% identical to a polynucleotide sequence of the vector and neo-antigen following homologous recombination, to confirm that a polynucleotide sequence of the vector comprises a deletion in an E1 region, an E2 region, an E2b region, an E3 region, an E4 region, or any combination thereof of a replication defective viral vector, to confirm that a polynucleotide sequence does not comprise any unintentional sequencing errors, to confirm that a polynucleotide sequence that comprises the vector and neo-antigen does not comprise one or more contaminating sequences, to confirm that a sequence of a neo-antigen produced after passaging the cells, or any combination thereof. In some embodiments, the sequencing methods of the present disclosure can be used to obtain a sequence-verified neo-antigen vector that can be used as a personalized cancer vaccine in a subject in need thereof. Sequence verification can be a pivotal step in producing personalized cancer vaccines, particularly for neo-antigens, which are specific to patients and are not commonly characterized in the art. Thus, the methods described herein can be used to obtain sequence-verified neo-antigen vectors, which can have superior efficacy and lower off-target effects as compared to non-sequence verified neo-antigen vectors, which may encode for erroneous or incorrect moieties. In some embodiments, any next generation sequencing (NGS) technique used herein to obtain the sequence-verified neo-antigen vector confirms that sequence-verified neo-antigen vector has at least 90%, 92%, 95%, 97%, 99%, or 99.5% sequence identity to the expected sequence. NGS techniques of the present disclosure are described in further detail below.

In some embodiments, the tissue obtained from a subject can be analyzed by any sequencing technique, including whole exome sequencing or whole genome sequencing. Non sequencing techniques can also be used to supplement sequencing data in order to identify neo-antigens with high binding affinity for MHC. For example, computer algorithms can be used to predict binding affinity of a given neo-antigen to MHC. In some embodiments, MHC multimer screens and functional T cell assays can be used to assess the immunogenicity of an identified neo-antigen. Any next-generation sequencing (NGS) method can be used herein to sequence a tumor tissue sample obtained from a subject. Said NGS methods can include, but are not limited to, those described below.

In some embodiments, GPS Cancer™ can be used to sequence-verify neo-antigen vectors or to sequence neo-antigens, as described above. GPS Cancer™ can include mass spectrometry, whole genome (DNA) sequencing, and whole transcriptome (RNA) sequencing. GPS Cancer™ sequencing methods and analyses can be used to provide personalized treatment strategies for a subject in need thereof, as further described at www.gpscancer.com.

Tumor neo-antigens can be identified using standard next-generation sequencing (NGS) methods including, but not limited to, genome sequencing and resequencing, RNA-sequencing, and ChIP sequencing.

Said techniques can be used identify mutations, such as missense mutations or frameshift mutations, in tumor cells as compared to host cells. DNA mutations can be identified using massively parallel sequencing (MPS) as described by Gubin et al. (J Clin Invest. 2015 Sep. 1; 125(9): 3413-3421) and Simpson et al. (Nat Rev Cancer. 2005 August; 5(8):615-25). RNA can also be analyzed by first obtaining corresponding cDNA and sequencing said cDNA. In some embodiments, exome-capture can be used to sequence and identify tumor neo-antigen genes as described in Gubin et al. (J Clin Invest. 2015 Sep. 1; 125(9): 3413-3421) by comparison of the resulting sequencing data to normal cells, which can serve as a reference sequence.

Further assays that can be used to identify tumor neo-antigens include, but are not limited to, proteomics (e.g., protein sequencing by tandem mass spectrometry (MS/MS) or meta-shotgun protein sequencing), array hybridization, solution hybridization, nucleic amplification, polymerase chain reaction, quantitative PCR, RT-PCR, in situ hybridization, Northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Oligo Ligation Assay (OLA), hybridization, and array analysis as described in US20170211074, which is incorporated herein by reference.

In some embodiments, a panomics-based test is performed to compare sequencing data between a tumor sample and a normal reference samples. Said panomics-based tests can comprise analyzing the whole genome, single nucleotide variances (SNVs), copy number variances, insertions, deletions, rearrangements, or any combination thereof. Samples that can be sequenced for identification of tumor neo-antigens can be any sample from a subject. Said samples can be extracted for DNA or RNA. In some embodiments, samples can be formalin fixed paraffin embedded (FFPE) or freshly frozen. In some embodiments, the RainStorm (Raindance Technologies) system or molecular inversion probes (MIP) can be used for DNA extraction from FFPE samples. In some embodiments, the sample can be whole blood. In some embodiments, the sample is a solid tumor tissue sample or a liquid tumor sample. Samples can be enriched, for example, using laser microdissection. The TruSeq™ DNA Sample Preparation Kit and the Exome Enrichment Kit TruSeq™ Exome Enrichment Kit can be used for sample preparation and enrichment prior to sequencing. In some embodiments, enrichment can comprise PCR-amplicon based methods or hybridization capture methods as described in Meldrum et al. (Clin Biochem Rev. 2011 November; 32(4): 177-195). In some embodiments, microfluidics-based methods can be used for PCR-based enrichment. For example, the Fluidigm system can be used to carry out multiple parallel PCR reactions.

In some embodiments, any suitable sequencing method can be used including, but not limited to, the classic Sanger sequencing method, high-throughput sequencing, pyrosequencing, sequencing-by-synthesis, single-molecule sequencing, nanopore sequencing, sequencing-by-ligation, sequencing-by-hybridization, RNA-Seq (Illumina), Digital Gene Expression (Helicos), next generation sequencing, single molecule sequencing by synthesis (SMSS) (Helicos), massively-parallel sequencing, clonal single molecule Array (Solexa), shotgun sequencing, Maxim-Gilbert sequencing, primer walking, next-generation sequencing, and any other sequencing methods known in the art. In some embodiments, sequencing methods and assays for obtaining a sequence-verified neo-antigen vector are carried out using Sanger sequencing to verify the insert and polymerase chain reaction (PCR) to test for mutations. In some embodiments, Sanger sequencing confirms that the neo-antigen vector obtained through the methods of making described herein has 97%, 98%, 99%, 99.1%, 99.2%, 99.3%, 99.4%, 99.5%, 99.6%, 99.7%, 99.8%, or 99.9% sequence identity to the expected sequence.

In some instances, next-generation sequencing, or “NGS,” can be used to sequence a molecule described herein. NGS techniques can include all novel high throughput sequencing technologies which, in contrast to the “conventional” sequencing methodology known as Sanger chemistry, read nucleic acid templates randomly in parallel along the entire genome by breaking the entire genome into small pieces.

Any NGS technique can be used to analyze the whole genome, exomes, transcriptomes, and/or methylomes, as described in WO2016128376 A1. Said NGS techniques can be carried out in less than 2 weeks, less than 1 week, less than 6 days, less than 5 days, less than 4 days, less than 3 days, less than 2 day, or less than 1 day. Commercially NGS platforms that can be used to sequence for neo-antigens of the present disclosure are described by Zhang et al. (J Genet Genomics. Author manuscript; available in PMC 2011 Apr. 13).

NGS methods used herein can include any method described in Masoudi-Nejad, Ali, Zahra Narimani, and Nazanin Hosseinkhan. Next generation sequencing and sequence assembly: methodologies and algorithms. Vol. 4. Springer Science & Business Media, 2013; Buermans et al., “Next Generation sequencing technology: Advances and applications,” Biochimica et Biophysica Acta, 1842:1931-1941, 2014.; and by Liu et al., Comparison of Next-Generation Sequencing Systems. Journal of Biomedicine and Biotechnology, 11 pages, 2012. NGS methods used herein can also include those described in US20160125129, each of which is incorporated herein by reference.

For example, in some embodiments, sequencing-by synthesis (Solexa, now Illumina) can be performed using the Illumina/Solexa Genome Analyzer™ and the Illumina HiSeq 2000 Genome Analyze.

In some embodiments, sequencing-by-ligation can be performed using SOLid™ platform of Applied Biosystems (Life Technologies) or the Polonator™ G.007 platform of Dover Systems (Salem, N.H.).

In some embodiments, single-molecule sequencing can be performed using the PacBio RS system of Pacific Biosciences (Menlo Park, Calif.), the HeliScope™ platform of Helicos Biosciences (Cambridge, Mass.), a fluorescence based systems from Visigen Biotechnology (Houston, Tex.), U.S. Genomics (GeneEngine™), or Genovoxx (AnyGene™).

In some embodiments, nanotechnology based single-molecule sequencing can be performed using GridON™ platform, hybridization-assisted nano-pore sequencing (HANS™) platforms, ligase-based DNA sequencing platform referred to as combinatorial probe-anchor ligation (cPAL™), and electron microscopy.

In some embodiments, the NGS method is ion semiconductor sequencing, which can be performed using Ion Torrent Systems.

Further methods are described in Teer et al. (Hum Mol Genet. 2010 Oct. 15; 19(R2):R145-51), Hodges et al. (Nat Genet. 2007 December; 39(12):1522-7), and Choi et al. (Proc Natl Acad Sci USA. 2009 Nov. 10; 106(45):19096-101).

Commercial kits for DNA sample preparation and subsequent exome capture are also available: for example, Illumina Inc. (San Diego, Calif.) offers the TruSeq™ DNA Sample Preparation Kit and the Exome Enrichment Kit TruSeq™ Exome Enrichment Kit.

In some embodiments, RNA sequencing can be used to identify tumor neo-antigens. RNA sequencing technologies can include any high-throughput sequencing method, for example, Illumina IG, Applied Biosystems SOLiD and Roche 454 Life Science systems, or a Helicos Biosciences tSMS system as described in Wang et al. (Nat Rev Genet. 2009 January; 10(1): 57-63). In some embodiments, extracted RNA can be converted to cDNA and subsequently sequenced at read lengths of 30-400 base pairs.

High-throughput sequencing methods can also be employed to characterize short stretches of sequence contiguity and genomic variation. U.S. Pat. No. 9,715,573 (Dovetail Genomics, LLC) discloses methods for rapid paired and/or grouped sequence reads, which can be used to assess sequence contiguity at the chromosomal level,

### Identification of Tumor Neo-Antigens and Neo-Epitopes

In some embodiments, sequencing analysis can be used to identify neo-antigens. The neo-antigen can be an 8 mer to a 50 mer. In other embodiments, the neo-antigen can be up to a 25 mer. Identified neo-antigens can be further analyzed for their affinity for binding HLA molecules of a subject. As described above, highly immunogenic neo-antigens can have high affinity for MHC (HLA in humans) molecules. In some embodiments, the present disclosure provides neo-antigen inserts, which can comprise one or more than one neo-antigen sequences, a linker, a tag, and other factors, and can therefore be up to 3 kilobases.

In some embodiments, the HLA type of a subject is identified and computer prediction algorithms are used to model mutations in neo-antigens that can result in high affinity for binding HLA and/or MHC molecules. Tools to predict neo-antigen binding to MHC molecules can include any of those available at http://cancerimmunity.org/resources/webtools, including but not limited to, PAProC, NetChop, MAPPP, TAPPred, RankPep, MHCBench, HLA Peptide Binding Predictions, PREDEP, nHLAPred-I, ProPred-1, SVMHC, EPIPREDICT, ProPred, NetMHC, NetMHCII, NetMHCpan, SMM, POPI, OptiTope, Mosaic Vaccine Tool Suite, HLABinding, Prediction of Antigenic Determinants, ANTIGENIC, BepiPred, DiscoTope, ElliPro, Antibody Epitope Prediction, CTLPred, NetCTL, MHC-I processing predictions, Epitope Cluster Analysis, Epitope Conservancy Analysis, VaxiJen, or combinations thereof. Programs such as SYFPEITHI, as described in Rammensee et al. (Immunogenetics. 1999 November; 50(3-4):213-9), Rankpep, as described in Reche et al. (Hum Immunol. 2002 September; 63(9):701-9), or BIMAS, as described in Parker et al (J Immunol. 1994 Jan. 1; 152(1):163-75) can also be used. In some embodiments, neo-antigens can also be identified using the Immune Epitope Database and Analysis Resource (IEDB), as described in Vita et al. (Nucleic Acids Res. 2015 January; 43(Database issue):D405-12). In some embodiments, said algorithms can predict peptide binding to MIIC class I variants using artificial neural networks (ANN). These algorithms can yield IC50 values as a metric of neo-antigen binding to MHC. NetMHC (Lundegaard et al. Nucleic Acids Res. 2008 Jul. 1; 36(Web Server issue): W509-W512. Published online 2008 May 7), or SMM (Peters et al. BMC Bioinformatics. 2005 May 31; 6:132) and SMMPMBEC (Kim et al. BMC Bioinformatics. 2009 Nov. 30; 10:394) can also be used. MIIC tetramer based assays can also be used to identify tumor neo-antigens with high binding affinity for MIIC molecules as described in Lu et al. (Semin Immunol. 2016 February; 28(1): 22-27). In some embodiments, SNPs can be removed from neo-antigens.

In some embodiments, tumor neo-antigens can also be identified by pulsing antigen presenting cells with relatively long synthetic peptides that encompass minimal T cell epitopes, as described by Lu et al. (Semin Immunol. 2016 February; 28(1): 22-27). In other embodiments, tumor neo-antigens can also be identified using tandem minigene screening or sequencing analysis of the whole-exome or the transcriptome, as described by Lu et al.

### Tumor Neo-Epitope Prioritization

In some embodiments, methods are provided for prioritizing tumor neo-antigens that can stimulate robust immune response after vaccination in an Ad5 [E1−, E2b−] viral vector of the present disclosure. For example, tumor neo-antigens identified by sequencing methods can be subsequently classified and prioritized by MIIC binding affinity. Tumor neo-antigens can be further classified and prioritized by epitope abundance, as determined by mass spectrometry, RNA expression levels, or RNA sequencing. Tumor neo-antigens can be further classified and prioritized by antigen processing, including antigen degradation and transport to MHC processing pathways.

Neo-antigen prioritization can be further refined by eliminating false positives and can be further subject to algorithms described in Gubin et al. (J Clin Invest. 2015 Sep. 1; 125(9): 3413-3421), including NetChop, NetCTL, and NetCTLpan (Nielsen M, et al. Immunogenetics, 2005; 57(1-2):33-41, Peters B, et al. J. Immunol., 2003; 171(4):1741-1749).

MIIC Class II binding affinities can be assessed using prediction algorithms such as those described in Gubin et al. (J Clin Invest. 2015 Sep. 1; 125(9): 3413-3421), including TEPITOPE (Hammer J, et al. J. Exp. Med., 1994; 180(6):2353-2358), netMHCII (Nielsen M, et al. BMC Bioinformatics. 2009; 10:296), and SMM-align (Nielsen M, et al. BMC Bioinformatics 2007; 8:238). Known programs such as the NetMHCpan program can be used to identify neo-antigens with high binding affinity for MHC.

In some embodiments, the affinity of a neo-antigen of the present disclosure for an MHC molecules can be less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500 nmol/L. In some embodiments, a neo-antigen that has strong affinity for MHC can have an IC50 value of less than 50 nmol/L. In some embodiments, a neo-antigen that has moderate affinity for MHC can have an IC 50 value from 50 to 150 nmol/L. In some embodiments, a neo-antigen that has weak affinity for MHC can have an IC50 value from 150 to 500 nmol/L. In some embodiments, a neo-antigen that has low or no affinity for MHC can have an IC50 value greater than 500 nmol/L.

In some embodiments, functional T cell responses can be further examined to prioritize neo-antigens. For example, neo-antigen pulsed antigen presenting cells can be co-cultured with CD4+ or CD8+ T cells and T-cell proliferation and cytokine release can be examined. Neo-antigens that elicit the highest functional T cell response can be prioritized for incorporation into a vector of the present disclosure

In some embodiments, the present disclosure provides methods of making and administering an individual, personalized neo-antigen/neo-epitope vaccine. For example, the present disclosure provides methods for obtaining a sample from a subject and analyzing the sample for the presence of tumor neo-epitopes or neo-antigens that are unique to that subject or to a subset of individuals. The tumor neo-epitopes or neo-antigens can be then sequenced and inserted into a vector of the present disclosure as shown in FIG. 1 at the insert design stage. Vectors are then subject to the manufacturing process of the present disclosure, which includes the step of utilizing a SARTOBIND® Q Membrane for purification, yielding efficient and high purity adenovirus vectors encoding for the neo-antigen or neo-epitope of interest. In some embodiments, the resulting neo-antigen vaccine can be sequence verified using high throughput sequencing methods, such as any next generation sequencing technique. The resulting neo-antigen/neo-epitope personalized vaccine can be administered back to the subject in need thereof.

Combination Immunotherapy with Ad5 Vaccines and Calreticulin

In some embodiments, any antigen described herein can be expressed as a fusion protein with calreticulin (CRT). CRT can serve as an immunologic adjuvant in cancer vaccines immunizing against tumor associated antigens, such as those described herein. In some embodiments, any antigen described herein, such as CEA (SEQ ID NO: 1, SEQ ID NO: 3, SEQ ID NO: 4, or SEQ ID NO: 100), MUC1-C(SEQ ID NO: 5, SEQ ID NO: 6, SEQ ID NO: 7, or SEQ ID NO: 101), or Brachyury (SEQ ID NO: 9, SEQ ID NO: 10, or SEQ ID NO: 102) are expressed as a fusion protein with CRT. In other embodiments, a neo-antigen is identified in a subject using the methods described herein and the neo-antigen is expressed as a fusion protein with CRT. The present disclosure provides compositions and methods for making Ad5 [E1−, E2b−] vectors encoding for any one of the above described fusions of an antigen with CRT.

CRT can be expressed on a tumor cell and can serve as a cancer marker to antigen presenting cells, which can subsequently phagocytose and cross-present tumor associated antigens from the tumor cell. CRT is a 60 kDa protein that can bind to calcium ions and is located in the endoplasmic reticulum. However, translocation of CRT from the endoplasmic reticulum to the cell surface can result in inducement of apoptosis and serve as a signal to antigen presenting cells to phagocytose said cell. In some embodiments, CRT can translocate from the endoplasmic reticulum to the cell surface on its own. In some embodiments, treatment with any chemotherapeutic agent can trigger CRT translocation from the endoplasmic reticulum to the cell surface. In some embodiments, CRT can have a sequence as set forth in SEQ ID NO: 107 (GCGGCGTCCGTCCGTACTGCAGAGCCGCTGCCGGAGGGTCGTTTTAAAGGGCCCGC GCGTTGCCGCCCCCTCGGCCCGCCATGCTGCTATCCGTGCCGCTGCTGCTCGGCCTCC TCGGCCTGGCCGTCGCCGAGCCTGCCGTCTACTTCAAGGAGCAGTTTCTGGACGGAG ACGGGTGGACTTCCCGCTGGATCGAATCCAAACACAAGTCAGATTTTGGCAAATTCG TTCTCAGTTCCGGCAAGTTCTACGGTGACGAGGAGAAAGATAAAGGTTTGCAGACA AGCCAGGATGCACGCTTTTATGCTCTGTCGGCCAGTTTCGAGCCTTTCAGCAACAAA GGCCAGACGCTGGTGGTGCAGTTCACGGTGAAACATGAGCAGAACATCGACTGTGG GGGCGGCTATGTGAAGCTGTTTCCTAATAGTTTGGACCAGACAGACATGCACGGAG ACTCAGAATACAACATCATGTTTGGTCCCGACATCTGTGGCCCTGGCACCAAGAAGG TTCATGTCATCTTCAACTACAAGGGCAAGAACGTGCTGATCAACAAGGACATCCGTT GCAAGGATGATGAGTTTACACACCTGTACACACTGATTGTGCGGCCAGACAACACCT ATGAGGTGAAGATTGACAACAGCCAGGTGGAGTCCGGCTCCTTGGAAGACGATTGG GACTTCCTGCCACCCAAGAAGATAAAGGATCCTGATGCTTCAAAACCGGAAGACTG GGATGAGCGGGCCAAGATCGATGATCCCACAGACTCCAAGCCTGAGGACTGGGACA AGCCCGAGCATATCCCTGACCCTGATGCTAAGAAGCCCGAGGACTGGGATGAAGAG ATGGACGGAGAGTGGGAACCCCCAGTGATTCAGAACCCTGAGTACAAGGGTGAGTG GAAGCCCCGGCAGATCGACAACCCAGATTACAAGGGCACTTGGATCCACCCAGAAA TTGACAACCCCGAGTATTCTCCCGATCCCAGTATCTATGCCTATGATAACTTTGGCGT GCTGGGCCTGGACCTCTGGCAGGTCAAGTCTGGCACCATCTTTGACAACTTCCTCAT CACCAACGATGAGGCATACGCTGAGGAGTTTGGCAACGAGACGTGGGGCGTAACAA AGGCAGCAGAGAAACAAATGAAGGACAAACAGGACGAGGAGCAGAGGCTTAAGGA GGAGGAAGAAGACAAGAAACGCAAAGAGGAGGAGGAGGCAGAGGACAAGGAGGA TGATGAGGACAAAGATGAGGATGAGGAGGATGAGGAGGACAAGGAGGAAGATGAG GAGGAAGATGTCCCCGGCCAGGCCAAGGACGAGCTGTAGAGAGGCCTGCCTCCAGG GCTGGACTGAGGCCTGAGCGCTCCTGCCGCAGAGCTGGCCGCGCCAAATAATGTCTC TGTGAGACTCGAGAACTTTCATTTTTTTCCAGGCTGGTTCGGATTTGGGGTGGATTTT GGTTTTGTTCCCCTCCTCCACTCTCCCCCACCCCCTCCCCGCCCTTTTTTTTTTTTTTTT TTAAACTGGTATTTTATCTTTGATTCTCCTTCAGCCCTCACCCCTGGTTCTCATCTTTC TTGATCAACATCTTTTCTTGCCTCTGTCCCCTTCTCTCATCTCTTAGCTCCCCTCCAAC CTGGGGGGCAGTGGTGTGGAGAAGCCACAGGCCTGAGATTTCATCTGCTCTCCTTCC TGGAGCCCAGAGGAGGGCAGCAGAAGGGGGTGGTGTCTCCAACCCCCCAGCACTGA GGAAGAACGGGGCTCTTCTCATTTCACCCCTCCCTTTCTCCCCTGCCCCCAGGACTGG GCCACTTCTGGGTGGGGCAGTGGGTCCCAGATTGGCTCACACTGAGAATGTAAGAA CTACAAACAAAATTTCTATTAAATTAAATTTTGTGTCTCCAAAAAAAAAAAAAAAAAA).

In some embodiments, the present disclosure provides a CRT fused to an antigen, wherein said antigen is a tumor associated antigen. When encoded for by an adenovirus vector of the present disclosure, the CRT-antigen fusion is expressed in cells. CRT, being capable of translocation to the cell surface, can subsequently move itself and the fused antigen to the cell surface, thereby signaling for phagocytosis of the CRT-antigen complex by a dendritic cell, which can lead to presentation of the antigen by the antigen presenting cell. Thus, in some embodiments, vectors of the present disclosure encoding for a fusion of CRT and an antigen are administered in a subject in need thereof and target tumor cells directly.

In some embodiments, the present disclosure provides a vector encoding for CRT fused to an antigen, wherein the target cell is an antigen presenting cell, such as a dendritic cell. CRT is also capable of functioning as a general adjuvant and can boost immune responses in vaccines. For example, when an adenovirus vector of the present disclosure encodes for a CRT-antigen fusion for vaccinating against a cancer, the resulting immune response is significantly greater than if the antigen alone was present in the adenovirus. For example, adenovirus vectors encoding for CRT-antigen fusions can induce greater levels of cytokine production (e.g., IFN-γ and TNF-α production), which can result in increased CD4+ and CD8+ T cell proliferation. Thus, compositions and methods provided herein provide a superior immunologic fusion of CRT with any antigen disclosed herein to induce robust protective immune responses.

In some embodiments, calreticulin would be directly fused to any antigen of the present disclosure (e.g., any one of SEQ ID NO: 1-SEQ ID NO: 15 or SEQ ID NO: 100-SEQ ID NO: 106). In some embodiments, CRT and the antigen would be separated by a linker, such as any one of SEQ ID NO: 84-SEQ ID NO: 98.

Combination Immunotherapy with Ad5-CEA Vaccines and IL-15 Superagonists

Certain embodiments provide combination immunotherapy compositions for the treatment of cancers. In some aspects, combination immunotherapies provided herein can comprise a multi-targeted immunotherapeutic approach against antigens associated with the development of cancer such as tumor associated antigen (TAA) or antigens know to be involved in a particular infectious disease, such as infectious disease associated antigen (IDAA). In some aspects, combination immunotherapies and vaccines provided herein can comprise a multi-targeted antigen signature immunotherapeutic approach against antigens associated with the development of cancer. The compositions and methods, in various embodiments, provide viral based vectors expressing CEA or a variant of CEA for immunization of a disease, as provided herein. These vectors can raise an immune response against CEA.

### Ad5-Based Vaccines in Combination Therapy

In some aspects, the vector can comprise at least one antigen, such as CEA. In some aspects, the vector can comprise at least two antigens. In some aspects, the vector can comprise at least three antigens. In some aspects, the vector can comprise more than three antigens. In some aspects, the vaccine formulation can comprise 1:1 ratio of vector to antigen. In some aspects, the vaccine can comprise 1:2 ratio of vector to antigen. In some aspects, the vaccine can comprise 1:3 ratio of vector to antigen. In some aspects, the vaccine can comprise 1:4 ratio of vector to antigen. In some aspects, the vaccine can comprise 1:5 ratio of vector to antigen. In some aspects, the vaccine can comprise 1:6 ratio of vector to antigen. In some aspects, the vaccine can comprise 1:7 ratio of vector to antigen. In some aspects, the vaccine can comprise 1:8 ratio of vector to antigen. In some aspects, the vaccine can comprise 1:9 ratio of vector to antigen. In some aspects, the vaccine can comprise 1:10 ratio of vector to antigen.

In some aspects, the vaccine can be a single-antigen vaccine, for example and Ad5[E1−, E2b−]-CEA vaccine. In some aspects, the vaccine can comprise a combination vaccine, wherein the vaccine can comprise at least two vectors each containing at least a single antigen. In some aspects the vaccine can be a combination vaccine, wherein the vaccine can comprise at least three vectors each containing at least a single antigen target. In some aspects the vaccine can comprise a combination vaccine, wherein the vaccine comprises more than three vectors each containing at least a single antigen.

In some aspects, the vaccine can be a combination vaccine, wherein the vaccine can comprise at least two vectors, wherein a first vector of the at least two vectors can comprise at least a single antigen and wherein a second vector of the at least two vectors can comprise at least two antigens. In some aspects, the vaccine can comprise a combination vaccine, wherein the vaccine can comprise at least three vectors, wherein a first vector of the at least three vectors can comprise at least a single antigen and wherein a second vector of the at least three vectors can comprise at least two antigens. In some aspects, the vaccine can be a combination vaccine, wherein the vaccine can comprise three or more vectors, wherein a first vector of the three or more vectors can comprise at least a single antigen and wherein a second vector of the three or more vectors can comprise at least two antigens. In some aspects, the vaccine can be a combination vaccine, wherein the vaccine can comprise more than three vectors each containing at least two antigens.

When a mixture of different antigens are simultaneously administered or expressed from a same or different vector in an individual, they may compete with one another. As a result the formulations comprising different concentration and ratios of expressed antigens in a combination immunotherapy or vaccine must be evaluated and tailored to the individual or group of individuals to ensure that effective and sustained immune responses occur after administration.

Composition that comprises multiple antigens can be present at various ratios. For example, formulations with more than vector can have various ratios. For example, immunotherapies or vaccines can have two different vectors in a stoichiometry of 1:1, 1:2, 1:3, 1:4, 1:5, 1:6, 1:7, 1:8, 1:9, 1:10, 1:15, 1:20, 1:30, 2:1, 2:3, 2:4, 2:5, 2:6, 2:7, 2:8, 3:1, 3:3, 3:4, 3:5, 3:6, 3:7, 3:8, 3:1, 3:3, 3:4, 3:5, 3:6, 3:7, 3:8, 4:1, 4:3, 4:5, 4:6, 4:7, 4:8, 5:1, 5:3, 5:4, 5:6, 5:7, 5:8, 6:1, 6:3, 6:4, 6:5, 6:7, 6:8, 7:1, 7:3, 7:4, 7:5, 7:6, 7:8, 8:1, 8:3, 8:4, 8:5, 8:6, or 8:7. For example, immunotherapies or vaccines can have three different vectors in a stoichiometry of: 1:1:1, 1:2:1, 1:3:1, 1:4:1, 1:5:1, 1:6:1, 1:7:1, 1:8:1, 2:1:1, 2:3:1, 2:4:1, 2:5:1, 2:6:1, 2:7:1, 2:8:1, 3:1, 3:3:1, 3:4:1, 3:5:1, 3:6:1, 3:7:1, 3:8:1, 3:1:1, 3:3:1, 3:4:1, 3:5:1, 3:6:1, 3:7:1, 3:8:1, 4:1:1, 4:3:1, 4:4:1, 4:5:1, 4:6:1, 4:7:1, 4:8:1, 5:1:1, 5:3:1, 5:4:1, 5:5:1, 5:6:1, 5:7:1, 5:8:1, 6:1:1, 6:3:1, 6:4:1, 6:5:1, 6:6:1, 6:7:1, 6:8:1, 7:1:1, 7:3:1, 7:4:1, 7:5:1, 7:6:1, 7:7:1, 7:8:1, 8:1:1, 8:3:1, 8:4:1, 8:5:1, 8:6:1, 8:7:1, 8:8:1, 1:1:2, 1:2:2, 1:3:2, 1:4:2, 1:5:2, 1:6:2, 1:7:2, 1:8:2, 2:1:2, 2:3:2, 2:4:2, 2:5:2, 2:6:2, 2:7:2, 2:8:2, 3:1:2, 3:3:2, 3:4:2, 3:5:2, 3:6:2, 3:7:2, 3:8:2, 3:1:2, 3:3:2, 3:4:2, 3:5:2, 3:6:2, 3:7:2, 3:8:2, 4:1:2, 4:3:2, 4:4:2, 4:5:2, 4:6:2, 4:7:2, 4:8:2, 5:1:2, 5:3:2, 5:4:2, 5:5:2, 5:6:2, 5:7:2, 5:8:2, 6:1:2, 6:3:2, 6:4:2, 6:5:2, 6:6:2, 6:7:2, 6:8:2, 7:1:2, 7:3:2, 7:4:2, 7:5:2, 7:6:2, 7:7:2, 7:8:2, 8:1:2, 8:3:2, 8:4:2, 8:5:2, 8:6:2, 8:7:2, 8:8:2, 1:1:3, 1:2:3, 1:3:3, 1:4:3, 1:5:3, 1:6:3, 1:7:3, 1:8:3, 2:1:3, 2:3:3, 2:4:3, 2:5:3, 2:6:3, 2:7:3, 2:8:3, 3:1:3, 3:3:3, 3:4:3, 3:5:3, 3:6:3, 3:7:3, 3:8:3, 3:1:3, 3:3:3, 3:4:3, 3:5:3, 3:6:3, 3:7:3, 3:8:3, 4:1:3, 4:3:3, 4:4:3, 4:5:3, 4:6:3, 4:7:3, 4:8:3, 5:1:3, 5:3:3, 5:4:3, 5:5:3, 5:6:3, 5:7:3, 5:8:3, 6:1:3, 6:3:3, 6:4:3, 6:5:3, 6:6:3, 6:7:3, 6:8:3, 7:1:3, 7:3:3, 7:4:3, 7:5:3, 7:6:3, 7:7:3, 7:8:3, 8:1:3, 8:3:3, 8:4:3, 8:5:3, 8:6:3, 8:7:3, 8:8:3, 1:1:4, 1:2:4, 1:3:4, 1:4:4, 1:5:4, 1:6:4, 1:7:4, 1:8:4, 2:1:4, 2:3:4, 2:4:4, 2:5:4, 2:6:4, 2:7:4, 2:8:4, 3:1:4, 3:3:4, 3:4:4, 3:5:4, 3:6:4, 3:7:4, 3:8:4, 3:1:4, 3:3:4, 3:4:4, 3:5:4, 3:6:4, 3:7:4, 3:8:4, 4:1:4, 4:3:4, 4:4:4, 4:5:4, 4:6:4, 4:7:4, 4:8:4, 5:1:4, 5:3:4, 5:4:4, 5:5:4, 5:6:4, 5:7:4, 5:8:4, 6:1:4, 6:3:4, 6:4:4, 6:5:4, 6:6:4, 6:7:4, 6:8:4, 7:1:4, 7:3:4, 7:4:4, 7:5:4, 7:6:4, 7:7:4, 7:8:4, 8:1:4, 8:3:4, 8:4:3, 8:5:4, 8:6:4, 8:7:4, 8:8:4, 1:1:5, 1:2:5, 1:3:5, 1:4:5, 1:5:5, 1:6:5, 1:7:5, 1:8:5, 2:1:5, 2:3:5, 2:4:5, 2:5:5, 2:6:5, 2:7:5, 2:8:5, 3:1:5, 3:3:5, 3:4:5, 3:5:5, 3:6:5, 3:7:5, 3:8:5, 3:1:5, 3:3:5, 3:4:5, 3:5:5, 3:6:5, 3:7:5, 3:8:5, 4:1:5, 4:3:5, 4:4:5, 4:5:5, 4:6:5, 4:7:5, 4:8:5, 5:1:5, 5:3:5, 5:4:5, 5:5:5, 5:6:5, 5:7:5, 5:8:5, 6:1:5, 6:3:5, 6:4:5, 6:5:5, 6:6:5, 6:7:5, 6:8:5, 7:1:5, 7:3:5, 7:4:5, 7:5:5, 7:6:5, 7:7:5, 7:8:5, 8:1:5, 8:3:5, 8:4:5, 8:5:5, 8:6:5, 8:7:5, 8:8:5, 1:1:6, 1:2:6, 1:3:6, 1:4:6, 1:5:6, 1:6:6, 1:7:6, 1:8:6, 2:1:6, 2:3:6, 2:4:6, 2:5:6, 2:6:6, 2:7:6, 2:8:6, 3:1:6, 3:3:6, 3:4:6, 3:5:6, 3:6:6, 3:7:6, 3:8:6, 3:1:6, 3:3:6, 3:4:6, 3:5:6, 3:6:6, 3:7:6, 3:8:6, 4:1:6, 4:3:6, 4:4:6, 4:5:6, 4:6:6, 4:7:6, 4:8:6, 5:1:6, 5:3:6, 5:4:6, 5:5:6, 5:6:6, 5:7:6, 5:8:6, 6:1:6, 6:3:6, 6:4:6, 6:5:6, 6:6:6, 6:7:6, 6:8:6, 7:1:6, 7:3:6, 7:4:6, 7:5:6, 7:6:6, 7:7:6, 7:8:6, 8:1:6, 8:3:6, 8:4:6, 8:5:6, 8:6:5, 8:7:6, 8:8:6, 1:1:7, 1:2:7, 1:3:7, 1:4:7, 1:5:7, 1:6:7, 1:7:7, 1:8:7, 2:1:7, 2:3:7, 2:4:7, 2:5:7, 2:6:7, 2:7:7, 2:8:7, 3:1:7, 3:3:7, 3:4:7, 3:5:7, 3:6:7, 3:7:7, 3:8:7, 3:1:7, 3:3:7, 3:4:7, 3:5:7, 3:6:7, 3:7:7, 3:8:7, 4:1:7, 4:3:7, 4:4:7, 4:5:7, 4:6:7, 4:7:7, 4:8:7, 5:1:7, 5:3:7, 5:4:7, 5:5:7, 5:6:7, 5:7:7, 5:8:7, 6:1:7, 6:3:7, 6:4:7, 6:5:7, 6:6:7, 6:7:7, 6:8:7, 7:1:7, 7:3:7, 7:4:7, 7:5:7, 7:6:7, 7:7:7, 7:8:7, 8:1:7, 8:3:7, 8:4:7, 8:5:7, 8:6:5, 8:7:7, or 8:8:7.

Certain embodiments provide combination immunotherapies comprising multi-targeted immunotherapeutic directed TAAs. Certain embodiments provide combination immunotherapies comprising multi-targeted immunotherapeutic directed to IDAAs.

Certain embodiments provide a combination immunotherapies or vaccines comprising: at least two, at least three, or more than three different target antigens comprising a sequence encoding a modified CEA. For example, a combination immunotherapy or vaccine can comprise at least two, at least three, or more than three different target antigens comprising a sequence encoding a modified CEA, wherein the modified CEA comprises a sequence with an identity value of at least 70%, 75%, 80%, 85%, 90%, 95%, 98%, 99%, 99.5%, or 99.9% to SEQ ID NO: 1 or SEQ ID NO: 100. In some embodiments, the modified CEA comprises a sequence with an identity value of at least 70%, 75%, 80%, 85%, 90%, 95%, 98%, 99%, 99.5%, 99.9%, or 100% SEQ ID NO: 1 and has a Asn->Asp substitution at position 610. In some embodiments, the CEA comprises a sequence of YLSGANLNL (SEQ ID NO: 3), a CAP1 epitope of CEA or YLSGADLNL (SEQ ID NO: 4), a mutated CAP1 epitope. The Ad5-CEA expressing vector can have a sequence as set forth in SEQ ID NO: 2.

IL-15 Superagonist in Combination Therapy with Ad5 Vaccines

The present invention provides compositions for combination therapy including an Ad5 [E1−, E2b−]-CEA vaccine and an IL-15 super-agonist complex. In certain embodiments, the present invention provides a method of treating a CEA-expressing cancer in a subject, the method comprising: administering to the individual a first pharmaceutical composition comprising a replication-defective vector comprising a nucleic acid sequence encoding a CEA antigen or any suitable antigen; and administering to the individual an IL-15 super-agonist. In some embodiments, the IL-15 super-agonist is any molecule or molecular complex that binds to and activates IL-15 receptors. In certain embodiments, the IL-15 super-agonist is ALT-803, a molecular complex of IL-15N72D, an IL-15RαSu domain, and an IgG1 Fc domain. The composition of ALT-803 and methods of producing and using ALT-803 are described in U.S. Patent Application Publication 2015/0374790, which is herein incorporated by reference.

Interleukin 15 (IL-15) is a naturally occurring inflammatory cytokine secreted after viral infections. Secreted IL-15 can carry out its function by signaling via the its cognate receptor on effector immune cells, and thus, can lead to overall enhancement of effector immune cell activity.

Based on IL-15's broad ability to stimulate and maintain cellular immune responses, it is believed to be a promising immunotherapeutic drug that could potentially cure certain cancers. However, major limitations in clinical development of IL-15 can include low production yields in standard mammalian cell expression systems and short serum half-life. Moreover, the IL-15:IL-15Rα complex, comprising proteins co-expressed by the same cell, rather than the free IL-15 cytokine, can be responsible for stimulating immune effector cells bearing IL-15 βγc receptor.

To contend with these shortcomings, a novel IL-15 superagonist mutant (IL-15N72D) was identified that has increased ability to bind IL-15Rβγc and enhanced biological activity. Addition of either mouse or human IL-15Rα and Fc fusion protein (the Fc region of immunoglobulin) to equal molar concentrations of IL-15N72D can provide a further increase in IL-15 biologic activity, such that IL-15N72D:IL-15Rα/Fc super-agonist complex exhibits a median effective concentration (EC₅₀) for supporting IL-15-dependent cell growth that was greater than 10-fold lower than that of free IL-15 cytokine.

Thus, in some embodiments, the present disclosure provides a IL-15N72D:IL-15Rα/Fc super-agonist complex with an EC₅₀ for supporting IL-15-dependent cell growth that is greater than 2-fold lower, greater than 3-fold lower, greater than 4-fold lower, greater than 5-fold lower, greater than 6-fold lower, greater than 7-fold lower, greater than 8-fold lower, greater than 9-fold lower, greater than 10-fold lower, greater than 15-fold lower, greater than 20-fold lower, greater than 25-fold lower, greater than 30-fold lower, greater than 35-fold lower, greater than 40-fold lower, greater than 45-fold lower, greater than 50-fold lower, greater than 55-fold lower, greater than 60-fold lower, greater than 65-fold lower, greater than 70-fold lower, greater than 75-fold lower, greater than 80-fold lower, greater than 85-fold lower, greater than 90-fold lower, greater than 95-fold lower, or greater than 100-fold lower than that of free IL-15 cytokine.

In some embodiments, the interaction of IL-15N72D, soluble IL-15Rα, and Fc fusion protein have been exploited to create a biologically active protein complex, ALT-803. It is known that a soluble IL-15Rα fragment, containing the so-called “sushi” domain at the N terminus (Su), bears most of the structural elements responsible for high affinity cytokine binding. A soluble fusion protein can be generated by linking the human IL-15RαSu domain (amino acids 1-65 of the mature human IL-15Rα protein) with the human IgG1 CH2-CH3 region containing the Fc domain (232 amino acids). This IL-15RαSu/IgG1 Fc fusion protein has the advantages of dimer formation through disulfide bonding via IgG1 domains and ease of purification using standard Protein A affinity chromatography methods.

ALT-803 is a soluble complex consisting of 2 protein subunits of a human IL-15 variant (two IL-15N72D subunits) associated with high affinity to a dimeric IL-15Rα sushi domain/human IgG1 Fcfusion protein and. The IL-15 variant is a 114-amino acid polypeptide comprising the mature human IL-15 cytokine sequence with an Asn to Asp substitution at position 72 of helix C N72D). The human IL-15R sushi domain/human IgG1 Fc fusion protein comprises the sushi domain of the IL-15R subunit (amino acids 1-65 of the mature human IL-15Rα protein) linked with the human IgG1 CH2-CH3 region containing the Fc domain (232 amino acids). Aside from the N72D substitution, all of the protein sequences are human. Based on the amino acid sequence of the subunits, the calculated molecular weight of the complex comprising two IL-15N72D polypeptides and a disulfide linked homodimeric IL-15RαSu/IgG1 Fc protein is 92.4 kDa. Each IL-15N720 polypeptide has a calculated molecular weight of approximately 12.8 kDa and the IL-15RαSu/IgG 1 Fc fusion protein has a calculated molecular weight of approximately 33.4 kDa. Both the IL-15N72D and IL-15RαSu/IgG 1 Fc proteins are glycosylated resulting in an apparent molecular weight of ALT-803 as approximately 114 kDa by size exclusion chromatography. The isoelectric point (pI) determined for ALT-803 can range from approximately 5.6 to 6.5. Thus, the fusion protein can be negatively charged at pH 7. The calculated molar extinction coefficient at A280 for ALT-803 is 116,540 M or, in other words, one OD280 is equivalent to 0.79 mg/mL solution of ALT-803.

Additionally, it has been demonstrated that intracellular complex formation with IL-15Rα prevents IL-15 degradation in the endoplasm reticulum and facilitates its secretion. Using a co-expression strategy in Chinese hamster ovary (CHO) cells, the IL-15N72D and IL-15RαSu/IgG Fc proteins can be produced at high levels and formed a soluble, stable complex. The biological activity of CHO-produced ALT-803 complex can be equivalent to in-vitro assembled IL-15N72D:IL-15RαSu/IgG Fc complexes in standard cell-based potency assays using IL-15-dependent cell lines. The methods provided herein, thus represent a better approach for generating active, fully characterized cGMP grade IL-15:IL-15Rα complex than current strategies employing in vitro assembly of individually produced and, in some cases, refolded proteins.

Recent studies show that ALT-803 (1) can promote the development of high effector NK cells and CD8+ T cell responders of the innate phenotype, (2) can enhance the function of NK cells, and (3) can play a vital role in reducing tumor metastasis and ultimately survival, especially in combination with checkpoint inhibitors, which are further described below.

In some embodiments, an IL-15 super-agonist or an IL-15 super-agonist complex, ALT-803, can be administered parenterally, subcutaneously, intramuscularly, by intravenous infusion, by implantation, intraperitoneally, or intravesicularly. In some embodiments 0.1-5 μg of the IL-15 superagonist can be administered in a single dose. In some embodiments, 0.1-0.2 μg, 0.2-0.3 μg, 0.3-0.4 μg, 0.4-0.5 μg, 0.5-0.6 μg, 0.6-0.7 μg, 0.7-0.8 μg, 0.8-0.9 μg, 0.9-1 μg, 1-1.5 μg, 1.5-2 μg, 2-2.5 μg, 2.5-3 μg, 3-3.5 μg, 3.5-4 μg, 4-4.5 μg, or 4.5-5 μg of the IL-15 superagonistcan be administered in a single dose. In certain embodiments, 1 μg of the ALT-803 can be administered in a single dose. In some embodiments, ALT-803 can be administered at an effective dose of from about 0.1 μg/kg to abut 100 mg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 μg/kg body weight or 1, 2, 3, 4, 5, 6, 7, 8, 9, 10, 20, 30, 40, 50, 60, 70, 80, 90, 95, 99, or 100 mg/kg body weight. In some embodiments, an IL-15 superagonist can be administered with an Ad5 [E1−, E2b−]-CEA vaccine. In some embodiments, an IL-15 superagonist can be administered as a mixture with the Ad5 [E1−, E2b−]-CEA vaccine. In other embodiments, an IL-15 superagonist can be administered as a separate dose immediately before or after the Ad5 [E1−, E2b−]-CEA vaccine. In other embodiments, an ALT-803 is administered within 1 day, within 2 days, within 3 days, within 4 days, within 5 days, or within 6 days of administration of an Ad5 [E1−, E2b−]-CEA vaccine. In some embodiments, an ALT-803 is administered 3 days after an Ad5 [E1−, E2b−]-CEA vaccine. In some embodiments, ALT-803 is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours. Daily effective doses of ALT-803 can include from 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight. In some embodiments, ALT-803 is administered once per week, twice per week, three times per week, four times per week, five times per week, six times per week, or seven times per week. Effective weekly doses of ALT-803 include between 0.0001 mg/kg and 4 mg/kg body weight, e.g., 0.001, 0.003, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, or 4 mg/kg body weight. ALT-803 can be administered at a dose from from about 0.1 μg/kg body weight to about 5000 g/kg body weight; or from about 1 g/kg body weight to about 4000 μg/kg body weight or from about 10 μg/kg body weight to about 3000 μg/kg body weight. In other embodiments, ALT-803 can be administered at a dose of about 0.1, 0.3, 0.5, 1, 3, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 μg/kg. In some embodiments, ALT-803 can be administered at a dose from about 0.5 μg compound/kg body weight to about 20 g compound/kg body weight. In other embodiments, the doses may be about 0.5, 1, 3, 6, 10, or 20 mg/kg body weight. In some embodiments, or example in parenteral administration, ALT-803 can be administered at a dose of about 0.5 μg/kg-about 15 μg/kg (e.g., 0.5, 1, 3, 5, 10, or 15 g/kg).

In some embodiments, a subject in need thereof receiving combination therapy with the Ad5 [E1−, E2b−]-CEA vaccine and ALT-803 is administered one or more dose of the Ad5 [E1−, E2b−]-CEA vaccine and ALT-803 over a 21-day period. For example, a subject in need thereof can be administered the Ad-CEA vaccine on Day 7, Day 14, and Day 21. Additionally, a subject in need thereof can be administered the IL-15 superagonist (ALT-803) on Day 10 and Day 17. Thus, in some embodiments, the subject is administered more than one dose of ALT-803 in a complete dosing regimen. In some embodiments, the subject can be administered at least 1 dose, at least 2 doses, at least 3 doses, at least 4 doses, or at least 5 doses of the IL-15 superagonist. In certain embodiments, the subject can be administered one less dose of ALT-803 than the Ad5 [E1−, E2b-]-CEA vaccine.

In some embodiments, the IL-15 superagonist, such as ALT-803, can be encoded as an immunological fusion with the CEA antigen. For example, in some embodiments the Ad5 [E1−, E2b−] vaccine can encode for CEA and ALT-803 (Ad5 [E1−, E2b−]-CEA/ALT-803). In these embodiments, upon administration to a subject in need thereof, Ad5 [E1−, E2b−] vectors encoding for CEA and ALT-803 induce expression of CEA and ALT-803 as an immunological fusion, which is therapeutically active.

Combination therapy with Ad5[E1−, E2b−] vectors encoding for CEA and ALT-803 can result in boosting the immune response, such that the combination of both therapeutic moieties acts to synergistically boost the immune response than either therapy alone. For example, combination therapy with Ad5[E1−, E2b−] vectors encoding for CEA and ALT-803 can result in synergistic enhancement of stimulation of antigen-specific effector CD4+ and CD8+ T cells, stimulation of NK cell response directed towards killing infected cells, stimulation of neutrophils or monocyte cell responses directed towards killing infected cells via antibody dependent cell-mediated cytotoxicity (ADCC) or antibody dependent cellular phagocytosis (ADCP) mechanisms. Combination therapy with Ad5[E1−, E2b−] vectors encoding for CEA and ALT-803 can synergistically boost any one of the above responses, or a combination of the above responses, to vastly improve survival outcomes after administration to a subject in need thereof.

Combination Therapies of Ad5-Vaccines with Further Immunotherapies

In further embodiments, the present invention provides compositions for further combination therapies which include the Ad5 [E1−, E2b−] vector encoding for a calreticulin-antigen fusion, wherein the antigen can be any antigen disclosed herein (e.g., CEA or a neo-antigen), and one or more of the following agents: a chemotherapeutic agent, costimulatory molecules, checkpoint inhibitors, antibodies against a specific antigen (e.g., CEA), engineered NK cells, or any combination thereof. For example, the present invention provides a method of treating a CEA-expressing cancer in an individual in need thereof, the method comprising: administering to the individual a first pharmaceutical composition comprising a replication-defective vector comprising a nucleic acid sequence encoding a CEA antigen or any suitable antigen fused to calreticulin, and administering to the individual an anti-CEA antibody and engineered NK cells. In some embodiments, the method can further comprise administering to the individual a VEGF inhibitor, a chemotherapy, or a combination thereof. In other embodiments, the method can further comprise administering to the individual engineered NK cells and a checkpoint inhibitor. Any combination of chemotherapeutic agents, costimulatory molecules, checkpoint inhibitors, antibodies against a specific antigen (e.g., CEA), or engineered NK cells can be included in combination therapy with the Ad5 [E1−, E2b−] vaccine encoding for an antigen, such as CEA, fused to CRT.

In certain embodiments, the chemotherapy used herein is capecitabine, leucovorin, fluorouracil, oxaliplatin, fluoropyrimidine, irinotecan, mitomycin, regorafenib, cetuxinab, panitumumab, acetinophen, or a combination thereof. In particular embodiments, the chemotherapy used herein is FOLFOX (leucovorin, fluorouracil and oxaliplatin) or capecitabine. In certain embodiments, the immune checkpoint inhibitor is an anti-PD-1 or anti-PD-L1 antibody, such as avelumab. In certain embodiments, the VEGF inhibitor is an anti-VEGF antibody, such as bevacizumab. The agents which can be used in combination therapy alongside the replication defective vector encoding for the CRT-antigen fusion are described in further detail below.

FOLFOX (5-Fluorouracil, Leucovorin, Oxaliplatin)

A randomized trial comparing irinotecan and bolus fluorouracil plus leucovorin (IFL, control combination), oxaliplatin and infused fluorouracil plus leucovorin (FOLFOX), or irinotecan and oxaliplatin (IROX) established the FOLFOX combination, given for a total of 6 months, as the standard of care for first line treatment in patients with metastatic colorectal cancer (mCRC). Though multiple infusion schedules of FOLFOX have been validated, typically denominated as ‘modified FOLFOX, there are no essential changes in the constituent cytotoxic agents of the regimen. Of these, mFOLFOX6 is one of the most widely used.

Oxaliplatin, however, is very difficult for patients to receive for greater than 6 months (12 cycles) due to progressive neurotoxicity. Though 6 months of combination therapy remains the standard of care in mCRC, clinical judgment may influence the decision to limit the number of oxaliplatin-containing cycles towards the end of treatment Other trials, including the CAIRO3 study, have demonstrated the feasibility and benefit of discontinuation of oxaliplatin after a 3 month “induction” period with continuation of 5-FU and leucovorin as “maintenance” therapy.

Bevacizumab (AVASTIN®)

Addition of bevacizumab to first-line 5-FU and Oxaliplatin containing regimens was demonstrated to increase time to progression in mCRC patients with a manageable side effect profile and non-overlapping toxicities. Later trials indicated that continuing bevacizumab beyond first progression (in combination with subsequent chemotherapy) improved overall survival in an unselected group of patients by KRAS mutational status, which has led to its approved use in the maintenance setting.

Capecitabine

This agent is a prodrug that is enzymatically converted to 5-fluorouracil by 3 enzymatic steps following oral ingestion. As an orally active fluoropyrimidine, capecitabine has been approved for use in the adjuvant setting. In the advanced colon cancer setting, it has been shown to be equally efficacious as 5-fluorouracil, though with more reported rates of hand-foot syndrome. This agent offers the convenience of the oral route with its benefits of reducing infusion commitments for patients in the maintenance setting, while achieving high concentrations intratumorally, given the higher concentrations of thymidine phosphorylase in tumor as compared to normal tissues.

Costimulatory Molecules

In addition to the use of a recombinant adenovirus-based vector vaccine containing target antigens such as a CEA antigen or epitope, co-stimulatory molecules can be incorporated into said vaccine to increase immunogenicity. Initiation of an immune response requires at least two signals for the activation of naive T cells by APCs (Damle, et al. J Immunol 148: 1985-92 (1992); Guinan, et al. Blood 84: 3261-82 (1994); Hellstrom, et al. Cancer Chemother Pharmacol 38: S40-44 (1996); Hodge, et al. Cancer Res 39: 5800-07 (1999)). An antigen specific first signal is delivered through the T cell receptor (TCR) via the peptide/major histocompatability complex (MHC) and causes the T cell to enter the cell cycle. A second, or costimulatory, signal may be delivered for cytokine production and proliferation.

At least three distinct molecules normally found on the surface of professional antigen presenting cells (APCs) have been reported as capable of providing the second signal critical for T cell activation: B7-1 (CD80), ICAM-1 (CD54), and LFA-3 (human CD58) (Damle, et al. J Immunol 148: 1985-92 (1992); Guinan, et al. Blood 84: 3261-82 (1994); Wingren, et al. Crit Rev Immunol 15: 235-53 (1995); Parra, et al. Scand. J Immunol 38: 508-14 (1993); Hellstrom, et al. Ann NY Acad Sci 690: 225-30 (1993); Parra, et al. J Immunol 158: 637-42 (1997); Sperling, et al. J Immunol 157: 3909-17 (1996); Dubey, et al. J Immunol 155: 45-57 (1995); Cavallo, et al. Eur J Immunol 25: 1154-62 (1995)).

These costimulatory molecules have distinct T cell ligands. B7-1 interacts with the CD28 and CTLA-4 molecules, ICAM-1 interacts with the CD11a/CD18 (LFA-1/β2 integrin) complex, and LFA-3 interacts with the CD2 (LFA-2) molecules. Therefore, in a preferred embodiment, it would be desirable to have a recombinant adenovirus vector that contains B7-1, ICAM-1, and LFA-3, respectively, that, when combined with a recombinant adenovirus-based vector vaccine containing one or more nucleic acids encoding target antigens such as a HER2/neu antigen or epitope, will further increase/enhance anti-tumor immune responses directed to specific target antigens.

Natural Killer (NK) Cells

In certain embodiments, native or engineered NK cells may be provided to be administered to a subject in need thereof, in combination with adenoviral vector-based compositions and IL-15 superagonist or other immunotherapies as described herein.

The immune system is a tapestry of diverse families of immune cells each with its own distinct role in protecting from infections and diseases. Among these immune cells are the natural killer, or NK, cells as the body's first line of defense. NK cells have the innate ability to rapidly seek and destroy abnormal cells, such as cancer or virally-infected cells, without prior exposure or activation by other support molecules. In contrast to adaptive immune cells such as T cells, NK cells have been utilized as a cell-based “off-the-shelf” treatment in phase 1 clinical trials, and have demonstrated tumor killing abilities for cancer.

aNK Cells

In addition to native NK cells, there may be provided NK cells for administering to a patient that has do not express Killer Inhibitory Receptors (KR), which diseased cells often exploit to evade the killing function of NK cells. This unique activated NK, or aNK, cell lack these inhibitory receptors while retaining the broad array of activating receptors which enable the selective targeting and killing of diseased cells. aNK cells also carry a larger pay load of granzyme and perforin containing granules, thereby enabling them to deliver a far greater payload of lethal enzymes to multiple targets.

taNK Cells

Chimeric antigen receptor (CAR) technology is among the most novel cancer therapy approaches currently in development. CARs are proteins that allow immune effector cells to target cancer cells displaying specific surface antigen (target-activated Natural Killer) is a platform in which aNK cells are engineered with one or more CARs to target proteins found on cancers and is then integrated with a wide spectrum of CARs. This strategy has multiple advantages over other CAR approaches using patient or donor sourced effector cells such as autologous T-cells, especially in terms of scalability, quality control and consistency.

Much of the cancer cell killing relies upon ADCC (antibody dependent cell-mediated cytotoxicity) whereupon effector immune cells attach to antibodies, which are in turn bound to the target cancer cell, thereby facilitating killing of the cancer by the effector cell. NK cells are the key effector cell in the body for ADCC and utilize a specialized receptor (CD16) to bind antibodies.

haNK Cells

Studies have shown that perhaps only 20% of the human population uniformly expresses the “high-affinity” variant of CD16, which is strongly correlated with more favorable therapeutic outcomes compared to patients with the “low-affinity” CD16. Additionally, many cancer patients have severely weakened immune systems due to chemotherapy, the disease itself or other factors.

In certain aspects, haNK cells are modified to express high-affinity CD16. As such, haNK cells may potentiate the therapeutic efficacy of a broad spectrum of antibodies directed against cancer cells.

Anti-CEA Antibodies

In some embodiments, compositions are administered with one or more antibodies targeted to CEA, or anti-CEA antibodies. In some embodiments, the composition comprises a replication-defective vector comprising a nucleotide sequence encoding a target antigen, such as CEA, MUC1, Brachyury, or a combination thereof, or any suitable antigens.

Anti-CEA antibodies can be used to generate an immune response against a target antigen expressed and/or presented by a cell. In certain embodiments, the compositions and methods can be used to generate immune responses against a carcinoembryonic antigen (CEA), such as CEA expressed or presented by a cell. For example, the compositions and methods can be used to generate an immune response against CEA(6D) expressed or presented by a cell.

CEA has been shown to be overexpressed on a variety of cancers. In some embodiments, the targeted patient population administered anti-CEA antibody therapy may be individuals with CEA expressing colorectal cancer, head and neck cancer, liver cancer, breast cancer, lung cancer, bladder cancer, or pancreas cancer.

The present invention provides for a novel monoclonal antibody that specifically binds a CPAA. This monoclonal antibody, identified as “16C3”, which refers to the number assigned to its hybridoma clone. Herein, 16C3 also refers to the portion of the monoclonal antibody, the paratope or CDRs, that bind specifically with a CPAA epitope identified as 16C3 because of its ability to bind the 16C3 antibody. The several recombinant and humanized forms of 16C3 described herein may be referred to by the same name.

The present invention includes, within its scope, DNA sequences encoding the variable regions of the light and heavy chains of the anti-CPAA antibody of the present invention. A nucleic acid sequence encoding the variable region of the light chain of the 16C3 antibody is presented in SEQ ID NO: 16. A nucleic acid sequence encoding the variable region of the heavy chain of the 16C3 antibody is presented in SEQ ID NO: 17.

The present invention includes, within its scope, a peptide of the 16C3 light chain comprising the amino acid sequence of SEQ ID NO: 18 and SEQ ID NO: 19; and a peptide of the 16C3 heavy chain comprising the amino acid sequence depicted in SEQ ID NO: 99 and SEQ ID NO: 20. Further, the present invention includes the CDR regions depicted for the 16C3 kappa light chain which are the residues underlined in SEQ ID NO: 18, having the amino acids of CDR 1: GASENIYGALN (SEQ ID NO: 21); CDR 2: GASNLAD (SEQ ID NO: 22); and CDR 3: QNVLSSPYT (SEQ ID NO: 23); as well as the amino acids the light chain underlined in SEQ ID NO: 19, which include CDR 1: QASENIYGALN (SEQ ID NO: 24); CDR 2: GASNLAT (SEQ ID NO: 25); and CDR 3: QQVLSSPYT (SEQ ID NO: 26). The invention similarly identifies the CDR regions for the heavy chain, which include the amino acids for CDR 1: GYTFTDYAMH (SEQ ID NO: 27); CDR 2: LISTYSGDTKYNQNFKG (SEQ ID NO: 28); and CDR 3: GDYSGSRYWFAY (SEQ ID NO: 29); as well as the amino acids the heavy chain, which include CDR 1: GYTFTDYAMH (SEQ ID NO: 27); CDR 2: LISTYSGDTKYNQKFQG (SEQ ID NO: 30); and CDR 3: GDYSGSRYWFAY (SEQ ID NO: 31).

In the present application, the 16C3 antibody is also referred to as the NEO-201 antibody.

In certain embodiments, anti-CEA antibodies used can be COL1, COL2, COL3, COL4, COL5, COL6, COL7, COL8, COL9, COL10, COL11, COL12, COL13, COL14, COL15, arcitumomab, besilesomab, labetuzumab, altumomab, or NEO-201. In certain embodiments, the anti-CEA antibody can be murine, chimeric, or humanized.

In certain embodiments, the anti-CEA antibody binds to a CEA overexpressing cell 2, 3, 4, 5, 6, 7, 8, 9, or 10 times or more over a baseline CEA expression in a non-cancer cell.

Immune Pathway Checkpoint Modulators

In some embodiments, compositions are administered with one or more immune checkpoint modulator, such as immune checkpoint inhibitors. In some embodiments, the composition comprises a replication-defective vector comprising a nucleotide sequence encoding a target antigen, such as CEA, or any suitable antigens.

A balance between activation and inhibitory signals regulates the interaction between T lymphocytes and disease cells, wherein T-cell responses are initiated through antigen recognition by the T-cell receptor (TCR). The inhibitory pathways and signals are referred to as immune checkpoints. In normal circumstances, immune checkpoints play a critical role in control and prevention of autoimmunity and also protect from tissue damage in response to pathogenic infection.

In certain aspects, there are provided combination immunotherapies comprising viral vector based vaccines and compositions for modulating immune checkpoint inhibitory pathways for the treatment of cancer and infectious diseases. In some embodiments, modulating is increasing expression or activity of a gene or protein. In some embodiments, modulating is decreasing expression or activity of a gene or protein. In some embodiments, modulating affects a family of genes or proteins.

Certain embodiments provide combination immunotherapies comprising multi-targeted immunotherapeutic directed to TAAs and molecular compositions comprising an immune pathway checkpoint modulator that targets at least one immune checkpoint protein of the immune inhibitory pathway. Certain embodiments provide combination immunotherapies comprising multi-targeted immunotherapeutic directed to IDAAs and molecular compositions comprising an immune pathway checkpoint modulator that targets at least one immune checkpoint protein of the immune inhibitory pathway. Certain embodiments provide a combination immunotherapies or vaccines comprising: at least two, at least three, or more than three different target antigens comprising a sequence encoding a modified CEA, and at least one molecular composition comprising an immune pathway checkpoint modulator. For example, a combination immunotherapy or vaccine can comprise at least two, at least three, or more than three different target antigens comprising a sequence encoding a modified CEA, wherein the modified CEA comprises a sequence with an identity value of at least 70%, 75%, 80%, 85%, 90%, 95%, 98%, 99%, 99.5%, or 99.9% to SEQ ID NO: 1 or SEQ ID NO: 100 and at least one molecular composition comprising an immune pathway checkpoint modulator. In some embodiments, the modified CEA comprises a sequence with an identity value of at least 70%, 75%, 80%, 85%, 90%, 95%, 98%, 99%, 99.5%, 99.9% or 100% SEQ ID NO: 1 has a Asn->Asp substitution at position 610 or SEQ ID NO: 100.

In general, the immune inhibitory pathways are initiated by ligand-receptor interactions. It is now clear that in diseases, the disease can co-opt immune-checkpoint pathways as mechanism for inducing immune resistance in a subject.

The induction of immune resistance or immune inhibitory pathways in a subject by a given disease can be blocked by molecular compositions such as siRNAs, antisense, small molecules, mimic, a recombinant form of ligand, receptor or protein, or antibodies (which can be an Ig fusion protein) that are known to modulate one or more of the Immune Inhibitory Pathways, or any combination thereof. For example, preliminary clinical findings with blockers of immune-checkpoint proteins, such as Cytotoxic T-lymphocyte-associated antigen 4 (CTLA4) and programmed cell death protein 1 (PD1) have shown promise for enhancing antitumor immunity.

Because diseased cells can express multiple inhibitory ligands, and disease-infiltrating lymphocytes express multiple inhibitory receptors, dual or triple blockade of immune checkpoints proteins may enhance anti-disease immunity. Combination immunotherapies as provide herein can comprise one or more molecular compositions of the following immune-checkpoint proteins: PD1, PDL1, PDL2, CD28, CD80, CD86, CTLA4, B7RP1, ICOS, B7RPI, B7-H3 (also known as CD276), B7-H4 (also known as B7-S1, B7x and VCTN1), BTLA (also known as CD272), HVEM, KIR, TCR, LAG3 (also known as CD223), CD137, CD137L, OX40, OX40L, CD27, CD70, CD40, CD40L, TIM3 (also known as HAVcr2), GAL9, and A2aR. In some embodiments, the molecular composition comprises siRNAs. In some embodiments, the molecular composition comprises a small molecule. In some embodiments, the molecular composition comprises a recombinant form of a ligand. In some embodiments, the molecular composition comprises a recombinant form of a receptor. In some embodiments, the molecular composition comprises an antibody. In some embodiments, the combination therapy comprises more than one molecular composition and/or more than one type of molecular composition. As it will be appreciated by those in the art, future discovered proteins of the immune checkpoint inhibitory pathways are also envisioned to be encompassed in certain aspects.

In some embodiments, combination immunotherapies comprise molecular compositions for the modulation of CTLA4. In some embodiments, combination immunotherapies comprise molecular compositions for the modulation PD1. In some embodiments, combination immunotherapies comprise molecular compositions for the modulation PDL1. In some embodiments, combination immunotherapies comprise molecular compositions for the modulation LAG3. In some embodiments, combination immunotherapies comprise molecular compositions for the modulation B7-H3. In some embodiments, combination immunotherapies comprise molecular compositions for the modulation B7-H4. In some embodiments, combination immunotherapies comprise molecular compositions for the modulation TIM3. In some embodiments, modulation is an increase or enhancement of expression. In other embodiments, modulation is the decrease of absence of expression.

Two exemplary immune checkpoint inhibitors include the cytotoxic T lymphocyte associated antigen-4 (CTLA-4) and the programmed cell death protein-1 (PD1). CTLA-4 can be expressed exclusively on T-cells where it regulates early stages of T-cell activation. CTLA-4 interacts with the co-stimulatory T-cell receptor CD28 which can result in signaling that inhibits T-cell activity. Once TCR antigen recognition occurs, CD28 signaling may enhance TCR signaling, in some cases leading to activated T-cells, and CTLA-4 inhibits the signaling activity of CD28. Certain embodiments provide immunotherapies as provided herein in combination with anti-CTLA-4 monoclonal antibody for the treatment of proliferative disease and cancer. Certain embodiments provide immunotherapies as provided herein in combination with CTLA-4 molecular compositions for the treatment of proliferative disease and cancer.

Programmed death cell protein ligand-1 (PDL1) is a member of the B7 family and is distributed in various tissues and cell types. PDL1 can interact with PD1 inhibiting T-cell activation and CTL mediated lysis. Significant expression of PDL1 has been demonstrated on various human tumors and PDL1 expression is one of the key mechanisms in which tumors evade host antitumor immune responses. Programmed death-ligand 1 (PDL1) and programmed cell death protein-1 (PD1) interact as immune checkpoints. This interaction can be a major tolerance mechanism which results in the blunting of anti-tumor immune responses and subsequent tumor progression. PD1 is present on activated T cells and PDL1, the primary ligand of PD1, is often expressed on tumor cells and antigen-presenting cells (APC) as well as other cells, including B cells. PDL1 interacts with PD1 on T cells inhibiting T cell activation and cytotoxic T lymphocyte (CTL) mediated lysis. Certain embodiments provide immunotherapies as provided herein in combination with anti-PD1 or anti-PDL1 monoclonal antibody for the treatment of proliferative disease and cancer. Certain embodiments provide immunotherapies as provided herein in combination with PD1 or anti-PDL1 molecular compositions for the treatment of proliferative disease and cancer. Certain embodiments provide immunotherapies as provided herein in combination with anti-CTLA-4 and anti-PD1 monoclonal antibodies for the treatment of proliferative disease and cancer. Certain embodiments provide immunotherapies as provided herein in combination with anti-CTLA-4 and PDL1 monoclonal antibodies for the treatment of proliferative disease and cancer. Certain embodiments provide immunotherapies as provided herein in combination with anti-CTLA-4, anti-PD1, PDL1, monoclonal antibodies, or a combination thereof, for the treatment of proliferative disease and cancer.

Certain embodiments provide immunotherapies as provided herein in combination with several antibodies directed against the PD-L1/PD-1 pathway that are in clinical development for cancer treatment. In certain embodiments, anti-PD-L1 antibodies may be used. Compared with anti-PD-1 antibodies that target T-cells, anti-PDL1 antibodies that target tumor cells are expected to have less side effects, including a lower risk of autoimmune-related safety issues, as blockade of PD-L1 leaves the PD-L2/PD-1 pathway intact to promote peripheral self-tolerance.

To this end, avelumab, a fully human IgG1 anti-PDL1 antibody (drug code MSB0010718C) has been produced. Avelumab selectively binds to PD-L1 and competitively blocks its interaction with PD-1.

Avelumab is also cross-reactive with murine PD-L1, thus allowing in vivo pharmacology studies to be conducted in normal laboratory mice. However, due to immunogenicity directed against the fully human avelumab molecule, the dosing regimen was limited to three doses given within a week. In some embodiments, avelumab can be administered at a dose of 1 mg/kg-20 mg/kg. In some embodiments, avelumab can also be administered at 1 mg/kg, 3 mg/kg, 10 mg/kg, and 20 mg/kg. In some embodiments, the addition of Avelumab, or any other immune pathway checkpoint modulator, in the dosing regimen can increase the immune response by at least 2, at least 3, at least 4, at least 5, at least 6, at least 7, at least 8, at least 9, at least 10, at least 15, at least 20, or at least 25-fold.

The key preclinical pharmacology findings for avelumab are summarized below. Avelumab showed functional enhancement of primary T cell activation in vitro in response to antigen-specific and antigen non-specific stimuli; and significant inhibition of in vivo tumor growth (PD-L1 expressing MC38 colon carcinoma) as a monotherapy. Its in vivo efficacy is driven by CD8+ T cells, as evidenced by complete abrogation of anti-tumor activity when this cell type was systemically depleted. Its combination with localized, fractionated radiotherapy resulted in complete regression of established tumors with generation of anti-tumor immune memory. Its use in chemotherapy combinations also showed promising activity: additive combination effect when partnered with oxaliplatin and 5-fluorouracil (5-FU) (core components of FOLFOX [oxaliplatin, 5-FU, and folinic acid]) against MC38 colon tumors; significant increase in survival when partnered with gemcitabine against PANC02 pancreatic tumors. Its antibody-dependent cell-mediated cytotoxicity (ADCC) was demonstrated against human tumor cells in vitro; furthermore, studies in ADCC deficient settings in vivo support a contribution of ADCC to anti-tumor efficacy. Additional findings of Avelumab include: no complement-dependent cytotoxicity was observed in vitro. Immunomonitoring assays with translational relevance for the clinic further support an immunological mechanism of action: consistent increases in CD8+PD-1+ T cells and CD8+ effector memory T cells as measured by fluorescence-activated cell sorter (FACS); enhanced tumor-antigen specific CD8+ T cell responses as measured by pentamer staining and enzyme-linked immunosorbent spot (ELISPOT) assays.

Despite reports indicating that anti-tumor radiographic responses were unlikely using agents that interfere with PD-1-PD-L1 binding in colorectal cancer, there have been reports of radiographic responses. Additionally, a correlation has been demonstrated in multiple clinical trials indicating that PD-L1 expression levels on tumor tissue predict the likelihood of radiographic response. However, it has become clear that PD-L1 expression, as it is currently measured, is not a definitive requirement for anti-tumor efficacy. It has been noted that colorectal tumors rarely express PD-L1 compared with other tumors that are more likely to respond to PD-1-PD-L1 blockade. However, it is known that a strong anti-tumor T cell response, producing IFN-gamma, will induce PD-L1 expression.

In some embodiments, without being bound by theory, it was contemplated that an underlying immune response is necessary for PD-1-PD-L1 blockade to have an anti-tumor effect. Without being bound by theory, it was further contemplated that this combination of an immune checkpoint inhibitor with the standard therapy and an adenoviral vector composition such as Ad-CEA immunizations or Ad-CEA immunizations may be capable of induction of PD-L1 expression and thereby increases the anti-tumor activity of PD-1-PD-L1 blockade.

Immune checkpoint molecules can be expressed by T cells. Immune checkpoint molecules can effectively serve as “brakes” to down-modulate or inhibit an immune response. Immune checkpoint molecules include, but are not limited to Programmed Death 1 (PD1, also known as PDCD1 or CD279, accession number: NM_005018), Cytotoxic T-Lymphocyte Antigen 4 (CTLA-4, also known as CD152, GenBank accession number AF414120.1), LAG3 (also known as CD223, accession number: NM_002286.5), Tim3 (also known as HAVCR2, GenBank accession number: JX049979.1), BTLA (also known as CD272, accession number: NM_181780.3), BY55 (also known as CD160, GenBank accession number: CR541888.1), TIGIT (also known as IVSTM3, accession number: NM_173799), LAIR1 (also known as CD305, GenBank accession number: CR542051.1), SIGLECIO (GeneBank accession number: AY358337.1), 2B4 (also known as CD244, accession number: NM_001166664.1), PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, ILIORA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3 which directly inhibit immune cells. For example, PD1 can be combined with an adenoviral vaccine to treat a patient in need thereof. TABLE 1, without being exhaustive, shows exemplary immune checkpoint genes that can be inactivated to improve the efficiency of the adenoviral vaccine. Immune checkpoints gene can be selected from such genes listed in TABLE 1 and others involved in co-inhibitory receptor function, cell death, cytokine signaling, arginine tryptophan starvation, TCR signaling, Induced T-reg repression, transcription factors controlling exhaustion or anergy, and hypoxia mediated tolerance.

TABLE 1
Exemplary Immune Checkpoint Genes

| Gene | NCBI # | | | Genome |
|---|---|---|---|---|
| Symbol | (GRCh38.p2) | Start | Stop | location |
| ADORA2A | 135 | 24423597 | 24442360 | 22q11.23 |
| CD276 | 80381 | 73684281 | 73714518 | 15q23-q24 |
| VTCN1 | 79679 | 117143587 | 117270368 | 1p13.1 |
| BTLA | 151888 | 112463966 | 112499702 | 3q13.2 |
| CTLA4 | 1493 | 203867788 | 203873960 | 2q33 |
| IDO1 | 3620 | 39913809 | 39928790 | 8p12-p11 |
| KIR3DL1 | 3811 | 54816438 | 54830778 | 19q13.4 |
| LAG3 | 3902 | 6772483 | 6778455 | 12p13.32 |
| PDCD1 | 5133 | 241849881 | 241858908 | 2q37.3 |
| HAVCR2 | 84868 | 157085832 | 157109237 | 5q33.3 |
| VISTA | 64115 | 71747556 | 71773580 | 10q22.1 |
| CD244 | 51744 | 160830158 | 160862902 | 1q23.3 |
| CISH | 1154 | 50606454 | 50611831 | 3p21.3 |

The combination of an adenoviral-based vaccine and an immune pathway checkpoint modulator may result in reduction in cancer recurrences in treated patients, as compared to either agent alone. In yet another embodiment the combination of an adenoviral-based vaccine and an immune pathway checkpoint modulator may result in reduction in the presence or appearance of metastases or micro metastases in treated patients, as compared to either agent alone. In another embodiment, the combination of an adenoviral-based vaccine and an immune pathway checkpoint modulator may result improved overall survival of treated patients, as compared to either agent alone. In some cases, the combination of an adenoviral vaccine and an immune pathway checkpoint modulator may increase the frequency or intensity of tumor-specific T cell responses in patients compared to either agent alone.

Some embodiments also disclose the use of immune checkpoint inhibition to improve performance of an adenoviral vector-based vaccine. The immune checkpoint inhibition may be administered at the time of the vaccine. The immune checkpoint inhibition may also be administered after a vaccine. Immune checkpoint inhibition may occur simultaneously to an adenoviral vaccine administration. Immune checkpoint inhibition may occur 1, 2, 3, 4, 5, 6, 7, 8, 9, 10, 15, 20, 30, 40, 50, or 60 minutes after vaccination. Immune checkpoint inhibition may also occur 1, 2, 3, 4, 5, 6, 7, 8, 9, 10, 11, 12, 13, 14, 15, 16, 17, 18, 19, 20, 21, 22, 23, or 24 hours post vaccination. In some cases, immune inhibition may occur 1, 2, 3, 4, 5, 6, or 7 days after vaccination. Immune checkpoint inhibition may occur at any time before or after vaccination.

In another aspect, there is provided a vaccine comprising an antigen and an immune pathway checkpoint modulator. Some embodiments pertain to a method for treating a subject having a condition that would benefit from downregulation of an immune checkpoint, PD1 for example, and its natural binding partner(s) on cells of the subject.

An immune pathway checkpoint modulator may be combined with an adenoviral vaccine comprising nucleotide sequences encoding any antigen. For example, an antigen can be MUC1c, HER3, Brachyury, HER2NEU, CEA, PMSA, or PSA. An immune pathway checkpoint modulator may produce a synergistic effect when combined with a vaccine. An immune pathway checkpoint modulator may also produce an additive effect when combined with a vaccine.

In particular embodiments, a checkpoint immune inhibitor may be combined with a vector comprising nucleotide sequences encoding any antigen, optionally with a chemotherapy or any other cancer care or therapy, such as VEGF inhibitors, angiogenesis inhibitors, radiation, other immune therapy, or any suitable cancer care or therapy.

### Immunological Fusion Partner Antigen Targets

The viral vectors or composition described herein may further comprise nucleic acid sequences that encode proteins, or an “immunological fusion partner,” that can increase the immunogenicity of the target antigen such as a tumor neo-antigen or neo-epitope. In this regard, the protein produced following immunization with the viral vector containing such a protein may be a fusion protein comprising the target antigen of interest fused to a protein that increases the immunogenicity of the target antigen of interest.

In one embodiment, such an immunological fusion partner is derived from a Mycobacterium sp., such as a Mycobacterium tuberculosis-derived Ra12 fragment. The immunological fusion partner derived from Mycobacterium sp. can be any one of the sequences set forth in SEQ ID NO: 32-SEQ ID NO: 40. Ra12 compositions and methods for their use in enhancing the expression and/or immunogenicity of heterologous polynucleotide/polypeptide sequences are described in U.S. Pat. No. 7,009,042, which is herein incorporated by reference in its entirety. Briefly, Ra12 refers to a polynucleotide region that is a subsequence of a Mycobacterium tuberculosis MTB32A nucleic acid. MTB32A is a serine protease of 32 kDa encoded by a gene in virulent and avirulent strains of M. tuberculosis. The nucleotide sequence and amino acid sequence of MTB32A have been described (see, e.g., U.S. Pat. No. 7,009,042; Skeiky et al., Infection and Immun. 67:3998-4007 (1999), incorporated herein by reference in their entirety). C-terminal fragments of the MTB32A coding sequence can be expressed at high levels and remain as soluble polypeptides throughout the purification process. Moreover, Ra12 may enhance the immunogenicity of heterologous immunogenic polypeptides with which it is fused. A Ral2 fusion polypeptide can comprise a 14 kDa C-terminal fragment corresponding to amino acid residues 192 to 323 of MTB32A. Other Ra12 polynucleotides generally can comprise at least about 15, 30, 60, 100, 200, 300, or more nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants can have at least about 70%, 80%, or 90% identity, or more, to a polynucleotide sequence that encodes a native Ra12 polypeptide or a portion thereof.

In certain aspects, an immunological fusion partner can be derived from protein D, a surface protein of the gram-negative bacterium Haemophilus influenzae B. The immunological fusion partner derived from protein D can be the sequence set forth in SEQ ID NO: 41. In some cases, a protein D derivative comprises approximately the first third of the protein (e.g., the first N-terminal 100-110 amino acids). A protein D derivative may be lipidated. Within certain embodiments, the first 109 residues of a Lipoprotein D fusion partner is included on the N-terminus to provide the polypeptide with additional exogenous T-cell epitopes, which may increase the expression level in E. coli and may function as an expression enhancer. The lipid tail may ensure optimal presentation of the antigen to antigen presenting cells. Other fusion partners can include the non-structural protein from influenza virus, NS1 (hemagglutinin). Typically, the N-terminal 81 amino acids are used, although different fragments that include T-helper epitopes may be used.

In certain aspects, the immunological fusion partner can be the protein known as LYTA, or a portion thereof (particularly a C-terminal portion). The immunological fusion partner derived from LYTA can the sequence set forth in SEQ ID NO: 42. LYTA is derived from Streptococcus pneumoniae, which synthesizes an N-acetyl-L-alanine amidase known as amidase LYTA (encoded by the LytA gene). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus can be employed. Within another embodiment, a repeat portion of LYTA may be incorporated into a fusion polypeptide. A repeat portion can, for example, be found in the C-terminal region starting at residue 178. One particular repeat portion incorporates residues 188-305.

In some embodiments, the target antigen is fused to an immunological fusion partner, also referred to herein as an “immunogenic component,” comprising a cytokine selected from the group of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-01, and MIF. The target antigen fusion can produce a protein with substantial identity to one or more of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. The target antigen fusion can encode a nucleic acid encoding a protein with substantial identity to one or more of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. In some embodiments, the target antigen fusion further comprises one or more immunological fusion partner, also referred to herein as an “immunogenic components,” comprising a cytokine selected from the group of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. The sequence of IFN-γ can be, but is not limited to, a sequence as set forth in SEQ ID NO: 43. The sequence of TNFα can be, but is not limited to, a sequence as set forth in SEQ ID NO: 44. The sequence of IL-2 can be, but is not limited to, a sequence as set forth in SEQ ID NO: 45. The sequence of IL-8 can be, but is not limited to, a sequence as set forth in SEQ ID NO: 46. The sequence of IL-12 can be, but is not limited to, a sequence as set forth in SEQ ID NO: 47. The sequence of IL-18 can be, but is not limited to, a sequence as set forth in SEQ ID NO: 48. The sequence of IL-7 can be, but is not limited to, a sequence as set forth in SEQ ID NO: 49. The sequence of IL-3 can be, but is not limited to, a sequence as set forth in SEQ ID NO: 50. The sequence of IL-4 can be, but is not limited to, a sequence as set forth in SEQ ID NO: 51. The sequence of IL-5 can be, but is not limited to, a sequence as set forth in SEQ ID NO: 52. The sequence of IL-6 can be, but is not limited to, a sequence as set forth in SEQ ID NO: 53. The sequence of IL-9 can be, but is not limited to, a sequence as set forth in SEQ ID NO: 54. The sequence of IL-10 can be, but is not limited to, a sequence as set forth in SEQ ID NO: 55. The sequence of IL-13 can be, but is not limited to, a sequence as set forth in SEQ ID NO: 56. The sequence of IL-15 can be, but is not limited to, a sequence as set forth in SEQ ID NO: 57. The sequence of IL-16 can be, but is not limited to, a sequence as set forth in SEQ ID NO: 103. The sequence of IL-17 can be, but is not limited to, a sequence as set forth in SEQ ID NO: 104. The sequence of IL-23 can be, but is not limited to, a sequence as set forth in SEQ ID NO: 105. The sequence of IL-32 can be, but is not limited to, a sequence as set forth in SEQ ID NO: 106.

In some embodiments, the target antigen is fused or linked to an immunological fusion partner, also referred to herein as an “immunogenic component,” comprising a cytokine selected from the group of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-01, and MIF. In some embodiments, the target antigen is co-expressed in a cell with an immunological fusion partner, also referred to herein as an “immunogenic component,” comprising a cytokine selected from the group of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. In some embodiments, the immunogenic component is selected from the group consisting of IL-7, a nucleic acid encoding IL-7, a protein with substantial identity to IL-7, and a nucleic acid encoding a protein with substantial identity to IL-7. In some embodiments, the adjuvant is selected from the group consisting of IL-15, a nucleic acid encoding IL-15, a protein with substantial identity to IL-15, and a nucleic acid encoding a protein with substantial identity to IL-15.

In some embodiments, the target antigen is fused or linked to an immunological fusion partner, comprising CpG ODN (a non-limiting example sequence is shown in SEQ ID NO: 58), cholera toxin (a non-limiting example sequence is shown in SEQ ID NO: 59), a truncated A subunit coding region derived from a bacterial ADP-ribosylating exotoxin (a non-limiting example sequence is shown in (a non-limiting example sequence is shown in SEQ ID NO: 60), a truncated B subunit coding region derived from a bacterial ADP-ribosylating exotoxin (a non-limiting example sequence is shown in SEQ ID NO: 61), Hp91 (a non-limiting example sequence is shown in SEQ ID NO: 62), CCL20 (a non-limiting example sequence is shown in SEQ ID NO: 63), CCL3 (a non-limiting example sequence is shown in SEQ ID NO: 64), GM-CSF (a non-limiting example sequence is shown in SEQ ID NO: 65), G-CSF (a non-limiting example sequence is shown in SEQ ID NO: 66), LPS peptide mimic (non-limiting example sequences are shown in SEQ ID NO: 67-SEQ ID NO: 78), shiga toxin (a non-limiting example sequence is shown in SEQ ID NO: 79), diphtheria toxin (a non-limiting example sequence is shown in SEQ ID NO: 80), or CRM₁₉₇ (a non-limiting example sequence is shown in SEQ ID NO: 83).

In some embodiments, the target antigen is fused or linked to an immunological fusion partner, comprising an IL-15 superagonist. In some embodiments, the IL-15 superagonist can be a novel IL-15 superagonist mutant (IL-15N72D). In certain embodiments, addition of either mouse or human IL-15Rα and Fc fusion protein (the Fc region of immunoglobulin) to equal molar concentrations of IL-15N72D can provide a further increase in IL-15 biologic activity, such that IL-15N72D:IL-15Rα/Fc super-agonist complex exhibits a median effective concentration (EC₅₀) for supporting IL-15-dependent cell growth that can be greater than 10-fold lower than that of free IL-15 cytokine.

In some embodiments, the IL-15 super agonist is a biologically active protein complex of IL-15N72D, soluble IL-15Rα, and Fc fusion protein, also known as ALT-803. It is known that a soluble IL-15Rα fragment, containing the so-called “sushi” domain at the N terminus (Su), can bear most of the structural elements responsible for high affinity cytokine binding. A soluble fusion protein can be generated by linking the human IL-15RαSu domain (amino acids 1-65 of the mature human IL-15Rα protein) with the human IgG1 CH2-CH3 region containing the Fc domain (232 amino acids). This IL-15RαSu/IgG1 Fc fusion protein can have the advantages of dimer formation through disulfide bonding via IgG1 domains and ease of purification using standard Protein A affinity chromatography methods.

In some embodiments, ALT-803 can have a soluble complex consisting of 2 protein subunits of a human IL-15 variant associated with high affinity to a dimeric IL-15Rα sushi domain/human IgG1 Fc fusion protein. The IL-15 variant is a 114 amino acid polypeptide comprising the mature human IL-15 cytokine sequence with an Asn to Asp substitution at position 72 of helix C N72D). The human IL-15R sushi domain/human IgG1 Fc fusion protein comprises the sushi domain of the IL-15R subunit (amino acids 1-65 of the mature human IL-15Rα protein) linked with the human IgG1 CH2-CH3 region containing the Fc domain (232 amino acids). Aside from the N72D substitution, all of the protein sequences are human. Based on the amino acid sequence of the subunits, the calculated molecular weight of the complex comprising two IL-15N72D polypeptides (an example IL-15N72D sequence is shown in SEQ ID NO: 81) and a disulfide linked homodimeric IL-15RαSu/IgG1 Fc protein (an example IL-15RαSu/Fc domain is shown in SEQ ID NO: 82) is 92.4 kDa. In some embodiments, a recombinant vector encoding for a target antigen and for ALT-803 can have any sequence described herein to encode for the target antigen and can have SEQ ID NO: 81, SEQ ID NO: 81, SEQ ID NO: 82, and SEQ ID NO: 82 in any order, to encode for ALT-803.

Each IL-15N720 polypeptide has a calculated molecular weight of approximately 12.8 kDa and the IL-15RαSu/IgG 1 Fc fusion protein has a calculated molecular weight of approximately 33.4 kDa. Both the IL-15N72D and IL-15RαSu/IgG 1 Fc proteins can be glycosylated resulting in an apparent molecular weight of ALT-803 of approximately 114 kDa by size exclusion chromatography. The isoelectric point (pI) determined for ALT-803 can range from approximately 5.6 to 6.5. Thus, the fusion protein can be negatively charged at pH 7.

Any of the immunogenicity enhancing agents described herein can be fused or linked to a target antigen by expressing the immunogenicity enhancing agents and the target antigen in the same recombinant vector, using any recombinant vector described herein.

Nucleic acid sequences that encode for such immunogenicity enhancing agents can be any one of SEQ ID NO: 32-SEQ ID NO: 83 and are summarized in TABLE 2.

TABLE 2
Sequences of Immunogenicity Enhancing Agents

| SEQ ID NO | Sequence |
|---|---|
| SEQ ID NO: 32 | TAASDNFQLSQGGQGFAIPIGQAMAIAGQIRSGGGSPTVHIGPTAFL |
| | GLGVVDNNGNGARVQRVVGSAPAASLGISTGDVITAVDGAPINSAT |
| | AMADALNGHHPGDVISVTWQTKSGGTRTGNVTLAEGPPA |
| SEQ ID NO: 33 | MHHHHHHTAASDNFQLSQGGQGFAIPIGQAMAIAGQIRSGGGSPTV |
| | HIGPTAFLGLGVVDNNGNGARVQRVVGSAPAASLGISTGDVITAVD |
| | GAPINSATAMADALNGHHPGDVISVTWQTKSGGTRTGNVTLAEGP |
| | PAEFDDDDKDPPDPHQPDMTKGYCPGGRWGFGDLAVCDGEKYPD |
| | GSFWHQWMQTWFTGPQFYFDCVSGGEPLPGPPPPGGCGGAIPSEQP |
| | NAP |
| SEQ ID NO: 34 | MHHHHHHTAASDNFQLSQGGQGFAIPIGQAMAIAGQIRSGGGSPTV |
| | HIGPTAFLGLGVVDNNGNGARVQRVVGSAPAASLGISTGDVITAVD |
| | GAPINSATAMADALNGHHPGDVISVTWQTKSGGTRTGNVTLAEGP |
| | PAEFPLVPRGSPMGSDVRDLNALLPAVPSLGGGGGCALPVSGAAQ |
| | WAPVLDFAPPGASAYGSLGGPAPPPAPPPPPPPPPHSFIKQEPSWGGA |
| | EPHEEQCLSAFTVHFSGQFTGTAGACRYGPFGPPPPSQASSGQARMF |
| | PNAPYLPSCLESQPAIRNQGYSTVTFDGTPSYGHTPSHHAAQFPNHS |
| | FKHEDPMGQQGSLGEQQYSVPPPVYGCHTPTDSCTGSQALLLRTPY |
| | SSDNLYQMTSQLECMTWNQMNLGATLKGHSTGYESDNHTTPILCG |
| | AQYRIHTHGVFRGIQDVRRVPGVAPTLVRSASETSEKRPFMCAYSG |
| | CNKRYFKLSHLQMHSRKHTGEKPYQCDFKDCERRFFRSDQLKRHQ |
| | RRHTGVKPFQCKTCQRKFSRSDHLKTHTRTHTGEKPFSCRWPSCQK |
| | KFARSDELVRHHNMHQRNMTKLQLAL |
| SEQ ID NO: 35 | MHHHHHHTAASDNFQLSQGGQGFAIPIGQAMAIAGQIRSGGGSPTV |
| | HIGPTAFLGLGVVDNNGNGARVQRVVGSAPAASLGISTGDVITAVD |
| | GAPINSATAMADALNGHHPGDVISVTWQTKSGGTRTGNVTLAEGP |
| | PAEFIEGRGSGCPLLENVISKTINPQVSKTEYKELLQEFIDDNATTNAI |
| | DELKECFLNQTDETLSNVEVFMQLIYDSSLCDLF |
| SEQ ID NO: 36 | MHHHHHHTAASDNFQLSQGGQGFAIPIGQAMAIAGQIRSGGGSPTV |
| | HIGPTAFLGLGVVDNNGNGARVQRVVGSAPAASLGISTGDVITAVD |
| | GAPINSATAMADALNGHHPGDVISVTWQTKSGGTRTGNVTLAEGP |
| | PAEFMVDFGALPPEINSARMYAGPGSASLVAAAQMWDSVASDLFS |
| | AASAFQSVVWGLTVGSWIGSSAGLMVAAASPYVAWMSVTAGQAE |
| | LTAAQVRVAAAAYETAYGLTVPPPVIAENRAELMILIATNLLGQNT |
| | PAIAVNEAEYGEMWAQDAAAMFGYAAATATATATLLPFEEAPEMT |
| | SAGGLLEQAAAVEEASDTAAANQLMNNVPQALQQLAQPTQGTTPS |
| | SKLGGLWKTVSPHRSPISNMVSMANNHMSMTNSGVSMTNTLSSML |
| | KGFAPAAAAQAVQTAAQNGVRAMSSLGSSLGSSGLGGGVAANLG |
| | RAASVGSLSVPQAWAAANQAVTPAARALPLTSLTSAAERGPGQML |
| | GGLPVGQMGARAGGGLSGVLRVPPRPYVMPHSPAAGDIAPPALSQ |
| | DRFADFPALPLDPSAMVAQVGPQVVNINTKLGYNNAVGAGTGIVID |
| | PNGVVLTNNHVIAGATDINAFSVGSGQTYGVDVVGYDRTQDVAVL |
| | QLRGAGGLPSAAIGGGVAVGEPVVAMGNSGGQGGTPRAVPGRVV |
| | ALGQTVQASDSLTGAEETLNGLIQFDAAIQPGDSGGPVVNGLGQVV |
| | GMNTAAS |
| SEQ ID NO: 37 | TAASDNFQLSQGGQGFAIPIGQAMAIAGQI |
| SEQ ID NO: 38 | TAASDNFQLSQGGQGFAIPIGQAMAIAGQIKLPTVHIGPTAFLGLGV |
| | VDNNGNGARVQRVVGSAPAASLGISTGDVITAVDGAPINSATAMA |
| | DALNGHHPGDVISVTWQTKSGGTRTGNVTLAEGPPA |
| SEQ ID NO: 39 | TAASDNFQLSQGGQGFAIPIGQAMAIAGQIRSGGGSPTVHIGPTAFL |
| | GLGVVDNNGNGARVQRVVGSAPAASLGISTGDVITAVDGAPINSAT |
| | AMADALNGHHPGDVISVTWQTKSGGTRTGNVTLAE |
| SEQ ID NO: 40 | MSNSRRRSLRWSWLLSVLAAVGLGLATAPAQAAPPALSQDRFADF |
| | PALPLDPSAMVAQVGPQVVNINTKLGYNNAVGAGTGIVIDPNGVVL |
| | TNNHVIAGATDINAFSVGSGQTYGVDVVGYDRTQDVAVLQLRGAG |
| | GLPSAAIGGGVAVGEPVVAMGNSGGQGGTPRAVPGRVVALGQTV |
| | QASDSLTGAEETLNGLIQFDAAIQPGDSGGPVVNGLGQVVGMNTA |
| | ASDNFQLSQGGQGFAIPIGQAMAIAGQIRSGGGSPTVHIGPTAFLGL |
| | GVVDNNGNGARVQRVVGSAPAASLGISTGDVITAVDGAPINSATA |
| | MADALNGHHPGDVISVTWQTKSGGTRTGNVTLAEGPPA |
| SEQ ID NO: 41 | MKLKTLALSLLAAGVLAGCSSHSSNMANTQMKSDKIIIAHRGASGY |
| | LPEHTLESKALAFAQQADYLEQDLAMTKDGRLVVIHDHFLDGLTD |
| | VAKKFPHRHRKDGRYYVIDFTLKEIQSLEMTENFETKDGKQAQVYP |
| | NRFPLWKSHFRIHTFEDEIEFIQGLEKSTGKKVGIYPEIKAPWFHHQN |
| | GKDIAAETLKVLKKYGYDKKTDMVYLQTFDFNELKRIKTELLPQM |
| | GMDLKLVQLIAYTDWKETQEKDPKGYWVNYNYDWMFKPGAMAE |
| | VVKYADGVGPGWYMLVNKEESKPDNIVYTPLVKELAQYNVEVHP |
| | YTVRKDALPAFFTDVNQMYDVLLNKSGATGVFTDFPDTGVEFLKGI |
| | K |
| SEQ ID NO: 42 | MEINVSKLRTDLPQVGVQPYRQVHAHSTGNPHSTVQNEADYHWRK |
| | DPELGFFSHIVGNGCIMQVGPVDNGAWDVGGGWNAETYAAVELIE |
| | SHSTKEEFMTDYRLYIELLRNLADEAGLPKTLDTGSLAGIKTHEYCT |
| | NNQPNNHSDHVDPYPYLAKWGISREQFKHDIENGLTIETGWQKNDT |
| | GYWYVHSDGSYPKDKFEKINGTWYYFDSSGYMLADRWRKHTDGN |
| | WYWFDNSGEMATGWKKIADKWYYFNEEGAMKTGWVKYKDTWY |
| | YLDAKEGAMVSNAFIQSADGTGWYYLKPDGTLADRPEFRMSQMA |
| SEQ ID NO: 43 | MKYTSYILAFQLCIVLGSLGCYCQDPYVKEAENLKKYFNAGHSDVA |
| | DNGTLFLGILKNWKEESDRKIMQSQIVSFYFKLFKNFKDDQSIQKSV |
| | ETIKEDMNVKFFNSNKKKRDDFEKLTNYSVTDLNVQRKAIHELIQV |
| | MAELSPAAKTGKRKRSQMLFRGRRASQ |
| SEQ ID NO: 44 | MSTESMIRDVELAEEALPKKTGGPQGSRRCLFLSLFSFLIVAGATTLF |
| | CLLHFGVIGPQREEFPRDLSLISPLAQAVRSSSRTPSDKPVAHVVANP |
| | QAEGQLQWLNRRANALLANGVELRDNQLVVPSEGLYLIYSQVLFK |
| | GQGCPSTHVLLTHTISRIAVSYQTKVNLLSAIKSPCQRETPEGAEAKP |
| | WYEPIYLGGVFQLEKGDRLSAEINRPDYLDFAESGQVYFGIIAL |
| SEQ ID NO: 45 | MYRMQLLSCIALSLALVTNSAPTSSSTKKTQLQLEHLLLDLQMILNG |
| | INNYKNPKLTRMLTFKFYMPKKATELKHLQCLEEELKPLEEVLNLA |
| | QSKNFHLRPRDLISNINVIVLELKGSETTFMCEYADETATIVEFLNRW |
| | ITFCQSIISTLT |
| SEQ ID NO: 46 | MTSKLAVALLAAFLISAALCEGAVLPRSAKELRCQCIKTYSKPFHPK |
| | FIKELRVIESGPHCANTEIIVKLSDGRELCLDPKENWVQRVVEKFLK |
| | RAENS |
| SEQ ID NO: 47 | MEPLVTWVVPLLFLFLLSRQGAACRTSECCFQDPPYPDADSGSASG |
| | PRDLRCYRISSDRYECSWQYEGPTAGVSHFLRCCLSSGRCCYFAAG |
| | SATRLQFSDQAGVSVLYTVTLWVESWARNQTEKSPEVTLQLYNSV |
| | KYEPPLGDIKVSKLAGQLRMEWETPDNQVGAEVQFRHRTPSSPWK |
| | LGDCGPQDDDTESCLCPLEMNVAQEFQLRRRQLGSQGSSWSKWSS |
| | PVCVPPENPPQPQVRFSVEQLGQDGRRRLTLKEQPTQLELPEGCQGL |
| | APGTEVTYRLQLHMLSCPCKAKATRTLHLGKMPYLSGAAYNVAVI |
| | SSNQFGPGLNQTWHIPADTHTEPVALNISVGTNGTTMYWPARAQS |
| | MTYCIEWQPVGQDGGLATCSLTAPQDPDPAGMATYSWSRESGAM |
| | GQEKCYYITIFASAHPEKLTLWSTVLSTYHFGGNASAAGTPHHVSV |
| | KNHSLDSVSVDWAPSLLSTCPGVLKEYVVRCRDEDSKQVSEHPVQP |
| | TETQVTLSGLRAGVAYTVQVRADTAWLRGVWSQPQRFSIEVQVSD |
| | WLIFFASLGSFLSILLVGVLGYLGLNRAARHLCPPLPTPCASSAIEFPG |
| | GKETWQWINPVDFQEEASLQEALVVEMSWDKGERTEPLEKTELPE |
| | GAPELALDTELSLEDGDRCKAKM |
| SEQ ID NO: 48 | MAAEPVEDNCINFVAMKFIDNTLYFIAEDDENLESDYFGKLESKLSV |
| | IRNLNDQVLFIDQGNRPLFEDMTDSDCRDNAPRTIFIISMYKDSQPRG |
| | MAVTISVKCEKISTLSCENKIISFKEMNPPDNIKDTKSDIIFFQRSVPG |
| | HDNKMQFESSSYEGYFLACEKERDLFKLILKKEDELGDRSIMFTVQ |
| | NED |
| SEQ ID NO: 49 | MFHVSFRYIFGLPPLILVLLPVASSDCDIEGKDGKQYESVLMVSIDQL |
| | LDSMKEIGSNCLNNEFNFFKRHICDANKEGMFLFRAARKLRQFLKM |
| | NSTGDFDLHLLKVSEGTTILLNCTGQVKGRKPAALGEAQPTKSLEE |
| | NKSLKEQKKLNDLCFLKRLLQEIKTCWNKILMGTKEH |
| SEQ ID NO: 50 | MSRLPVLLLLQLLVRPGLQAPMTQTTSLKTSWVNCSNMIDEIITHLK |
| | QPPLPLLDFNNLNGEDQDILMENNLRRPNLEAFNRAVKSLQNASAIE |
| | SILKNLLPCLPLATAAPTRHPIHIKDGDWNEFRRKLTFYLKTLENAQ |
| | AQQTTLSLAIF |
| SEQ ID NO: 51 | MGLTSQLLPPLFFLLACAGNFVHGHKCDITLQEIIKTLNSLTEQKTLC |
| | TELTVTDIFAASKNTTEKETFCRAATVLRQFYSHHEKDTRCLGATA |
| | QQFHRHKQLIRFLKRLDRNLWGLAGLNSCPVKEANQSTLENFLERL |
| | KTIMREKYSKCSS |
| SEQ ID NO: 52 | MRMLLHLSLLALGAAYVYAIPTEIPTSALVKETLALLSTHRTLLIAN |
| | ETLRIPVPVHKNHQLCTEEIFQGIGTLESQTVQGGTVERLFKNLSLIK |
| | KYIDGQKKKCGEERRRVNQFLDYLQEFLGVMNTEWIIES |
| SEQ ID NO: 53 | MNSFSTSAFGPVAFSLGLLLVLPAAFPAPVPPGEDSKDVAAPHRQPL |
| | TSSERIDKQIRYILDGISALRKETCNKSNMCESSKEALAENNLNLPK |
| | MAEKDGCFQSGFNEETCLVKIITGLLEFEVYLEYLQNRFESSEEQAR |
| | AVQMSTKVLIQFLQKKAKNLDAITTPDPTTNASLLTKLQAQNQWLQ |
| | DMTTHLILRSFKEFLQSSLRALRQM |
| SEQ ID NO: 54 | MVLTSALLLCSVAGQGCPTLAGILDINFLINKMQEDPASKCHCSAN |
| | VTSCLCLGIPSDNCTRPCFSERLSQMTNTTMQTRYPLIFSRVKKSVE |
| | VLKNNKCPYFSCEQPCNQTTAGNALTFLKSLLEIFQKEKMRGMRGK |
| | I |
| SEQ ID NO: 55 | MHSSALLCCLVLLTGVRASPGQGTQSENSCTHFPGNLPNMLRDLRD |
| | AFSRVKTFFQMKDQLDNLLLKESLLEDFKGYLGCQALSEMIQFYLE |
| | EVMPQAENQDPDIKAHVNSLGENLKTLRLRLRRCHRFLPCENKSKA |
| | VEQVKNAFNKLQEKGIYKAMSEFDIFINYIEAYMTMKIRN |
| SEQ ID NO: 56 | MALLLTTVIALTCLGGFASPGPVPPSTALRELIEELVNITQNQKAPLC |
| | NGSMVWSINLTAGMYCAALESLINVSGCSAIEKTQRMLSGFCPHKV |
| | SAGQFSSLHVRDTKIEVAQFVKDLLLHLKKLEREGQENRNEESIIICR |
| | DRT |
| SEQ ID NO: 57 | MDEQVQIFSFLLISASVIMSRANWVNVISDLKKIEDLIQSMHIDATLY |
| | TESDVHPSCKVTAMKCFLLELQVISLESGDASIHDTVENLIILANNSL |
| | SSNGNVTESGCKECEELEEKNIKEFLQSFVHIVQMFINTS |
| SEQ ID NO: 58 | MEGDGSDPEPPDAGEDSKSENGENAPIYCICRKPDINCFMIGCDNCN |
| | EWFHGDCIRITEKMAKAIREWYCRECREKDPKLEIRYRHKKSRERD |
| | GNERDSSEPRDEGGGRKRPVPDPNLQRRAGSGTGVGAMLARGSAS |
| | PHKSSPQPLVATPSQHHQQQQQQIKRSARMCGECEACRRTEDCGHC |
| | DFCRDMKKFGGPNKIRQKCRLRQCQLRARESYKYFPSSLSPVTPSES |
| | LPRPRRPLPTQQQPQPSQKLGRIREDEGAVASSTVKEPPEATATPEPL |
| | SDEDLPLDPDLYQDFCAGAFDDNGLPWMSDTEESPFLDPALRKRAV |
| | KVKHVKRREKKSEKKKEERYKRHRQKQKHKDKWKHPERADAKD |
| | PASLPQCLGPGCVRPAQPSSKYCSDDCGMKLAANRIYEILPQRIQQW |
| | QQSPCIAEEHGKKLLERIRREQQSARTRLQEMERRFHELEAIILRAKQ |
| | QAVREDEESNEGDSDDTDLQIFCVSCGHPINPRVALRHMERCYAKY |
| | ESQTSFGSMYPTRIEGATRLFCDVYNPQSKTYCKRLQVLCPEHSRDP |
| | KVPADEVCGCPLVRDVFELTGDFCRLPKRQCNRHYCWEKLRRAEV |
| | DLERVRVWYKLDELFEQERNVRTAMTNRAGLLALMLHQTIQHDPL |
| | TTDLRSSADR |
| SEQ ID NO: 59 | MIKLKFGVFFTVLLSSAYAHGTPQNITDLCAEYHNTQIYTLNDKIFS |
| | YTESLAGKREMAIITFKNGAIFQVEVPGSQHIDSQKKAIERMKDTLRI |
| | AYLTEAKVEKLCVWNNKTPHAIAAISMAN |
| SEQ ID NO: 60 | MVKIIFVFFIFLSSFSYANDDKLYRADSRPPDEIKQSGGLMPRGQNEY |
| | FDRGTQMNINLYDHARGTQTGFVRHDDGYVSTSISLRSAHLVGQTI |
| | LSGHSTYYIYVIATAPNMFNVNDVLGAYSPHPDEQEVSALGGIPYSQ |
| | IYGWYRVHFGVLDEQLHRNRGYRDRYYSNLDIAPAADGYGLAGFP |
| | PEHRAWREEPWIHHAPPGCGNAPRSSMSNTCDEKTQSLGVKFLDEY |
| | QSKVKRQIFSGYQSDIDTHNRIKDEL |
| SEQ ID NO: 61 | MIKLKFGVFFTVLLSSAYAHGTPQNITDLCAEYHNTQIHTLNDKILS |
| | YTESLAGNREMAIITFKNGATFQVEVPGSQHIDSQKKAIERMKDTLR |
| | IAYLTEAKVEKLCVWNNKTPHAIAAISMAN |
| SEQ ID NO: 62 | DPNAPKRPPSAFFLFCSE |
| SEQ ID NO: 63 | MCCTKSLLLAALMSVLLLHLCGESEAASNFDCCLGYTDRILHPKFIV |
| | GFTRQLANEGCDINAIIFHTKKKLSVCANPKQTWVKYIVRLLSKKV |
| | KNM |
| SEQ ID NO: 64 | MQVSTAALAVLLCTMALCNQFSASLAADTPTACCFSYTSRQIPQNFI |
| | ADYFETSSQCSKPGVIFLTKRSRQVCADPSEEWVQKYVSDLELSA |
| SEQ ID NO: 65 | MWLQSLLLLGTVACSISAPARSPSPSTQPWEHVNAIQEARRLLNLSR |
| | DTAAEMNETVEVISEMFDLQEPTCLQTRLELYKQGLRGSLTKLKGP |
| | LTMMASHYKQHCPPTPETSCATQIITFESFKENLKDFLLVIPFDCWEP |
| | VQE |
| SEQ ID NO: 66 | MAGPATQSPMKLMALQLLLWHSALWTVQEATPLGPASSLPQSFLL |
| | KCLEQVRKIQGDGAALQEKLCATYKLCHPEELVLLGHSLGIPWAPL |
| | SSCPSQALQLAGCLSQLHSGLFLYQGLLQALEGISPELGPTLDTLQL |
| | DVADFATTIWQQMEELGMAPALQPTQGAMPAFASAFQRRAGGVL |
| | VASHLQSFLEVSYRVLRHLAQP |
| SEQ ID NO: 67 | QEINSSY |
| SEQ ID NO: 68 | SHPRLSA |
| SEQ ID NO: 69 | SMPNPMV |
| SEQ ID NO: 70 | GLQQVLL |
| SEQ ID NO: 71 | HELSVLL |
| SEQ ID NO: 72 | YAPQRLP |
| SEQ ID NO: 73 | TPRTLPT |
| SEQ ID NO: 74 | APVHSSI |
| SEQ ID NO: 75 | APPHALS |
| SEQ ID NO: 76 | TFSNRFI |
| SEQ ID NO: 77 | VVPTPPY |
| SEQ ID NO: 78 | ELAPDSP |
| SEQ ID NO: 79 | TPDCVTGKVEYTKYNDDDTFTVKVGDKELFTNRWNLQSLLLSAQIT |
| | GMTVTIKQNACHNGGGFSEVIFR |
| SEQ ID NO: 80 | MSRKLFASILIGALLGIGAPPSAHAGADDVVDSSKSFVMENFSSYHG |
| | TKPGYVDSIQKGIQKPKSGTQGNYDDDWKGFYSTDNKYDAAGYSV |
| | DNENPLSGKAGGVVKVTYPGLTKVLALKVDNAETIKKELGLSLTEP |
| | LMEQVGTEEFIKRFGDGASRVVLSLPFAEGSSSVEYINNWEQAKALS |
| | VELEINFETRGKRGQDAMYEYMAQACAGNRVRRSVGSSLSCINLD |
| | WDVIRDKTKTKIESLKEHGPIKNKMSESPNKTVSEEKAKQYLEEFH |
| | QTALEHPELSELKTVTGTNPVFAGANYAAWAVNVAQVIDSETADN |
| | LEKTTAALSILPGIGSVMGIADGAVHHNTEEIVAQSIALSSLMVAQAI |
| | PLVGELVDIGFAAYNFVESIINLFQVVHNSYNRPAYSPGHKTQPFLH |
| | DGYAVSWNTVEDSIIRTGFQGESGHDIKITAENTPLPIAGVLLPTIPG |
| | KLDVNKSKTHISVNGRKIRMRCRAIDGDVTFCRPKSPVYVGNGVHA |
| | NLHVAFHRSSSEKIHSNEISSDSIGVLGYQKTVDHTKVNSKLSLFFEI |
| | KS |
| SEQ ID NO: 81 | NWVNVISDLKKIEDLIQSMHIDATLYTESDVHPSCKVTAMKCFLLEL |
| | QVISLESGDASIHDTVENLIILANDSLSSNGNVTESGCKECEELEEKNI |
| | KEFLQSFVHIVQMFINTS |
| SEQ ID NO: 82 | ITCPPPMSVEHADIWVKSYSLYSRERYICNSGFKRKAGTSSLTECVL |
| | NKATNVAHWTTPSLKCIREPKSCDKTHTCPPCPAPELLGGPSVFLFP |
| | PKPKDTLMISRTPEVTCVVVDVSHEDPEVKFNWYVDGVEVHNAKT |
| | KPREEQYNSTYRVVSVLTVLHQDWLNGKEYKCKVSNKALPAPIEK |
| | TISKAKGQPREPQVYTLPPSRDELTKNQVSLTCLVKGFYPSDIAVEW |
| | ESNGQPENNYKTTPPVLDSDGSFFLYSKLTVDKSRWQQGNVFSCSV |
| | MHEALHNHYTQKSLSLSPGK |
| SEQ ID NO: 83 | GADDVVDSSKSFVMENFSSYHGTKPGYVDSIQKGIQKPKSGTQGNY |
| | DDDWKEFYSTDNKYDAAGYSVDNENPLSGKAGGVVKVTYPGLTK |
| | VLALKVDNAETIKKELGLSLTEPLMEQVGTEEFIKRFGDGASRVVLS |
| | LPFAEGSSSVEYINNWEQAKALSVELEINFETRGKRGQDAMYEYMA |
| | QACAGNRVRRSVGSSLSCINLDWDVIRDKTKTKIESLKEHGPIKNK |
| | MSESPNKTVSEEKAKQYLEEFHQTALEHPELSELKTVTGTNPVFAG |
| | ANYAAWAVNVAQVIDSETADNLEKTTAALSILPGIGSVMGIADGAV |
| | HHNTEEIVAQSIALSSLMVAQAIPLVGELVDIGFAAYNFVESIINLFQ |
| | VVHNSYNRPAYSPGHKTQPFLHDGYAVSWNTVEDSIIRTGFQGESG |
| | HDIKITAENTPLPIAGVLLPTIPGKLDVNKSKTHISVNGRKIRMRCRAI |
| | DGDVTFCRPKSPVYVGNGVHANLHVAFHRSSSEKIHSNEISSDSIGV |
| | LGYQKTVDHTKVNSKLSLFFEIKS |
| SEQ ID NO: 103 | MESHSRAGKSRKSAKFRSISRSLMLCNAKTSDDGSSPDEKYPDPFEI |
| | SLAQGKEGIFHSSVQLADTSEAGPSSVPDLALASEAAQLQAAGNDR |
| | GKTCRRIFFMKESSTASSREKPGKLEAQSSNFLFPKACHQRARSNST |
| | SVNPYCTREIDFPMTKKSAAPTDRQPYSLCSNRKSLSQQLDCPAGK |
| | AAGTSRPTRSLSTAQLVQPSGGLQASVISNIVLMKGQAKGLGFSIVG |
| | GKDSIYGPIGIYVKTIFAGGAAAADGRLQEGDEILELNGESMAGLTH |
| | QDALQKFKQAKKGLLTLTVRTRLTAPPSLCSHLSPPLCRSLSSSTCIT |
| | KDSSSFALESPSAPISTAKPNYRIMVEVSLQKEAGVGLGIGLCSVPYF |
| | QCISGIFVHTLSPGSVAHLDGRLRCGDEIVEISDSPVHCLTLNEVYTIL |
| | SRCDPGPVPIIVSRHPDPQVSEQQLKEAVAQAVENTKFGKERHQWS |
| | LEGVKRLESSWHGRPTLEKEREKNSAPPHRRAQKVMIRSSSDSSYM |
| | SGSPGGSPGSGSAEKPSSDVDISTHSPSLPLAREPVVLSIASSRLPQES |
| | PPLPESRDSHPPLRLKKSFEILVRKPMSSKPKPPPRKYFKSDSDPQKS |
| | LEERENSSCSSGHTPPTCGQEARELLPLLLPQEDTAGRSPSASAGCPG |
| | PGIGPQTKSSTEGEPGWRRASPVTQTSPIKHPLLKRQARMDYSFDTT |
| | AEDPWVRISDCIKNLFSPIMSENHGHMPLQPNASLNEEEGTQGHPDG |
| | TPPKLDTANGTPKVYKSADSSTVKKGPPVAPKPAWFRQSLKGLRNR |
| | ASDPRGLPDPALSTQPAPASREHLGSHIRASSSSSSIRQRISSFETFGSS |
| | QLPDKGAQRLSLQPSSGEAAKPLGKHEEGRFSGLLGRGAAPTLVPQ |
| | QPEQVLSSGSPAASEARDPGVSESPPPGRQPNQKTLPPGPDPLLRLLS |
| | TQAEESQGPVLKMPSQRARSFPLTRSQSCETKLLDEKTSKLYSISSQ |
| | VSSAVMKSLLCLPSSISCAQTPCIPKEGASPTSSSNEDSAANGSAETS |
| | ALDTGFSLNLSELREYTEGLTEAKEDDDGDHSSLQSGQSVISLLSSEE |
| | LKKLIEEVKVLDEATLKQLDGIHVTILHKEEGAGLGFSLAGGADLEN |
| | KVITVHRVFPNGLASQEGTIQKGNEVLSINGKSLKGTTHHDALAILR |
| | QAREPRQAVIVTRKLTPEAMPDLNSSTDSAASASAASDVSVESTEAT |
| | VCTVTLEKMSAGLGFSLEGGKGSLHGDKPLTINRIFKGAASEQSETV |
| | QPGDEILQLGGTAMQGLTRFEAWNIIKALPDGPVTIVIRRKSLQSKE |
| | TTAAGDS |
| SEQ ID NO: 104 | MTPGKTSLVSLLLLLSLEAIVKAGITIPRNPGCPNSEDKNFPRTVMVN |
| | LNIHNRNTNTNPKRSSDYYNRSTSPWNLHRNEDPERYPSVIWEAKC |
| | RHLGCINADGNVDYHMNSVPIQQEILVLRREPPHCPNSFRLEKILVS |
| | VGCTCVTPIVHHVA |
| SEQ ID NO: 105 | RAVPGGSSPAWTQCQQLSQKLCTLAWSAHPLVGHMDLREEGDEET |
| | TNDVPHIQCGDGCDPQGLRDNSQFCLQRIHQGLIFYEKLLGSDIFTG |
| | EPSLLPDSPVGQLHASLLGLSQLLQPEGHHWETQQIPSLSPSQPWQR |
| | LLLRFKILRSLQAFVAVAARVFAHGAATLSPIWELKKDVYVVELDW |
| | YPDAPGEMVVLTCDTPEEDGITWTLDQSSEVLGSGKTLTIQVKEFG |
| | DAGQYTCHKGGEVLSHSLLLLHKKEDGIWSTDILKDQKEPKNKTFL |
| | RCEAKNYSGRFTCWWLTTISTDLTFSVKSSRGSSDPQGVTCGAATL |
| | SAERVRGDNKEYEYSVECQEDSACPAAEESLPIEVMVDAVHKLKYE |
| | NYTSSFFIRDIIKPDPPKNLQLKPLKNSRQVEVSWEYPDTWSTPHSYF |
| | SLTFCVQVQGKSKREKKDRVFTDKTSATVICRKNASISVRAQDRYY |
| | SSSWSEWASVPCS |
| SEQ ID NO: 106 | MCFPKVLSDDMKKLKARMVMLLPTSAQGLGAWVSACDTEDTVGH |
| | LGPWRDKDPALWCQLCLSSQHQAIERFYDKMQNAESGRGQVMSSL |
| | AELEDDFKEGYLETVAAYYEEQHPELTPLLEKERDGLRCRGNRSPV |
| | PDVEDPATEEPGESFCDKVMRWFQAMLQRLQTWWHGVLAWVKE |
| | KVVALVHAVQALWKQFQSFCCSLSELFMSSFQSYGAPRGDKEELTP |
| | QKCSEPQSSK |

In some embodiments, the nucleic acid sequences for the target antigen and the immunological fusion partner are not separated by any nucleic acids. In other embodiments, a nucleic acid sequence that encodes for a linker can be inserted between the nucleic acid sequence encoding for any target antigen described herein and the nucleic acid sequence encoding for any immunological fusion partner described herein. Thus, in certain embodiments, the protein produced following immunization with the viral vector containing a target antigen, a linker, and an immunological fusion partner can be a fusion protein comprising the target antigen of interest followed by the linker and ending with the immunological fusion partner, thus linking the target antigen to an immunological fusion partner that increases the immunogenicity of the target antigen of interest via a linker. In some embodiments, the sequence of linker nucleic acids can be from about 1 to about 150 nucleic acids long, from about 5 to about 100 nucleic acids along, or from about 10 to about 50 nucleic acids in length. In some embodiments, the nucleic acid sequences may encode one or more amino acid residues. In some embodiments, the amino acid sequence of the linker can be from about 1 to about 50, or about 5 to about 25 amino acid residues in length. In some embodiments, the sequence of the linker comprises less than 10 amino acids. In some embodiments, the linker can be a polyalanine linker, a polyglycine linker, or a linker with both alanines and glycines.

Nucleic acid sequences that encode for such linkers can be any one of SEQ ID NO: 84-SEQ ID NO: 98 and are summarized in TABLE 3.

TABLE 3
Sequences of Linkers

| SEQ ID NO | Sequence |
|---|---|
| SEQ ID NO: 84 | MAVPMQLSCSR |
| SEQ ID NO: 85 | RSTG |
| SEQ ID NO: 86 | TR |
| SEQ ID NO: 87 | RSQ |
| SEQ ID NO: 88 | RSAGE |
| SEQ ID NO: 89 | RS |
| SEQ ID NO: 90 | GG |
| SEQ ID NO: 91 | GSGGSGGSG |
| SEQ ID NO: 92 | GGSGGSGGSGG |
| SEQ ID NO: 93 | GGSGGSGGSGGSGG |
| SEQ ID NO: 94 | GGSGGSGGSGGSGGSGG |
| SEQ ID NO: 95 | GGSGGSGGSGGSGGSGGSGG |
| SEQ ID NO: 96 | GGSGGSGGSGGSGGSGGSGGSGG |
| SEQ ID NO: 97 | GGSGGSGGSGGSGGSG |
| SEQ ID NO: 98 | GSGGSGGSGGSGGSGG |

### Formulations of Vaccines or ALT-803

Some embodiments provide pharmaceutical compositions comprising a vaccination and ALT-803 regimen that can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, in drug delivery devices for implantation and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. The compositions described throughout can be formulated into a pharmaceutical medicament and be used to treat a human or mammal, in need thereof, diagnosed with a disease, e.g., cancer.

For administration, viral vector or ALT-803 stock can be combined with an appropriate buffer, physiologically acceptable carrier, excipient or the like. In certain embodiments, an appropriate number of virus vector particles (VP) or ALT-803 proteins are administered in an appropriate buffer, such as, sterile PBS or saline. In certain embodiment, vector compositions and ALT-803 compositions disclosed herein are provided in specific formulations for subcutaneously, parenterally, intravenously, intramuscularly, or even intraperitoneally administration. In certain embodiments, formulations in a solution of the active compounds as free base or pharmacologically acceptable salts may be prepared in water suitably mixed with a surfactant, such as hydroxypropylcellulose. Dispersions may also be prepared in glycerol, liquid polyethylene glycols, squalene-based emulsion, Squalene-based oil-in-water emulsions, water-in-oil emulsions, oil-in-water emulsions, nonaqueous emulsions, water-in-paraffin oil emulsion, and mixtures thereof and in oils. In other embodiments, viral vectors may are provided in specific formulations for pill form administration by swallowing or by suppository.

Illustrative pharmaceutical forms suitable for injectable use include sterile aqueous solutions or dispersions and sterile powders for the extemporaneous preparation of sterile injectable solutions or dispersions (see, e.g., U.S. Pat. No. 5,466,468). Fluid forms to the extent that easy syringability exists may be preferred. Forms that are stable under the conditions of manufacture and storage are provided in some embodiments. In various embodiments, forms are preserved against the contaminating action of microorganisms, such as bacteria, molds and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, and thimerosal. It may be suitable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution can be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see, e.g., “Remington's Pharmaceutical Sciences” 15th Edition, pages 1035-1038 and 1570-1580). Some variation in dosage may occur depending on the condition of the subject being treated.

Carriers of formulation can comprise any and all solvents, dispersion media, vehicles, coatings, diluents, antibacterial and antifungal agents, isotonic and absorption delaying agents, buffers, carrier solutions, suspensions, colloids, suspending agents, solubilizing agents, stabilizing agents, pH-adjusting agent (such as hydrochloric id, sodium hydroxide or a suitable buffer, 1,3-butanediol, Ringer's solution, and isotonic sodium chloride solution and dextrose solution), tonicity adjusting agents, preservatives (e.g., methyl, ethyl or n-propyl p-hydroxybenzoate) and the like. Except insofar as any conventional media or agent is incompatible with the active ingredient, its use in the therapeutic compositions is contemplated. Supplementary active ingredients can also be incorporated into the compositions.

Pharmaceutical formulations can be provided as a unit dose, (e.g., in single-dose ampoules, syringes or bags), or in vials containing several doses and in which a suitable preservative may be added (see below). Therapeutic moieties can be formulated in microspheres, microcapsules, nanoparticles, or liposomes.

Formulation of Viral Vectors with Immunostimulants

In certain embodiments, the viral vectors may be administered in conjunction with one or more immunostimulants, such as an adjuvant. An immunostimulant refers to essentially any substance that enhances or potentiates an immune response (antibody and/or cell-mediated) to an antigen. One type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories); Merck Adjuvant 65 (Merck and Company, Inc.) AS-2 (SmithKline Beecham); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, MIF and others, like growth factors, may also be used as adjuvants.

In some embodiments, the adjuvant is selected from the group consisting of IL-15, a nucleic acid encoding IL-15, a protein with substantial identity to IL-15, and a nucleic acid encoding a protein with substantial identity to IL-15.

Within certain embodiments, the adjuvant composition can be one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient may support an immune response that includes Th1- and/or Th2-type responses. Within certain embodiments, in which a response is predominantly Th1-type, the level of Th1-type cytokines will increase to a greater extent than the level of Th2-type cytokines. The levels of these cytokines may be readily assessed using standard assays. Thus, various embodiments relate to therapies raising an immune response against a target antigen, for example CEA, using cytokines, e.g., IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and/or MIF supplied concurrently with a replication defective viral vector treatment. In some embodiments, a cytokine or a nucleic acid encoding a cytokine, is administered together with a replication defective viral described herein. In some embodiments, cytokine administration is performed prior or subsequent to viral vector administration. In some embodiments, a replication defective viral vector capable of raising an immune response against a target antigen, for example CEA, further comprises a sequence encoding a cytokine.

Certain illustrative adjuvants for eliciting a predominantly Th1-type response include, for example, a combination of monophosphoryl lipid A, such as 3-de-O-acylated monophosphoryl lipid A, together with an aluminum salt. MPL® adjuvants are commercially available (see, e.g., U.S. Pat. Nos. 4,436,727; 4,877,611; 4,866,034 and 4,912,094). CpG-containing oligonucleotides (in which the CpG dinucleotide is unmethylated) also induce a predominantly Th1 response. (see, e.g., WO 96/02555, WO 99/33488 and U.S. Pat. Nos. 6,008,200 and 5,856,462). Immunostimulatory DNA sequences can also be used. Another adjuvant for use comprises a saponin, such as Quil A, or derivatives thereof, including QS21 and QS7 (Aquila Biopharmaceuticals Inc.), Escin; Digitonin; or Gypsophila or Chenopodium quinoa saponins. Other formulations may include more than one saponin in the adjuvant combinations, e.g., combinations of at least two of the following group comprising QS21, QS7, Quil A, β-escin, or digitonin.

In some embodiments, the compositions may be delivered by intranasal sprays, inhalation, and/or other aerosol delivery vehicles. The delivery of drugs using intranasal microparticle resins and lysophosphatidyl-glycerol compounds can be employed (see, e.g., U.S. Pat. No. 5,725,871). Likewise, illustrative transmucosal drug delivery in the form of a polytetrafluoroetheylene support matrix can be employed (see, e.g., U.S. Pat. No. 5,780,045).

Liposomes, nanocapsules, microparticles, lipid particles, vesicles, and the like, can be used for the introduction of the compositions into suitable hot cells/organisms. Compositions as described herein may be formulated for delivery either encapsulated in a lipid particle, a liposome, a vesicle, a nanosphere, or a nanoparticle or the like. Alternatively, compositions as described herein can be bound, either covalently or non-covalently, to the surface of such carrier vehicles. Liposomes can be used effectively to introduce genes, various drugs, radiotherapeutic agents, enzymes, viruses, transcription factors, allosteric effectors and the like, into a variety of cultured cell lines and animals. Furthermore, the use of liposomes does not appear to be associated with autoimmune responses or unacceptable toxicity after systemic delivery. In some embodiments, liposomes are formed from phospholipids dispersed in an aqueous medium and spontaneously form multilamellar concentric bilayer vesicles (i.e. multilamellar vesicles (MLVs)).

In some embodiments, pharmaceutically-acceptable nanocapsule formulations of the compositions are provided. Nanocapsules can generally entrap compounds in a stable and reproducible way. To avoid side effects due to intracellular polymeric overloading, such ultrafine particles (sized around 0.1 μm) may be designed using polymers able to be degraded in vivo.

The compositions in some embodiments comprise or are administered with a chemotherapeutic agent (e.g., a chemical compound useful in the treatment of cancer). Chemotherapeutic cancer agents that can be used in combination with the disclosed T cell include, but are not limited to, mitotic inhibitors (vinca alkaloids), such as vincristine, vinblastine, vindesine and Navelbine™ (vinorelbine, 5′-noranhydroblastine); topoisomerase I inhibitors, such as camptothecin compounds (e.g., Camptosar™ (irinotecan HCL), Hycamtin™ (topotecan HCL) and other compounds derived from camptothecin and its analogues); podophyllotoxin derivatives, such as etoposide, teniposide and mitopodozide; alkylating agents such as cisplatin, cyclophosphamide, nitrogen mustard, trimethylene thiophosphoramide, carmustine, busulfan, chlorambucil, belustine, uracil mustard, chlomaphazin, and dacarbazine; antimetabolites such as cytosine arabinoside, fluorouracil, methotrexate, mercaptopurine, azathioprime, and procarbazine; antibiotics, such as doxorubicin, bleomycin, dactinomycin, daunorubicin, mithramycin, mitomycin, mytomycin C, and daunomycin; anti-tumor antibodies; dacarbazine; azacytidine; amsacrine; melphalan; ifosfamide; and mitoxantrone.

Compositions disclosed herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2 (TIMP-1 and -2). Small molecules, including topoisomerases such as razoxane, a topoisomerase II inhibitor with anti-angiogenic activity, can also be used.

### Methods of Preparation of Ad5 Vaccines

In some embodiments, compositions and methods make use of human cytolytic T-cells (CTLs), such as those that recognize CEAs epitopes which bind to selected MHC molecules, e.g., HLA-A2, A3, and A24. Individuals expressing MHC molecules of certain serotypes, e.g., HLA-A2, A3, and A24 may be selected for therapy using the methods and compositions as described herein. For example, individuals expressing MHC molecules of certain serotypes, e.g., HLA-A2, A3, and A24, may be selected for a therapy including raising an immune response against CEAs, using the methods and compositions described herein.

In various embodiments, these T-cells can be generated by in vitro cultures using antigen-presenting cells pulsed with the epitope of interest to stimulate peripheral blood mononuclear cells. In addition, T-cell lines can also be generated after stimulation with CEA latex beads, CEA protein-pulsed plastic adherent peripheral blood mononuclear cells, or DCs sensitized with CEAsRNA. T-cells can also be generated from patients immunized with a vaccine vector encoding CEAs immunogen. HLA A2-presented peptides from CEAs can further be found in primary gastrointestinal tumors.

Some embodiments relate to an HLA A2 restricted epitope of CEAs, CAP-1, a nine amino acid sequence (YLSGANLNL; SEQ ID NO: 4), with ability to stimulate CTLs from cancer patients immunized with vaccine—CEAs. Cap-1(6D) (YLSGADLNL; SEQ ID NO: 4) is a peptide analog of CAP-1. Its sequence includes a heteroclitic (nonanchor position) mutation, resulting in an amino acid change from Asn to Asp, enhancing recognition by the T-cell receptor. The Asn to Asp mutation appears to not cause any change in the binding of the peptide to HLA A2. Compared with the non-mutated CAP-1 epitope, Cap-1(6D) can enhance the sensitization of CTLs by 100 to 1,000 times. CTL lines can be elicited from peripheral blood mononuclear cells of healthy volunteers by in vitro sensitization to the Cap-1(6D) peptide, but not significantly to the CAP-1 peptide. These cell lines can lyse human tumor cells expressing endogenous CEA. Thus, polypeptide sequences comprising CAP-1 or CAP-1(6D), nucleic acid sequences encoding such sequences, an adenovirus vectors; for example replication defective adenovirus vectors, comprising such nucleic acid sequences are provided in some embodiments.

Methods of Treatment with Ad5 Vaccines

The adenovirus vectors can be used in a number of vaccine settings for generating an immune response against one or more target antigens as described herein. Some embodiments provide methods of generating an immune response against any target antigen, such as those described elsewhere herein. The adenovirus vectors are of particular importance because of the unexpected finding that they can be used to generate immune responses in subjects who have preexisting immunity to Ad and can be used in vaccination regimens that include multiple rounds of immunization using the adenovirus vectors, regimens not possible using previous generation adenovirus vectors.

In some embodiments, a first or a second replication defective adenovirus infects dendritic cells in the human and wherein the infected dendritic cells present the antigen, thereby inducing the immune response.

Generally, generating an immune response comprises an induction of a humoral response and/or a cell-mediated response. It may desirable to increase an immune response against a target antigen of interest. Generating an immune response may involve a decrease in the activity and/or number of certain cells of the immune system or a decrease in the level and/or activity of certain cytokines or other effector molecules. Any suitable methods for detecting alterations in an immune response (e.g., cell numbers, cytokine expression, cell activity) can be used in some embodiments. Illustrative methods useful in this context include intracellular cytokine staining (ICS), ELISpot, proliferation assays, cytotoxic T-cell assays including chromium release or equivalent assays, and gene expression analysis using any number of polymerase chain reaction (PCR) or RT-PCR based assays.

Generating an immune response can comprise an increase in target antigen-specific CTL activity of between 1.5 and 5-fold in a subject administered the adenovirus vectors as described herein as compared to a control. In another embodiment, generating an immune response comprises an increase in target-specific CTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered the adenovirus vectors as compared to a control.

Generating an immune response can comprise an increase in target antigen-specific HTL activity, such as proliferation of helper T-cells, of between 1.5 and 5-fold in a subject administered the adenovirus vectors that comprise nucleic acid encoding the target antigen as compared to an appropriate control. In another embodiment, generating an immune response comprises an increase in target-specific HTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold as compared to a control. In this context, HTL activity may comprise an increase as described above, or decrease, in production of a particular cytokine, such as interferon-γ (IFN-γ), interleukin-1 (IL-1), IL-2, IL-3, IL-6, IL-7, IL-12, IL-15, tumor necrosis factor-α (TNF-α), granulocyte macrophage colony-stimulating factor (GM-CSF), granulocyte-colony stimulating factor (G-CSF), or other cytokines. In this regard, generating an immune response may comprise a shift from a Th2 type response to a Th1 type response or in certain embodiments a shift from a Th1 type response to a Th2 type response. In other embodiments, generating an immune response may comprise the stimulation of a predominantly Th1 or a Th2 type response.

Generating an immune response can comprise an increase in target-specific antibody production of between 1.5 and 5-fold in a subject administered the adenovirus vectors as compared to an appropriate control. In another embodiment, generating an immune response comprises an increase in target-specific antibody production of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered the adenovirus vector as compared to a control.

In some embodiments, the recombinant viral vector affects overexpression of the antigen in transfected cells. In some embodiments, the recombinant viral induces a specific immune response against cells expressing the antigen in a human that is at least 2, 3, 4, 5, 6, 7, 8, 9, 10, 15, 20, or 25-fold over basal. In some embodiments, the human has an inverse Ad5 neutralizing antibody titer of greater than 50, 75, 100, 125, 150, 160, 175, 200, 225, 250, 275, or 300 prior to the administering step. In some embodiments, the human has an inverse Ad5 neutralizing antibody titer of greater than 250, 500, 750, 1000, 1500, 2000, 2500, 3000, 3500, 4000, 4500, or 4767. In some embodiments, the immune response is measured as antigen specific antibody response.

In some embodiments, the immune response is measured as antigen specific cell-mediated immunity (CMI). In some embodiments, the immune response is measured as antigen specific IFN-γ secretion. In some embodiments, the immune response is measured as antigen specific IL-2 secretion. In some embodiments, the immune response against the antigen is measured by ELISpot assay. In some embodiments, the antigen specific CMI is greater than 25, 50, 75, 100, 150, 200, 250, or 300 IFN-γ spot forming cells (SFC) per 10⁶ peripheral blood mononuclear cells (PBMC). In some embodiments, the immune response is measured by T-cell lysis of CAP-1 pulsed antigen-presenting cells, allogeneic antigen expressing cells from a tumor cell line or from an autologous tumor.

Thus, some embodiments provide methods for generating an immune response against a target antigen of interest comprising administering to the individual an adenovirus vector comprising: a) a replication defective adenovirus vector, wherein the adenovirus vector has a deletion in the E2b region, and b) a nucleic acid encoding the target antigen; and readministering the adenovirus vector at least once to the individual; thereby generating an immune response against the target antigen. In certain embodiments, the vector administered to the individual is not a gutted vector. In particular embodiments, the target antigen may be a wild-type protein, a fragment, a variant, or a variant fragment thereof. In some embodiments, the target antigen comprises CEA, a fragment, a variant, or a variant fragment thereof.

In a further embodiment, there is provided methods for generating an immune response against a target antigen in an individual, wherein the individual has preexisting immunity to Ad, by administering to the individual an adenovirus vector comprising: a) a replication defective adenovirus vector, wherein the adenovirus vector has a deletion in the E2b region, and b) a nucleic acid encoding the target antigen; and readministering the adenovirus vector at least once to the individual; thereby generating an immune response against the target antigen. In particular embodiments, the target antigen may be a wild-type protein, a fragment, a variant, or a variant fragment thereof. In some embodiments, the target antigen comprises CEA, a fragment, a variant, or a variant fragment thereof.

With regard to preexisting immunity to Ad, this can be determined using any suitable methods, such as antibody-based assays to test for the presence of Ad antibodies. Further, in certain embodiments, the methods include first determining that an individual has preexisting immunity to Ad then administering the E2b deleted adenovirus vectors as described herein.

One embodiment provides a method of generating an immune response against one or more target antigens in an individual comprising administering to the individual a first adenovirus vector comprising a replication defective adenovirus vector, wherein the adenovirus vector has a deletion in the E2b region, and a nucleic acid encoding at least one target antigen; administering to the individual a second adenovirus vector comprising a replication defective adenovirus vector, wherein the adenovirus vector has a deletion in the E2b region, and a nucleic acid encoding at least one target antigen, wherein the at least one target antigen of the second adenovirus vector is the same or different from the at least one target antigen of the first adenovirus vector. In particular embodiments, the target antigen may be a wild-type protein, a fragment, a variant, or a variant fragment thereof. In some embodiments, the target antigen comprises CEA, a fragment, a variant, or a variant fragment thereof.

Thus, multiple immunizations with the same E2b deleted adenovirus vector or multiple immunizations with different E2b deleted adenovirus vectors are contemplated in some embodiments. In each case, the adenovirus vectors may comprise nucleic acid sequences that encode one or more target antigens as described elsewhere herein. In certain embodiments, the methods comprise multiple immunizations with an E2b deleted adenovirus encoding one target antigen, and re-administration of the same adenovirus vector multiple times, thereby inducing an immune response against the target antigen. In some embodiments, the target antigen comprises CEA, a fragment, a variant, or a variant fragment thereof.

In a further embodiment, the methods comprise immunization with a first adenovirus vector that encodes one or more target antigens, and then administration with a second adenovirus vector that encodes one or more target antigens that may be the same or different from those antigens encoded by the first adenovirus vector. In this regard, one of the encoded target antigens may be different or all of the encoded antigens may be different, or some may be the same and some may be different. Further, in certain embodiments, the methods include administering the first adenovirus vector multiple times and administering the second adenovirus multiple times. In this regard, the methods comprise administering the first adenovirus vector 1, 2, 3, 4, 5, 6, 7, 8, 9, 10, 11, 12, 13, 14, 15, or more times and administering the second adenovirus vector 1, 2, 3, 4, 5, 6, 7, 8, 9, 10, 11, 12, 13, 14, 15, or more times. The order of administration may comprise administering the first adenovirus one or multiple times in a row followed by administering the second adenovirus vector one or multiple times in a row. In certain embodiments, the methods include alternating administration of the first and the second adenovirus vectors as one administration each, two administrations each, three administrations each, and so on. In certain embodiments, the first and the second adenovirus vectors are administered simultaneously. In other embodiments, the first and the second adenovirus vectors are administered sequentially. In some embodiments, the target antigen comprises CEA, a fragment, a variant, or a variant fragment thereof.

As would be readily understood by the skilled artisan, more than two adenovirus vectors may be used in the methods. Three, 4, 5, 6, 7, 8, 9, 10, or more different adenovirus vectors may be used in the methods as described herein. In certain embodiments, the methods comprise administering more than one E2b deleted adenovirus vector at a time. In this regard, immune responses against multiple target antigens of interest can be generated by administering multiple different adenovirus vectors simultaneously, each comprising nucleic acid sequences encoding one or more target antigens.

The adenovirus vectors can be used to generate an immune response against a cancer, such as carcinomas or sarcomas (e.g., solid tumors, lymphomas and leukemia). The adenovirus vectors can be used to generate an immune response against an infectious disease, such as a cancer, such as any CEA-expressing cancer, Brachyury-expressing cancer, MUC1-expressing cancer, an epithelial cancer, a neurologic cancer, melanoma, non-Hodgkin's lymphoma, Hodgkin's disease, leukemia, plasmocytomas, adenomas, gliomas, thymomas, breast cancer, prostate cancer, colorectal cancer, kidney cancer, renal cell carcinoma, uterine cancer, pancreatic cancer, esophageal cancer, lung cancer, ovarian cancer, cervical cancer, testicular cancer, gastric cancer, multiple myeloma, hepatoma, acute lymphoblastic leukemia (ALL), acute myelogenous leukemia (AML), chronic myelogenous leukemia (CML), and chronic lymphocytic leukemia (CLL), gastrointestinal cancer, or other cancers.

In one aspect, a method of selecting a human for administration of the compositions is provided comprising: determining a HLA subtype of the human; and administering the composition to the human, if the HLA subtype is determined to be one of a preselected subgroup of HLA subtypes. In some embodiments, the preselected subgroup of HLA subtypes comprises one or more of HLA-A2, HLA-A3, and HLA-A24.

In some embodiments, the human is not concurrently being treated by any one of steroids, corticosteroids, and immunosuppressive agents. In some embodiments, the human does not have an autoimmune disease. In some embodiments, the human does not have inflammatory bowel disease, systemic lupus erythematosus, ankylosing spondylitis, scleroderma, multiple sclerosis, viral hepatitis, or HIV. In some embodiments, the human has or may have in the future an infectious disease. In some embodiments, the human has autoimmune related thyroid disease or vitiligo. In some embodiments, the human has or may have in the future a proliferative disease cancer. In some embodiments, the human has colorectal adenocarcinoma, metastatic colorectal cancer, advanced CEA expressing colorectal cancer, advanced MUC1-C, Brachyury, or CEA expressing colorectal cancer, breast cancer, lung cancer, bladder cancer, or pancreas cancer. In some embodiments, the human has at least 1, 2, or 3 sites of metastatic disease. In some embodiments, the human comprises cells overexpressing CEA. In some embodiments, the cells overexpressing CEA, overexpress the CEA by at least 2, 3, 4, 5, 6, 7, 8, 9, or 10 times over a baseline CEA expression in a non-cancer cell. In some embodiments, the cells overexpressing CEA comprise cancer cells. In some embodiments, the human comprises cells overexpressing MUC1-C, Brachyury, or CEA. In some embodiments, the cells overexpressing MUC1-C, Brachyury, or CEA, overexpress the MUC1-C, Brachyury, or CEA by at least 2, 3, 4, 5, 6, 7, 8, 9, or 10 times over a baseline MUC1-C, Brachyury, or CEA expression in a non-cancer cell. In some embodiments, the cells overexpressing MUC1-C, Brachyury, or CEA comprise cancer cells. In some embodiments, the subject has a diagnosed disease predisposition. In some embodiments, the subject has a stable disease. In some embodiments, the subject has a genetic predisposition for a disease. In some embodiments, the disease is a cancer. In some embodiments, the cancer is selected from the group consisting of prostate cancer, colon cancer, breast cancer, or gastric cancer. In some embodiments, the cancer is prostate cancer.

Some embodiments provide combination multi-targeted vaccines, immunotherapies and methods for enhanced therapeutic response to complex diseases such as infectious diseases and cancers. For example, in some embodiments, a subject can be administered a combination Ad5 vaccine as apart of the immunization strategy during treatment. For example, in some embodiments, a first and second replication defective adenovirus vector can be administered, each encoding for a different antigen. In some embodiments, the first or the second replication defective adenovirus vector comprises a sequence with at least 80% sequence identity to SEQ ID NO: 2. In some embodiments, the first or the second replication defective adenovirus vector comprises a region with at least 80% sequence identity to a region in SEQ ID NO: 2 selected from 26048-26177, 26063-26141, 1-103, 54-103, 32214-32315, and 32214-32262. In some embodiments, the first or the second replication defective adenovirus vector comprises a region with at least 80% sequence identity to a region in SEQ ID NO: 2 between positions 1057 and 3165. In some embodiments, the first or second replication defective adenovirus vector comprises a sequence encoding a MUC1-C, Brachyury, or CEA antigen; wherein the MUC1-C antigen is encoded by a sequence with at least 80% sequence identity to SEQ ID NO: 5, SEQ ID NO: 6, or SEQ ID NO: 101; wherein the Brachyury antigen is encoded by a sequence with at least 80% sequence identity to SEQ ID NO: 9, SEQ ID NO: 10, or SEQ ID NO: 102; wherein the CEA antigen is encoded by a sequence with at least 80% sequence identity to SEQ ID NO: 1, SEQ ID NO: 2, or SEQ ID NO: 100.

Methods are also provided for treating or ameliorating the symptoms of any of the infectious diseases or cancers as described herein. The methods of treatment comprise administering the adenovirus vectors one or more times to individuals suffering from or at risk from suffering from an infectious disease or cancer as described herein. As such, some embodiments provide methods for vaccinating against infectious diseases or cancers in individuals who are at risk of developing such a disease. Individuals at risk may be individuals who may be exposed to an infectious agent at some time or have been previously exposed but do not yet have symptoms of infection or individuals having a genetic predisposition to developing a cancer or being particularly susceptible to an infectious agent. Individuals suffering from an infectious disease or cancer described herein may be determined to express and/or present a target antigen, which may be use to guide the therapies herein. For example, an example can be found to express and/or present a target antigen and an adenovirus vector encoding the target antigen, a variant, a fragment or a variant fragment thereof may be administered subsequently.

Some embodiments contemplate the use of adenovirus vectors for the in vivo delivery of nucleic acids encoding a target antigen, or a fragment, a variant, or a variant fragment thereof. Once injected into a subject, the nucleic acid sequence is expressed resulting in an immune response against the antigen encoded by the sequence. The adenovirus vector vaccine can be administered in an “effective amount”, that is, an amount of adenovirus vector that is effective in a selected route or routes of administration to elicit an immune response as described elsewhere herein. An effective amount can induce an immune response effective to facilitate protection or treatment of the host against the target infectious agent or cancer. The amount of vector in each vaccine dose is selected as an amount which induces an immune, immunoprotective or other immunotherapeutic response without significant adverse effects generally associated with typical vaccines. Once vaccinated, subjects may be monitored to determine the efficacy of the vaccine treatment. Monitoring the efficacy of vaccination may be performed by any method known to a person of ordinary skill in the art. In some embodiments, blood or fluid samples may be assayed to detect levels of antibodies. In other embodiments, ELISpot assays may be performed to detect a cell-mediated immune response from circulating blood cells or from lymphoid tissue cells.

Routes and frequency of administration of the therapeutic compositions described herein, as well as dosage, may vary from individual to individual, and from disease to disease, and may be readily established using standard techniques. In general, the pharmaceutical compositions and vaccines may be administered by injection (e.g., intracutaneous, intramuscular, intravenous or subcutaneous), intranasally (e.g., by aspiration), in pill form (e.g., swallowing, suppository for vaginal or rectal delivery). In certain embodiments, between 1 and 10 doses may be administered over a 52-week period. In certain embodiments, 6 doses are administered, at intervals of 1 month, and further booster vaccinations may be given periodically thereafter. Alternate protocols may be appropriate for individual patients. As such, 1, 2, 3, 4, 5, 6, 7, 8, 9, 10, 11, 12, 13, 14, 15, 16, 17, 18, 19, 20, or more doses may be administered over a 1 year period or over shorter or longer periods, such as over 35, 40, 45, 50, 55, 60, 65, 70, 75, 80, 85, 90, 95 or 100 week periods. Doses may be administered at 1, 2, 3, 4, 5, or 6 week intervals or longer intervals.

A vaccine can be infused over a period of less than about 4 hours, and more preferably, over a period of less than about 3 hours. For example, the first 25-50 mg could be infused within 30 minutes, preferably within 15 min, and the remainder infused over the next 2-3 hrs. More generally, the dosage of an administered vaccine construct may be administered as one dosage every 2 or 3 weeks, repeated for a total of at least 3 dosages. Or, the construct may be administered twice per week for 4-6 weeks. The dosing schedule can optionally be repeated at other intervals and dosage may be given through various parenteral routes, with appropriate adjustment of the dose and schedule. Compositions can be administered to a patient in conjunction with (e.g., before, simultaneously, or following) any number of relevant treatment modalities.

A suitable dose is an amount of an adenovirus vector that, when administered as described above, is capable of promoting a target antigen immune response as described elsewhere herein. In certain embodiments, the immune response is at least 10-50% above the basal (i.e., untreated) level. In certain embodiments, the immune response is at least 2, 3, 4, 5, 6, 7, 8, 9, 10, 12, 15, 20, 25, 30, 35, 40, 45, 50, 55, 60, 65, 70, 75, 80, 85, 90, 100, 110, 125, 150, 200, 250, 300, 400, 500, or more over the basal level. Such response can be monitored by measuring the target antigen(s) antibodies in a patient or by vaccine-dependent generation of cytolytic effector cells capable of killing patient tumor or infected cells in vitro, or other methods known in the art for monitoring immune responses. Such vaccines should also be capable of causing an immune response that leads to an improved clinical outcome of the disease in question in vaccinated patients as compared to non-vaccinated patients. In some embodiments, the improved clinical outcome comprises treating disease, reducing the symptoms of a disease, changing the progression of a disease, or extending life.

In general, an appropriate dosage and treatment regimen provides the adenovirus vectors in an amount sufficient to provide therapeutic and/or prophylactic benefit. Such a response can be monitored by establishing an improved clinical outcome for the particular disease being treated in treated patients as compared to non-treated patients. The monitoring data can be evaluated over time. The progression of a disease over time can be altered. Such improvements in clinical outcome would be readily recognized by a treating physician. Increases in preexisting immune responses to a target protein can generally correlate with an improved clinical outcome. Such immune responses may generally be evaluated using standard proliferation, cytotoxicity or cytokine assays, which may be performed using samples obtained from a patient before and after treatment.

While one advantage is the capability to administer multiple vaccinations with the same or different adenovirus vectors, particularly in individuals with preexisting immunity to Ad, the adenoviral vaccines may also be administered as part of a prime and boost regimen. A mixed modality priming and booster inoculation scheme may result in an enhanced immune response. Thus, one aspect is a method of priming a subject with a plasmid vaccine, such as a plasmid vector comprising a target antigen of interest, by administering the plasmid vaccine at least one time, allowing a predetermined length of time to pass, and then boosting by administering the adenovirus vector. Multiple primings, e.g., 1-4, may be employed, although more may be used. The length of time between priming and boost may typically vary from about four months to a year, but other time frames may be used. In certain embodiments, subjects may be primed 1, 2, 3, 4, 5, 6, 7, 8, 9, 10, or more times with plasmid vaccines, and then boosted 4 months later with the adenovirus vector.

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In some cases, the compositions provided herein are administered to a cell ex vivo. In some cases, the compositions provided herein are administered to an individual as a method of treating a disease or disorder. In some embodiments, the individual has a genetic disease. In some cases, the individual is at risk of having the disease, such as any of the diseases described herein. In some embodiments, the individual is at increased risk of having a disease or disorder caused by insufficient amount of a protein or insufficient activity of a protein. If an individual is “at an increased risk” of having a disease or disorder, the method involves preventative or prophylactic treatment. For example, an individual can be at an increased risk of having such a disease or disorder because of family history of the disease. Typically, individuals at an increased risk of having such a disease or disorder benefit from prophylactic treatment (e.g., by preventing or delaying the onset or progression of the disease or disorder).

In some cases, a subject does not have a disease. In some cases, the treatment is administered before onset of a disease. A subject may have undetected disease. A subject may have a low disease burden. A subject may also have a high disease burden. In certain cases, a subject may be administered a treatment as described herein according to a grading scale. A grading scale can be a Gleason classification. A Gleason classification reflects how different tumor tissue is from normal prostate tissue. It uses a scale from 1 to 5. A physician gives a cancer a number based on the patterns and growth of the cancer cells. The lower the number, the more normal the cancer cells look and the lower the grade. The higher the number, the less normal the cancer cells look and the higher the grade. In certain cases, a treatment may be administered to a patient with a low Gleason score. Particularly, a patient with a Gleason score of 3 or below may be administered a treatment as described herein. In some embodiments, the subject has a Gleason score of 6 or less. In some embodiments, the subject has a Gleason score greater than 6.

Various embodiments relate to compositions and methods for raising an immune response against CEA antigens in selected patient populations. Accordingly, methods and compositions may target patients with a cancer including, but not limited to, carcinomas or sarcomas such as neurologic cancers, melanoma, non-Hodgkin's lymphoma, Hodgkin's disease, leukemia, plasmocytomas, adenomas, gliomas, thymomas, breast cancer, gastrointestinal cancer, prostate cancer, colorectal cancer, kidney cancer, renal cell carcinoma, uterine cancer, pancreatic cancer, esophageal cancer, lung cancer, ovarian cancer, cervical cancer, testicular cancer, gastric cancer, multiple myeloma, hepatoma, acute lymphoblastic leukemia (ALL), acute myelogenous leukemia (AML), chronic myelogenous leukemia (CML), and chronic lymphocytic leukemia (CLL), or other cancers can be targeted for therapy. In some cases, the targeted patient population may be limited to individuals having colorectal adenocarcinoma, metastatic colorectal cancer, advanced CEA expressing colorectal cancer, head and neck cancer, liver cancer, breast cancer, lung cancer, bladder cancer, or pancreas cancer. A histologically confirmed diagnosis of a selected cancer, for example colorectal adenocarcinoma, may be used. A particular disease stage or progression may be selected, for example, patients with one or more of a metastatic, recurrent, stage III, or stage IV cancer may be selected for therapy with the methods and compositions. In some embodiments, patients may be required to have received and, optionally, progressed through other therapies including but not limited to fluoropyrimidine, irinotecan, oxaliplatin, bevacizumab, cetuximab, or panitumumab containing therapies. In some cases, individual's refusal to accept such therapies may allow the patient to be included in a therapy eligible pool with methods and compositions. In some embodiments, individuals to receive therapy using the methods and compositions may be required to have an estimated life expectancy of at least, 1, 2, 3, 4, 5, 6, 7, 8, 9, 10, 11, 12, 14, 15, 18, 21, or 24 months. The patient pool to receive a therapy using the methods and compositions may be limited by age. For example, individuals who are older than 2, 3, 4, 5, 6, 7, 8, 9, 10, 11, 12, 13, 14, 15, 16, 17, 18, 19, 20, 21, 25, 30, 35, 40, 50, 60, or more years old can be eligible for therapy with methods and compositions. For another example, individuals who are younger than 75, 70, 65, 60, 55, 50, 40, 35, 30, 25, 20, or fewer years old can be eligible for therapy with methods and compositions.

In some embodiments, patients receiving therapy using the methods and compositions are limited to individuals with adequate hematologic function, for example with one or more of a WBC count of at least 1000, 1500, 2000, 2500, 3000, 3500, 4000, 4500, 5000 or more per microliter, a hemoglobin level of at least 5, 6, 7, 8, 9, 10, 11, 12, 13, 14 or higher g/dL, a platelet count of at least 50,000; 60,000; 70,000; 75,000; 90,000; 100,000; 110,000; 120,000; 130,000; 140,000; 150,000 or more per microliter; with a PT-INR value of less than or equal to 0.8, 1.0, 1.2, 1.3, 1.4, 1.5, 1.6, 1.8, 2.0, 2.5, 3.0, or higher, a PTT value of less than or equal to 1.2, 1.4, 1.5, 1.6, 1.8, 2.0×ULN or more. In various embodiments, hematologic function indicator limits are chosen differently for individuals in different gender and age groups, for example 0-5, 5-10, 10-15, 15-18, 18-21, 21-30, 30-40, 40-50, 50-60, 60-70, 70-80 or older than 80.

In some embodiments, patients receiving therapy using the methods and compositions are limited to individuals with adequate renal and/or hepatic function, for example with one or more of a serum creatinine level of less than or equal to 0.8, 0.9, 1.0, 1.1, 1.2, 1.3, 1.4, 1.5, 1.6, 1.7, 1.8, 1.9, 2.0, 2.1, 2.2 mg/dL or more, a bilirubin level of 0.8, 0.9, 1.0, 1.1, 1.2, 1.3, 1.4, 1.5, 1.6, 1.7, 1.8, 1.9, 2.0, 2.1, 2.2 mg/dL or more, while allowing a higher limit for Gilbert's syndrome, for example, less than or equal to 1.5, 1.6, 1.8, 1.9, 2.0, 2.1, 2.2, 2.3, or 2.4 mg/dL, an ALT and AST value of less than or equal to less than or equal to 1.5, 2.0, 2.5, 3.0× upper limit of normal (ULN) or more. In various embodiments, renal or hepatic function indicator limits are chosen differently for individuals in different gender and age groups, for example 0-5, 5-10, 10-15, 15-18, 18-21, 21-30, 30-40, 40-50, 50-60, 60-70, 70-80 or older than 80.

In some embodiments, the K-ras mutation status of individuals who are candidates for a therapy using the methods and compositions as described herein can be determined. Individuals with a preselected K-ras mutational status can be included in an eligible patient pool for therapies using the methods and compositions as described herein.

In various embodiments, patients receiving therapy using the methods and compositions as described herein are limited to individuals without concurrent cytotoxic chemotherapy or radiation therapy, a history of, or current, brain metastases, a history of autoimmune disease, such as but not restricted to, inflammatory bowel disease, systemic lupus erythematosus, ankylosing spondylitis, scleroderma, multiple sclerosis, thyroid disease and vitiligo, serious intercurrent chronic or acute illness, such as cardiac disease (NYHA class III or IV), or hepatic disease, a medical or psychological impediment to probable compliance with the protocol, concurrent (or within the last 5 years) second malignancy other than non-melanoma skin cancer, cervical carcinoma in situ, controlled superficial bladder cancer, or other carcinoma in situ that has been treated, an active acute or chronic infection including: a urinary tract infection, HIV (e.g., as determined by ELISA and confirmed by Western Blot), and chronic hepatitis, or concurrent steroid therapy (or other immuno-suppressives, such as azathioprine or cyclosporin A). In some cases, patients with at least 3, 4, 5, 6, 7, 8, 9, or 10 weeks of discontinuation of any steroid therapy (except that used as pre-medication for chemotherapy or contrast-enhanced studies) may be included in a pool of eligible individuals for therapy using the methods and compositions as described herein.

In some embodiments, patients receiving therapy using the methods and compositions as described herein include individuals with thyroid disease and vitiligo.

In various embodiments, samples, for example serum or urine samples, from the individuals or candidate individuals for a therapy using the methods and compositions as described herein may be collected. Samples may be collected before, during, and/or after the therapy for example, within 2, 4, 6, 8, 10 weeks prior to the start of the therapy, within 1 week, 10 day, 2 weeks, 3 weeks, 4 weeks, 6 weeks, 8 weeks, or 12 weeks from the start of the therapy, within 2, 4, 6, 8, 10 weeks prior to the start of the therapy, within 1 week, 10 day, 2 weeks, 3 weeks, 4 weeks, 6 weeks, 8 weeks, 9 weeks, or 12 weeks from the start of the therapy, in 1 week, 10 day, 2 weeks, 3 weeks, 4 weeks, 6 weeks, 8 weeks, 9 weeks, or 12 weeks intervals during the therapy, in 1 month, 3 month, 6 month, 1 year, 2 year intervals after the therapy, within 1 month, 3 months, 6 months, 1 year, 2 years, or longer after the therapy, for a duration of 6 months, 1, 2, 3, 4, 5, 6, 7, 8, 9, 10 years or longer. The samples may be tested for any of the hematologic, renal, or hepatic function indicators described herein as well as suitable others known in the art, for example a 8-HCG for women with childbearing potential. In that regard, hematologic and biochemical tests, including cell blood counts with differential, PT, INR and PTT, tests measuring Na, K, Cl, CO₂, BUN, creatinine, Ca, total protein, albumin, total bilirubin, alkaline phosphatase, AST, ALT and glucose may be used in some embodiments. In some embodiments, the presence or the amount of HIV antibody, Hepatitis BsAg, or Hepatitis C antibody are determined in a sample from individuals or candidate individuals for a therapy using the methods and compositions as described herein. Biological markers, such as antibodies to CEA or the neutralizing antibodies to Ad5 vector can be tested in a sample, such as serum, from individuals or candidate individuals for a therapy using the methods and compositions as described herein. In some cases, one or more samples, such as a blood sample can be collected and archived from an individuals or candidate individuals for a therapy using the methods and compositions as described herein. Collected samples can be assayed for immunologic evaluation. Individuals or candidate individuals for a therapy using the methods and compositions as described herein can be evaluated in imaging studies, for example using CT scans or MRI of the chest, abdomen, or pelvis. Imaging studies can be performed before, during, or after therapy using the methods and compositions as described herein, during, and/or after the therapy, for example, within 2, 4, 6, 8, 10 weeks prior to the start of the therapy, within 1 week, 10 day, 2 weeks, 3 weeks, 4 weeks, 6 weeks, 8 weeks, or 12 weeks from the start of the therapy, within 2, 4, 6, 8, 10 weeks prior to the start of the therapy, within 1 week, 10 day, 2 weeks, 3 weeks, 4 weeks, 6 weeks, 8 weeks, 9 weeks, or 12 weeks from the start of the therapy, in 1 week, 10 day, 2 week, 3 week, 4 week, 6 week, 8 week, 9 week, or 12 week intervals during the therapy, in 1 month, 3 month, 6 month, 1 year, 2 year intervals after the therapy, within 1 month, 3 months, 6 months, 1 year, 2 years, or longer after the therapy, for a duration of 6 months, 1, 2, 3, 4, 5, 6, 7, 8, 9, 10 years or longer.

With regard to treatment of a condition with Ad5 vectors encoding for CEA, MUC1-C, and Brachyury, in one aspect, a method of generating an immune response in a human to each antigen, or any combination thereof is provided comprising administering to the human the composition. In some embodiments, the administering step is repeated at least once. In some embodiments, the administering step is repeated after about 2, 3, 4, 5, or 6 weeks following a previous administering step. In some embodiments, the administering step is repeated after about 2, 3, 4, 5, or 6 months following a previous administering step. In some embodiments, the administering step is repeated twice.

In one aspect, a method of treatment is provided comprising: selecting a first phase of treatment and a second phase of treatment; during the first phase, administering to a human a total of 3 times, in about 3 week intervals, a first composition comprising a first replication defective adenovirus vector encoding a MUC1-C antigen; and during the second phase, administering to the human a total of 3 times, in about 3 month intervals, a second composition comprising a second replication defective adenovirus vector encoding an antigen that induces an immune response in a human against cells expressing the MUC1-C antigen.

In one aspect, a method of treatment is provided comprising: selecting a first phase and a second phase of treatment; during the first phase, administering to a human a total of 3 times, in about 3 week intervals, a first composition comprising a first replication defective adenovirus vector encoding a Brachyury antigen; and during the second phase, administering to the human a total of 3 times, in about 3 month intervals, a second composition comprising a second replication defective adenovirus vector encoding an antigen that induces an immune response in a human against cells expressing the Brachyury antigen.

In one aspect, a method of treatment is provided comprising: selecting a first phase of treatment and a second phase of treatment; during the first phase, administering to a human a total of 3 times, in about 3 week intervals, a first composition comprising a first replication defective adenovirus vector encoding at least two antigens selected from the group consisting of a MUC1-C antigen, a Brachyury antigen, and a CEA antigen; and during the second phase, administering to the human a total of 3 times, in about 3 month intervals, a second composition comprising a second replication defective adenovirus vector encoding an antigen that induces an immune response in a human against cells expressing the at least two antigens. In some embodiments, the second phase starts about 3 months after the end of the first phase.

In one aspect, a method of treatment is provided comprising: selecting a first phase of treatment and a second phase of treatment; during the first phase, administering to a human, a total of n times, a first composition comprising a first replication defective adenovirus vector encoding a Brachyury antigen; during the second phase, administering the human, a total of m times, a second composition comprising a second replication defective adenovirus vector encoding an antigen that induces an immune response in a human against cells expressing the Brachyury antigen.

In one aspect, a method of treatment is provided comprising: selecting a first phase of treatment and a second phase of treatment; during the first phase, administering to a human, a total of n times, a first composition comprising a first replication defective adenovirus vector encoding a MUC1-C antigen; during the second phase, administering the human, a total of m times, a second composition comprising a second replication defective adenovirus vector encoding an antigen that induces an immune response in a human against cells expressing the MUC1-C antigen.

In one aspect, a method of treatment is provided comprising: selecting a first phase of treatment and a second phase of treatment; during the first phase, administering to a human, a total of n times, a first composition comprising a first replication defective adenovirus vector encoding at least two antigens selected from the group consisting of a MUC1-C antigen, a Brachyury antigen, and a CEA antigen; during the second phase, administering the human, a total of m times, a second composition comprising a second replication defective adenovirus vector encoding the at least two antigens that induces an immune response in a human against cells expressing the at least two antigens. In some embodiments, n is greater than 1. In some embodiments, n is 3. In some embodiments, m is greater than 1. In some embodiments, m is 3. In some embodiments, the first phase is at least 2, 3, 4, 5, 6, 7, or 8 weeks. In some embodiments, the second phase is at least 2, 3, 4, 5, 6, 7, or 8 months. In some embodiments, the second phase starts 3-16 weeks after first phase ends. In some embodiments, in the first phase two administrations of the replication defective adenovirus are at least 18 days apart. In some embodiments, in the first phase two administrations of the replication defective adenovirus are about 21 days apart. In some embodiments, in the first phase two administrations of the replication defective adenovirus are at most 24 days apart. In some embodiments, in the second phase two administrations of the replication defective adenovirus are at least 10 weeks apart. In some embodiments, in the second phase two administrations of the replication defective adenovirus are about 13 weeks apart. In some embodiments, in the second phase two administrations of the replication defective adenovirus are at most 16 weeks apart. In some embodiments, the method further comprises administering a molecular composition comprising an immune pathway checkpoint modulator.

In one aspect, a method of treatment is provided comprising: selecting a first phase of treatment and a second phase of treatment; during the first phase, administering to a human, a total of n times, a first composition comprising a first replication defective adenovirus vector encoding an antigen that induces an immune response in a human against cells expressing a MUC1-C, Brachyury, or CEA antigen; and during the second phase, administering the human, a total of m times, a second composition comprising a second replication defective adenovirus vector encoding an antigen that is capable of inducing an immune response directed towards cells expressing MUC1-C, Brachyury, or CEA antigen in a human; wherein a molecular composition comprising and an immune pathway checkpoint modulator is administered during the first phase, the second phase, or both.

In one aspect, a method of treating a subject in need thereof is provided, comprising administering to the subject: (a) a recombinant replication deficient adenovirus vector encoding (i) a MUC1-C antigen, (ii) a Brachyury antigen, or (iii) at least two antigens selected from the group consisting of a MUC1-C antigen, a Brachyury antigen, and a CEA antigen; and (b) a molecular composition comprising an immune pathway checkpoint modulator; thereby generating an immune response in the subject. In some embodiments, (a) and (b) are administered in series. In some embodiments, (a) and (b) are administered at the same time. In some embodiments, (a) and (b) are administered a month apart.

### Dosages and Administration of Ad5 Vaccines

Compositions and methods as described herein contemplate various dosage and administration regimens during therapy. Patients may receive one or more replication defective adenovirus or adenovirus vector, for example Ad5 [E1−, E2B−]-CEA(6D), that is capable of raising an immune response in an individual against a target antigen described herein. Patients can also receive one or more replication defective adenovirus or adenovirus vector, for example Ad5 [E1−, E2B−]-CEA(6D), Ad5 [E1−, E2b−]-MUC1, Ad5 [E1−, E2b−]-MUC1c, Ad5 [E1−, E2b−]-MUC1n, or Ad5 [E1−, E2b−]-T (i.e., Ad5 [E1−, E2b−]-Brachyury) that is capable of raising an immune response in an individual against a target antigen described herein. In various embodiments, the replication defective adenovirus is administered at a dose that suitable for effecting such immune response. In some cases, the replication defective adenovirus is administered at a dose that is greater than or equal to 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×¹⁰, 6×¹⁰, 7×¹⁰, 8×¹⁰, 9×¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 3×10¹², 4×10¹², 5×10¹² or more virus particles (VP) per immunization. In some cases, the replication defective adenovirus is administered at a dose that is less than or equal to 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 3×10¹², 4×10¹², 5×10¹², or more virus particles per immunization. In some embodiments, the replication defective adenovirus is administered at a dose of 1×10⁹-5×10¹² virus particles per immunization. In some embodiments, the composition comprises at least 1.0×10¹¹, 2.0×10¹¹, 3.0×10¹¹, 3.5×10¹¹, 4.0×10¹¹, 4.5×10¹¹, 4.8×10¹¹, 4.9×10¹¹, 4.95×10¹¹, or 4.99×10¹¹ virus particles comprising the recombinant nucleic acid vector. In some embodiments, the composition comprises at most 7.0×10¹¹, 6.5×10¹¹, 6.0×10¹¹, 5.5×10¹¹, 5.2×10¹¹, 5.1×10¹¹, 5.05×10¹¹, or 5.01×10¹¹ virus particles. In some embodiments, the composition comprises 1.0×10¹¹-7.0×10¹¹ or 1.0-5.5×10¹¹ virus particles. In some embodiments, the composition comprises 4.5×10¹¹-5.5×10¹¹ virus particles. In some embodiments, the composition comprises 4.8×10¹¹-5.2×10¹¹ virus particles. In some embodiments, the composition comprises 4.9×10¹¹-5.1×10¹¹ virus particles. In some embodiments, the composition comprises 4.95×10¹¹-5.05×10¹¹ virus particles. In some embodiments, the composition comprises 4.99×10¹¹-5.01×10¹¹ virus particles.

In various embodiments, a desired dose described herein is administered in a suitable volume of formulation buffer, for example a volume of about 0.1-10 mL, 0.2-8 mL, 0.3-7 mL, 0.4-6 mL, 0.5-5 mL, 0.6-4 mL, 0.7-3 mL, 0.8-2 mL, 0.9-1.5 mL, 0.95-1.2 mL, or 1.0-1.1 mL. Those of skill in the art appreciate that the volume may fall within any range bounded by any of these values (e.g., about 0.5 mL to about 1.1 mL). Administration of virus particles can be through a variety of suitable paths for delivery, for example it can be by injection (e.g., intradermally, intracutaneously, intramuscularly, intravenously or subcutaneously), intranasally (e.g., by aspiration), in pill form (e.g., swallowing, suppository for vaginal or rectal delivery. In some embodiments, a subcutaneous delivery may be preferred and can offer greater access to dendritic cells.

Administration of virus particles to an individual may be repeated. Repeated deliveries of virus particles may follow a schedule or alternatively, may be performed on an as needed basis. For example, an individual's immunity against a target antigen, for example CEA, may be tested and replenished as necessary with additional deliveries. In some embodiments, schedules for delivery include administrations of virus particles at regular intervals. Joint delivery regimens may be designed comprising one or more of a period with a schedule and/or a period of need based administration assessed prior to administration. For example, a therapy regimen may include an administration, such as subcutaneous administration once every three weeks then another immunotherapy treatment every three months until removed from therapy for any reason including death. Another example regimen comprises three administrations every three weeks then another set of three immunotherapy treatments every three months. Another example regimen comprises a first period with a first number of administrations at a first frequency, a second period with a second number of administrations at a second frequency, a third period with a third number of administrations at a third frequency, etc., and optionally one or more periods with undetermined number of administrations on an as needed basis. The number of administrations in each period can be independently selected and can for example be 1, 2, 3, 4, 5, 6, 7, 8, 9, 10, 11, 12, 13, 14, 15, 16, 17, 18, 19, 20 or more. The frequency of the administration in each period can also be independently selected, can for example be about every day, every other day, every third day, twice a week, once a week, once every other week, every three weeks, every month, every six weeks, every other month, every third month, every fourth month, every fifth month, every sixth month, once a year etc. The therapy can take a total period of up to 1, 2, 3, 4, 5, 6, 7, 8, 9, 10, 11, 12, 13, 14, 15, 16, 17, 18, 19, 20, 21, 22, 23, 24, 30, 36 months or more. The scheduled interval between immunizations may be modified so that the interval between immunizations is revised by up to a fifth, a fourth, a third, or half of the interval. For example, for a 3-week interval schedule, an immunization may be repeated between 20 and 28 days (3 weeks−1 day to 3 weeks+7 days). For the first 3 immunizations, if the second and/or third immunization is delayed, the subsequent immunizations may be shifted allowing a minimum amount of buffer between immunizations. For example, for a three week interval schedule, if an immunization is delayed, the subsequent immunization may be scheduled to occur no earlier than 17, 18, 19, or 20 days after the previous immunization.

Compositions, such as Ad5 [E1−, E2B−]-CEA(6D) virus particles, can be provided in various states, for example, at room temperature, on ice, or frozen. Compositions may be provided in a container of a suitable size, for example a vial of 2 mL vial. In one embodiment, a 2-ml vial with 1.0 mL of extractable vaccine contains 5×10¹¹ total virus particles/mL. Storage conditions including temperature and humidity may vary. For example, compositions for use in therapy may be stored at room temperature, 4° C., −20° C., or lower.

In various embodiments, general evaluations are performed on the individuals receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization.

General evaluations may include one or more of medical history, ECOG Performance Score, Karnofsky performance status, and complete physical examination with weight by the attending physician. Any other treatments, medications, biologics, or blood products that the patient is receiving or has received since the last visit may be recorded. Patients may be followed at the clinic for a suitable period, for example approximately 30 minutes, following receipt of vaccine to monitor for any adverse reactions. Local and systemic reactogenicity after each dose of vaccine will may be assessed daily for a selected time, for example for 3 days (on the day of immunization and 2 days thereafter). Diary cards may be used to report symptoms and a ruler may be used to measure local reactogenicity. Immunization injection sites may be assessed. CT scans or MRI of the chest, abdomen, and pelvis may be performed.

In various embodiments, hematological and biochemical evaluations are performed on the individuals receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization. Hematological and biochemical evaluations may include one or more of blood test for chemistry and hematology, CBC with differential, Na, K, Cl, CO₂, BUN, creatinine, Ca, total protein, albumin, total bilirubin, alkaline phosphatase, AST, ALT, glucose, and ANA

In various embodiments, biological markers are evaluated on individuals receiving treatment according to the methods and compositions as described herein. One or more of any tests may be performed as needed or in a scheduled basis, such as on weeks 0, 3, 6 etc. A different set of tests may be performed concurrent with immunization vs. at time points without immunization.

Biological marker evaluations may include one or more of measuring antibodies to CEA or the Ad5 vector, from a serum sample of adequate volume, for example about 5 ml Biomarkers (e.g., CEA or CA15-3) may be reviewed if determined and available.

In various embodiments, an immunological assessment is performed on individuals receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization.

Peripheral blood, for example about 90 mL may be drawn prior to each immunization and at a time after at least some of the immunizations, to determine whether there is an effect on the immune response at specific time points during the study and/or after a specific number of immunizations. Immunological assessment may include one or more of assaying peripheral blood mononuclear cells (PBMC) for T-cell responses to CEA using ELISpot, proliferation assays, multi-parameter flow cytometric analysis, and cytoxicity assays. Serum from each blood draw may be archived and sent and determined.

In various embodiments, a tumor assessment is performed on individuals receiving treatment according to the methods and compositions as described herein. One or more of any tests may be performed as needed or in a scheduled basis, such as prior to treatment, on weeks 0, 3, 6 etc. A different set of tests may be performed concurrent with immunization vs. at time points without immunization. Tumor assessment may include one or more of CT or MRI scans of chest, abdomen, or pelvis performed prior to treatment, at a time after at least some of the immunizations and at approximately every three months following the completion of a selected number, for example 2, 3, or 4, of first treatments and for example until removal from treatment.

Immune responses against a target antigen described herein, such as CEA, may be evaluated from a sample, such as a peripheral blood sample of an individual using one or more suitable tests for immune response, such as ELISpot, cytokine flow cytometry, or antibody response. A positive immune response can be determined by measuring a T-cell response. A T-cell response can be considered positive if the mean number of spots adjusted for background in six wells with antigen exceeds the number of spots in six control wells by 10 and the difference between single values of the six wells containing antigen and the six control wells is statistically significant at a level of p≤0.05 using the Student's t-test. Immunogenicity assays may occur prior to each immunization and at scheduled time points during the period of the treatment. For example, a time point for an immunogenicity assay at around week 1, 2, 3, 4, 5, 6, 7, 8, 9, 10, 11, 12, 13, 14, 15, 18, 20, 24, 30, 36, or 48 of a treatment may be scheduled even without a scheduled immunization at this time. In some cases, an individual may be considered evaluable for immune response if they receive at least a minimum number of immunizations, for example 1, 2, 3, 4, 5, 6, 7, 8, 9, or more immunizations.

In some embodiments, disease progression or clinical response determination is made according to the RECIST 1.1 criteria among patients with measurable/evaluable disease. In some embodiments, therapies using the methods and compositions as described herein affect a Complete Response (CR; disappearance of all target lesions for target lesions or disappearance of all non-target lesions and normalization of tumor marker level for non-target lesions) in an individual receiving the therapy. In some embodiments, therapies using the methods and compositions affect a Partial Response (PR; at least a 30% decrease in the sum of the LD of target lesions, taking as reference the baseline sum LD for target lesions) in an individual receiving the therapy.

In some embodiments, therapies using the methods and compositions affect a Stable Disease (SD; neither sufficient shrinkage to qualify for PR nor sufficient increase to qualify for PD, taking as reference the smallest sum LD since the treatment started for target lesions) in an individual receiving the therapy. In some embodiments, therapies using the methods and compositions as described herein affect an Incomplete Response/Stable Disease (SD; persistence of one or more non-target lesion(s) or/and maintenance of tumor marker level above the normal limits for non-target lesions) in an individual receiving the therapy. In some embodiments, therapies using the methods and compositions as described herein affect a Progressive Disease (PD; at least a 20% increase in the sum of the LD of target lesions, taking as reference the smallest sum LD recorded since the treatment started or the appearance of one or more new lesions for target lesions or persistence of one or more non-target lesion(s) or/and maintenance of tumor marker level above the normal limits for non-target lesions) in an individual receiving the therapy.

### Kits for Combination Therapy Using Ad5 Vaccines Comprising Antigen-Calreticulin Fusions

The compositions, immunotherapy, or vaccines may be supplied in the form of a kit. Certain embodiments provide compositions, methods and kits for generating an immune response in an individual to fight infectious diseases and cancer. Certain embodiments provide compositions, methods and kits for generating an immune response against a target antigen or cells expressing or presenting a target antigen or a target antigen signature comprising at least one target antigen. The kits may further comprise instructions regarding the dosage and or administration including treatment regimen information. In some embodiments, the instructions are for the treatment of a proliferative disease or cancer. In some embodiments, the instructions are for the treatment of an infectious disease.

In some embodiments, kits comprise the compositions and methods for providing combination Ad5-CEA-CRT vaccines. In some embodiment's kits may further comprise components useful in administering the kit components and instructions on how to prepare the components. In some embodiments, the kit can further comprise software for conducting monitoring patient before and after treatment with appropriate laboratory tests, or communicating results and patient data with medical staff. In some embodiments, the kit comprises multiple effective doses of Ad5[E1−, E2b−]-CEA-CRT vaccines.

In one aspect, a kit for inducing an immune response in a human is provided comprising: a composition comprising a therapeutic solution of a volume in the range of 0.8-1.2 mL, the therapeutic solution comprising at least 1.0×10¹¹ virus particles; wherein the virus particles comprise a recombinant replication defective adenovirus vector; a composition comprising of a therapeutic solution of a molecular composition comprising an immune pathway checkpoint modulator and; instructions.

In some embodiments, the therapeutic solution comprises 1.0×10¹¹-5.5×10¹¹ virus particles. In some embodiments, adenovirus vector is capable of effecting overexpression of the modified CEA in transfected cells. In some embodiments, therapeutic solution comprises a first, second and third replication defective adenovirus vector each comprising an antigen selected from the group consisting of a CEA antigen, and combinations thereof. In some embodiments, the adenovirus vector comprises a nucleic acid sequence encoding an antigen that induces a specific immune response against CEA expressing cells in a human.

In some embodiments, the kit further comprises an immunogenic component. In some embodiments, the immunogenic component comprises a cytokine selected from the group of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-0, CD40 ligand, Fas ligand, CD27 ligand, CD30 ligand, 4-1BBL, Trail, OPG-L, APRIL, LIGHT, TWEAK, BAFF, TGF-β1, and MIF. In some embodiments, the immunogenic component is selected from the group consisting of IL-7, a nucleic acid encoding IL-7, a protein with substantial identity to IL-7, and a nucleic acid encoding a protein with substantial identity to IL-7. In some embodiments, the kit further comprises IL-15, a nucleic acid encoding for IL-15, a protein with substantial identity to IL-14, or a nucleic acid encoding a protein with substantial identity to IL-15.

The components comprising the kit may be in dry or liquid form. If they are in dry form, the kit may include a solution to solubilize the dried material. The kit may also include transfer factor in liquid or dry form. If the transfer factor is in dry form, the kit will include a solution to solubilize the transfer factor. The kit may also include containers for mixing and preparing the components. The kit may also include instrument for assisting with the administration such for example needles, tubing, applicator, inhalant, syringe, pipette, forceps, measured spoon, eye dropper or any such medically approved delivery vehicle. In some embodiments, the kits or drug delivery systems as described herein also include a means for containing compositions disclosed herein in close confinement for commercial sale and distribution.

### EXAMPLES

The following examples are included to further describe some aspects of the present disclosure, and should not be used to limit the scope of the invention.

### Example 1

### Peptides and Vectors

This example describes peptides and vectors. The following HLA-A2 and HLA-A24 binding peptides were used in this and other examples: (a) the HLA-A2 binding CEA agonist peptide CAP1-6D (YLSGADLNL). All peptides were greater than 96% pure.

Ad5 [E1−, E2b−]-CEA was constructed and produced. Briefly, the transgene was sub-cloned into the E1 region of the Ad5 [E1−, E2b−] vector using a homologous recombination-based approach. The replication deficient virus was propagated in the E.C7 packaging cell line, CsCl₂ purified, and titered. Viral infectious titer was determined as plaque-forming units (PFUs) on an E.C7 cell monolayer. The VP concentration was determined by sodium dodecyl sulfate (SDS) disruption and spectrophotometry at 260 nm and 280 nm. The CEA transgene also contained a modified CEA containing the highly immunogenic epitope CAP1-6D.

### Example 2

### GLP Production of Clinical Grade Multi-Targeted Vaccine

This example shows the production of clinical-grade multi-target vaccine using good laboratory practice (GLP) standards. Previously, the Ad5 [E1−, E2b−]-CEA(6D) product was produced using a 5 L Cell Bioreactor under GLP conditions in accordance with good manufacturing practice standards. This example shows that the Ad5 [E1−, E2b−]-mMUC1-C and the Ad5 [E1−, E2b−]-Brachyury products can be produced in a 5 L Cell Bioreactor using a similar approach.

Briefly, vials of the E.C7 manufacturing cell line are thawed, transferred into a T225 flasks, and initially cultured at 37° C. in 5% C02 in DMEM containing 10% FBS/4 mM L-glutamine. After expansion, the E.C7 cells will be expanded using 10-layered CellSTACKS (CS-10) and transitioned to FreeStyle serum-free medium (SFM). The E.C7 cells will be cultured in SFM for 24 hours at 37° C. in 5% C02 to a target density of 5×10⁵ cells/mL in the Cell Bioreactor. The E.C7 cells will then be infected with Ad5 [E1−, E2b−]-mMUC1-C or Ad5 [E1−, E2b−]-Brachyury, respectively, and cultured for 48 hours.

Mid-stream processing will be performed in an identical manner as that used to prepare clinical grade Ad5 [E1−, E2b−]-CEA(6D) product under IND14325. Thirty minutes before harvest, Benzonase nuclease will be added to the culture to promote better cell pelleting for concentration. After pelleting by centrifugation, the supernatant will be discarded and the pellets re-suspended in Lysis Buffer containing 1% Polysorbate-20 for 90 minutes at room temperature. The lysate will then be treated with Benzonase and the reaction quenched by addition of 5M NaCl. The slurry will be centrifuged and the pellet discarded. The lysate will be clarified by filtration and subjected to a two-column ion exchange procedure.

To purify the vaccine products, a two-column anion exchange procedure will be performed. A first column will be packed with Q Sepharose XL resin, sanitized, and equilibrated with loading buffer. The clarified lysate will be loaded onto the column and washed with loading buffer. The vaccine product will be eluted and the main elution peak (eluate) containing Ad5 [E1−, E2b−]-mMUC1-C or Ad5 [E1−, E2b−]-Brachyury is carried forward to the next step. A second column will be packed with Source 15Q resin, sanitized, and equilibrated with loading buffer. The eluate from the first anion exchange column will be loaded onto the second column and the vaccine product eluted with a gradient starting at 100% Buffer A (20 mM Tris, 1 mM MgCl₂, pH 8.0) running to 50% Buffer B (20 mM Tris, 1 mM MgCl₂, 2M NaCl, pH 8.0). The elution peak containing Ad5 [E1−, E2b−]-mMUC1-C or Ad5 [E1−, E2b−]-Brachyury will be collected and stored overnight at 2-8° C. The peak elution fraction will be processed through a tangential flow filtration (TFF) system for concentration and diafiltration against formulation buffer (20 mM Tris, 25 mM NaCl, 2.5% (v/v) glycerol, pH 8.0). After processing, the final vaccine product will be sterile filtered, dispensed into aliquots, and stored at ≤−60° C. A highly purified product approaching 100% purity is typically produced and similar results for these products are predicted.

The concentration and total number of VP product produced will be determined spectrophotometrically. Product purity is assessed by HPLC. Infectious activity is determined by performing an Ad5 hexon-staining assay for infectious particles using kits.

Western blots will be performed using lysates from vector transfected A549 cells to verify mMUC1-C or Brachyury expression. Quality control tests will be performed to determine that the final vaccine products are Mycoplasma-free, have no microbial bioburden, and exhibit endotoxin levels less than 2.5 endotoxin units (EU) per mL. To confirm immunogenicity, the individual vectors will tested in mice as described below (Example 8).

### Example 3

### Treatment of Cancer with Ad5 [E1− E2b−]-CEA(6D)-CRT Vaccine

This example describes treatment of cancer in a subject in need thereof with Ad5 [E1−, E2b−]-CEA(6D)-calreticulin (CRT) vaccine. Subjects with CEA-expressing tumors are immunized with the Ad5[E1−, E2b−]-CEA-CRT vaccine. The Ad5[E1−, E2b−]-CEA-CRT vaccine is administered at a dose of 5×10¹¹ virus particles (VPs) by subcutaneous (SC) injection. Vaccinations are repeated up to 3 times total over a 3-week period. The Ad5[E−, E2b−]-CEA-CRT vaccine is administered on days 7, 14, and 21, respectively.

Subjects in need thereof have CEA-expressing cancer cells, such as CEA-expressing colorectal cancer. Subjects are any mammal, such as a human or a non-human primate.

### Example 4

### Treatment of Cancer with Ad5 [E1− E2b−]-CEA(6D)-CRT Vaccine in Combination with Engineered NK Cells

This example describes treatment of cancer in a subject in need thereof with Ad5 [E1−, E2b−]-CEA(6D)-calreticulin (CRT) vaccine in combination with engineered NK cells. Subjects with CEA-expressing tumors are immunized with the Ad5[E1−, E2b−]-CEA-CRT vaccine. The Ad5[E1−, E2b−]-CEA-CRT vaccine is administered at a dose of 5×10¹¹ virus particles (VPs) by subcutaneous (SC) injection. The Ad5[E1−, E2b−]-CEA-CRT vaccine is administered on days 7, 14, and 21, respectively.

Subjects are additionally administered aNK cells. aNK cells are infused intravenously on days 9, 11, 18, 22, 27, and 33 at a dose of 2×10⁹ cells per treatment. Subjects in need thereof have CEA-expressing cancer cells, such as colorectal cancer. Subjects are any mammal, such as a human or a non-human primate.

### Example 5

### Treatment of Cancer with Ad5 [E1− E2b−]-CEA(6D)-CRT Vaccine in Combination with an Anti-CEA Antibody

This example describes treatment of cancer in a subject in need thereof with Ad5 [E1−, E2b−]-CEA(6D)-calreticulin (CRT) vaccine in combination with an anti-CEA antibody. Subjects with CEA-expressing tumors are immunized with the Ad5[E1−, E2b−]-CEA-CRT vaccine. The Ad5[E1−, E2b−]-CEA-CRT vaccine is administered at a dose of 5×10¹¹ virus particles (VPs) by subcutaneous (SC) injection. The Ad5[E1−, E2b−]-CEA-CRT vaccine is administered on days 7, 14, and 21, respectively.

Subjects are additionally administered an anti-CEA antibody, such as a NEO-201 antibody. NEO-201 antibody is infused in subjects at a dose of 3 mg/kg administered IV every on days 1, 15, and 22 after infusions with haNK cells delivered to patients above. This occurs over a 2 to 3-month period. Subjects in need thereof have CEA-expressing cancer cells, such as colorectal cancer. Subjects are any mammal, such as a human or a non-human primate.

### Example 6

### Treatment of Cancer with Ad5 [E1−, E2b−]-CEA(6D)-CRT Vaccine in Combination with FOLFOX-B, Avelumab, and NK Cell Therapy

This example describes treatment of cancer with Ad5 [E1−, E2b−]-CEA(6D)-calreticulin (CRT) vaccine in combination with FOLFOX-B, Avelumab, NEO-201 antibody, and NK cell therapy. Subjects with CEA-expressing tumors are immunized with the Ad5[E1−, E2b−]-CEA-CRT vaccine. The Ad5[E1−, E2b−]-CEA-CRT vaccine is administered at a dose of 5×10¹¹ virus particles (VPs) by subcutaneous (SC) injection. Vaccinations are repeated up to 3 times total over a 3-week period. The Ad5[E1−, E2b−]-CEA-CRT vaccine is administered on days 7, 14, and 21, respectively.

Anti-PD-1 monoclonal antibody, a checkpoint inhibitor, is (avelumab) infused in in order to enhance the vaccine effect. As a routine precaution, subjects enrolled in this trial are observed for 1 hour post infusion, in an area with resuscitation equipment and emergency agents. At all times during avelumab treatment, immediate emergency treatment of an infusion-related reaction or a severe hypersensitivity reaction according to institutional standards must be assured. In order to treat possible anaphylactic reactions, for instance, dexamethasone 10 mg and epinephrine in a 1:1000 dilution or equivalents are available along with equipment for assisted ventilation. Subjects receive intravenous infusion of avelumab over 1 hour (−10 minutes/+20 minutes, i.e., 50 to 80 minutes) as applicable at a dose of 10 mg/kg. Treatment with avelumab starts on the second vaccine treatment 3 weeks after the first vaccine injection. An immune response against the CEA tumor-associated antigens (TAAs) is induced and then enhanced by injections with anti-PD-1 that will interfere with the inhibitory effect of the immune checkpoint pathway. Anti-PD-1 antibody is injected into subjects at a dose of 3 mg/kg administered IV after a vaccination beginning on week 3. This infusion (injection) procedure is repeated on weeks 9 and 12.

Following Avelumab administration, FOLFOX therapy is administered intravenously. Oxaliplatin 85 mg/m² is administered IV over 2 hours on day 1 or 2, Leucovorin* 400 mg/m² is administered IV over 2 hours on day 1 or 2, 5-FU* 400 mg/m² is administered IV bolus on day 1 or 2, and 5-FU* 2400 mg/m² is administered IV over 46 hours to start on day 1 or 2. 5-Fluorouracil and leucovorin should be administered separately to avoid the formation of a precipitate. Per package insert, leucovorin is administered first.

Engineered NK cells, specifically aNK cells, are infused on days 9, 11, 18, 22, 27, and 33 at a dose of 2×10⁹ cells per treatment.

A NEO-201 antibody is infused in subjects at a dose of 3 mg/kg administered IV every on days 1, 15, and 22 after infusions with haNK cells delivered to patients above. This occurs over a 2 to 3-month period.

A subject in need thereof has any stage of disease progression, including metastatic colorectal cancer or advanced stage colorectal cancer. Subjects are any mammal, such as a human or a non-human primate. Administration is performed intravenously by infusion or subcutaneously. Administration of each therapy is given or days, weeks, or months. Therapies are administered once or multiple types, depending on the agent being delivered.

### Example 7

### Treatment of Cancer with Ad5 [E1−, E2b−]-CEA(6D)-CRT Vaccine in Combination with Ad5 [E1−, E2b−]-Brachyury-CRT and Ad5 [E1−, E2b−]-MUC1-CRT

This example describes treatment of cancer with Ad5 [E1−, E2b−]-CEA(6D)-calreticulin (CRT) vaccine in combination with Ad5 [E1−, E2b−]-Brachyury-CRT and Ad5 [E1−, E2b−]-MUC1-CRT. The following HLA-A2 and HLA-A24 binding peptides were used in this and other examples: (a) the HLA-A2 binding CEA agonist peptide CAP1-6D (YLSGADLNL), (b) the HLA-A2 MUC1 agonist peptide P93L (ALWGQDVTSV), (c) the HLA-A24 binding MUC1 agonist peptide C6A (KYHPMSEYAL), and (d) the HLA-A2 binding brachyury agonist peptide (WLLPGTSTV). All peptides were greater than 96% pure. Ad5 [E1−, E2b−]-Brachyury-CRT, Ad5 [E1−, E2b−]-CEA-CRT and Ad5 [E1−, E2b−]-MUC1-CRT were constructed and produced. Constructs were designed such that each of the antigens was followed by a nucleic acid sequence encoding for calreticulin (CRT) to generate the CEA-CRT, Brachyury-CRT, and MUC1-CRT inserts. Briefly, the transgenes were sub-cloned into the E1 region of the Ad5 [E1−, E2b−] vector using a homologous recombination-based approach. The replication deficient virus was propagated in the E.C7 packaging cell line, CsCl₂ purified, and titered. Viral infectious titer was determined as plaque-forming units (PFUs) on an E.C7 cell monolayer. The VP concentration was determined by sodium dodecyl sulfate (SDS) disruption and spectrophotometry at 260 nm and 280 nm. The CEA transgene also contained a modified CEA containing the highly immunogenic epitope CAP1-6D. The sequence encoding for the human Brachyury protein (T, NM_003181.3) was modified by introducing the enhancer T-cell HLA-A2 epitope (WLLPGTSTV; SEQ ID NO: 15) and removal of a 25-amino acid fragment involved in DNA binding. The resulting construct was subsequently subcloned into the Ad5 vector to generate the Ad5 [E1−, E2b−]-Brachyury-CRT construct. The MUC1 molecule consisted of two regions: the N-terminus (MUC1-n), which is the large extracellular domain of MUC1, and the C-terminus (MUC1-c), which has three regions: a small extracellular domain, a single transmembrane domain, and a cytoplasmic tail. The cytoplasmic tail contained sites for interaction with signaling proteins and acts as an oncogene and a driver of cancer motility, invasiveness and metastasis. For construction of the Ad5 [E1−, E2b−]-MUC1-CRT, the entire MUC1 transgene, including eight agonist epitopes, was subcloned into the Ad5 vector. The agonist epitopes included in the Ad5 [E1−, E2b−]-MUC1-CRT vector bind to HLA-A2 (epitope P93L in the N-terminus, V1A and V2A in the VNTR region, and C1A, C2A and C3A in the C-terminus), HLA-A3 (epitope C5A), and HLA-A24 (epitope C6A in the C-terminus). The Tri-Ad5 vaccine was produced by combining of 10¹⁰ VP of Ad5 [E1−, E2b−]-Brachyury-CRT, Ad5 [E1−, E2b−]-CEA-CRT and Ad5 [E1−, E2b−]-MUC1-CRT at a ratio of 1:1:1 (3×10¹⁰ VP total).

Subjects with CEA-expressing tumors are immunized by subcutaneous injection with a mixture of 5×10¹¹ virus particles (VPs) of the Ad5[E1−, E2b−]-CEA-CRT vaccine, 5×10¹¹ VPs of the Ad5[E1−, E2b−]-Brachyury-CRT vaccine, and 5×10¹¹ VPs of the Ad5[E1−, E2b−]-MUC1-CRT vaccine. Vaccinations are repeated up to 3 times total over a 3-week period. The Ad5[E1−, E2b−]-CEA-CRT, Ad5[E1−, E2b−]-Brachyury-CRT, Ad5[E1−, E2b−]-MUC1-CRT vaccine mixture is administered on days 7, 14, and 21, respectively. Subjects in need thereof have CEA-expressing cancer cells, such as CEA-expressing colorectal cancer. Subjects are any mammal, such as a human or a non-human primate.

### Example 8

### Treatment of Cancer with Ad5 [E1−, E2b−]-CEA(6D)-CRT Vaccine in Combination with a Checkpoint Inhibitor

This example describes treatment of cancer with Ad5 [E1−, E2b−]-CEA(6D)-calreticulin (CRT) vaccine in combination with a checkpoint inhibitor. Subjects with CEA-expressing tumors are immunized with the Ad5[E1−, E2b−]-CEA-CRT vaccine. The Ad5[E1−, E2b−]-CEA-CRT vaccine is administered at a dose of 5×10¹¹ virus particles (VPs) by subcutaneous (SC) injection. Vaccinations are repeated up to 3 times total over a 3-week period. The Ad5[E1−, E2b−]-CEA-CRT vaccine is administered on days 7, 14, and 21, respectively.

The checkpoint inhibitor administered in combination therapy is an anti-PD-1 monoclonal antibody, such as Avelumab. An anti-PD-1 monoclonal antibody (avelumab) is infused in in order to enhance the vaccine effect. As a routine precaution, subjects enrolled in this trial are observed for 1 hour post infusion, in an area with resuscitation equipment and emergency agents. At all times during avelumab treatment, immediate emergency treatment of an infusion-related reaction or a severe hypersensitivity reaction according to institutional standards must be assured. In order to treat possible anaphylactic reactions, for instance, dexamethasone 10 mg and epinephrine in a 1:1000 dilution or equivalents are available along with equipment for assisted ventilation. Subjects receive intravenous infusion of avelumab over 1 hour (−10 minutes/+20 minutes, i.e., 50 to 80 minutes) as applicable at a dose of 10 mg/kg. Treatment with avelumab starts on the second vaccine treatment 3 weeks after the first vaccine injection. An immune response against the CEA tumor-associated antigens (TAAs) is induced and then enhanced by injections with anti-PD-1 that will interfere with the inhibitory effect of the immune checkpoint pathway. Anti-PD-1 antibody is injected into subjects at a dose of 3 mg/kg administered IV after a vaccination beginning on week 3. This infusion (injection) procedure is repeated on weeks 9 and 12.

A subject in need thereof has any stage of disease progression, including metastatic colorectal cancer or advanced stage colorectal cancer. Subjects are any mammal, such as a human or a non-human primate. Administration is performed intravenously by infusion or subcutaneously. Administration of each therapy is given or days, weeks, or months. Therapies are administered once or multiple types, depending on the agent being delivered.

### Example 9

### Treatment of Cancer with Ad5 [E1−, E2b−]-Neo-Antigen-CRT Vaccine

This example describes treatment of cancer with an Ad5 [E1−, E2b−]-neo-antigen-calreticulin (CRT) vaccine. A tumor tissue sample is obtained from a subject in need of cancer treatment. The sample is analyze for identification of tumor neo-antigens or tumor neo-epitopes. Tumor neo-antigens are encoded for as a fusion with CRT in an Ad5 [E1−, E2b−] viral vector. The final vector is sequenced using next generation sequencing techniques in order to verify the neo-antigen and the CRT moieties. As shown in FIG. 1, the construct is cloned, transfected in EC.7 cells, purified, and concentrated. Ad5 [E1−, E2b−]-neo-antigen-CRT vectors are formulated for vaccination. Subjects in need thereof are vaccinated with a personalized neo-antigen vaccine, in which the neo-antigen is fused to CRT. CRT boosts the immune response and administration of the Ad5 [E1−, E2b−]-neo-antigen-CRT vectors results in elimination of cancer cells.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 4
Additional Sequences

| SEQ ID NO | Sequence |
|---|---|
| SEQ ID NO: 1 | ATGGAGTCTCCCTCGGCCCCTCCCCACAGATGGTGCATCCCCTG |
| | GCAGAGGCTCCTGCTCACAGCCTCACTTCTAACCTTCTGGAACC |
| | CGCCCACCACTGCCAAGCTCACTATTGAATCCACGCCGTTCAAT |
| | GTCGCAGAGGGGAAGGAGGTGCTTCTACTTGTCCACAATCTGCC |
| | CCAGCATCTTTTTGGCTACAGCTGGTACAAAGGTGAAAGAGTGG |
| | ATGGCAACCGTCAAATTATAGGATATGTAATAGGAACTCAACA |
| | AGCTACCCCAGGGCCCGCATACAGTGGTCGAGAGATAATATAC |
| | CCCAATGCATCCCTGCTGATCCAGAACATCATCCAGAATGACAC |
| | AGGATTCTACACCCTACACGTCATAAAGTCAGATCTTGTGAATG |
| | AAGAAGCAACTGGCCAGTTCCGGGTATACCCGGAGCTGCCCAA |
| | GCCCTCCATCTCCAGCAACAACTCCAAACCCGTGGAGGACAAG |
| | GATGCTGTGGCCTTCACCTGTGAACCTGAGACTCAGGACGCAAC |
| | CTACCTGTGGTGGGTAAACAATCAGAGCCTCCCGGTCAGTCCCA |
| | GGCTGCAGCTGTCCAATGGCAACAGGACCCTCACTCTATTCAAT |
| | GTCACAAGAAATGACACAGCAAGCTACAAATGTGAAACCCAGA |
| | ACCCAGTGAGTGCCAGGCGCAGTGATTCAGTCATCCTGAATGTC |
| | CTCTATGGCCCGGATGCCCCCACCATTTCCCCTCTAAACACATCT |
| | TACAGATCAGGGGAAAATCTGAACCTCTCCTGCCACGCAGCCTC |
| | TAACCCACCTGCACAGTACTCTTGGTTTGTCAATGGGACTTTCC |
| | AGCAATCCACCCAAGAGCTCTTTATCCCCAACATCACTGTGAAT |
| | AATAGTGGATCCTATACGTGCCAAGCCCATAACTCAGACACTGG |
| | CCTCAATAGGACCACAGTCACGACGATCACAGTCTATGCAGAG |
| | CCACCCAAACCCTTCATCACCAGCAACAACTCCAACCCCGTGGA |
| | GGATGAGGATGCTGTAGCCTTAACCTGTGAACCTGAGATTCAGA |
| | ACACAACCTACCTGTGGTGGGTAAATAATCAGAGCCTCCCGGTC |
| | AGTCCCAGGCTGCAGCTGTCCAATGACAACAGGACCCTCACTCT |
| | ACTCAGTGTCACAAGGAATGATGTAGGACCCTATGAGTGTGGA |
| | ATCCAGAACGAATTAAGTGTTGACCACAGCGACCCAGTCATCCT |
| | GAATGTCCTCTATGGCCCAGACGACCCCACCATTTCCCCCTCAT |
| | ACACCTATTACCGTCCAGGGGTGAACCTCAGCCTCTCCTGCCAT |
| | GCAGCCTCTAACCCACCTGCACAGTATTCTTGGCTGATTGATGG |
| | GAACATCCAGCAACACACACAAGAGCTCTTTATCTCCAACATCA |
| | CTGAGAAGAACAGCGGACTCTATACCTGCCAGGCCAATAACTC |
| | AGCCAGTGGCCACAGCAGGACTACAGTCAAGACAATCACAGTC |
| | TCTGCGGAGCTGCCCAAGCCCTCCATCTCCAGCAACAACTCCAA |
| | ACCCGTGGAGGACAAGGATGCTGTGGCCTTCACCTGTGAACCTG |
| | AGGCTCAGAACACAACCTACCTGTGGTGGGTAAATGGTCAGAG |
| | CCTCCCAGTCAGTCCCAGGCTGCAGCTGTCCAATGGCAACAGGA |
| | CCCTCACTCTATTCAATGTCACAAGAAATGACGCAAGAGCCTAT |
| | GTATGTGGAATCCAGAACTCAGTGAGTGCAAACCGCAGTGACC |
| | CAGTCACCCTGGATGTCCTCTATGGGCCGGACACCCCCATCATT |
| | TCCCCCCCAGACTCGTCTTACCTTTCGGGAGCGAACCTCAACCT |
| | CTCCTGCCACTCGGCCTCTAACCCATCCCCGCAGTATTCTTGGCG |
| | TATCAATGGGATACCGCAGCAACACACACAAGTTCTCTTTATCG |
| | CCAAAATCACGCCAAATAATAACGGGACCTATGCCTGTTTTGTC |
| | TCTAACTTGGCTACTGGCCGCAATAATTCCATAGTCAAGAGCAT |
| | CACAGTCTCTGCATCTGGAACTTCTCCTGGTCTCTCAGCTGGGG |
| | CCACTGTCGGCATCATGATTGGAGTGCTGGTTGGGGTTGCTCTG |
| | ATATAG |
| SEQ ID NO: 2 | CATCATCAATAATATACCTTATTTTGGATTGAAGCCAATATGAT |
| | AATGAGGGGGTGGAGTTTGTGACGTGGCGCGGGGCGTGGGAAC |
| | GGGGCGGGTGACGTAGTAGTGTGGCGGAAGTGTGATGTTGCAA |
| | GTGTGGCGGAACACATGTAAGCGACGGATGTGGCAAAAGTGAC |
| | GTTTTTGGTGTGCGCCGGTGTACACAGGAAGTGACAATTTTCGC |
| | GCGGTTTTAGGCGGATGTTGTAGTAAATTTGGGCGTAACCGAGT |
| | AAGATTTGGCCATTTTCGCGGGAAAACTGAATAAGAGGAAGTG |
| | AAATCTGAATAATTTTGTGTTACTCATAGCGCGTAATACTGTAA |
| | TAGTAATCAATTACGGGGTCATTAGTTCATAGCCCATATATGGA |
| | GTTCCGCGTTACATAACTTACGGTAAATGGCCCGCCTGGCTGAC |
| | CGCCCAACGACCCCCGCCCATTGACGTCAATAATGACGTATGTT |
| | CCCATAGTAACGCCAATAGGGACTTTCCATTGACGTCAATGGGT |
| | GGAGTATTTACGGTAAACTGCCCACTTGGCAGTACATCAAGTGT |
| | ATCATATGCCAAGTACGCCCCCTATTGACGTCAATGACGGTAAA |
| | TGGCCCGCCTGGCATTATGCCCAGTACATGACCTTATGGGACTT |
| | TCCTACTTGGCAGTACATCTACGTATTAGTCATCGCTATTACCAT |
| | GGTGATGCGGTTTTGGCAGTACATCAATGGGCGTGGATAGCGGT |
| | TTGACTCACGGGGATTTCCAAGTCTCCACCCCATTGACGTCAAT |
| | GGGAGTTTGTTTTGGCACCAAAATCAACGGGACTTTCCAAAATG |
| | TCGTAACAACTCCGCCCCATTGACGCAAATGGGCGGTAGGCGTG |
| | TACGGTGGGAGGTCTATATAAGCAGAGCTGGTTTAGTGAACCGT |
| | CAGATCCGCTAGAGATCTGGTACCGTCGACGCGGCCGCTCGAGC |
| | CTAAGCTTGGTACCGAGCTCGGATCCACTAGTAACGGCCGCCAG |
| | TGTGCTGGAATTCGGCTTAAAGGTACCCAGAGCAGACAGCCGC |
| | CACCATGGAGTCTCCCTCGGCCCCTCCCCACAGATGGTGCATCC |
| | CCTGGCAGAGGCTCCTGCTCACAGCCTCACTTCTAACCTTCTGG |
| | AACCCGCCCACCACTGCCAAGCTCACTATTGAATCCACGCCGTT |
| | CAATGTCGCAGAGGGGAAGGAGGTGCTTCTACTTGTCCACAATC |
| | TGCCCCAGCATCTTTTTGGCTACAGCTGGTACAAAGGTGAAAGA |
| | GTGGATGGCAACCGTCAAATTATAGGATATGTAATAGGAACTC |
| | AACAAGCTACCCCAGGGCCCGCATACAGTGGTCGAGAGATAAT |
| | ATACCCCAATGCATCCCTGCTGATCCAGAACATCATCCAGAATG |
| | ACACAGGATTCTACACCCTACACGTCATAAAGTCAGATCTTGTG |
| | AATGAAGAAGCAACTGGCCAGTTCCGGGTATACCCGGAGCTGC |
| | CCAAGCCCTCCATCTCCAGCAACAACTCCAAACCCGTGGAGGAC |
| | AAGGATGCTGTGGCCTTCACCTGTGAACCTGAGACTCAGGACGC |
| | AACCTACCTGTGGTGGGTAAACAATCAGAGCCTCCCGGTCAGTC |
| | CCAGGCTGCAGCTGTCCAATGGCAACAGGACCCTCACTCTATTC |
| | AATGTCACAAGAAATGACACAGCAAGCTACAAATGTGAAACCC |
| | AGAACCCAGTGAGTGCCAGGCGCAGTGATTCAGTCATCCTGAAT |
| | GTCCTCTATGGCCCGGATGCCCCCACCATTTCCCCTCTAAACAC |
| | ATCTTACAGATCAGGGGAAAATCTGAACCTCTCCTGCCACGCAG |
| | CCTCTAACCCACCTGCACAGTACTCTTGGTTTGTCAATGGGACTT |
| | TCCAGCAATCCACCCAAGAGCTCTTTATCCCCAACATCACTGTG |
| | AATAATAGTGGATCCTATACGTGCCAAGCCCATAACTCAGACAC |
| | TGGCCTCAATAGGACCACAGTCACGACGATCACAGTCTATGCAG |
| | AGCCACCCAAACCCTTCATCACCAGCAACAACTCCAACCCCGTG |
| | GAGGATGAGGATGCTGTAGCCTTAACCTGTGAACCTGAGATTCA |
| | GAACACAACCTACCTGTGGTGGGTAAATAATCAGAGCCTCCCG |
| | GTCAGTCCCAGGCTGCAGCTGTCCAATGACAACAGGACCCTCAC |
| | TCTACTCAGTGTCACAAGGAATGATGTAGGACCCTATGAGTGTG |
| | GAATCCAGAACGAATTAAGTGTTGACCACAGCGACCCAGTCAT |
| | CCTGAATGTCCTCTATGGCCCAGACGACCCCACCATTTCCCCCT |
| | CATACACCTATTACCGTCCAGGGGTGAACCTCAGCCTCTCCTGC |
| | CATGCAGCCTCTAACCCACCTGCACAGTATTCTTGGCTGATTGA |
| | TGGGAACATCCAGCAACACACACAAGAGCTCTTTATCTCCAACA |
| | TCACTGAGAAGAACAGCGGACTCTATACCTGCCAGGCCAATAA |
| | CTCAGCCAGTGGCCACAGCAGGACTACAGTCAAGACAATCACA |
| | GTCTCTGCGGAGCTGCCCAAGCCCTCCATCTCCAGCAACAACTC |
| | CAAACCCGTGGAGGACAAGGATGCTGTGGCCTTCACCTGTGAA |
| | CCTGAGGCTCAGAACACAACCTACCTGTGGTGGGTAAATGGTCA |
| | GAGCCTCCCAGTCAGTCCCAGGCTGCAGCTGTCCAATGGCAACA |
| | GGACCCTCACTCTATTCAATGTCACAAGAAATGACGCAAGAGCC |
| | TATGTATGTGGAATCCAGAACTCAGTGAGTGCAAACCGCAGTG |
| | ACCCAGTCACCCTGGATGTCCTCTATGGGCCGGACACCCCCATC |
| | ATTTCCCCCCCAGACTCGTCTTACCTTTCGGGAGCGGACCTCAA |
| | CCTCTCCTGCCACTCGGCCTCTAACCCATCCCCGCAGTATTCTTG |
| | GCGTATCAATGGGATACCGCAGCAACACACACAAGTTCTCTTTA |
| | TCGCCAAAATCACGCCAAATAATAACGGGACCTATGCCTGTTTT |
| | GTCTCTAACTTGGCTACTGGCCGCAATAATTCCATAGTCAAGAG |
| | CATCACAGTCTCTGCATCTGGAACTTCTCCTGGTCTCTCAGCTGG |
| | GGCCACTGTCGGCATCATGATTGGAGTGCTGGTTGGGGTTGCTC |
| | TGATATAGCAGCCCTGGTGTAGTTTCTTCATTTCAGGAAGACTG |
| | ACAGTTGTTTTGCTTCTTCCTTAAAGCATTTGCAACAGCTACAGT |
| | CTAAAATTGCTTCTTTACCAAGGATATTTACAGAAAAGACTCTG |
| | ACCAGAGATCGAGACCATCCTCTAGATAAGATATCCGATCCACC |
| | GGATCTAGATAACTGATCATAATCAGCCATACCACATTTGTAGA |
| | GGTTTTACTTGCTTTAAAAAACCTCCCACACCTCCCCCTGAACCT |
| | GAAACATAAAATGAATGCAATTGTTGTTGTTAACTTGTTTATTG |
| | CAGCTTATAATGGTTACAAATAAAGCAATAGCATCACAAATTTC |
| | ACAAATAAAGCATTTTTTTCACTGCATTCTAGTTGTGGTTTGTCC |
| | AAACTCATCAATGTATCTTAACGCGGATCTGGGCGTGGTTAAGG |
| | GTGGGAAAGAATATATAAGGTGGGGGTCTTATGTAGTTTTGTAT |
| | CTGTTTTGCAGCAGCCGCCGCCGCCATGAGCACCAACTCGTTTG |
| | ATGGAAGCATTGTGAGCTCATATTTGACAACGCGCATGCCCCCA |
| | TGGGCCGGGGTGCGTCAGAATGTGATGGGCTCCAGCATTGATG |
| | GTCGCCCCGTCCTGCCCGCAAACTCTACTACCTTGACCTACGAG |
| | ACCGTGTCTGGAACGCCGTTGGAGACTGCAGCCTCCGCCGCCGC |
| | TTCAGCCGCTGCAGCCACCGCCCGCGGGATTGTGACTGACTTTG |
| | CTTTCCTGAGCCCGCTTGCAAGCAGTGCAGCTTCCCGTTCATCC |
| | GCCCGCGATGACAAGTTGACGGCTCTTTTGGCACAATTGGATTC |
| | TTTGACCCGGGAACTTAATGTCGTTTCTCAGCAGCTGTTGGATCT |
| | GCGCCAGCAGGTTTCTGCCCTGAAGGCTTCCTCCCCTCCCAATG |
| | CGGTTTAAAACATAAATAAAAAACCAGACTCTGTTTGGATTTGG |
| | ATCAAGCAAGTGTCTTGCTGTCTTTATTTAGGGGTTTTGCGCGCG |
| | CGGTAGGCCCGGGACCAGCGGTCTCGGTCGTTGAGGGTCCTGTG |
| | TATTTTTTCCAGGACGTGGTAAAGGTGACTCTGGATGTTCAGAT |
| | ACATGGGCATAAGCCCGTCTCTGGGGTGGAGGTAGCACCACTG |
| | CAGAGCTTCATGCTGCGGGGTGGTGTTGTAGATGATCCAGTCGT |
| | AGCAGGAGCGCTGGGCGTGGTGCCTAAAAATGTCTTTCAGTAGC |
| | AAGCTGATTGCCAGGGGCAGGCCCTTGGTGTAAGTGTTTACAAA |
| | GCGGTTAAGCTGGGATGGGTGCATACGTGGGGATATGAGATGC |
| | ATCTTGGACTGTATTTTTAGGTTGGCTATGTTCCCAGCCATATCC |
| | CTCCGGGGATTCATGTTGTGCAGAACCACCAGCACAGTGTATCC |
| | GGTGCACTTGGGAAATTTGTCATGTAGCTTAGAAGGAAATGCGT |
| | GGAAGAACTTGGAGACGCCCTTGTGACCTCCAAGATTTTCCATG |
| | CATTCGTCCATAATGATGGCAATGGGCCCACGGGCGGCGGCCTG |
| | GGCGAAGATATTTCTGGGATCACTAACGTCATAGTTGTGTTCCA |
| | GGATGAGATCGTCATAGGCCATTTTTACAAAGCGCGGGCGGAG |
| | GGTGCCAGACTGCGGTATAATGGTTCCATCCGGCCCAGGGGCGT |
| | AGTTACCCTCACAGATTTGCATTTCCCACGCTTTGAGTTCAGATG |
| | GGGGGATCATGTCTACCTGCGGGGCGATGAAGAAAACGGTTTC |
| | CGGGGTAGGGGAGATCAGCTGGGAAGAAAGCAGGTTCCTGAGC |
| | AGCTGCGACTTACCGCAGCCGGTGGGCCCGTAAATCACACCTAT |
| | TACCGGCTGCAACTGGTAGTTAAGAGAGCTGCAGCTGCCGTCAT |
| | CCCTGAGCAGGGGGGCCACTTCGTTAAGCATGTCCCTGACTCGC |
| | ATGTTTTCCCTGACCAAATCCGCCAGAAGGCGCTCGCCGCCCAG |
| | CGATAGCAGTTCTTGCAAGGAAGCAAAGTTTTTCAACGGTTTGA |
| | GACCGTCCGCCGTAGGCATGCTTTTGAGCGTTTGACCAAGCAGT |
| | TCCAGGCGGTCCCACAGCTCGGTCACCTGCTCTACGGCATCTCG |
| | ATCCAGCATATCTCCTCGTTTCGCGGGTTGGGGCGGCTTTCGCT |
| | GTACGGCAGTAGTCGGTGCTCGTCCAGACGGGCCAGGGTCATGT |
| | CTTTCCACGGGCGCAGGGTCCTCGTCAGCGTAGTCTGGGTCACG |
| | GTGAAGGGGTGCGCTCCGGGCTGCGCGCTGGCCAGGGTGCGCT |
| | TGAGGCTGGTCCTGCTGGTGCTGAAGCGCTGCCGGTCTTCGCCC |
| | TGCGCGTCGGCCAGGTAGCATTTGACCATGGTGTCATAGTCCAG |
| | CCCCTCCGCGGCGTGGCCCTTGGCGCGCAGCTTGCCCTTGGAGG |
| | AGGCGCCGCACGAGGGGCAGTGCAGACTTTTGAGGGCGTAGAG |
| | CTTGGGCGCGAGAAATACCGATTCCGGGGAGTAGGCATCCGCG |
| | CCGCAGGCCCCGCAGACGGTCTCGCATTCCACGAGCCAGGTGA |
| | GCTCTGGCCGTTCGGGGTCAAAAACCAGGTTTCCCCCATGCTTT |
| | TTGATGCGTTTCTTACCTCTGGTTTCCATGAGCCGGTGTCCACGC |
| | TCGGTGACGAAAAGGCTGTCCGTGTCCCCGTATACAGACTTGAG |
| | AGGCCTGTCCTCGAGCGGTGTTCCGCGGTCCTCCTCGTATAGAA |
| | ACTCGGACCACTCTGAGACAAAGGCTCGCGTCCAGGCCAGCAC |
| | GAAGGAGGCTAAGTGGGAGGGGTAGCGGTCGTTGTCCACTAGG |
| | GGGTCCACTCGCTCCAGGGTGTGAAGACACATGTCGCCCTCTTC |
| | GGCATCAAGGAAGGTGATTGGTTTGTAGGTGTAGGCCACGTGA |
| | CCGGGTGTTCCTGAAGGGGGGCTATAAAAGGGGGTGGGGGCGC |
| | GTTCGTCCTCACTCTCTTCCGCATCGCTGTCTGCGAGGGCCAGCT |
| | GTTGGGGTGAGTACTCCCTCTGAAAAGCGGGCATGACTTCTGCG |
| | CTAAGATTGTCAGTTTCCAAAAACGAGGAGGATTTGATATTCAC |
| | CTGGCCCGCGGTGATGCCTTTGAGGGTGGCCGCATCCATCTGGT |
| | CAGAAAAGACAATCTTTTTGTTGTCAAGCTTGGTGGCAAACGAC |
| | CCGTAGAGGGCGTTGGACAGCAACTTGGCGATGGAGCGCAGGG |
| | TTTGGTTTTTGTCGCGATCGGCGCGCTCCTTGGCCGCGATGTTTA |
| | GCTGCACGTATTCGCGCGCAACGCACCGCCATTCGGGAAAGAC |
| | GGTGGTGCGCTCGTCGGGCACCAGGTGCACGCGCCAACCGCGG |
| | TTGTGCAGGGTGACAAGGTCAACGCTGGTGGCTACCTCTCCGCG |
| | TAGGCGCTCGTTGGTCCAGCAGAGGCGGCCGCCCTTGCGCGAGC |
| | AGAATGGCGGTAGGGGGTCTAGCTGCGTCTCGTCCGGGGGGTCT |
| | GCGTCCACGGTAAAGACCCCGGGCAGCAGGCGCGCGTCGAAGT |
| | AGTCTATCTTGCATCCTTGCAAGTCTAGCGCCTGCTGCCATGCG |
| | CGGGCGGCAAGCGCGCGCTCGTATGGGTTGAGTGGGGGACCCC |
| | ATGGCATGGGGTGGGTGAGCGCGGAGGCGTACATGCCGCAAAT |
| | GTCGTAAACGTAGAGGGGCTCTCTGAGTATTCCAAGATATGTAG |
| | GGTAGCATCTTCCACCGCGGATGCTGGCGCGCACGTAATCGTAT |
| | AGTTCGTGCGAGGGAGCGAGGAGGTCGGGACCGAGGTTGCTAC |
| | GGGCGGGCTGCTCTGCTCGGAAGACTATCTGCCTGAAGATGGCA |
| | TGTGAGTTGGATGATATGGTTGGACGCTGGAAGACGTTGAAGCT |
| | GGCGTCTGTGAGACCTACCGCGTCACGCACGAAGGAGGCGTAG |
| | GAGTCGCGCAGCTTGTTGACCAGCTCGGCGGTGACCTGCACGTC |
| | TAGGGCGCAGTAGTCCAGGGTTTCCTTGATGATGTCATACTTAT |
| | CCTGTCCCTTTTTTTTCCACAGCTCGCGGTTGAGGACAAACTCTT |
| | CGCGGTCTTTCCAGTACTCTTGGATCGGAAACCCGTCGGCCTCC |
| | GAACGGTAAGAGCCTAGCATGTAGAACTGGTTGACGGCCTGGT |
| | AGGCGCAGCATCCCTTTTCTACGGGTAGCGCGTATGCCTGCGCG |
| | GCCTTCCGGCATGACCAGCATGAAGGGCACGAGCTGCTTCCCAA |
| | AGGCCCCCATCCAAGTATAGGTCTCTACATCGTAGGTGACAAAG |
| | AGACGCTCGGTGCGAGGATGCGAGCCGATCGGGAAGAACTGGA |
| | TCTCCCGCCACCAATTGGAGGAGTGGCTATTGATGTGGTGAAAG |
| | TAGAAGTCCCTGCGACGGGCCGAACACTCGTGCTGGCTTTTGTA |
| | AAAACGTGCGCAGTACTGGCAGCGGTGCACGGGCTGTACATCC |
| | TGCACGAGGTTGACCTGACGACCGCGCACAAGGAAGCAGAGTG |
| | GGAATTTGAGCCCCTCGCCTGGCGGGTTTGGCTGGTGGTCTTCT |
| | ACTTCGGCTGCTTGTCCTTGACCGTCTGGCTGCTCGAGGGGAGT |
| | TACGGTGGATCGGACCACCACGCCGCGCGAGCCCAAAGTCCAG |
| | ATGTCCGCGCGCGGCGGTCGGAGCTTGATGACAACATCGCGCA |
| | GATGGGAGCTGTCCATGGTCTGGAGCTCCCGCGGCGTCAGGTCA |
| | GGCGGGAGCTCCTGCAGGTTTACCTCGCATAGACGGGTCAGGG |
| | CGCGGGCTAGATCCAGGTGATACCTAATTTCCAGGGGCTGGTTG |
| | GTGGCGGCGTCGATGGCTTGCAAGAGGCCGCATCCCCGCGGCG |
| | CGACTACGGTACCGCGCGGCGGGCGGTGGGCCGCGGGGGTGTC |
| | CTTGGATGATGCATCTAAAAGCGGTGACGCGGGCGAGCCCCCG |
| | GAGGTAGGGGGGGCTCCGGACCCGCCGGGAGAGGGGGCAGGG |
| | GCACGTCGGCGCCGCGCGCGGGCAGGAGCTGGTGCTGCGCGCG |
| | TAGGTTGCTGGCGAACGCGACGACGCGGCGGTTGATCTCCTGAA |
| | TCTGGCGCCTCTGCGTGAAGACGACGGGCCCGGTGAGCTTGAAC |
| | CTGAAAGAGAGTTCGACAGAATCAATTTCGGTGTCGTTGACGGC |
| | GGCCTGGCGCAAAATCTCCTGCACGTCTCCTGAGTTGTCTTGAT |
| | AGGCGATCTCGGCCATGAACTGCTCGATCTCTTCCTCCTGGAGA |
| | TCTCCGCGTCCGGCTCGCTCCACGGTGGCGGCGAGGTCGTTGGA |
| | AATGCGGGCCATGAGCTGCGAGAAGGCGTTGAGGCCTCCCTCG |
| | TTCCAGACGCGGCTGTAGACCACGCCCCCTTCGGCATCGCGGGC |
| | GCGCATGACCACCTGCGCGAGATTGAGCTCCACGTGCCGGGCG |
| | AAGACGGCGTAGTTTCGCAGGCGCTGAAAGAGGTAGTTGAGGG |
| | TGGTGGCGGTGTGTTCTGCCACGAAGAAGTACATAACCCAGCGT |
| | CGCAACGTGGATTCGTTGATAATTGTTGTGTAGGTACTCCGCCG |
| | CCGAGGGACCTGAGCGAGTCCGCATCGACCGGATCGGAAAACC |
| | TCTCGAGAAAGGCGTCTAACCAGTCACAGTCGCAAGGTAGGCT |
| | GAGCACCGTGGCGGGCGGCAGCGGGCGGCGGTCGGGGTTGTTT |
| | CTGGCGGAGGTGCTGCTGATGATGTAATTAAAGTAGGCGGTCTT |
| | GAGACGGCGGATGGTCGACAGAAGCACCATGTCCTTGGGTCCG |
| | GCCTGCTGAATGCGCAGGCGGTCGGCCATGCCCCAGGCTTCGTT |
| | TTGACATCGGCGCAGGTCTTTGTAGTAGTCTTGCATGAGCCTTTC |
| | TACCGGCACTTCTTCTTCTCCTTCCTCTTGTCCTGCATCTCTTGCA |
| | TCTATCGCTGCGGCGGCGGCGGAGTTTGGCCGTAGGTGGCGCCC |
| | TCTTCCTCCCATGCGTGTGACCCCGAAGCCCCTCATCGGCTGAA |
| | GCAGGGCTAGGTCGGCGACAACGCGCTCGGCTAATATGGCCTG |
| | CTGCACCTGCGTGAGGGTAGACTGGAAGTCATCCATGTCCACAA |
| | AGCGGTGGTATGCGCCCGTGTTGATGGTGTAAGTGCAGTTGGCC |
| | ATAACGGACCAGTTAACGGTCTGGTGACCCGGCTGCGAGAGCT |
| | CGGTGTACCTGAGACGCGAGTAAGCCCTCGAGTCAAATACGTA |
| | GTCGTTGCAAGTCCGCACCAGGTACTGGTATCCCACCAAAAAGT |
| | GCGGCGGCGGCTGGCGGTAGAGGGGCCAGCGTAGGGTGGCCGG |
| | GGCTCCGGGGGCGAGATCTTCCAACATAAGGCGATGATATCCGT |
| | AGATGTACCTGGACATCCAGGTGATGCCGGCGGCGGTGGTGGA |
| | GGCGCGCGGAAAGTCGCGGACGCGGTTCCAGATGTTGCGCAGC |
| | GGCAAAAAGTGCTCCATGGTCGGGACGCTCTGGCCGGTCAGGC |
| | GCGCGCAATCGTTGACGCTCTAGCGTGCAAAAGGAGAGCCTGT |
| | AAGCGGGCACTCTTCCGTGGTCTGGTGGATAAATTCGCAAGGGT |
| | ATCATGGCGGACGACCGGGGTTCGAGCCCCGTATCCGGCCGTCC |
| | GCCGTGATCCATGCGGTTACCGCCCGCGTGTCGAACCCAGGTGT |
| | GCGACGTCAGACAACGGGGGAGTGCTCCTTTTGGCTTCCTTCCA |
| | GGCGCGGCGGCTGCTGCGCTAGCTTTTTTGGCCACTGGCCGCGC |
| | GCAGCGTAAGCGGTTAGGCTGGAAAGCGAAAGCATTAAGTGGC |
| | TCGCTCCCTGTAGCCGGAGGGTTATTTTCCAAGGGTTGAGTCGC |
| | GGGACCCCCGGTTCGAGTCTCGGACCGGCCGGACTGCGGCGAA |
| | CGGGGGTTTGCCTCCCCGTCATGCAAGACCCCGCTTGCAAATTC |
| | CTCCGGAAACAGGGACGAGCCCCTTTTTTGCTTTTCCCAGATGC |
| | ATCCGGTGCTGCGGCAGATGCGCCCCCCTCCTCAGCAGCGGCAA |
| | GAGCAAGAGCAGCGGCAGACATGCAGGGCACCCTCCCCTCCTC |
| | CTACCGCGTCAGGAGGGGCGACATCCGCGGTTGACGCGGCAGC |
| | AGATGGTGATTACGAACCCCCGCGGCGCCGGGCCCGGCACTAC |
| | CTGGACTTGGAGGAGGGCGAGGGCCTGGCGCGGCTAGGAGCGC |
| | CCTCTCCTGAGCGGCACCCAAGGGTGCAGCTGAAGCGTGATAC |
| | GCGTGAGGCGTACGTGCCGCGGCAGAACCTGTTTCGCGACCGC |
| | GAGGGAGAGGAGCCCGAGGAGATGCGGGATCGAAAGTTCCACG |
| | CAGGGCGCGAGCTGCGGCATGGCCTGAATCGCGAGCGGTTGCT |
| | GCGCGAGGAGGACTTTGAGCCCGACGCGCGAACCGGGATTAGT |
| | CCCGCGCGCGCACACGTGGCGGCCGCCGACCTGGTAACCGCAT |
| | ACGAGCAGACGGTGAACCAGGAGATTAACTTTCAAAAAAGCTT |
| | TAACAACCACGTGCGTACGCTTGTGGCGCGCGAGGAGGTGGCT |
| | ATAGGACTGATGCATCTGTGGGACTTTGTAAGCGCGCTGGAGCA |
| | AAACCCAAATAGCAAGCCGCTCATGGCGCAGCTGTTCCTTATAG |
| | TGCAGCACAGCAGGGACAACGAGGCATTCAGGGATGCGCTGCT |
| | AAACATAGTAGAGCCCGAGGGCCGCTGGCTGCTCGATTTGATA |
| | AACATCCTGCAGAGCATAGTGGTGCAGGAGCGCAGCTTGAGCC |
| | TGGCTGACAAGGTGGCCGCCATCAACTATTCCATGCTTAGCCTG |
| | GGCAAGTTTTACGCCCGCAAGATATACCATACCCCTTACGTTCC |
| | CATAGACAAGGAGGTAAAGATCGAGGGGTTCTACATGCGCATG |
| | GCGCTGAAGGTGCTTACCTTGAGCGACGACCTGGGCGTTTATCG |
| | CAACGAGCGCATCCACAAGGCCGTGAGCGTGAGCCGGCGGCGC |
| | GAGCTCAGCGACCGCGAGCTGATGCACAGCCTGCAAAGGGCCC |
| | TGGCTGGCACGGGCAGCGGCGATAGAGAGGCCGAGTCCTACTT |
| | TGACGCGGGCGCTGACCTGCGCTGGGCCCCAAGCCGACGCGCC |
| | CTGGAGGCAGCTGGGGCCGGACCTGGGCTGGCGGTGGCACCCG |
| | CGCGCGCTGGCAACGTCGGCGGCGTGGAGGAATATGACGAGGA |
| | CGATGAGTACGAGCCAGAGGACGGCGAGTACTAAGCGGTGATG |
| | TTTCTGATCAGATGATGCAAGACGCAACGGACCCGGCGGTGCG |
| | GGCGGCGCTGCAGAGCCAGCCGTCCGGCCTTAACTCCACGGAC |
| | GACTGGCGCCAGGTCATGGACCGCATCATGTCGCTGACTGCGCG |
| | CAATCCTGACGCGTTCCGGCAGCAGCCGCAGGCCAACCGGCTCT |
| | CCGCAATTCTGGAAGCGGTGGTCCCGGCGCGCGCAAACCCCAC |
| | GCACGAGAAGGTGCTGGCGATCGTAAACGCGCTGGCCGAAAAC |
| | AGGGCCATCCGGCCCGACGAGGCCGGCCTGGTCTACGACGCGC |
| | TGCTTCAGCGCGTGGCTCGTTACAACAGCGGCAACGTGCAGACC |
| | AACCTGGACCGGCTGGTGGGGGATGTGCGCGAGGCCGTGGCGC |
| | AGCGTGAGCGCGCGCAGCAGCAGGGCAACCTGGGCTCCATGGT |
| | TGCACTAAACGCCTTCCTGAGTACACAGCCCGCCAACGTGCCGC |
| | GGGGACAGGAGGACTACACCAACTTTGTGAGCGCACTGCGGCT |
| | AATGGTGACTGAGACACCGCAAAGTGAGGTGTACCAGTCTGGG |
| | CCAGACTATTTTTTCCAGACCAGTAGACAAGGCCTGCAGACCGT |
| | AAACCTGAGCCAGGCTTTCAAAAACTTGCAGGGGCTGTGGGGG |
| | GTGCGGGCTCCCACAGGCGACCGCGCGACCGTGTCTAGCTTGCT |
| | GACGCCCAACTCGCGCCTGTTGCTGCTGCTAATAGCGCCCTTCA |
| | CGGACAGTGGCAGCGTGTCCCGGGACACATACCTAGGTCACTTG |
| | CTGACACTGTACCGCGAGGCCATAGGTCAGGCGCATGTGGACG |
| | AGCATACTTTCCAGGAGATTACAAGTGTCAGCCGCGCGCTGGGG |
| | CAGGAGGACACGGGCAGCCTGGAGGCAACCCTAAACTACCTGC |
| | TGACCAACCGGCGGCAGAAGATCCCCTCGTTGCACAGTTTAAAC |
| | AGCGAGGAGGAGCGCATTTTGCGCTACGTGCAGCAGAGCGTGA |
| | GCCTTAACCTGATGCGCGACGGGGTAACGCCCAGCGTGGCGCT |
| | GGACATGACCGCGCGCAACATGGAACCGGGCATGTATGCCTCA |
| | AACCGGCCGTTTATCAACCGCCTAATGGACTACTTGCATCGCGC |
| | GGCCGCCGTGAACCCCGAGTATTTCACCAATGCCATCTTGAACC |
| | CGCACTGGCTACCGCCCCCTGGTTTCTACACCGGGGGATTCGAG |
| | GTGCCCGAGGGTAACGATGGATTCCTCTGGGACGACATAGACG |
| | ACAGCGTGTTTTCCCCGCAACCGCAGACCCTGCTAGAGTTGCAA |
| | CAGCGCGAGCAGGCAGAGGCGGCGCTGCGAAAGGAAAGCTTCC |
| | GCAGGCCAAGCAGCTTGTCCGATCTAGGCGCTGCGGCCCCGCG |
| | GTCAGATGCTAGTAGCCCATTTCCAAGCTTGATAGGGTCTCTTA |
| | CCAGCACTCGCACCACCCGCCCGCGCCTGCTGGGCGAGGAGGA |
| | GTACCTAAACAACTCGCTGCTGCAGCCGCAGCGCGAAAAAAAC |
| | CTGCCTCCGGCATTTCCCAACAACGGGATAGAGAGCCTAGTGGA |
| | CAAGATGAGTAGATGGAAGACGTACGCGCAGGAGCACAGGGAC |
| | GTGCCAGGCCCGCGCCCGCCCACCCGTCGTCAAAGGCACGACC |
| | GTCAGCGGGGTCTGGTGTGGGAGGACGATGACTCGGCAGACGA |
| | CAGCAGCGTCCTGGATTTGGGAGGGAGTGGCAACCCGTTTGCGC |
| | ACCTTCGCCCCAGGCTGGGGAGAATGTTTTAAAAAAAAAAAAG |
| | CATGATGCAAAATAAAAAACTCACCAAGGCCATGGCACCGAGC |
| | GTTGGTTTTCTTGTATTCCCCTTAGTATGCGGCGCGCGGCGATGT |
| | ATGAGGAAGGTCCTCCTCCCTCCTACGAGAGTGTGGTGAGCGCG |
| | GCGCCAGTGGCGGCGGCGCTGGGTTCTCCCTTCGATGCTCCCCT |
| | GGACCCGCCGTTTGTGCCTCCGCGGTACCTGCGGCCTACCGGGG |
| | GGAGAAACAGCATCCGTTACTCTGAGTTGGCACCCCTATTCGAC |
| | ACCACCCGTGTGTACCTGGTGGACAACAAGTCAACGGATGTGG |
| | CATCCCTGAACTACCAGAACGACCACAGCAACTTTCTGACCACG |
| | GTCATTCAAAACAATGACTACAGCCCGGGGGAGGCAAGCACAC |
| | AGACCATCAATCTTGACGACCGGTCGCACTGGGGCGGCGACCT |
| | GAAAACCATCCTGCATACCAACATGCCAAATGTGAACGAGTTC |
| | ATGTTTACCAATAAGTTTAAGGCGCGGGTGATGGTGTCGCGCTT |
| | GCCTACTAAGGACAATCAGGTGGAGCTGAAATACGAGTGGGTG |
| | GAGTTCACGCTGCCCGAGGGCAACTACTCCGAGACCATGACCAT |
| | AGACCTTATGAACAACGCGATCGTGGAGCACTACTTGAAAGTG |
| | GGCAGACAGAACGGGGTTCTGGAAAGCGACATCGGGGTAAAGT |
| | TTGACACCCGCAACTTCAGACTGGGGTTTGACCCCGTCACTGGT |
| | CTTGTCATGCCTGGGGTATATACAAACGAAGCCTTCCATCCAGA |
| | CATCATTTTGCTGCCAGGATGCGGGGTGGACTTCACCCACAGCC |
| | GCCTGAGCAACTTGTTGGGCATCCGCAAGCGGCAACCCTTCCAG |
| | GAGGGCTTTAGGATCACCTACGATGATCTGGAGGGTGGTAACAT |
| | TCCCGCACTGTTGGATGTGGACGCCTACCAGGCGAGCTTGAAAG |
| | ATGACACCGAACAGGGCGGGGGTGGCGCAGGCGGCAGCAACA |
| | GCAGTGGCAGCGGCGCGGAAGAGAACTCCAACGCGGCAGCCGC |
| | GGCAATGCAGCCGGTGGAGGACATGAACGATCATGCCATTCGC |
| | GGCGACACCTTTGCCACACGGGCTGAGGAGAAGCGCGCTGAGG |
| | CCGAAGCAGCGGCCGAAGCTGCCGCCCCCGCTGCGCAACCCGA |
| | GGTCGAGAAGCCTCAGAAGAAACCGGTGATCAAACCCCTGACA |
| | GAGGACAGCAAGAAACGCAGTTACAACCTAATAAGCAATGACA |
| | GCACCTTCACCCAGTACCGCAGCTGGTACCTTGCATACAACTAC |
| | GGCGACCCTCAGACCGGAATCCGCTCATGGACCCTGCTTTGCAC |
| | TCCTGACGTAACCTGCGGCTCGGAGCAGGTCTACTGGTCGTTGC |
| | CAGACATGATGCAAGACCCCGTGACCTTCCGCTCCACGCGCCAG |
| | ATCAGCAACTTTCCGGTGGTGGGCGCCGAGCTGTTGCCCGTGCA |
| | CTCCAAGAGCTTCTACAACGACCAGGCCGTCTACTCCCAACTCA |
| | TCCGCCAGTTTACCTCTCTGACCCACGTGTTCAATCGCTTTCCCG |
| | AGAACCAGATTTTGGCGCGCCCGCCAGCCCCCACCATCACCACC |
| | GTCAGTGAAAACGTTCCTGCTCTCACAGATCACGGGACGCTACC |
| | GCTGCGCAACAGCATCGGAGGAGTCCAGCGAGTGACCATTACT |
| | GACGCCAGACGCCGCACCTGCCCCTACGTTTACAAGGCCCTGGG |
| | CATAGTCTCGCCGCGCGTCCTATCGAGCCGCACTTTTTGAGCAA |
| | GCATGTCCATCCTTATATCGCCCAGCAATAACACAGGCTGGGGC |
| | CTGCGCTTCCCAAGCAAGATGTTTGGCGGGGCCAAGAAGCGCTC |
| | CGACCAACACCCAGTGCGCGTGCGCGGGCACTACCGCGCGCCC |
| | TGGGGCGCGCACAAACGCGGCCGCACTGGGCGCACCACCGTCG |
| | ATGACGCCATCGACGCGGTGGTGGAGGAGGCGCGCAACTACAC |
| | GCCCACGCCGCCACCAGTGTCCACAGTGGACGCGGCCATTCAG |
| | ACCGTGGTGCGCGGAGCCCGGCGCTATGCTAAAATGAAGAGAC |
| | GGCGGAGGCGCGTAGCACGTCGCCACCGCCGCCGACCCGGCAC |
| | TGCCGCCCAACGCGCGGCGGCGGCCCTGCTTAACCGCGCACGTC |
| | GCACCGGCCGACGGGCGGCCATGCGGGCCGCTCGAAGGCTGGC |
| | CGCGGGTATTGTCACTGTGCCCCCCAGGTCCAGGCGACGAGCGG |
| | CCGCCGCAGCAGCCGCGGCCATTAGTGCTATGACTCAGGGTCGC |
| | AGGGGCAACGTGTATTGGGTGCGCGACTCGGTTAGCGGCCTGC |
| | GCGTGCCCGTGCGCACCCGCCCCCCGCGCAACTAGATTGCAAGA |
| | AAAAACTACTTAGACTCGTACTGTTGTATGTATCCAGCGGCGGC |
| | GGCGCGCAACGAAGCTATGTCCAAGCGCAAAATCAAAGAAGAG |
| | ATGCTCCAGGTCATCGCGCCGGAGATCTATGGCCCCCCGAAGAA |
| | GGAAGAGCAGGATTACAAGCCCCGAAAGCTAAAGCGGGTCAAA |
| | AAGAAAAAGAAAGATGATGATGATGAACTTGACGACGAGGTGG |
| | AACTGCTGCACGCTACCGCGCCCAGGCGACGGGTACAGTGGAA |
| | AGGTCGACGCGTAAAACGTGTTTTGCGACCCGGCACCACCGTAG |
| | TCTTTACGCCCGGTGAGCGCTCCACCCGCACCTACAAGCGCGTG |
| | TATGATGAGGTGTACGGCGACGAGGACCTGCTTGAGCAGGCCA |
| | ACGAGCGCCTCGGGGAGTTTGCCTACGGAAAGCGGCATAAGGA |
| | CATGCTGGCGTTGCCGCTGGACGAGGGCAACCCAACACCTAGC |
| | CTAAAGCCCGTAACACTGCAGCAGGTGCTGCCCGCGCTTGCACC |
| | GTCCGAAGAAAAGCGCGGCCTAAAGCGCGAGTCTGGTGACTTG |
| | GCACCCACCGTGCAGCTGATGGTACCCAAGCGCCAGCGACTGG |
| | AAGATGTCTTGGAAAAAATGACCGTGGAACCTGGGCTGGAGCC |
| | CGAGGTCCGCGTGCGGCCAATCAAGCAGGTGGCGCCGGGACTG |
| | GGCGTGCAGACCGTGGACGTTCAGATACCCACTACCAGTAGCA |
| | CCAGTATTGCCACCGCCACAGAGGGCATGGAGACACAAACGTC |
| | CCCGGTTGCCTCAGCGGTGGCGGATGCCGCGGTGCAGGCGGTC |
| | GCTGCGGCCGCGTCCAAGACCTCTACGGAGGTGCAAACGGACC |
| | CGTGGATGTTTCGCGTTTCAGCCCCCCGGCGCCCGCGCCGTTCG |
| | AGGAAGTACGGCGCCGCCAGCGCGCTACTGCCCGAATATGCCC |
| | TACATCCTTCCATTGCGCCTACCCCCGGCTATCGTGGCTACACCT |
| | ACCGCCCCAGAAGACGAGCAACTACCCGACGCCGAACCACCAC |
| | TGGAACCCGCCGCCGCCGTCGCCGTCGCCAGCCCGTGCTGGCCC |
| | CGATTTCCGTGCGCAGGGTGGCTCGCGAAGGAGGCAGGACCCT |
| | GGTGCTGCCAACAGCGCGCTACCACCCCAGCATCGTTTAAAAGC |
| | CGGTCTTTGTGGTTCTTGCAGATATGGCCCTCACCTGCCGCCTCC |
| | GTTTCCCGGTGCCGGGATTCCGAGGAAGAATGCACCGTAGGAG |
| | GGGCATGGCCGGCCACGGCCTGACGGGCGGCATGCGTCGTGCG |
| | CACCACCGGCGGCGGCGCGCGTCGCACCGTCGCATGCGCGGCG |
| | GTATCCTGCCCCTCCTTATTCCACTGATCGCCGCGGCGATTGGC |
| | GCCGTGCCCGGAATTGCATCCGTGGCCTTGCAGGCGCAGAGAC |
| | ACTGATTAAAAACAAGTTGCATGTGGAAAAATCAAAATAAAAA |
| | GTCTGGACTCTCACGCTCGCTTGGTCCTGTAACTATTTTGTAGAA |
| | TGGAAGACATCAACTTTGCGTCTCTGGCCCCGCGACACGGCTCG |
| | CGCCCGTTCATGGGAAACTGGCAAGATATCGGCACCAGCAATA |
| | TGAGCGGTGGCGCCTTCAGCTGGGGCTCGCTGTGGAGCGGCATT |
| | AAAAATTTCGGTTCCACCGTTAAGAACTATGGCAGCAAGGCCTG |
| | GAACAGCAGCACAGGCCAGATGCTGAGGGATAAGTTGAAAGAG |
| | CAAAATTTCCAACAAAAGGTGGTAGATGGCCTGGCCTCTGGCAT |
| | TAGCGGGGTGGTGGACCTGGCCAACCAGGCAGTGCAAAATAAG |
| | ATTAACAGTAAGCTTGATCCCCGCCCTCCCGTAGAGGAGCCTCC |
| | ACCGGCCGTGGAGACAGTGTCTCCAGAGGGGCGTGGCGAAAAG |
| | CGTCCGCGCCCCGACAGGGAAGAAACTCTGGTGACGCAAATAG |
| | ACGAGCCTCCCTCGTACGAGGAGGCACTAAAGCAAGGCCTGCC |
| | CACCACCCGTCCCATCGCGCCCATGGCTACCGGAGTGCTGGGCC |
| | AGCACACACCCGTAACGCTGGACCTGCCTCCCCCCGCCGACACC |
| | CAGCAGAAACCTGTGCTGCCAGGCCCGACCGCCGTTGTTGTAAC |
| | CCGTCCTAGCCGCGCGTCCCTGCGCCGCGCCGCCAGCGGTCCGC |
| | GATCGTTGCGGCCCGTAGCCAGTGGCAACTGGCAAAGCACACT |
| | GAACAGCATCGTGGGTCTGGGGGTGCAATCCCTGAAGCGCCGA |
| | CGATGCTTCTGATAGCTAACGTGTCGTATGTGTGTCATGTATGC |
| | GTCCATGTCGCCGCCAGAGGAGCTGCTGAGCCGCCGCGCGCCC |
| | GCTTTCCAAGATGGCTACCCCTTCGATGATGCCGCAGTGGTCTT |
| | ACATGCACATCTCGGGCCAGGACGCCTCGGAGTACCTGAGCCCC |
| | GGGCTGGTGCAGTTTGCCCGCGCCACCGAGACGTACTTCAGCCT |
| | GAATAACAAGTTTAGAAACCCCACGGTGGCGCCTACGCACGAC |
| | GTGACCACAGACCGGTCCCAGCGTTTGACGCTGCGGTTCATCCC |
| | TGTGGACCGTGAGGATACTGCGTACTCGTACAAGGCGCGGTTCA |
| | CCCTAGCTGTGGGTGATAACCGTGTGCTGGACATGGCTTCCACG |
| | TACTTTGACATCCGCGGCGTGCTGGACAGGGGCCCTACTTTTAA |
| | GCCCTACTCTGGCACTGCCTACAACGCCCTGGCTCCCAAGGGTG |
| | CCCCAAATCCTTGCGAATGGGATGAAGCTGCTACTGCTCTTGAA |
| | ATAAACCTAGAAGAAGAGGACGATGACAACGAAGACGAAGTA |
| | GACGAGCAAGCTGAGCAGCAAAAAACTCACGTATTTGGGCAGG |
| | CGCCTTATTCTGGTATAAATATTACAAAGGAGGGTATTCAAATA |
| | GGTGTCGAAGGTCAAACACCTAAATATGCCGATAAAACATTTCA |
| | ACCTGAACCTCAAATAGGAGAATCTCAGTGGTACGAAACAGAA |
| | ATTAATCATGCAGCTGGGAGAGTCCTAAAAAAGACTACCCCAA |
| | TGAAACCATGTTACGGTTCATATGCAAAACCCACAAATGAAAAT |
| | GGAGGGCAAGGCATTCTTGTAAAGCAACAAAATGGAAAGCTAG |
| | AAAGTCAAGTGGAAATGCAATTTTTCTCAACTACTGAGGCAGCC |
| | GCAGGCAATGGTGATAACTTGACTCCTAAAGTGGTATTGTACAG |
| | TGAAGATGTAGATATAGAAACCCCAGACACTCATATTTCTTACA |
| | TGCCCACTATTAAGGAAGGTAACTCACGAGAACTAATGGGCCA |
| | ACAATCTATGCCCAACAGGCCTAATTACATTGCTTTTAGGGACA |
| | ATTTTATTGGTCTAATGTATTACAACAGCACGGGTAATATGGGT |
| | GTTCTGGCGGGCCAAGCATCGCAGTTGAATGCTGTTGTAGATTT |
| | GCAAGACAGAAACACAGAGCTTTCATACCAGCTTTTGCTTGATT |
| | CCATTGGTGATAGAACCAGGTACTTTTCTATGTGGAATCAGGCT |
| | GTTGACAGCTATGATCCAGATGTTAGAATTATTGAAAATCATGG |
| | AACTGAAGATGAACTTCCAAATTACTGCTTTCCACTGGGAGGTG |
| | TGATTAATACAGAGACTCTTACCAAGGTAAAACCTAAAACAGG |
| | TCAGGAAAATGGATGGGAAAAAGATGCTACAGAATTTTCAGAT |
| | AAAAATGAAATAAGAGTTGGAAATAATTTTGCCATGGAAATCA |
| | ATCTAAATGCCAACCTGTGGAGAAATTTCCTGTACTCCAACATA |
| | GCGCTGTATTTGCCCGACAAGCTAAAGTACAGTCCTTCCAACGT |
| | AAAAATTTCTGATAACCCAAACACCTACGACTACATGAACAAG |
| | CGAGTGGTGGCTCCCGGGCTAGTGGACTGCTACATTAACCTTGG |
| | AGCACGCTGGTCCCTTGACTATATGGACAACGTCAACCCATTTA |
| | ACCACCACCGCAATGCTGGCCTGCGCTACCGCTCAATGTTGCTG |
| | GGCAATGGTCGCTATGTGCCCTTCCACATCCAGGTGCCTCAGAA |
| | GTTCTTTGCCATTAAAAACCTCCTTCTCCTGCCGGGCTCATACAC |
| | CTACGAGTGGAACTTCAGGAAGGATGTTAACATGGTTCTGCAGA |
| | GCTCCCTAGGAAATGACCTAAGGGTTGACGGAGCCAGCATTAA |
| | GTTTGATAGCATTTGCCTTTACGCCACCTTCTTCCCCATGGCCCA |
| | CAACACCGCCTCCACGCTTGAGGCCATGCTTAGAAACGACACCA |
| | ACGACCAGTCCTTTAACGACTATCTCTCCGCCGCCAACATGCTC |
| | TACCCTATACCCGCCAACGCTACCAACGTGCCCATATCCATCCC |
| | CTCCCGCAACTGGGCGGCTTTCCGCGGCTGGGCCTTCACGCGCC |
| | TTAAGACTAAGGAAACCCCATCACTGGGCTCGGGCTACGACCCT |
| | TATTACACCTACTCTGGCTCTATACCCTACCTAGATGGAACCTTT |
| | TACCTCAACCACACCTTTAAGAAGGTGGCCATTACCTTTGACTC |
| | TTCTGTCAGCTGGCCTGGCAATGACCGCCTGCTTACCCCCAACG |
| | AGTTTGAAATTAAGCGCTCAGTTGACGGGGAGGGTTACAACGTT |
| | GCCCAGTGTAACATGACCAAAGACTGGTTCCTGGTACAAATGCT |
| | AGCTAACTATAACATTGGCTACCAGGGCTTCTATATCCCAGAGA |
| | GCTACAAGGACCGCATGTACTCCTTCTTTAGAAACTTCCAGCCC |
| | ATGAGCCGTCAGGTGGTGGATGATACTAAATACAAGGACTACC |
| | AACAGGTGGGCATCCTACACCAACACAACAACTCTGGATTTGTT |
| | GGCTACCTTGCCCCCACCATGCGCGAAGGACAGGCCTACCCTGC |
| | TAACTTCCCCTATCCGCTTATAGGCAAGACCGCAGTTGACAGCA |
| | TTACCCAGAAAAAGTTTCTTTGCGATCGCACCCTTTGGCGCATC |
| | CCATTCTCCAGTAACTTTATGTCCATGGGCGCACTCACAGACCT |
| | GGGCCAAAACCTTCTCTACGCCAACTCCGCCCACGCGCTAGACA |
| | TGACTTTTGAGGTGGATCCCATGGACGAGCCCACCCTTCTTTAT |
| | GTTTTGTTTGAAGTCTTTGACGTGGTCCGTGTGCACCAGCCGCA |
| | CCGCGGCGTCATCGAAACCGTGTACCTGCGCACGCCCTTCTCGG |
| | CCGGCAACGCCACAACATAAAGAAGCAAGCAACATCAACAACA |
| | GCTGCCGCCATGGGCTCCAGTGAGCAGGAACTGAAAGCCATTG |
| | TCAAAGATCTTGGTTGTGGGCCATATTTTTTGGGCACCTATGAC |
| | AAGCGCTTTCCAGGCTTTGTTTCTCCACACAAGCTCGCCTGCGC |
| | CATAGTCAATACGGCCGGTCGCGAGACTGGGGGCGTACACTGG |
| | ATGGCCTTTGCCTGGAACCCGCACTCAAAAACATGCTACCTCTT |
| | TGAGCCCTTTGGCTTTTCTGACCAGCGACTCAAGCAGGTTTACC |
| | AGTTTGAGTACGAGTCACTCCTGCGCCGTAGCGCCATTGCTTCT |
| | TCCCCCGACCGCTGTATAACGCTGGAAAAGTCCACCCAAAGCGT |
| | ACAGGGGCCCAACTCGGCCGCCTGTGGACTATTCTGCTGCATGT |
| | TTCTCCACGCCTTTGCCAACTGGCCCCAAACTCCCATGGATCAC |
| | AACCCCACCATGAACCTTATTACCGGGGTACCCAACTCCATGCT |
| | CAACAGTCCCCAGGTACAGCCCACCCTGCGTCGCAACCAGGAA |
| | CAGCTCTACAGCTTCCTGGAGCGCCACTCGCCCTACTTCCGCAG |
| | CCACAGTGCGCAGATTAGGAGCGCCACTTCTTTTTGTCACTTGA |
| | AAAACATGTAAAAATAATGTACTAGAGACACTTTCAATAAAGG |
| | CAAATGCTTTTATTTGTACACTCTCGGGTGATTATTTACCCCCAC |
| | CCTTGCCGTCTGCGCCGTTTAAAAATCAAAGGGGTTCTGCCGCG |
| | CATCGCTATGCGCCACTGGCAGGGACACGTTGCGATACTGGTGT |
| | TTAGTGCTCCACTTAAACTCAGGCACAACCATCCGCGGCAGCTC |
| | GGTGAAGTTTTCACTCCACAGGCTGCGCACCATCACCAACGCGT |
| | TTAGCAGGTCGGGCGCCGATATCTTGAAGTCGCAGTTGGGGCCT |
| | CCGCCCTGCGCGCGCGAGTTGCGATACACAGGGTTGCAGCACTG |
| | GAACACTATCAGCGCCGGGTGGTGCACGCTGGCCAGCACGCTCT |
| | TGTCGGAGATCAGATCCGCGTCCAGGTCCTCCGCGTTGCTCAGG |
| | GCGAACGGAGTCAACTTTGGTAGCTGCCTTCCCAAAAAGGGCG |
| | CGTGCCCAGGCTTTGAGTTGCACTCGCACCGTAGTGGCATCAAA |
| | AGGTGACCGTGCCCGGTCTGGGCGTTAGGATACAGCGCCTGCAT |
| | AAAAGCCTTGATCTGCTTAAAAGCCACCTGAGCCTTTGCGCCTT |
| | CAGAGAAGAACATGCCGCAAGACTTGCCGGAAAACTGATTGGC |
| | CGGACAGGCCGCGTCGTGCACGCAGCACCTTGCGTCGGTGTTGG |
| | AGATCTGCACCACATTTCGGCCCCACCGGTTCTTCACGATCTTG |
| | GCCTTGCTAGACTGCTCCTTCAGCGCGCGCTGCCCGTTTTCGCTC |
| | GTCACATCCATTTCAATCACGTGCTCCTTATTTATCATAATGCTT |
| | CCGTGTAGACACTTAAGCTCGCCTTCGATCTCAGCGCAGCGGTG |
| | CAGCCACAACGCGCAGCCCGTGGGCTCGTGATGCTTGTAGGTCA |
| | CCTCTGCAAACGACTGCAGGTACGCCTGCAGGAATCGCCCCATC |
| | ATCGTCACAAAGGTCTTGTTGCTGGTGAAGGTCAGCTGCAACCC |
| | GCGGTGCTCCTCGTTCAGCCAGGTCTTGCATACGGCCGCCAGAG |
| | CTTCCACTTGGTCAGGCAGTAGTTTGAAGTTCGCCTTTAGATCGT |
| | TATCCACGTGGTACTTGTCCATCAGCGCGCGCGCAGCCTCCATG |
| | CCCTTCTCCCACGCAGACACGATCGGCACACTCAGCGGGTTCAT |
| | CACCGTAATTTCACTTTCCGCTTCGCTGGGCTCTTCCTCTTCCTC |
| | TTGCGTCCGCATACCACGCGCCACTGGGTCGTCTTCATTCAGCC |
| | GCCGCACTGTGCGCTTACCTCCTTTGCCATGCTTGATTAGCACCG |
| | GTGGGTTGCTGAAACCCACCATTTGTAGCGCCACATCTTCTCTTT |
| | CTTCCTCGCTGTCCACGATTACCTCTGGTGATGGCGGGCGCTCG |
| | GGCTTGGGAGAAGGGCGCTTCTTTTTCTTCTTGGGCGCAATGGC |
| | CAAATCCGCCGCCGAGGTCGATGGCCGCGGGCTGGGTGTGCGC |
| | GGCACCAGCGCGTCTTGTGATGAGTCTTCCTCGTCCTCGGACTC |
| | GATACGCCGCCTCATCCGCTTTTTTGGGGGCGCCCGGGGAGGCG |
| | GCGGCGACGGGGACGGGGACGACACGTCCTCCATGGTTGGGGG |
| | ACGTCGCGCCGCACCGCGTCCGCGCTCGGGGGTGGTTTCGCGCT |
| | GCTCCTCTTCCCGACTGGCCATTTCCTTCTCCTATAGGCAGAAAA |
| | AGATCATGGAGTCAGTCGAGAAGAAGGACAGCCTAACCGCCCC |
| | CTCTGAGTTCGCCACCACCGCCTCCACCGATGCCGCCAACGCGC |
| | CTACCACCTTCCCCGTCGAGGCACCCCCGCTTGAGGAGGAGGAA |
| | GTGATTATCGAGCAGGACCCAGGTTTTGTAAGCGAAGACGACG |
| | AGGACCGCTCAGTACCAACAGAGGATAAAAAGCAAGACCAGGA |
| | CAACGCAGAGGCAAACGAGGAACAAGTCGGGCGGGGGGACGA |
| | AAGGCATGGCGACTACCTAGATGTGGGAGACGACGTGCTGTTG |
| | AAGCATCTGCAGCGCCAGTGCGCCATTATCTGCGACGCGTTGCA |
| | AGAGCGCAGCGATGTGCCCCTCGCCATAGCGGATGTCAGCCTTG |
| | CCTACGAACGCCACCTATTCTCACCGCGCGTACCCCCCAAACGC |
| | CAAGAAAACGGCACATGCGAGCCCAACCCGCGCCTCAACTTCT |
| | ACCCCGTATTTGCCGTGCCAGAGGTGCTTGCCACCTATCACATC |
| | TTTTTCCAAAACTGCAAGATACCCCTATCCTGCCGTGCCAACCG |
| | CAGCCGAGCGGACAAGCAGCTGGCCTTGCGGCAGGGCGCTGTC |
| | ATACCTGATATCGCCTCGCTCAACGAAGTGCCAAAAATCTTTGA |
| | GGGTCTTGGACGCGACGAGAAGCGCGCGGCAAACGCTCTGCAA |
| | CAGGAAAACAGCGAAAATGAAAGTCACTCTGGAGTGTTGGTGG |
| | AACTCGAGGGTGACAACGCGCGCCTAGCCGTACTAAAACGCAG |
| | CATCGAGGTCACCCACTTTGCCTACCCGGCACTTAACCTACCCC |
| | CCAAGGTCATGAGCACAGTCATGAGTGAGCTGATCGTGCGCCGT |
| | GCGCAGCCCCTGGAGAGGGATGCAAATTTGCAAGAACAAACAG |
| | AGGAGGGCCTACCCGCAGTTGGCGACGAGCAGCTAGCGCGCTG |
| | GCTTCAAACGCGCGAGCCTGCCGACTTGGAGGAGCGACGCAAA |
| | CTAATGATGGCCGCAGTGCTCGTTACCGTGGAGCTTGAGTGCAT |
| | GCAGCGGTTCTTTGCTGACCCGGAGATGCAGCGCAAGCTAGAG |
| | GAAACATTGCACTACACCTTTCGACAGGGCTACGTACGCCAGGC |
| | CTGCAAGATCTCCAACGTGGAGCTCTGCAACCTGGTCTCCTACC |
| | TTGGAATTTTGCACGAAAACCGCCTTGGGCAAAACGTGCTTCAT |
| | TCCACGCTCAAGGGCGAGGCGCGCCGCGACTACGTCCGCGACT |
| | GCGTTTACTTATTTCTATGCTACACCTGGCAGACGGCCATGGGC |
| | GTTTGGCAGCAGTGCTTGGAGGAGTGCAACCTCAAGGAGCTGC |
| | AGAAACTGCTAAAGCAAAACTTGAAGGACCTATGGACGGCCTT |
| | CAACGAGCGCTCCGTGGCCGCGCACCTGGCGGACATCATTTTCC |
| | CCGAACGCCTGCTTAAAACCCTGCAACAGGGTCTGCCAGACTTC |
| | ACCAGTCAAAGCATGTTGCAGAACTTTAGGAACTTTATCCTAGA |
| | GCGCTCAGGAATCTTGCCCGCCACCTGCTGTGCACTTCCTAGCG |
| | ACTTTGTGCCCATTAAGTACCGCGAATGCCCTCCGCCGCTTTGG |
| | GGCCACTGCTACCTTCTGCAGCTAGCCAACTACCTTGCCTACCA |
| | CTCTGACATAATGGAAGACGTGAGCGGTGACGGTCTACTGGAG |
| | TGTCACTGTCGCTGCAACCTATGCACCCCGCACCGCTCCCTGGT |
| | TTGCAATTCGCAGCTGCTTAACGAAAGTCAAATTATCGGTACCT |
| | TTGAGCTGCAGGGTCCCTCGCCTGACGAAAAGTCCGCGGCTCCG |
| | GGGTTGAAACTCACTCCGGGGCTGTGGACGTCGGCTTACCTTCG |
| | CAAATTTGTACCTGAGGACTACCACGCCCACGAGATTAGGTTCT |
| | ACGAAGACCAATCCCGCCCGCCTAATGCGGAGCTTACCGCCTGC |
| | GTCATTACCCAGGGCCACATTCTTGGCCAATTGCAAGCCATCAA |
| | CAAAGCCCGCCAAGAGTTTCTGCTACGAAAGGGACGGGGGGTT |
| | TACTTGGACCCCCAGTCCGGCGAGGAGCTCAACCCAATCCCCCC |
| | GCCGCCGCAGCCCTATCAGCAGCAGCCGCGGGCCCTTGCTTCCC |
| | AGGATGGCACCCAAAAAGAAGCTGCAGCTGCCGCCGCCACCCA |
| | CGGACGAGGAGGAATACTGGGACAGTCAGGCAGAGGAGGTTTT |
| | GGACGAGGAGGAGGAGGACATGATGGAAGACTGGGAGAGCCT |
| | AGACGAGGAAGCTTCCGAGGTCGAAGAGGTGTCAGACGAAACA |
| | CCGTCACCCTCGGTCGCATTCCCCTCGCCGGCGCCCCAGAAATC |
| | GGCAACCGGTTCCAGCATGGCTACAACCTCCGCTCCTCAGGCGC |
| | CGCCGGCACTGCCCGTTCGCCGACCCAACCGTAGATGGGACACC |
| | ACTGGAACCAGGGCCGGTAAGTCCAAGCAGCCGCCGCCGTTAG |
| | CCCAAGAGCAACAACAGCGCCAAGGCTACCGCTCATGGCGCGG |
| | GCACAAGAACGCCATAGTTGCTTGCTTGCAAGACTGTGGGGGC |
| | AACATCTCCTTCGCCCGCCGCTTTCTTCTCTACCATCACGGCGTG |
| | GCCTTCCCCCGTAACATCCTGCATTACTACCGTCATCTCTACAGC |
| | CCATACTGCACCGGCGGCAGCGGCAGCAACAGCAGCGGCCACA |
| | CAGAAGCAAAGGCGACCGGATAGCAAGACTCTGACAAAGCCCA |
| | AGAAATCCACAGCGGCGGCAGCAGCAGGAGGAGGAGCGCTGC |
| | GTCTGGCGCCCAACGAACCCGTATCGACCCGCGAGCTTAGAAA |
| | CAGGATTTTTCCCACTCTGTATGCTATATTTCAACAGAGCAGGG |
| | GCCAAGAACAAGAGCTGAAAATAAAAAACAGGTCTCTGCGATC |
| | CCTCACCCGCAGCTGCCTGTATCACAAAAGCGAAGATCAGCTTC |
| | GGCGCACGCTGGAAGACGCGGAGGCTCTCTTCAGTAAATACTG |
| | CGCGCTGACTCTTAAGGACTAGTTTCGCGCCCTTTCTCAAATTTA |
| | AGCGCGAAAACTACGTCATCTCCAGCGGCCACACCCGGCGCCA |
| | GCACCTGTTGTCAGCGCCATTATGAGCAAGGAAATTCCCACGCC |
| | CTACATGTGGAGTTACCAGCCACAAATGGGACTTGCGGCTGGA |
| | GCTGCCCAAGACTACTCAACCCGAATAAACTACATGAGCGCGG |
| | GACCCCACATGATATCCCGGGTCAACGGAATACGCGCCCACCG |
| | AAACCGAATTCTCCTGGAACAGGCGGCTATTACCACCACACCTC |
| | GTAATAACCTTAATCCCCGTAGTTGGCCCGCTGCCCTGGTGTAC |
| | CAGGAAAGTCCCGCTCCCACCACTGTGGTACTTCCCAGAGACGC |
| | CCAGGCCGAAGTTCAGATGACTAACTCAGGGGCGCAGCTTGCG |
| | GGCGGCTTTCGTCACAGGGTGCGGTCGCCCGGGCAGGGTATAA |
| | CTCACCTGACAATCAGAGGGCGAGGTATTCAGCTCAACGACGA |
| | GTCGGTGAGCTCCTCGCTTGGTCTCCGTCCGGACGGGACATTTC |
| | AGATCGGCGGCGCCGGCCGCTCTTCATTCACGCCTCGTCAGGCA |
| | ATCCTAACTCTGCAGACCTCGTCCTCTGAGCCGCGCTCTGGAGG |
| | CATTGGAACTCTGCAATTTATTGAGGAGTTTGTGCCATCGGTCT |
| | ACTTTAACCCCTTCTCGGGACCTCCCGGCCACTATCCGGATCAA |
| | TTTATTCCTAACTTTGACGCGGTAAAGGACTCGGCGGACGGCTA |
| | CGACTGAATGTTAAGTGGAGAGGCAGAGCAACTGCGCCTGAAA |
| | CACCTGGTCCACTGTCGCCGCCACAAGTGCTTTGCCCGCGACTC |
| | CGGTGAGTTTTGCTACTTTGAATTGCCCGAGGATCATATCGAGG |
| | GCCCGGCGCACGGCGTCCGGCTTACCGCCCAGGGAGAGCTTGC |
| | CCGTAGCCTGATTCGGGAGTTTACCCAGCGCCCCCTGCTAGTTG |
| | AGCGGGACAGGGGACCCTGTGTTCTCACTGTGATTTGCAACTGT |
| | CCTAACCCTGGATTACATCAAGATCCTCTAGTTAATGTCAGGTC |
| | GCCTAAGTCGATTAACTAGAGTACCCGGGGATCTTATTCCCTTT |
| | AACTAATAAAAAAAAATAATAAAGCATCACTTACTTAAAATCA |
| | GTTAGCAAATTTCTGTCCAGTTTATTCAGCAGCACCTCCTTGCCC |
| | TCCTCCCAGCTCTGGTATTGCAGCTTCCTCCTGGCTGCAAACTTT |
| | CTCCACAATCTAAATGGAATGTCAGTTTCCTCCTGTTCCTGTCCA |
| | TCCGCACCCACTATCTTCATGTTGTTGCAGATGAAGCGCGCAAG |
| | ACCGTCTGAAGATACCTTCAACCCCGTGTATCCATATGACACGG |
| | AAACCGGTCCTCCAACTGTGCCTTTTCTTACTCCTCCCTTTGTAT |
| | CCCCCAATGGGTTTCAAGAGAGTCCCCCTGGGGTACTCTCTTTG |
| | CGCCTATCCGAACCTCTAGTTACCTCCAATGGCATGCTTGCGCT |
| | CAAAATGGGCAACGGCCTCTCTCTGGACGAGGCCGGCAACCTT |
| | ACCTCCCAAAATGTAACCACTGTGAGCCCACCTCTCAAAAAAAC |
| | CAAGTCAAACATAAACCTGGAAATATCTGCACCCCTCACAGTTA |
| | CCTCAGAAGCCCTAACTGTGGCTGCCGCCGCACCTCTAATGGTC |
| | GCGGGCAACACACTCACCATGCAATCACAGGCCCCGCTAACCG |
| | TGCACGACTCCAAACTTAGCATTGCCACCCAAGGACCCCTCACA |
| | GTGTCAGAAGGAAAGCTAGCCCTGCAAACATCAGGCCCCCTCA |
| | CCACCACCGATAGCAGTACCCTTACTATCACTGCCTCACCCCCT |
| | CTAACTACTGCCACTGGTAGCTTGGGCATTGACTTGAAAGAGCC |
| | CATTTATACACAAAATGGAAAACTAGGACTAAAGTACGGGGCT |
| | CCTTTGCATGTAACAGACGACCTAAACACTTTGACCGTAGCAAC |
| | TGGTCCAGGTGTGACTATTAATAATACTTCCTTGCAAACTAAAG |
| | TTACTGGAGCCTTGGGTTTTGATTCACAAGGCAATATGCAACTT |
| | AATGTAGCAGGAGGACTAAGGATTGATTCTCAAAACAGACGCC |
| | TTATACTTGATGTTAGTTATCCGTTTGATGCTCAAAACCAACTAA |
| | ATCTAAGACTAGGACAGGGCCCTCTTTTTATAAACTCAGCCCAC |
| | AACTTGGATATTAACTACAACAAAGGCCTTTACTTGTTTACAGC |
| | TTCAAACAATTCCAAAAAGCTTGAGGTTAACCTAAGCACTGCCA |
| | AGGGGTTGATGTTTGACGCTACAGCCATAGCCATTAATGCAGGA |
| | GATGGGCTTGAATTTGGTTCACCTAATGCACCAAACACAAATCC |
| | CCTCAAAACAAAAATTGGCCATGGCCTAGAATTTGATTCAAACA |
| | AGGCTATGGTTCCTAAACTAGGAACTGGCCTTAGTTTTGACAGC |
| | ACAGGTGCCATTACAGTAGGAAACAAAAATAATGATAAGCTAA |
| | CTTTGTGGACCACACCAGCTCCATCTCCTAACTGTAGACTAAAT |
| | GCAGAGAAAGATGCTAAACTCACTTTGGTCTTAACAAAATGTGG |
| | CAGTCAAATACTTGCTACAGTTTCAGTTTTGGCTGTTAAAGGCA |
| | GTTTGGCTCCAATATCTGGAACAGTTCAAAGTGCTCATCTTATT |
| | ATAAGATTTGACGAAAATGGAGTGCTACTAAACAATTCCTTCCT |
| | GGACCCAGAATATTGGAACTTTAGAAATGGAGATCTTACTGAA |
| | GGCACAGCCTATACAAACGCTGTTGGATTTATGCCTAACCTATC |
| | AGCTTATCCAAAATCTCACGGTAAAACTGCCAAAAGTAACATTG |
| | TCAGTCAAGTTTACTTAAACGGAGACAAAACTAAACCTGTAACA |
| | CTAACCATTACACTAAACGGTACACAGGAAACAGGAGACACAA |
| | CTCCAAGTGCATACTCTATGTCATTTTCATGGGACTGGTCTGGCC |
| | ACAACTACATTAATGAAATATTTGCCACATCCTCTTACACTTTTT |
| | CATACATTGCCCAAGAATAAAGAATCGTTTGTGTTATGTTTCAA |
| | CGTGTTTATTTTTCAATTGCAGAAAATTTCAAGTCATTTTTCATT |
| | CAGTAGTATAGCCCCACCACCACATAGCTTATACAGATCACCGT |
| | ACCTTAATCAAACTCACAGAACCCTAGTATTCAACCTGCCACCT |
| | CCCTCCCAACACACAGAGTACACAGTCCTTTCTCCCCGGCTGGC |
| | CTTAAAAAGCATCATATCATGGGTAACAGACATATTCTTAGGTG |
| | TTATATTCCACACGGTTTCCTGTCGAGCCAAACGCTCATCAGTG |
| | ATATTAATAAACTCCCCGGGCAGCTCACTTAAGTTCATGTCGCT |
| | GTCCAGCTGCTGAGCCACAGGCTGCTGTCCAACTTGCGGTTGCT |
| | TAACGGGCGGCGAAGGAGAAGTCCACGCCTACATGGGGGTAGA |
| | GTCATAATCGTGCATCAGGATAGGGCGGTGGTGCTGCAGCAGC |
| | GCGCGAATAAACTGCTGCCGCCGCCGCTCCGTCCTGCAGGAATA |
| | CAACATGGCAGTGGTCTCCTCAGCGATGATTCGCACCGCCCGCA |
| | GCATAAGGCGCCTTGTCCTCCGGGCACAGCAGCGCACCCTGATC |
| | TCACTTAAATCAGCACAGTAACTGCAGCACAGCACCACAATATT |
| | GTTCAAAATCCCACAGTGCAAGGCGCTGTATCCAAAGCTCATGG |
| | CGGGGACCACAGAACCCACGTGGCCATCATACCACAAGCGCAG |
| | GTAGATTAAGTGGCGACCCCTCATAAACACGCTGGACATAAAC |
| | ATTACCTCTTTTGGCATGTTGTAATTCACCACCTCCCGGTACCAT |
| | ATAAACCTCTGATTAAACATGGCGCCATCCACCACCATCCTAAA |
| | CCAGCTGGCCAAAACCTGCCCGCCGGCTATACACTGCAGGGAA |
| | CCGGGACTGGAACAATGACAGTGGAGAGCCCAGGACTCGTAAC |
| | CATGGATCATCATGCTCGTCATGATATCAATGTTGGCACAACAC |
| | AGGCACACGTGCATACACTTCCTCAGGATTACAAGCTCCTCCCG |
| | CGTTAGAACCATATCCCAGGGAACAACCCATTCCTGAATCAGCG |
| | TAAATCCCACACTGCAGGGAAGACCTCGCACGTAACTCACGTTG |
| | TGCATTGTCAAAGTGTTACATTCGGGCAGCAGCGGATGATCCTC |
| | CAGTATGGTAGCGCGGGTTTCTGTCTCAAAAGGAGGTAGACGAT |
| | CCCTACTGTACGGAGTGCGCCGAGACAACCGAGATCGTGTTGGT |
| | CGTAGTGTCATGCCAAATGGAACGCCGGACGTAGTCATATTTCC |
| | TGAAGCAAAACCAGGTGCGGGCGTGACAAACAGATCTGCGTCT |
| | CCGGTCTCGCCGCTTAGATCGCTCTGTGTAGTAGTTGTAGTATAT |
| | CCACTCTCTCAAAGCATCCAGGCGCCCCCTGGCTTCGGGTTCTA |
| | TGTAAACTCCTTCATGCGCCGCTGCCCTGATAACATCCACCACC |
| | GCAGAATAAGCCACACCCAGCCAACCTACACATTCGTTCTGCGA |
| | GTCACACACGGGAGGAGCGGGAAGAGCTGGAAGAACCATGTTT |
| | TTTTTTTTATTCCAAAAGATTATCCAAAACCTCAAAATGAAGAT |
| | CTATTAAGTGAACGCGCTCCCCTCCGGTGGCGTGGTCAAACTCT |
| | ACAGCCAAAGAACAGATAATGGCATTTGTAAGATGTTGCACAA |
| | TGGCTTCCAAAAGGCAAACGGCCCTCACGTCCAAGTGGACGTA |
| | AAGGCTAAACCCTTCAGGGTGAATCTCCTCTATAAACATTCCAG |
| | CACCTTCAACCATGCCCAAATAATTCTCATCTCGCCACCTTCTCA |
| | ATATATCTCTAAGCAAATCCCGAATATTAAGTCCGGCCATTGTA |
| | AAAATCTGCTCCAGAGCGCCCTCCACCTTCAGCCTCAAGCAGCG |
| | AATCATGATTGCAAAAATTCAGGTTCCTCACAGACCTGTATAAG |
| | ATTCAAAAGCGGAACATTAACAAAAATACCGCGATCCCGTAGG |
| | TCCCTTCGCAGGGCCAGCTGAACATAATCGTGCAGGTCTGCACG |
| | GACCAGCGCGGCCACTTCCCCGCCAGGAACCATGACAAAAGAA |
| | CCCACACTGATTATGACACGCATACTCGGAGCTATGCTAACCAG |
| | CGTAGCCCCGATGTAAGCTTGTTGCATGGGCGGCGATATAAAAT |
| | GCAAGGTGCTGCTCAAAAAATCAGGCAAAGCCTCGCGCAAAAA |
| | AGAAAGCACATCGTAGTCATGCTCATGCAGATAAAGGCAGGTA |
| | AGCTCCGGAACCACCACAGAAAAAGACACCATTTTTCTCTCAAA |
| | CATGTCTGCGGGTTTCTGCATAAACACAAAATAAAATAACAAA |
| | AAAACATTTAAACATTAGAAGCCTGTCTTACAACAGGAAAAAC |
| | AACCCTTATAAGCATAAGACGGACTACGGCCATGCCGGCGTGA |
| | CCGTAAAAAAACTGGTCACCGTGATTAAAAAGCACCACCGACA |
| | GCTCCTCGGTCATGTCCGGAGTCATAATGTAAGACTCGGTAAAC |
| | ACATCAGGTTGATTCACATCGGTCAGTGCTAAAAAGCGACCGA |
| | AATAGCCCGGGGGAATACATACCCGCAGGCGTAGAGACAACAT |
| | TACAGCCCCCATAGGAGGTATAACAAAATTAATAGGAGAGAAA |
| | AACACATAAACACCTGAAAAACCCTCCTGCCTAGGCAAAATAG |
| | CACCCTCCCGCTCCAGAACAACATACAGCGCTTCCACAGCGGCA |
| | GCCATAACAGTCAGCCTTACCAGTAAAAAAGAAAACCTATTAA |
| | AAAAACACCACTCGACACGGCACCAGCTCAATCAGTCACAGTG |
| | TAAAAAAGGGCCAAGTGCAGAGCGAGTATATATAGGACTAAAA |
| | AATGACGTAACGGTTAAAGTCCACAAAAAACACCCAGAAAACC |
| | GCACGCGAACCTACGCCCAGAAACGAAAGCCAAAAAACCCACA |
| | ACTTCCTCAAATCGTCACTTCCGTTTTCCCACGTTACGTCACTTC |
| | CCATTTTAAGAAAACTACAATTCCCAACACATACAAGTTACTCC |
| | GCCCTAAAACCTACGTCACCCGCCCCGTTCCCACGCCCCGCGCC |
| | ACGTCACAAACTCCACCCCCTCATTATCATATTGGCTTCAATCC |
| | AAAATAAGGTATATTATTGATGAT |
| SEQ ID NO: 3 | YLSGANLNL |
| SEQ ID NO: 4 | YLSGADLNL |
| SEQ ID NO: 5 | CGCTCCACCTCTCAAGCAGCCAGCGCCTGCCTGAATCTGTTCTG |
| | CCCCCTCCCCACCCATTTCACCACCACCATGACACCGGGCACCC |
| | AGTCTCCTTTCTTCCTGCTGCTGCTCCTCACAGTGCTTACAGTTG |
| | TTACGGGTTCTGGTCATGCAAGCTCTACCCCAGGTGGAGAAAAG |
| | GAGACTTCGGCTACCCAGAGAAGTTCAGTGCCCAGCTCTACTGA |
| | GAAGAATGCTGTGAGTATGACCAGCAGCGTACTCTCCAGCCAC |
| | AGCCCCGGTTCAGGCTCCTCCACCACTCAGGGACAGGATGTCAC |
| | TCTGGCCCCGGCCACGGAACCAGCTTCAGGTTCAGCTGCCACCT |
| | GGGGACAGGATGTCACCTCGGTCCCAGTCACCAGGCCAGCCCT |
| | GGGCTCCACCACCCCGCCAGCCCACGATGTCACCTCAGCCCCGG |
| | ACAACAAGCCAGCCCCGGGCTCCACCGCCCCCCCAGCCCACGG |
| | TGTCACCTCGGCCCCGGACACCAGGCCGGCCCCGGGCTCCACCG |
| | CCCCCCCAGCCCATGGTGTCACCTCGGCCCCGGACAACAGGCCC |
| | GCCTTGGGCTCCACCGCCCCTCCAGTCCACAATGTCACCTCGGC |
| | CTCAGGCTCTGCATCAGGCTCAGCTTCTACTCTGGTGCACAACG |
| | GCACCTCTGCCAGGGCTACCACAACCCCAGCCAGCAAGAGCAC |
| | TCCATTCTCAATTCCCAGCCACCACTCTGATACTCCTACCACCCT |
| | TGCCAGCCATAGCACCAAGACTGATGCCAGTAGCACTCACCATA |
| | GCACGGTACCTCCTCTCACCTCCTCCAATCACAGCACTTCTCCCC |
| | AGTTGTCTACTGGGGTCTCTTTCTTTTTCCTGTCTTTTCACATTTC |
| | AAACCTCCAGTTTAATTCCTCTCTGGAAGATCCCAGCACCGACT |
| | ACTACCAAGAGCTGCAGAGAGACATTTCTGAAATGTTTTTGCAG |
| | ATTTATAAACAAGGGGGTTTTCTGGGCCTCTCCAATATTAAGTT |
| | CAGGCCAGGATCTGTGGTGGTACAATTGACTCTGGCCTTCCGAG |
| | AAGGTACCATCAATGTCCACGACGTGGAGACACAGTTCAATCA |
| | GTATAAAACGGAAGCAGCCTCTCGATATAACCTGACGATCTCAG |
| | ACGTCAGCGTGAGTGATGTGCCATTTCCTTTCTCTGCCCAGTCTG |
| | GGGCTGGGGTGCCAGGCTGGGGCATCGCGCTGCTGGTGCTGGTC |
| | TGTGTTCTGGTTGCGCTGGCCATTGTCTATCTCATTGCCTTGGCT |
| | GTCTGTCAGTGCCGCCGAAAGAACTACGGGCAGCTGGACATCTT |
| | TCCAGCCCGGGATACCTACCATCCTATGAGCGAGTACCCCACCT |
| | ACCACACCCATGGGCGCTATGTGCCCCCTAGCAGTACCGATCGT |
| | AGCCCCTATGAGAAGGTTTCTGCAGGTAATGGTGGCAGCAGCCT |
| | CTCTTACACAAACCCAGCAGTGGCAGCCACTTCTGCCAACTTGT |
| | AGGGGCACGTCGCCCGCTGAGCTGAGTGGCCAGCCAGTGCCAT |
| | TCCACTCCACTCAGGTTCTTCAGGGCCAGAGCCCCTGCACCCTG |
| | TTTGGGCTGGTGAGCTGGGAGTTCAGGTGGGCTGCTCACAGCCT |
| | CCTTCAGAGGCCCCACCAATTTCTCGGACACTTCTCAGTGTGTG |
| | GAAGCTCATGTGGGCCCCTGAGGGCTCATGCCTGGGAAGTGTTG |
| | TGGTGGGGGCTCCCAGGAGGACTGGCCCAGAGAGCCCTGAGAT |
| | AGCGGGGATCCTGAACTGGACTGAATAAAACGTGGTCTCCCACT |
| | GCGCCAAAAAAAAAAAAAAAAA |
| SEQ ID NO: 6 | CGCTCCACCTCTCAAGCAGCCAGCGCCTGCCTGAATCTGTTCTG |
| | CCCCCTCCCCACCCATTTCACCACCACCATGACACCGGGCACCC |
| | AGTCTCCTTTCTTCCTGCTGCTGCTCCTCACAGTGCTTACAGTTG |
| | TTACGGGTTCTGGTCATGCAAGCTCTACCCCAGGTGGAGAAAAG |
| | GAGACTTCGGCTACCCAGAGAAGTTCAGTGCCCAGCTCTACTGA |
| | GAAGAATGCTGTGAGTATGACCAGCAGCGTACTCTCCAGCCAC |
| | AGCCCCGGTTCAGGCTCCTCCACCACTCAGGGACAGGATGTCAC |
| | TCTGGCCCCGGCCACGGAACCAGCTTCAGGTTCAGCTGCCCTTT |
| | GGGGACAGGATGTCACCTCGGTCCCAGTCACCAGGCCAGCCCT |
| | GGGCTCCACCACCCCGCCAGCCCACGATGTCACCTCAGCCCCGG |
| | ACAACAAGCCAGCCCCGGGCTCCACCGCCCCCCCAGCCCACGG |
| | TGTCACCTCGTATCTTGACACCAGGCCGGCCCCGGTTTATCTTGC |
| | CCCCCCAGCCCATGGTGTCACCTCGGCCCCGGACAACAGGCCCG |
| | CCTTGGGCTCCACCGCCCCTCCAGTCCACAATGTCACCTCGGCC |
| | TCAGGCTCTGCATCAGGCTCAGCTTCTACTCTGGTGCACAACGG |
| | CACCTCTGCCAGGGCTACCACAACCCCAGCCAGCAAGAGCACT |
| | CCATTCTCAATTCCCAGCCACCACTCTGATACTCCTACCACCCTT |
| | GCCAGCCATAGCACCAAGACTGATGCCAGTAGCACTCACCATA |
| | GCACGGTACCTCCTCTCACCTCCTCCAATCACAGCACTTCTCCCC |
| | AGTTGTCTACTGGGGTCTCTTTCTTTTTCCTGTCTTTTCACATTTC |
| | AAACCTCCAGTTTAATTCCTCTCTGGAAGATCCCAGCACCGACT |
| | ACTACCAAGAGCTGCAGAGAGACATTTCTGAAATGTTTTTGCAG |
| | ATTTATAAACAAGGGGGTTTTCTGGGCCTCTCCAATATTAAGTT |
| | CAGGCCAGGATCTGTGGTGGTACAATTGACTCTGGCCTTCCGAG |
| | AAGGTACCATCAATGTCCACGACGTGGAGACACAGTTCAATCA |
| | GTATAAAACGGAAGCAGCCTCTCGATATAACCTGACGATCTCAG |
| | ACGTCAGCGTGAGTGATGTGCCATTTCCTTTCTCTGCCCAGTCTG |
| | GGGCTGGGGTGCCAGGCTGGGGCATCGCGCTGCTGGTGCTGGTC |
| | TGTGTTCTGGTTTATCTGGCCATTGTCTATCTCATTGCCTTGGCT |
| | GTCGCTCAGGTTCGCCGAAAGAACTACGGGCAGCTGGACATCTT |
| | TCCAGCCCGGGATAAATACCATCCTATGAGCGAGTACGCTCTTT |
| | ACCACACCCATGGGCGCTATGTGCCCCCTAGCAGTCTTTTCCGT |
| | AGCCCCTATGAGAAGGTTTCTGCAGGTAATGGTGGCAGCTATCT |
| | CTCTTACACAAACCCAGCAGTGGCAGCCGCTTCTGCCAACTTGT |
| | AGGGGCACGTCGCCCGCTGAGCTGAGTGGCCAGCCAGTGCCAT |
| | TCCACTCCACTCAGGTTCTTCAGGGCCAGAGCCCCTGCACCCTG |
| | TTTGGGCTGGTGAGCTGGGAGTTCAGGTGGGCTGCTCACAGCCT |
| | CCTTCAGAGGCCCCACCAATTTCTCGGACACTTCTCAGTGTGTG |
| | GAAGCTCATGTGGGCCCCTGAGGGCTCATGCCTGGGAAGTGTTG |
| | TGGTGGGGGCTCCCAGGAGGACTGGCCCAGAGAGCCCTGAGAT |
| | AGCGGGGATCCTGAACTGGACTGAATAAAACGTGGTCTCCCACT |
| | GCGCCAAAAAAAAAAAAAAAAA |
| SEQ ID NO: 7 | MTPGTQSPFFLLLLLTVLTVVTGSGHASSTPGGEKETSATQRSSVPS |
| | STEKNAVSMTSSVLSSHSPGSGSSTTQGQDVTLAPATEPASGSAAL |
| | WGQDVTSVPVTRPALGSTTPPAHDVTSAPDNKPAPGSTAPPAHGV |
| | TSYLDTRPAPVYLAPPAHGVTSAPDNRPALGSTAPPVHNVTSASGS |
| | ASGSASTLVHNGTSARATTTPASKSTPFSIPSHEISDTPTTLASHSTKT |
| | DASSTHHSTVPPLTSSNHSTSPQLSTGVSFFFLSFHISNLQFNSSLEDP |
| | STDYYQELQRDISEMFLQIYKQGGFLGLSNIKFRPGSVVVQLTLAFR |
| | EGTINVHDVETQFNQYKTEAASRYNLTISDVSVSDVPFPFSAQSGA |
| | GVPGWGIALLVLVCVLVYLAIVYLIALAVAQVRRKNYGQLDIFPA |
| | RDKYHPMSEYALYHTHGRYVPPSSLFRSPYEKVSAGNGGSYLSYT |
| | NPAVAAASANL |
| SEQ ID NO: 8 | CATCATCAATAATATACCTTATTTTGGATTGAAGCCAATATGAT |
| | AATGAGGGGGTGGAGTTTGTGACGTGGCGCGGGGCGTGGGAAC |
| | GGGGCGGGTGACGTAGTAGTGTGGCGGAAGTGTGATGTTGCAA |
| | GTGTGGCGGAACACATGTAAGCGACGGATGTGGCAAAAGTGAC |
| | GTTTTTGGTGTGCGCCGGTGTACACAGGAAGTGACAATTTTCGC |
| | GCGGTTTTAGGCGGATGTTGTAGTAAATTTGGGCGTAACCGAGT |
| | AAGATTTGGCCATTTTCGCGGGAAAACTGAATAAGAGGAAGTG |
| | AAATCTGAATAATTTTGTGTTACTCATAGCGCGTAATACTGTAA |
| | TAGTAATCAATTACGGGGTCATTAGTTCATAGCCCATATATGGA |
| | GTTCCGCGTTACATAACTTACGGTAAATGGCCCGCCTGGCTGAC |
| | CGCCCAACGACCCCCGCCCATTGACGTCAATAATGACGTATGTT |
| | CCCATAGTAACGCCAATAGGGACTTTCCATTGACGTCAATGGGT |
| | GGAGTATTTACGGTAAACTGCCCACTTGGCAGTACATCAAGTGT |
| | ATCATATGCCAAGTACGCCCCCTATTGACGTCAATGACGGTAAA |
| | TGGCCCGCCTGGCATTATGCCCAGTACATGACCTTATGGGACTT |
| | TCCTACTTGGCAGTACATCTACGTATTAGTCATCGCTATTACCAT |
| | GGTGATGCGGTTTTGGCAGTACATCAATGGGCGTGGATAGCGGT |
| | TTGACTCACGGGGATTTCCAAGTCTCCACCCCATTGACGTCAAT |
| | GGGAGTTTGTTTTGGCACCAAAATCAACGGGACTTTCCAAAATG |
| | TCGTAACAACTCCGCCCCATTGACGCAAATGGGCGGTAGGCGTG |
| | TACGGTGGGAGGTCTATATAAGCAGAGCTGGTTTAGTGAACCGT |
| | CAGATCCGCTAGAGATCTGGTACCGTCGACGCGGCCGCTCGAGC |
| | CTAAGCTTCTAGATGCATGCTCGAGCGGCCGCCAGTGTGATGGA |
| | TATCTGCAGAATTCGCCCTTGCTCGCTCCACCTCTCAAGCAGCC |
| | AGCGCCTGCCTGAATCTGTTCTGCCCCCTCCCCACCCATTTCACC |
| | ACCACCATGACACCGGGCACCCAGTCTCCTTTCTTCCTGCTGCT |
| | GCTCCTCACAGTGCTTACAGTTGTTACGGGTTCTGGTCATGCAA |
| | GCTCTACCCCAGGTGGAGAAAAGGAGACTTCGGCTACCCAGAG |
| | AAGTTCAGTGCCCAGCTCTACTGAGAAGAATGCTGTGAGTATGA |
| | CCAGCAGCGTACTCTCCAGCCACAGCCCCGGTTCAGGCTCCTCC |
| | ACCACTCAGGGACAGGATGTCACTCTGGCCCCGGCCACGGAAC |
| | CAGCTTCAGGTTCAGCTGCCCTTTGGGGACAGGATGTCACCTCG |
| | GTCCCAGTCACCAGGCCAGCCCTGGGCTCCACCACCCCGCCAGC |
| | CCACGATGTCACCTCAGCCCCGGACAACAAGCCAGCCCCGGGC |
| | TCCACCGCCCCCCCAGCCCACGGTGTCACCTCGTATCTTGACAC |
| | CAGGCCGGCCCCGGTTTATCTTGCCCCCCCAGCCCATGGTGTCA |
| | CCTCGGCCCCGGACAACAGGCCCGCCTTGGGCTCCACCGCCCCT |
| | CCAGTCCACAATGTCACCTCGGCCTCAGGCTCTGCATCAGGCTC |
| | AGCTTCTACTCTGGTGCACAACGGCACCTCTGCCAGGGCTACCA |
| | CAACCCCAGCCAGCAAGAGCACTCCATTCTCAATTCCCAGCCAC |
| | CACTCTGATACTCCTACCACCCTTGCCAGCCATAGCACCAAGAC |
| | TGATGCCAGTAGCACTCACCATAGCACGGTACCTCCTCTCACCT |
| | CCTCCAATCACAGCACTTCTCCCCAGTTGTCTACTGGGGTCTCTT |
| | TCTTTTTCCTGTCTTTTCACATTTCAAACCTCCAGTTTAATTCCTC |
| | TCTGGAAGATCCCAGCACCGACTACTACCAAGAGCTGCAGAGA |
| | GACATTTCTGAAATGTTTTTGCAGATTTATAAACAAGGGGGTTT |
| | TCTGGGCCTCTCCAATATTAAGTTCAGGCCAGGATCTGTGGTGG |
| | TACAATTGACTCTGGCCTTCCGAGAAGGTACCATCAATGTCCAC |
| | GACGTGGAGACACAGTTCAATCAGTATAAAACGGAAGCAGCCT |
| | CTCGATATAACCTGACGATCTCAGACGTCAGCGTGAGTGATGTG |
| | CCATTTCCTTTCTCTGCCCAGTCTGGGGCTGGGGTGCCAGGCTG |
| | GGGCATCGCGCTGCTGGTGCTGGTCTGTGTTCTGGTTTATCTGGC |
| | CATTGTCTATCTCATTGCCTTGGCTGTCGCTCAGGTTCGCCGAAA |
| | GAACTACGGGCAGCTGGACATCTTTCCAGCCCGGGATAAATACC |
| | ATCCTATGAGCGAGTACGCTCTTTACCACACCCATGGGCGCTAT |
| | GTGCCCCCTAGCAGTCTTTTCCGTAGCCCCTATGAGAAGGTTTCT |
| | GCAGGTAATGGTGGCAGCTATCTCTCTTACACAAACCCAGCAGT |
| | GGCAGCCGCTTCTGCCAACTTGTAGGGGCACGTCGCCCGCTGAG |
| | CTGAGTGGCCAGCCAGTGCCATTCCACTCCACTCAGGTTCTTCA |
| | GGGCCAGAGCCCCTGCACCCTGTTTGGGCTGGTGAGCTGGGAGT |
| | TCAGGTGGGCTGCTCACAGCCTCCTTCAGAGGCCCCACCAATTT |
| | CTCGGACACTTCTCAGTGTGTGGAAGCTCATGTGGGCCCCTGAG |
| | GGCTCATGCCTGGGAAGTGTTGTGGTGGGGGCTCCCAGGAGGA |
| | CTGGCCCAGAGAGCCCTGAGATAGCGGGGATCCTGAACTGGAC |
| | TGAATAAAACGTGGTCTCCCACTGCGCCAAAAAAAAAAAAAAA |
| | AACGATCCACCGGATCTAGATAACTGATCATAATCAGCCATACC |
| | ACATTTGTAGAGGTTTTACTTGCTTTAAAAAACCTCCCACACCTC |
| | CCCCTGAACCTGAAACATAAAATGAATGCAATTGTTGTTGTTAA |
| | CTTGTTTATTGCAGCTTATAATGGTTACAAATAAAGCAATAGCA |
| | TCACAAATTTCACAAATAAAGCATTTTTTTCACTGCATTCTAGTT |
| | GTGGTTTGTCCAAACTCATCAATGTATCTTAACGCGGATCTGGA |
| | AGGTGCTGAGGTACGATGAGACCCGCACCAGGTGCAGACCCTG |
| | CGAGTGTGGCGGTAAACATATTAGGAACCAGCCTGTGATGCTG |
| | GATGTGACCGAGGAGCTGAGGCCCGATCACTTGGTGCTGGCCTG |
| | CACCCGCGCTGAGTTTGGCTCTAGCGATGAAGATACAGATTGAG |
| | GTACTGAAATGTGTGGGCGTGGCTTAAGGGTGGGAAAGAATAT |
| | ATAAGGTGGGGGTCTTATGTAGTTTTGTATCTGTTTTGCAGCAG |
| | CCGCCGCCGCCATGAGCACCAACTCGTTTGATGGAAGCATTGTG |
| | AGCTCATATTTGACAACGCGCATGCCCCCATGGGCCGGGGTGCG |
| | TCAGAATGTGATGGGCTCCAGCATTGATGGTCGCCCCGTCCTGC |
| | CCGCAAACTCTACTACCTTGACCTACGAGACCGTGTCTGGAACG |
| | CCGTTGGAGACTGCAGCCTCCGCCGCCGCTTCAGCCGCTGCAGC |
| | CACCGCCCGCGGGATTGTGACTGACTTTGCTTTCCTGAGCCCGC |
| | TTGCAAGCAGTGCAGCTTCCCGTTCATCCGCCCGCGATGACAAG |
| | TTGACGGCTCTTTTGGCACAATTGGATTCTTTGACCCGGGAACTT |
| | AATGTCGTTTCTCAGCAGCTGTTGGATCTGCGCCAGCAGGTTTC |
| | TGCCCTGAAGGCTTCCTCCCCTCCCAATGCGGTTTAAAACATAA |
| | ATAAAAAACCAGACTCTGTTTGGATTTGGATCAAGCAAGTGTCT |
| | TGCTGTCTTTATTTAGGGGTTTTGCGCGCGCGGTAGGCCCGGGA |
| | CCAGCGGTCTCGGTCGTTGAGGGTCCTGTGTATTTTTTCCAGGA |
| | CGTGGTAAAGGTGACTCTGGATGTTCAGATACATGGGCATAAGC |
| | CCGTCTCTGGGGTGGAGGTAGCACCACTGCAGAGCTTCATGCTG |
| | CGGGGTGGTGTTGTAGATGATCCAGTCGTAGCAGGAGCGCTGG |
| | GCGTGGTGCCTAAAAATGTCTTTCAGTAGCAAGCTGATTGCCAG |
| | GGGCAGGCCCTTGGTGTAAGTGTTTACAAAGCGGTTAAGCTGGG |
| | ATGGGTGCATACGTGGGGATATGAGATGCATCTTGGACTGTATT |
| | TTTAGGTTGGCTATGTTCCCAGCCATATCCCTCCGGGGATTCATG |
| | TTGTGCAGAACCACCAGCACAGTGTATCCGGTGCACTTGGGAAA |
| | TTTGTCATGTAGCTTAGAAGGAAATGCGTGGAAGAACTTGGAG |
| | ACGCCCTTGTGACCTCCAAGATTTTCCATGCATTCGTCCATAATG |
| | ATGGCAATGGGCCCACGGGCGGCGGCCTGGGCGAAGATATTTC |
| | TGGGATCACTAACGTCATAGTTGTGTTCCAGGATGAGATCGTCA |
| | TAGGCCATTTTTACAAAGCGCGGGCGGAGGGTGCCAGACTGCG |
| | GTATAATGGTTCCATCCGGCCCAGGGGCGTAGTTACCCTCACAG |
| | ATTTGCATTTCCCACGCTTTGAGTTCAGATGGGGGGATCATGTC |
| | TACCTGCGGGGCGATGAAGAAAACGGTTTCCGGGGTAGGGGAG |
| | ATCAGCTGGGAAGAAAGCAGGTTCCTGAGCAGCTGCGACTTAC |
| | CGCAGCCGGTGGGCCCGTAAATCACACCTATTACCGGCTGCAAC |
| | TGGTAGTTAAGAGAGCTGCAGCTGCCGTCATCCCTGAGCAGGG |
| | GGGCCACTTCGTTAAGCATGTCCCTGACTCGCATGTTTTCCCTGA |
| | CCAAATCCGCCAGAAGGCGCTCGCCGCCCAGCGATAGCAGTTCT |
| | TGCAAGGAAGCAAAGTTTTTCAACGGTTTGAGACCGTCCGCCGT |
| | AGGCATGCTTTTGAGCGTTTGACCAAGCAGTTCCAGGCGGTCCC |
| | ACAGCTCGGTCACCTGCTCTACGGCATCTCGATCCAGCATATCT |
| | CCTCGTTTCGCGGGTTGGGGCGGCTTTCGCTGTACGGCAGTAGT |
| | CGGTGCTCGTCCAGACGGGCCAGGGTCATGTCTTTCCACGGGCG |
| | CAGGGTCCTCGTCAGCGTAGTCTGGGTCACGGTGAAGGGGTGC |
| | GCTCCGGGCTGCGCGCTGGCCAGGGTGCGCTTGAGGCTGGTCCT |
| | GCTGGTGCTGAAGCGCTGCCGGTCTTCGCCCTGCGCGTCGGCCA |
| | GGTAGCATTTGACCATGGTGTCATAGTCCAGCCCCTCCGCGGCG |
| | TGGCCCTTGGCGCGCAGCTTGCCCTTGGAGGAGGCGCCGCACGA |
| | GGGGCAGTGCAGACTTTTGAGGGCGTAGAGCTTGGGCGCGAGA |
| | AATACCGATTCCGGGGAGTAGGCATCCGCGCCGCAGGCCCCGC |
| | AGACGGTCTCGCATTCCACGAGCCAGGTGAGCTCTGGCCGTTCG |
| | GGGTCAAAAACCAGGTTTCCCCCATGCTTTTTGATGCGTTTCTTA |
| | CCTCTGGTTTCCATGAGCCGGTGTCCACGCTCGGTGACGAAAAG |
| | GCTGTCCGTGTCCCCGTATACAGACTTGAGAGGCCTGTCCTCGA |
| | GCGGTGTTCCGCGGTCCTCCTCGTATAGAAACTCGGACCACTCT |
| | GAGACAAAGGCTCGCGTCCAGGCCAGCACGAAGGAGGCTAAGT |
| | GGGAGGGGTAGCGGTCGTTGTCCACTAGGGGGTCCACTCGCTCC |
| | AGGGTGTGAAGACACATGTCGCCCTCTTCGGCATCAAGGAAGG |
| | TGATTGGTTTGTAGGTGTAGGCCACGTGACCGGGTGTTCCTGAA |
| | GGGGGGCTATAAAAGGGGGTGGGGGCGCGTTCGTCCTCACTCT |
| | CTTCCGCATCGCTGTCTGCGAGGGCCAGCTGTTGGGGTGAGTAC |
| | TCCCTCTGAAAAGCGGGCATGACTTCTGCGCTAAGATTGTCAGT |
| | TTCCAAAAACGAGGAGGATTTGATATTCACCTGGCCCGCGGTGA |
| | TGCCTTTGAGGGTGGCCGCATCCATCTGGTCAGAAAAGACAATC |
| | TTTTTGTTGTCAAGCTTGGTGGCAAACGACCCGTAGAGGGCGTT |
| | GGACAGCAACTTGGCGATGGAGCGCAGGGTTTGGTTTTTGTCGC |
| | GATCGGCGCGCTCCTTGGCCGCGATGTTTAGCTGCACGTATTCG |
| | CGCGCAACGCACCGCCATTCGGGAAAGACGGTGGTGCGCTCGT |
| | CGGGCACCAGGTGCACGCGCCAACCGCGGTTGTGCAGGGTGAC |
| | AAGGTCAACGCTGGTGGCTACCTCTCCGCGTAGGCGCTCGTTGG |
| | TCCAGCAGAGGCGGCCGCCCTTGCGCGAGCAGAATGGCGGTAG |
| | GGGGTCTAGCTGCGTCTCGTCCGGGGGGTCTGCGTCCACGGTAA |
| | AGACCCCGGGCAGCAGGCGCGCGTCGAAGTAGTCTATCTTGCAT |
| | CCTTGCAAGTCTAGCGCCTGCTGCCATGCGCGGGCGGCAAGCGC |
| | GCGCTCGTATGGGTTGAGTGGGGGACCCCATGGCATGGGGTGG |
| | GTGAGCGCGGAGGCGTACATGCCGCAAATGTCGTAAACGTAGA |
| | GGGGCTCTCTGAGTATTCCAAGATATGTAGGGTAGCATCTTCCA |
| | CCGCGGATGCTGGCGCGCACGTAATCGTATAGTTCGTGCGAGGG |
| | AGCGAGGAGGTCGGGACCGAGGTTGCTACGGGCGGGCTGCTCT |
| | GCTCGGAAGACTATCTGCCTGAAGATGGCATGTGAGTTGGATGA |
| | TATGGTTGGACGCTGGAAGACGTTGAAGCTGGCGTCTGTGAGAC |
| | CTACCGCGTCACGCACGAAGGAGGCGTAGGAGTCGCGCAGCTT |
| | GTTGACCAGCTCGGCGGTGACCTGCACGTCTAGGGCGCAGTAGT |
| | CCAGGGTTTCCTTGATGATGTCATACTTATCCTGTCCCTTTTTTTT |
| | CCACAGCTCGCGGTTGAGGACAAACTCTTCGCGGTCTTTCCAGT |
| | ACTCTTGGATCGGAAACCCGTCGGCCTCCGAACGGTAAGAGCCT |
| | AGCATGTAGAACTGGTTGACGGCCTGGTAGGCGCAGCATCCCTT |
| | TTCTACGGGTAGCGCGTATGCCTGCGCGGCCTTCCGGCATGACC |
| | AGCATGAAGGGCACGAGCTGCTTCCCAAAGGCCCCCATCCAAG |
| | TATAGGTCTCTACATCGTAGGTGACAAAGAGACGCTCGGTGCGA |
| | GGATGCGAGCCGATCGGGAAGAACTGGATCTCCCGCCACCAAT |
| | TGGAGGAGTGGCTATTGATGTGGTGAAAGTAGAAGTCCCTGCG |
| | ACGGGCCGAACACTCGTGCTGGCTTTTGTAAAAACGTGCGCAGT |
| | ACTGGCAGCGGTGCACGGGCTGTACATCCTGCACGAGGTTGACC |
| | TGACGACCGCGCACAAGGAAGCAGAGTGGGAATTTGAGCCCCT |
| | CGCCTGGCGGGTTTGGCTGGTGGTCTTCTACTTCGGCTGCTTGTC |
| | CTTGACCGTCTGGCTGCTCGAGGGGAGTTACGGTGGATCGGACC |
| | ACCACGCCGCGCGAGCCCAAAGTCCAGATGTCCGCGCGCGGCG |
| | GTCGGAGCTTGATGACAACATCGCGCAGATGGGAGCTGTCCAT |
| | GGTCTGGAGCTCCCGCGGCGTCAGGTCAGGCGGGAGCTCCTGC |
| | AGGTTTACCTCGCATAGACGGGTCAGGGCGCGGGCTAGATCCA |
| | GGTGATACCTAATTTCCAGGGGCTGGTTGGTGGCGGCGTCGATG |
| | GCTTGCAAGAGGCCGCATCCCCGCGGCGCGACTACGGTACCGC |
| | GCGGCGGGCGGTGGGCCGCGGGGGTGTCCTTGGATGATGCATC |
| | TAAAAGCGGTGACGCGGGCGAGCCCCCGGAGGTAGGGGGGGCT |
| | CCGGACCCGCCGGGAGAGGGGGCAGGGGCACGTCGGCGCCGCG |
| | CGCGGGCAGGAGCTGGTGCTGCGCGCGTAGGTTGCTGGCGAAC |
| | GCGACGACGCGGCGGTTGATCTCCTGAATCTGGCGCCTCTGCGT |
| | GAAGACGACGGGCCCGGTGAGCTTGAACCTGAAAGAGAGTTCG |
| | ACAGAATCAATTTCGGTGTCGTTGACGGCGGCCTGGCGCAAAAT |
| | CTCCTGCACGTCTCCTGAGTTGTCTTGATAGGCGATCTCGGCCAT |
| | GAACTGCTCGATCTCTTCCTCCTGGAGATCTCCGCGTCCGGCTC |
| | GCTCCACGGTGGCGGCGAGGTCGTTGGAAATGCGGGCCATGAG |
| | CTGCGAGAAGGCGTTGAGGCCTCCCTCGTTCCAGACGCGGCTGT |
| | AGACCACGCCCCCTTCGGCATCGCGGGCGCGCATGACCACCTGC |
| | GCGAGATTGAGCTCCACGTGCCGGGCGAAGACGGCGTAGTTTC |
| | GCAGGCGCTGAAAGAGGTAGTTGAGGGTGGTGGCGGTGTGTTC |
| | TGCCACGAAGAAGTACATAACCCAGCGTCGCAACGTGGATTCG |
| | TTGATAATTGTTGTGTAGGTACTCCGCCGCCGAGGGACCTGAGC |
| | GAGTCCGCATCGACCGGATCGGAAAACCTCTCGAGAAAGGCGT |
| | CTAACCAGTCACAGTCGCAAGGTAGGCTGAGCACCGTGGCGGG |
| | CGGCAGCGGGCGGCGGTCGGGGTTGTTTCTGGCGGAGGTGCTG |
| | CTGATGATGTAATTAAAGTAGGCGGTCTTGAGACGGCGGATGGT |
| | CGACAGAAGCACCATGTCCTTGGGTCCGGCCTGCTGAATGCGCA |
| | GGCGGTCGGCCATGCCCCAGGCTTCGTTTTGACATCGGCGCAGG |
| | TCTTTGTAGTAGTCTTGCATGAGCCTTTCTACCGGCACTTCTTCT |
| | TCTCCTTCCTCTTGTCCTGCATCTCTTGCATCTATCGCTGCGGCG |
| | GCGGCGGAGTTTGGCCGTAGGTGGCGCCCTCTTCCTCCCATGCG |
| | TGTGACCCCGAAGCCCCTCATCGGCTGAAGCAGGGCTAGGTCG |
| | GCGACAACGCGCTCGGCTAATATGGCCTGCTGCACCTGCGTGAG |
| | GGTAGACTGGAAGTCATCCATGTCCACAAAGCGGTGGTATGCG |
| | CCCGTGTTGATGGTGTAAGTGCAGTTGGCCATAACGGACCAGTT |
| | AACGGTCTGGTGACCCGGCTGCGAGAGCTCGGTGTACCTGAGA |
| | CGCGAGTAAGCCCTCGAGTCAAATACGTAGTCGTTGCAAGTCCG |
| | CACCAGGTACTGGTATCCCACCAAAAAGTGCGGCGGCGGCTGG |
| | CGGTAGAGGGGCCAGCGTAGGGTGGCCGGGGCTCCGGGGGCGA |
| | GATCTTCCAACATAAGGCGATGATATCCGTAGATGTACCTGGAC |
| | ATCCAGGTGATGCCGGCGGCGGTGGTGGAGGCGCGCGGAAAGT |
| | CGCGGACGCGGTTCCAGATGTTGCGCAGCGGCAAAAAGTGCTC |
| | CATGGTCGGGACGCTCTGGCCGGTCAGGCGCGCGCAATCGTTGA |
| | CGCTCTAGCGTGCAAAAGGAGAGCCTGTAAGCGGGCACTCTTCC |
| | GTGGTCTGGTGGATAAATTCGCAAGGGTATCATGGCGGACGAC |
| | CGGGGTTCGAGCCCCGTATCCGGCCGTCCGCCGTGATCCATGCG |
| | GTTACCGCCCGCGTGTCGAACCCAGGTGTGCGACGTCAGACAAC |
| | GGGGGAGTGCTCCTTTTGGCTTCCTTCCAGGCGCGGCGGCTGCT |
| | GCGCTAGCTTTTTTGGCCACTGGCCGCGCGCAGCGTAAGCGGTT |
| | AGGCTGGAAAGCGAAAGCATTAAGTGGCTCGCTCCCTGTAGCC |
| | GGAGGGTTATTTTCCAAGGGTTGAGTCGCGGGACCCCCGGTTCG |
| | AGTCTCGGACCGGCCGGACTGCGGCGAACGGGGGTTTGCCTCCC |
| | CGTCATGCAAGACCCCGCTTGCAAATTCCTCCGGAAACAGGGAC |
| | GAGCCCCTTTTTTGCTTTTCCCAGATGCATCCGGTGCTGCGGCAG |
| | ATGCGCCCCCCTCCTCAGCAGCGGCAAGAGCAAGAGCAGCGGC |
| | AGACATGCAGGGCACCCTCCCCTCCTCCTACCGCGTCAGGAGGG |
| | GCGACATCCGCGGTTGACGCGGCAGCAGATGGTGATTACGAAC |
| | CCCCGCGGCGCCGGGCCCGGCACTACCTGGACTTGGAGGAGGG |
| | CGAGGGCCTGGCGCGGCTAGGAGCGCCCTCTCCTGAGCGGCAC |
| | CCAAGGGTGCAGCTGAAGCGTGATACGCGTGAGGCGTACGTGC |
| | CGCGGCAGAACCTGTTTCGCGACCGCGAGGGAGAGGAGCCCGA |
| | GGAGATGCGGGATCGAAAGTTCCACGCAGGGCGCGAGCTGCGG |
| | CATGGCCTGAATCGCGAGCGGTTGCTGCGCGAGGAGGACTTTG |
| | AGCCCGACGCGCGAACCGGGATTAGTCCCGCGCGCGCACACGT |
| | GGCGGCCGCCGACCTGGTAACCGCATACGAGCAGACGGTGAAC |
| | CAGGAGATTAACTTTCAAAAAAGCTTTAACAACCACGTGCGTAC |
| | GCTTGTGGCGCGCGAGGAGGTGGCTATAGGACTGATGCATCTGT |
| | GGGACTTTGTAAGCGCGCTGGAGCAAAACCCAAATAGCAAGCC |
| | GCTCATGGCGCAGCTGTTCCTTATAGTGCAGCACAGCAGGGACA |
| | ACGAGGCATTCAGGGATGCGCTGCTAAACATAGTAGAGCCCGA |
| | GGGCCGCTGGCTGCTCGATTTGATAAACATCCTGCAGAGCATAG |
| | TGGTGCAGGAGCGCAGCTTGAGCCTGGCTGACAAGGTGGCCGC |
| | CATCAACTATTCCATGCTTAGCCTGGGCAAGTTTTACGCCCGCA |
| | AGATATACCATACCCCTTACGTTCCCATAGACAAGGAGGTAAAG |
| | ATCGAGGGGTTCTACATGCGCATGGCGCTGAAGGTGCTTACCTT |
| | GAGCGACGACCTGGGCGTTTATCGCAACGAGCGCATCCACAAG |
| | GCCGTGAGCGTGAGCCGGCGGCGCGAGCTCAGCGACCGCGAGC |
| | TGATGCACAGCCTGCAAAGGGCCCTGGCTGGCACGGGCAGCGG |
| | CGATAGAGAGGCCGAGTCCTACTTTGACGCGGGCGCTGACCTGC |
| | GCTGGGCCCCAAGCCGACGCGCCCTGGAGGCAGCTGGGGCCGG |
| | ACCTGGGCTGGCGGTGGCACCCGCGCGCGCTGGCAACGTCGGC |
| | GGCGTGGAGGAATATGACGAGGACGATGAGTACGAGCCAGAGG |
| | ACGGCGAGTACTAAGCGGTGATGTTTCTGATCAGATGATGCAAG |
| | ACGCAACGGACCCGGCGGTGCGGGCGGCGCTGCAGAGCCAGCC |
| | GTCCGGCCTTAACTCCACGGACGACTGGCGCCAGGTCATGGACC |
| | GCATCATGTCGCTGACTGCGCGCAATCCTGACGCGTTCCGGCAG |
| | CAGCCGCAGGCCAACCGGCTCTCCGCAATTCTGGAAGCGGTGGT |
| | CCCGGCGCGCGCAAACCCCACGCACGAGAAGGTGCTGGCGATC |
| | GTAAACGCGCTGGCCGAAAACAGGGCCATCCGGCCCGACGAGG |
| | CCGGCCTGGTCTACGACGCGCTGCTTCAGCGCGTGGCTCGTTAC |
| | AACAGCGGCAACGTGCAGACCAACCTGGACCGGCTGGTGGGGG |
| | ATGTGCGCGAGGCCGTGGCGCAGCGTGAGCGCGCGCAGCAGCA |
| | GGGCAACCTGGGCTCCATGGTTGCACTAAACGCCTTCCTGAGTA |
| | CACAGCCCGCCAACGTGCCGCGGGGACAGGAGGACTACACCAA |
| | CTTTGTGAGCGCACTGCGGCTAATGGTGACTGAGACACCGCAAA |
| | GTGAGGTGTACCAGTCTGGGCCAGACTATTTTTTCCAGACCAGT |
| | AGACAAGGCCTGCAGACCGTAAACCTGAGCCAGGCTTTCAAAA |
| | ACTTGCAGGGGCTGTGGGGGGTGCGGGCTCCCACAGGCGACCG |
| | CGCGACCGTGTCTAGCTTGCTGACGCCCAACTCGCGCCTGTTGC |
| | TGCTGCTAATAGCGCCCTTCACGGACAGTGGCAGCGTGTCCCGG |
| | GACACATACCTAGGTCACTTGCTGACACTGTACCGCGAGGCCAT |
| | AGGTCAGGCGCATGTGGACGAGCATACTTTCCAGGAGATTACA |
| | AGTGTCAGCCGCGCGCTGGGGCAGGAGGACACGGGCAGCCTGG |
| | AGGCAACCCTAAACTACCTGCTGACCAACCGGCGGCAGAAGAT |
| | CCCCTCGTTGCACAGTTTAAACAGCGAGGAGGAGCGCATTTTGC |
| | GCTACGTGCAGCAGAGCGTGAGCCTTAACCTGATGCGCGACGG |
| | GGTAACGCCCAGCGTGGCGCTGGACATGACCGCGCGCAACATG |
| | GAACCGGGCATGTATGCCTCAAACCGGCCGTTTATCAACCGCCT |
| | AATGGACTACTTGCATCGCGCGGCCGCCGTGAACCCCGAGTATT |
| | TCACCAATGCCATCTTGAACCCGCACTGGCTACCGCCCCCTGGT |
| | TTCTACACCGGGGGATTCGAGGTGCCCGAGGGTAACGATGGATT |
| | CCTCTGGGACGACATAGACGACAGCGTGTTTTCCCCGCAACCGC |
| | AGACCCTGCTAGAGTTGCAACAGCGCGAGCAGGCAGAGGCGGC |
| | GCTGCGAAAGGAAAGCTTCCGCAGGCCAAGCAGCTTGTCCGAT |
| | CTAGGCGCTGCGGCCCCGCGGTCAGATGCTAGTAGCCCATTTCC |
| | AAGCTTGATAGGGTCTCTTACCAGCACTCGCACCACCCGCCCGC |
| | GCCTGCTGGGCGAGGAGGAGTACCTAAACAACTCGCTGCTGCA |
| | GCCGCAGCGCGAAAAAAACCTGCCTCCGGCATTTCCCAACAAC |
| | GGGATAGAGAGCCTAGTGGACAAGATGAGTAGATGGAAGACGT |
| | ACGCGCAGGAGCACAGGGACGTGCCAGGCCCGCGCCCGCCCAC |
| | CCGTCGTCAAAGGCACGACCGTCAGCGGGGTCTGGTGTGGGAG |
| | GACGATGACTCGGCAGACGACAGCAGCGTCCTGGATTTGGGAG |
| | GGAGTGGCAACCCGTTTGCGCACCTTCGCCCCAGGCTGGGGAG |
| | AATGTTTTAAAAAAAAAAAAGCATGATGCAAAATAAAAAACTC |
| | ACCAAGGCCATGGCACCGAGCGTTGGTTTTCTTGTATTCCCCTT |
| | AGTATGCGGCGCGCGGCGATGTATGAGGAAGGTCCTCCTCCCTC |
| | CTACGAGAGTGTGGTGAGCGCGGCGCCAGTGGCGGCGGCGCTG |
| | GGTTCTCCCTTCGATGCTCCCCTGGACCCGCCGTTTGTGCCTCCG |
| | CGGTACCTGCGGCCTACCGGGGGGAGAAACAGCATCCGTTACT |
| | CTGAGTTGGCACCCCTATTCGACACCACCCGTGTGTACCTGGTG |
| | GACAACAAGTCAACGGATGTGGCATCCCTGAACTACCAGAACG |
| | ACCACAGCAACTTTCTGACCACGGTCATTCAAAACAATGACTAC |
| | AGCCCGGGGGAGGCAAGCACACAGACCATCAATCTTGACGACC |
| | GGTCGCACTGGGGCGGCGACCTGAAAACCATCCTGCATACCAA |
| | CATGCCAAATGTGAACGAGTTCATGTTTACCAATAAGTTTAAGG |
| | CGCGGGTGATGGTGTCGCGCTTGCCTACTAAGGACAATCAGGTG |
| | GAGCTGAAATACGAGTGGGTGGAGTTCACGCTGCCCGAGGGCA |
| | ACTACTCCGAGACCATGACCATAGACCTTATGAACAACGCGATC |
| | GTGGAGCACTACTTGAAAGTGGGCAGACAGAACGGGGTTCTGG |
| | AAAGCGACATCGGGGTAAAGTTTGACACCCGCAACTTCAGACT |
| | GGGGTTTGACCCCGTCACTGGTCTTGTCATGCCTGGGGTATATA |
| | CAAACGAAGCCTTCCATCCAGACATCATTTTGCTGCCAGGATGC |
| | GGGGTGGACTTCACCCACAGCCGCCTGAGCAACTTGTTGGGCAT |
| | CCGCAAGCGGCAACCCTTCCAGGAGGGCTTTAGGATCACCTACG |
| | ATGATCTGGAGGGTGGTAACATTCCCGCACTGTTGGATGTGGAC |
| | GCCTACCAGGCGAGCTTGAAAGATGACACCGAACAGGGCGGGG |
| | GTGGCGCAGGCGGCAGCAACAGCAGTGGCAGCGGCGCGGAAG |
| | AGAACTCCAACGCGGCAGCCGCGGCAATGCAGCCGGTGGAGGA |
| | CATGAACGATCATGCCATTCGCGGCGACACCTTTGCCACACGGG |
| | CTGAGGAGAAGCGCGCTGAGGCCGAAGCAGCGGCCGAAGCTGC |
| | CGCCCCCGCTGCGCAACCCGAGGTCGAGAAGCCTCAGAAGAAA |
| | CCGGTGATCAAACCCCTGACAGAGGACAGCAAGAAACGCAGTT |
| | ACAACCTAATAAGCAATGACAGCACCTTCACCCAGTACCGCAG |
| | CTGGTACCTTGCATACAACTACGGCGACCCTCAGACCGGAATCC |
| | GCTCATGGACCCTGCTTTGCACTCCTGACGTAACCTGCGGCTCG |
| | GAGCAGGTCTACTGGTCGTTGCCAGACATGATGCAAGACCCCGT |
| | GACCTTCCGCTCCACGCGCCAGATCAGCAACTTTCCGGTGGTGG |
| | GCGCCGAGCTGTTGCCCGTGCACTCCAAGAGCTTCTACAACGAC |
| | CAGGCCGTCTACTCCCAACTCATCCGCCAGTTTACCTCTCTGACC |
| | CACGTGTTCAATCGCTTTCCCGAGAACCAGATTTTGGCGCGCCC |
| | GCCAGCCCCCACCATCACCACCGTCAGTGAAAACGTTCCTGCTC |
| | TCACAGATCACGGGACGCTACCGCTGCGCAACAGCATCGGAGG |
| | AGTCCAGCGAGTGACCATTACTGACGCCAGACGCCGCACCTGCC |
| | CCTACGTTTACAAGGCCCTGGGCATAGTCTCGCCGCGCGTCCTA |
| | TCGAGCCGCACTTTTTGAGCAAGCATGTCCATCCTTATATCGCC |
| | CAGCAATAACACAGGCTGGGGCCTGCGCTTCCCAAGCAAGATG |
| | TTTGGCGGGGCCAAGAAGCGCTCCGACCAACACCCAGTGCGCG |
| | TGCGCGGGCACTACCGCGCGCCCTGGGGCGCGCACAAACGCGG |
| | CCGCACTGGGCGCACCACCGTCGATGACGCCATCGACGCGGTG |
| | GTGGAGGAGGCGCGCAACTACACGCCCACGCCGCCACCAGTGT |
| | CCACAGTGGACGCGGCCATTCAGACCGTGGTGCGCGGAGCCCG |
| | GCGCTATGCTAAAATGAAGAGACGGCGGAGGCGCGTAGCACGT |
| | CGCCACCGCCGCCGACCCGGCACTGCCGCCCAACGCGCGGCGG |
| | CGGCCCTGCTTAACCGCGCACGTCGCACCGGCCGACGGGCGGC |
| | CATGCGGGCCGCTCGAAGGCTGGCCGCGGGTATTGTCACTGTGC |
| | CCCCCAGGTCCAGGCGACGAGCGGCCGCCGCAGCAGCCGCGGC |
| | CATTAGTGCTATGACTCAGGGTCGCAGGGGCAACGTGTATTGGG |
| | TGCGCGACTCGGTTAGCGGCCTGCGCGTGCCCGTGCGCACCCGC |
| | CCCCCGCGCAACTAGATTGCAAGAAAAAACTACTTAGACTCGTA |
| | CTGTTGTATGTATCCAGCGGCGGCGGCGCGCAACGAAGCTATGT |
| | CCAAGCGCAAAATCAAAGAAGAGATGCTCCAGGTCATCGCGCC |
| | GGAGATCTATGGCCCCCCGAAGAAGGAAGAGCAGGATTACAAG |
| | CCCCGAAAGCTAAAGCGGGTCAAAAAGAAAAAGAAAGATGAT |
| | GATGATGAACTTGACGACGAGGTGGAACTGCTGCACGCTACCG |
| | CGCCCAGGCGACGGGTACAGTGGAAAGGTCGACGCGTAAAACG |
| | TGTTTTGCGACCCGGCACCACCGTAGTCTTTACGCCCGGTGAGC |
| | GCTCCACCCGCACCTACAAGCGCGTGTATGATGAGGTGTACGGC |
| | GACGAGGACCTGCTTGAGCAGGCCAACGAGCGCCTCGGGGAGT |
| | TTGCCTACGGAAAGCGGCATAAGGACATGCTGGCGTTGCCGCTG |
| | GACGAGGGCAACCCAACACCTAGCCTAAAGCCCGTAACACTGC |
| | AGCAGGTGCTGCCCGCGCTTGCACCGTCCGAAGAAAAGCGCGG |
| | CCTAAAGCGCGAGTCTGGTGACTTGGCACCCACCGTGCAGCTGA |
| | TGGTACCCAAGCGCCAGCGACTGGAAGATGTCTTGGAAAAAAT |
| | GACCGTGGAACCTGGGCTGGAGCCCGAGGTCCGCGTGCGGCCA |
| | ATCAAGCAGGTGGCGCCGGGACTGGGCGTGCAGACCGTGGACG |
| | TTCAGATACCCACTACCAGTAGCACCAGTATTGCCACCGCCACA |
| | GAGGGCATGGAGACACAAACGTCCCCGGTTGCCTCAGCGGTGG |
| | CGGATGCCGCGGTGCAGGCGGTCGCTGCGGCCGCGTCCAAGAC |
| | CTCTACGGAGGTGCAAACGGACCCGTGGATGTTTCGCGTTTCAG |
| | CCCCCCGGCGCCCGCGCCGTTCGAGGAAGTACGGCGCCGCCAG |
| | CGCGCTACTGCCCGAATATGCCCTACATCCTTCCATTGCGCCTA |
| | CCCCCGGCTATCGTGGCTACACCTACCGCCCCAGAAGACGAGCA |
| | ACTACCCGACGCCGAACCACCACTGGAACCCGCCGCCGCCGTC |
| | GCCGTCGCCAGCCCGTGCTGGCCCCGATTTCCGTGCGCAGGGTG |
| | GCTCGCGAAGGAGGCAGGACCCTGGTGCTGCCAACAGCGCGCT |
| | ACCACCCCAGCATCGTTTAAAAGCCGGTCTTTGTGGTTCTTGCA |
| | GATATGGCCCTCACCTGCCGCCTCCGTTTCCCGGTGCCGGGATT |
| | CCGAGGAAGAATGCACCGTAGGAGGGGCATGGCCGGCCACGGC |
| | CTGACGGGCGGCATGCGTCGTGCGCACCACCGGCGGCGGCGCG |
| | CGTCGCACCGTCGCATGCGCGGCGGTATCCTGCCCCTCCTTATT |
| | CCACTGATCGCCGCGGCGATTGGCGCCGTGCCCGGAATTGCATC |
| | CGTGGCCTTGCAGGCGCAGAGACACTGATTAAAAACAAGTTGC |
| | ATGTGGAAAAATCAAAATAAAAAGTCTGGACTCTCACGCTCGCT |
| | TGGTCCTGTAACTATTTTGTAGAATGGAAGACATCAACTTTGCG |
| | TCTCTGGCCCCGCGACACGGCTCGCGCCCGTTCATGGGAAACTG |
| | GCAAGATATCGGCACCAGCAATATGAGCGGTGGCGCCTTCAGC |
| | TGGGGCTCGCTGTGGAGCGGCATTAAAAATTTCGGTTCCACCGT |
| | TAAGAACTATGGCAGCAAGGCCTGGAACAGCAGCACAGGCCAG |
| | ATGCTGAGGGATAAGTTGAAAGAGCAAAATTTCCAACAAAAGG |
| | TGGTAGATGGCCTGGCCTCTGGCATTAGCGGGGTGGTGGACCTG |
| | GCCAACCAGGCAGTGCAAAATAAGATTAACAGTAAGCTTGATC |
| | CCCGCCCTCCCGTAGAGGAGCCTCCACCGGCCGTGGAGACAGT |
| | GTCTCCAGAGGGGCGTGGCGAAAAGCGTCCGCGCCCCGACAGG |
| | GAAGAAACTCTGGTGACGCAAATAGACGAGCCTCCCTCGTACG |
| | AGGAGGCACTAAAGCAAGGCCTGCCCACCACCCGTCCCATCGC |
| | GCCCATGGCTACCGGAGTGCTGGGCCAGCACACACCCGTAACG |
| | CTGGACCTGCCTCCCCCCGCCGACACCCAGCAGAAACCTGTGCT |
| | GCCAGGCCCGACCGCCGTTGTTGTAACCCGTCCTAGCCGCGCGT |
| | CCCTGCGCCGCGCCGCCAGCGGTCCGCGATCGTTGCGGCCCGTA |
| | GCCAGTGGCAACTGGCAAAGCACACTGAACAGCATCGTGGGTC |
| | TGGGGGTGCAATCCCTGAAGCGCCGACGATGCTTCTGATAGCTA |
| | ACGTGTCGTATGTGTGTCATGTATGCGTCCATGTCGCCGCCAGA |
| | GGAGCTGCTGAGCCGCCGCGCGCCCGCTTTCCAAGATGGCTACC |
| | CCTTCGATGATGCCGCAGTGGTCTTACATGCACATCTCGGGCCA |
| | GGACGCCTCGGAGTACCTGAGCCCCGGGCTGGTGCAGTTTGCCC |
| | GCGCCACCGAGACGTACTTCAGCCTGAATAACAAGTTTAGAAA |
| | CCCCACGGTGGCGCCTACGCACGACGTGACCACAGACCGGTCC |
| | CAGCGTTTGACGCTGCGGTTCATCCCTGTGGACCGTGAGGATAC |
| | TGCGTACTCGTACAAGGCGCGGTTCACCCTAGCTGTGGGTGATA |
| | ACCGTGTGCTGGACATGGCTTCCACGTACTTTGACATCCGCGGC |
| | GTGCTGGACAGGGGCCCTACTTTTAAGCCCTACTCTGGCACTGC |
| | CTACAACGCCCTGGCTCCCAAGGGTGCCCCAAATCCTTGCGAAT |
| | GGGATGAAGCTGCTACTGCTCTTGAAATAAACCTAGAAGAAGA |
| | GGACGATGACAACGAAGACGAAGTAGACGAGCAAGCTGAGCA |
| | GCAAAAAACTCACGTATTTGGGCAGGCGCCTTATTCTGGTATAA |
| | ATATTACAAAGGAGGGTATTCAAATAGGTGTCGAAGGTCAAAC |
| | ACCTAAATATGCCGATAAAACATTTCAACCTGAACCTCAAATAG |
| | GAGAATCTCAGTGGTACGAAACAGAAATTAATCATGCAGCTGG |
| | GAGAGTCCTAAAAAAGACTACCCCAATGAAACCATGTTACGGT |
| | TCATATGCAAAACCCACAAATGAAAATGGAGGGCAAGGCATTC |
| | TTGTAAAGCAACAAAATGGAAAGCTAGAAAGTCAAGTGGAAAT |
| | GCAATTTTTCTCAACTACTGAGGCAGCCGCAGGCAATGGTGATA |
| | ACTTGACTCCTAAAGTGGTATTGTACAGTGAAGATGTAGATATA |
| | GAAACCCCAGACACTCATATTTCTTACATGCCCACTATTAAGGA |
| | AGGTAACTCACGAGAACTAATGGGCCAACAATCTATGCCCAAC |
| | AGGCCTAATTACATTGCTTTTAGGGACAATTTTATTGGTCTAATG |
| | TATTACAACAGCACGGGTAATATGGGTGTTCTGGCGGGCCAAGC |
| | ATCGCAGTTGAATGCTGTTGTAGATTTGCAAGACAGAAACACAG |
| | AGCTTTCATACCAGCTTTTGCTTGATTCCATTGGTGATAGAACCA |
| | GGTACTTTTCTATGTGGAATCAGGCTGTTGACAGCTATGATCCA |
| | GATGTTAGAATTATTGAAAATCATGGAACTGAAGATGAACTTCC |
| | AAATTACTGCTTTCCACTGGGAGGTGTGATTAATACAGAGACTC |
| | TTACCAAGGTAAAACCTAAAACAGGTCAGGAAAATGGATGGGA |
| | AAAAGATGCTACAGAATTTTCAGATAAAAATGAAATAAGAGTT |
| | GGAAATAATTTTGCCATGGAAATCAATCTAAATGCCAACCTGTG |
| | GAGAAATTTCCTGTACTCCAACATAGCGCTGTATTTGCCCGACA |
| | AGCTAAAGTACAGTCCTTCCAACGTAAAAATTTCTGATAACCCA |
| | AACACCTACGACTACATGAACAAGCGAGTGGTGGCTCCCGGGC |
| | TAGTGGACTGCTACATTAACCTTGGAGCACGCTGGTCCCTTGAC |
| | TATATGGACAACGTCAACCCATTTAACCACCACCGCAATGCTGG |
| | CCTGCGCTACCGCTCAATGTTGCTGGGCAATGGTCGCTATGTGC |
| | CCTTCCACATCCAGGTGCCTCAGAAGTTCTTTGCCATTAAAAAC |
| | CTCCTTCTCCTGCCGGGCTCATACACCTACGAGTGGAACTTCAG |
| | GAAGGATGTTAACATGGTTCTGCAGAGCTCCCTAGGAAATGACC |
| | TAAGGGTTGACGGAGCCAGCATTAAGTTTGATAGCATTTGCCTT |
| | TACGCCACCTTCTTCCCCATGGCCCACAACACCGCCTCCACGCT |
| | TGAGGCCATGCTTAGAAACGACACCAACGACCAGTCCTTTAACG |
| | ACTATCTCTCCGCCGCCAACATGCTCTACCCTATACCCGCCAAC |
| | GCTACCAACGTGCCCATATCCATCCCCTCCCGCAACTGGGCGGC |
| | TTTCCGCGGCTGGGCCTTCACGCGCCTTAAGACTAAGGAAACCC |
| | CATCACTGGGCTCGGGCTACGACCCTTATTACACCTACTCTGGC |
| | TCTATACCCTACCTAGATGGAACCTTTTACCTCAACCACACCTTT |
| | AAGAAGGTGGCCATTACCTTTGACTCTTCTGTCAGCTGGCCTGG |
| | CAATGACCGCCTGCTTACCCCCAACGAGTTTGAAATTAAGCGCT |
| | CAGTTGACGGGGAGGGTTACAACGTTGCCCAGTGTAACATGAC |
| | CAAAGACTGGTTCCTGGTACAAATGCTAGCTAACTATAACATTG |
| | GCTACCAGGGCTTCTATATCCCAGAGAGCTACAAGGACCGCATG |
| | TACTCCTTCTTTAGAAACTTCCAGCCCATGAGCCGTCAGGTGGT |
| | GGATGATACTAAATACAAGGACTACCAACAGGTGGGCATCCTA |
| | CACCAACACAACAACTCTGGATTTGTTGGCTACCTTGCCCCCAC |
| | CATGCGCGAAGGACAGGCCTACCCTGCTAACTTCCCCTATCCGC |
| | TTATAGGCAAGACCGCAGTTGACAGCATTACCCAGAAAAAGTTT |
| | CTTTGCGATCGCACCCTTTGGCGCATCCCATTCTCCAGTAACTTT |
| | ATGTCCATGGGCGCACTCACAGACCTGGGCCAAAACCTTCTCTA |
| | CGCCAACTCCGCCCACGCGCTAGACATGACTTTTGAGGTGGATC |
| | CCATGGACGAGCCCACCCTTCTTTATGTTTTGTTTGAAGTCTTTG |
| | ACGTGGTCCGTGTGCACCAGCCGCACCGCGGCGTCATCGAAACC |
| | GTGTACCTGCGCACGCCCTTCTCGGCCGGCAACGCCACAACATA |
| | AAGAAGCAAGCAACATCAACAACAGCTGCCGCCATGGGCTCCA |
| | GTGAGCAGGAACTGAAAGCCATTGTCAAAGATCTTGGTTGTGG |
| | GCCATATTTTTTGGGCACCTATGACAAGCGCTTTCCAGGCTTTGT |
| | TTCTCCACACAAGCTCGCCTGCGCCATAGTCAATACGGCCGGTC |
| | GCGAGACTGGGGGCGTACACTGGATGGCCTTTGCCTGGAACCC |
| | GCACTCAAAAACATGCTACCTCTTTGAGCCCTTTGGCTTTTCTGA |
| | CCAGCGACTCAAGCAGGTTTACCAGTTTGAGTACGAGTCACTCC |
| | TGCGCCGTAGCGCCATTGCTTCTTCCCCCGACCGCTGTATAACG |
| | CTGGAAAAGTCCACCCAAAGCGTACAGGGGCCCAACTCGGCCG |
| | CCTGTGGACTATTCTGCTGCATGTTTCTCCACGCCTTTGCCAACT |
| | GGCCCCAAACTCCCATGGATCACAACCCCACCATGAACCTTATT |
| | ACCGGGGTACCCAACTCCATGCTCAACAGTCCCCAGGTACAGCC |
| | CACCCTGCGTCGCAACCAGGAACAGCTCTACAGCTTCCTGGAGC |
| | GCCACTCGCCCTACTTCCGCAGCCACAGTGCGCAGATTAGGAGC |
| | GCCACTTCTTTTTGTCACTTGAAAAACATGTAAAAATAATGTAC |
| | TAGAGACACTTTCAATAAAGGCAAATGCTTTTATTTGTACACTC |
| | TCGGGTGATTATTTACCCCCACCCTTGCCGTCTGCGCCGTTTAAA |
| | AATCAAAGGGGTTCTGCCGCGCATCGCTATGCGCCACTGGCAGG |
| | GACACGTTGCGATACTGGTGTTTAGTGCTCCACTTAAACTCAGG |
| | CACAACCATCCGCGGCAGCTCGGTGAAGTTTTCACTCCACAGGC |
| | TGCGCACCATCACCAACGCGTTTAGCAGGTCGGGCGCCGATATC |
| | TTGAAGTCGCAGTTGGGGCCTCCGCCCTGCGCGCGCGAGTTGCG |
| | ATACACAGGGTTGCAGCACTGGAACACTATCAGCGCCGGGTGG |
| | TGCACGCTGGCCAGCACGCTCTTGTCGGAGATCAGATCCGCGTC |
| | CAGGTCCTCCGCGTTGCTCAGGGCGAACGGAGTCAACTTTGGTA |
| | GCTGCCTTCCCAAAAAGGGCGCGTGCCCAGGCTTTGAGTTGCAC |
| | TCGCACCGTAGTGGCATCAAAAGGTGACCGTGCCCGGTCTGGGC |
| | GTTAGGATACAGCGCCTGCATAAAAGCCTTGATCTGCTTAAAAG |
| | CCACCTGAGCCTTTGCGCCTTCAGAGAAGAACATGCCGCAAGAC |
| | TTGCCGGAAAACTGATTGGCCGGACAGGCCGCGTCGTGCACGC |
| | AGCACCTTGCGTCGGTGTTGGAGATCTGCACCACATTTCGGCCC |
| | CACCGGTTCTTCACGATCTTGGCCTTGCTAGACTGCTCCTTCAGC |
| | GCGCGCTGCCCGTTTTCGCTCGTCACATCCATTTCAATCACGTGC |
| | TCCTTATTTATCATAATGCTTCCGTGTAGACACTTAAGCTCGCCT |
| | TCGATCTCAGCGCAGCGGTGCAGCCACAACGCGCAGCCCGTGG |
| | GCTCGTGATGCTTGTAGGTCACCTCTGCAAACGACTGCAGGTAC |
| | GCCTGCAGGAATCGCCCCATCATCGTCACAAAGGTCTTGTTGCT |
| | GGTGAAGGTCAGCTGCAACCCGCGGTGCTCCTCGTTCAGCCAGG |
| | TCTTGCATACGGCCGCCAGAGCTTCCACTTGGTCAGGCAGTAGT |
| | TTGAAGTTCGCCTTTAGATCGTTATCCACGTGGTACTTGTCCATC |
| | AGCGCGCGCGCAGCCTCCATGCCCTTCTCCCACGCAGACACGAT |
| | CGGCACACTCAGCGGGTTCATCACCGTAATTTCACTTTCCGCTTC |
| | GCTGGGCTCTTCCTCTTCCTCTTGCGTCCGCATACCACGCGCCAC |
| | TGGGTCGTCTTCATTCAGCCGCCGCACTGTGCGCTTACCTCCTTT |
| | GCCATGCTTGATTAGCACCGGTGGGTTGCTGAAACCCACCATTT |
| | GTAGCGCCACATCTTCTCTTTCTTCCTCGCTGTCCACGATTACCT |
| | CTGGTGATGGCGGGCGCTCGGGCTTGGGAGAAGGGCGCTTCTTT |
| | TTCTTCTTGGGCGCAATGGCCAAATCCGCCGCCGAGGTCGATGG |
| | CCGCGGGCTGGGTGTGCGCGGCACCAGCGCGTCTTGTGATGAGT |
| | CTTCCTCGTCCTCGGACTCGATACGCCGCCTCATCCGCTTTTTTG |
| | GGGGCGCCCGGGGAGGCGGCGGCGACGGGGACGGGGACGACA |
| | CGTCCTCCATGGTTGGGGGACGTCGCGCCGCACCGCGTCCGCGC |
| | TCGGGGGTGGTTTCGCGCTGCTCCTCTTCCCGACTGGCCATTTCC |
| | TTCTCCTATAGGCAGAAAAAGATCATGGAGTCAGTCGAGAAGA |
| | AGGACAGCCTAACCGCCCCCTCTGAGTTCGCCACCACCGCCTCC |
| | ACCGATGCCGCCAACGCGCCTACCACCTTCCCCGTCGAGGCACC |
| | CCCGCTTGAGGAGGAGGAAGTGATTATCGAGCAGGACCCAGGT |
| | TTTGTAAGCGAAGACGACGAGGACCGCTCAGTACCAACAGAGG |
| | ATAAAAAGCAAGACCAGGACAACGCAGAGGCAAACGAGGAAC |
| | AAGTCGGGCGGGGGGACGAAAGGCATGGCGACTACCTAGATGT |
| | GGGAGACGACGTGCTGTTGAAGCATCTGCAGCGCCAGTGCGCC |
| | ATTATCTGCGACGCGTTGCAAGAGCGCAGCGATGTGCCCCTCGC |
| | CATAGCGGATGTCAGCCTTGCCTACGAACGCCACCTATTCTCAC |
| | CGCGCGTACCCCCCAAACGCCAAGAAAACGGCACATGCGAGCC |
| | CAACCCGCGCCTCAACTTCTACCCCGTATTTGCCGTGCCAGAGG |
| | TGCTTGCCACCTATCACATCTTTTTCCAAAACTGCAAGATACCCC |
| | TATCCTGCCGTGCCAACCGCAGCCGAGCGGACAAGCAGCTGGC |
| | CTTGCGGCAGGGCGCTGTCATACCTGATATCGCCTCGCTCAACG |
| | AAGTGCCAAAAATCTTTGAGGGTCTTGGACGCGACGAGAAGCG |
| | CGCGGCAAACGCTCTGCAACAGGAAAACAGCGAAAATGAAAGT |
| | CACTCTGGAGTGTTGGTGGAACTCGAGGGTGACAACGCGCGCCT |
| | AGCCGTACTAAAACGCAGCATCGAGGTCACCCACTTTGCCTACC |
| | CGGCACTTAACCTACCCCCCAAGGTCATGAGCACAGTCATGAGT |
| | GAGCTGATCGTGCGCCGTGCGCAGCCCCTGGAGAGGGATGCAA |
| | ATTTGCAAGAACAAACAGAGGAGGGCCTACCCGCAGTTGGCGA |
| | CGAGCAGCTAGCGCGCTGGCTTCAAACGCGCGAGCCTGCCGAC |
| | TTGGAGGAGCGACGCAAACTAATGATGGCCGCAGTGCTCGTTA |
| | CCGTGGAGCTTGAGTGCATGCAGCGGTTCTTTGCTGACCCGGAG |
| | ATGCAGCGCAAGCTAGAGGAAACATTGCACTACACCTTTCGAC |
| | AGGGCTACGTACGCCAGGCCTGCAAGATCTCCAACGTGGAGCT |
| | CTGCAACCTGGTCTCCTACCTTGGAATTTTGCACGAAAACCGCC |
| | TTGGGCAAAACGTGCTTCATTCCACGCTCAAGGGCGAGGCGCGC |
| | CGCGACTACGTCCGCGACTGCGTTTACTTATTTCTATGCTACACC |
| | TGGCAGACGGCCATGGGCGTTTGGCAGCAGTGCTTGGAGGAGT |
| | GCAACCTCAAGGAGCTGCAGAAACTGCTAAAGCAAAACTTGAA |
| | GGACCTATGGACGGCCTTCAACGAGCGCTCCGTGGCCGCGCACC |
| | TGGCGGACATCATTTTCCCCGAACGCCTGCTTAAAACCCTGCAA |
| | CAGGGTCTGCCAGACTTCACCAGTCAAAGCATGTTGCAGAACTT |
| | TAGGAACTTTATCCTAGAGCGCTCAGGAATCTTGCCCGCCACCT |
| | GCTGTGCACTTCCTAGCGACTTTGTGCCCATTAAGTACCGCGAA |
| | TGCCCTCCGCCGCTTTGGGGCCACTGCTACCTTCTGCAGCTAGC |
| | CAACTACCTTGCCTACCACTCTGACATAATGGAAGACGTGAGCG |
| | GTGACGGTCTACTGGAGTGTCACTGTCGCTGCAACCTATGCACC |
| | CCGCACCGCTCCCTGGTTTGCAATTCGCAGCTGCTTAACGAAAG |
| | TCAAATTATCGGTACCTTTGAGCTGCAGGGTCCCTCGCCTGACG |
| | AAAAGTCCGCGGCTCCGGGGTTGAAACTCACTCCGGGGCTGTG |
| | GACGTCGGCTTACCTTCGCAAATTTGTACCTGAGGACTACCACG |
| | CCCACGAGATTAGGTTCTACGAAGACCAATCCCGCCCGCCTAAT |
| | GCGGAGCTTACCGCCTGCGTCATTACCCAGGGCCACATTCTTGG |
| | CCAATTGCAAGCCATCAACAAAGCCCGCCAAGAGTTTCTGCTAC |
| | GAAAGGGACGGGGGGTTTACTTGGACCCCCAGTCCGGCGAGGA |
| | GCTCAACCCAATCCCCCCGCCGCCGCAGCCCTATCAGCAGCAGC |
| | CGCGGGCCCTTGCTTCCCAGGATGGCACCCAAAAAGAAGCTGC |
| | AGCTGCCGCCGCCACCCACGGACGAGGAGGAATACTGGGACAG |
| | TCAGGCAGAGGAGGTTTTGGACGAGGAGGAGGAGGACATGATG |
| | GAAGACTGGGAGAGCCTAGACGAGGAAGCTTCCGAGGTCGAAG |
| | AGGTGTCAGACGAAACACCGTCACCCTCGGTCGCATTCCCCTCG |
| | CCGGCGCCCCAGAAATCGGCAACCGGTTCCAGCATGGCTACAA |
| | CCTCCGCTCCTCAGGCGCCGCCGGCACTGCCCGTTCGCCGACCC |
| | AACCGTAGATGGGACACCACTGGAACCAGGGCCGGTAAGTCCA |
| | AGCAGCCGCCGCCGTTAGCCCAAGAGCAACAACAGCGCCAAGG |
| | CTACCGCTCATGGCGCGGGCACAAGAACGCCATAGTTGCTTGCT |
| | TGCAAGACTGTGGGGGCAACATCTCCTTCGCCCGCCGCTTTCTT |
| | CTCTACCATCACGGCGTGGCCTTCCCCCGTAACATCCTGCATTA |
| | CTACCGTCATCTCTACAGCCCATACTGCACCGGCGGCAGCGGCA |
| | GCAACAGCAGCGGCCACACAGAAGCAAAGGCGACCGGATAGC |
| | AAGACTCTGACAAAGCCCAAGAAATCCACAGCGGCGGCAGCAG |
| | CAGGAGGAGGAGCGCTGCGTCTGGCGCCCAACGAACCCGTATC |
| | GACCCGCGAGCTTAGAAACAGGATTTTTCCCACTCTGTATGCTA |
| | TATTTCAACAGAGCAGGGGCCAAGAACAAGAGCTGAAAATAAA |
| | AAACAGGTCTCTGCGATCCCTCACCCGCAGCTGCCTGTATCACA |
| | AAAGCGAAGATCAGCTTCGGCGCACGCTGGAAGACGCGGAGGC |
| | TCTCTTCAGTAAATACTGCGCGCTGACTCTTAAGGACTAGTTTC |
| | GCGCCCTTTCTCAAATTTAAGCGCGAAAACTACGTCATCTCCAG |
| | CGGCCACACCCGGCGCCAGCACCTGTTGTCAGCGCCATTATGAG |
| | CAAGGAAATTCCCACGCCCTACATGTGGAGTTACCAGCCACAA |
| | ATGGGACTTGCGGCTGGAGCTGCCCAAGACTACTCAACCCGAAT |
| | AAACTACATGAGCGCGGGACCCCACATGATATCCCGGGTCAAC |
| | GGAATACGCGCCCACCGAAACCGAATTCTCCTGGAACAGGCGG |
| | CTATTACCACCACACCTCGTAATAACCTTAATCCCCGTAGTTGG |
| | CCCGCTGCCCTGGTGTACCAGGAAAGTCCCGCTCCCACCACTGT |
| | GGTACTTCCCAGAGACGCCCAGGCCGAAGTTCAGATGACTAACT |
| | CAGGGGCGCAGCTTGCGGGCGGCTTTCGTCACAGGGTGCGGTC |
| | GCCCGGGCAGGGTATAACTCACCTGACAATCAGAGGGCGAGGT |
| | ATTCAGCTCAACGACGAGTCGGTGAGCTCCTCGCTTGGTCTCCG |
| | TCCGGACGGGACATTTCAGATCGGCGGCGCCGGCCGCTCTTCAT |
| | TCACGCCTCGTCAGGCAATCCTAACTCTGCAGACCTCGTCCTCT |
| | GAGCCGCGCTCTGGAGGCATTGGAACTCTGCAATTTATTGAGGA |
| | GTTTGTGCCATCGGTCTACTTTAACCCCTTCTCGGGACCTCCCGG |
| | CCACTATCCGGATCAATTTATTCCTAACTTTGACGCGGTAAAGG |
| | ACTCGGCGGACGGCTACGACTGAATGTTAAGTGGAGAGGCAGA |
| | GCAACTGCGCCTGAAACACCTGGTCCACTGTCGCCGCCACAAGT |
| | GCTTTGCCCGCGACTCCGGTGAGTTTTGCTACTTTGAATTGCCCG |
| | AGGATCATATCGAGGGCCCGGCGCACGGCGTCCGGCTTACCGC |
| | CCAGGGAGAGCTTGCCCGTAGCCTGATTCGGGAGTTTACCCAGC |
| | GCCCCCTGCTAGTTGAGCGGGACAGGGGACCCTGTGTTCTCACT |
| | GTGATTTGCAACTGTCCTAACCCTGGATTACATCAAGATCCTCT |
| | AGTTAATGTCAGGTCGCCTAAGTCGATTAACTAGAGTACCCGGG |
| | GATCTTATTCCCTTTAACTAATAAAAAAAAATAATAAAGCATCA |
| | CTTACTTAAAATCAGTTAGCAAATTTCTGTCCAGTTTATTCAGCA |
| | GCACCTCCTTGCCCTCCTCCCAGCTCTGGTATTGCAGCTTCCTCC |
| | TGGCTGCAAACTTTCTCCACAATCTAAATGGAATGTCAGTTTCC |
| | TCCTGTTCCTGTCCATCCGCACCCACTATCTTCATGTTGTTGCAG |
| | ATGAAGCGCGCAAGACCGTCTGAAGATACCTTCAACCCCGTGTA |
| | TCCATATGACACGGAAACCGGTCCTCCAACTGTGCCTTTTCTTA |
| | CTCCTCCCTTTGTATCCCCCAATGGGTTTCAAGAGAGTCCCCCTG |
| | GGGTACTCTCTTTGCGCCTATCCGAACCTCTAGTTACCTCCAATG |
| | GCATGCTTGCGCTCAAAATGGGCAACGGCCTCTCTCTGGACGAG |
| | GCCGGCAACCTTACCTCCCAAAATGTAACCACTGTGAGCCCACC |
| | TCTCAAAAAAACCAAGTCAAACATAAACCTGGAAATATCTGCA |
| | CCCCTCACAGTTACCTCAGAAGCCCTAACTGTGGCTGCCGCCGC |
| | ACCTCTAATGGTCGCGGGCAACACACTCACCATGCAATCACAGG |
| | CCCCGCTAACCGTGCACGACTCCAAACTTAGCATTGCCACCCAA |
| | GGACCCCTCACAGTGTCAGAAGGAAAGCTAGCCCTGCAAACAT |
| | CAGGCCCCCTCACCACCACCGATAGCAGTACCCTTACTATCACT |
| | GCCTCACCCCCTCTAACTACTGCCACTGGTAGCTTGGGCATTGA |
| | CTTGAAAGAGCCCATTTATACACAAAATGGAAAACTAGGACTA |
| | AAGTACGGGGCTCCTTTGCATGTAACAGACGACCTAAACACTTT |
| | GACCGTAGCAACTGGTCCAGGTGTGACTATTAATAATACTTCCT |
| | TGCAAACTAAAGTTACTGGAGCCTTGGGTTTTGATTCACAAGGC |
| | AATATGCAACTTAATGTAGCAGGAGGACTAAGGATTGATTCTCA |
| | AAACAGACGCCTTATACTTGATGTTAGTTATCCGTTTGATGCTC |
| | AAAACCAACTAAATCTAAGACTAGGACAGGGCCCTCTTTTTATA |
| | AACTCAGCCCACAACTTGGATATTAACTACAACAAAGGCCTTTA |
| | CTTGTTTACAGCTTCAAACAATTCCAAAAAGCTTGAGGTTAACC |
| | TAAGCACTGCCAAGGGGTTGATGTTTGACGCTACAGCCATAGCC |
| | ATTAATGCAGGAGATGGGCTTGAATTTGGTTCACCTAATGCACC |
| | AAACACAAATCCCCTCAAAACAAAAATTGGCCATGGCCTAGAA |
| | TTTGATTCAAACAAGGCTATGGTTCCTAAACTAGGAACTGGCCT |
| | TAGTTTTGACAGCACAGGTGCCATTACAGTAGGAAACAAAAAT |
| | AATGATAAGCTAACTTTGTGGACCACACCAGCTCCATCTCCTAA |
| | CTGTAGACTAAATGCAGAGAAAGATGCTAAACTCACTTTGGTCT |
| | TAACAAAATGTGGCAGTCAAATACTTGCTACAGTTTCAGTTTTG |
| | GCTGTTAAAGGCAGTTTGGCTCCAATATCTGGAACAGTTCAAAG |
| | TGCTCATCTTATTATAAGATTTGACGAAAATGGAGTGCTACTAA |
| | ACAATTCCTTCCTGGACCCAGAATATTGGAACTTTAGAAATGGA |
| | GATCTTACTGAAGGCACAGCCTATACAAACGCTGTTGGATTTAT |
| | GCCTAACCTATCAGCTTATCCAAAATCTCACGGTAAAACTGCCA |
| | AAAGTAACATTGTCAGTCAAGTTTACTTAAACGGAGACAAAACT |
| | AAACCTGTAACACTAACCATTACACTAAACGGTACACAGGAAA |
| | CAGGAGACACAACTCCAAGTGCATACTCTATGTCATTTTCATGG |
| | GACTGGTCTGGCCACAACTACATTAATGAAATATTTGCCACATC |
| | CTCTTACACTTTTTCATACATTGCCCAAGAATAAAGAATCGTTTG |
| | TGTTATGTTTCAACGTGTTTATTTTTCAATTGCAGAAAATTTCAA |
| | GTCATTTTTCATTCAGTAGTATAGCCCCACCACCACATAGCTTAT |
| | ACAGATCACCGTACCTTAATCAAACTCACAGAACCCTAGTATTC |
| | AACCTGCCACCTCCCTCCCAACACACAGAGTACACAGTCCTTTC |
| | TCCCCGGCTGGCCTTAAAAAGCATCATATCATGGGTAACAGACA |
| | TATTCTTAGGTGTTATATTCCACACGGTTTCCTGTCGAGCCAAAC |
| | GCTCATCAGTGATATTAATAAACTCCCCGGGCAGCTCACTTAAG |
| | TTCATGTCGCTGTCCAGCTGCTGAGCCACAGGCTGCTGTCCAAC |
| | TTGCGGTTGCTTAACGGGCGGCGAAGGAGAAGTCCACGCCTAC |
| | ATGGGGGTAGAGTCATAATCGTGCATCAGGATAGGGCGGTGGT |
| | GCTGCAGCAGCGCGCGAATAAACTGCTGCCGCCGCCGCTCCGTC |
| | CTGCAGGAATACAACATGGCAGTGGTCTCCTCAGCGATGATTCG |
| | CACCGCCCGCAGCATAAGGCGCCTTGTCCTCCGGGCACAGCAGC |
| | GCACCCTGATCTCACTTAAATCAGCACAGTAACTGCAGCACAGC |
| | ACCACAATATTGTTCAAAATCCCACAGTGCAAGGCGCTGTATCC |
| | AAAGCTCATGGCGGGGACCACAGAACCCACGTGGCCATCATAC |
| | CACAAGCGCAGGTAGATTAAGTGGCGACCCCTCATAAACACGC |
| | TGGACATAAACATTACCTCTTTTGGCATGTTGTAATTCACCACCT |
| | CCCGGTACCATATAAACCTCTGATTAAACATGGCGCCATCCACC |
| | ACCATCCTAAACCAGCTGGCCAAAACCTGCCCGCCGGCTATACA |
| | CTGCAGGGAACCGGGACTGGAACAATGACAGTGGAGAGCCCAG |
| | GACTCGTAACCATGGATCATCATGCTCGTCATGATATCAATGTT |
| | GGCACAACACAGGCACACGTGCATACACTTCCTCAGGATTACA |
| | AGCTCCTCCCGCGTTAGAACCATATCCCAGGGAACAACCCATTC |
| | CTGAATCAGCGTAAATCCCACACTGCAGGGAAGACCTCGCACG |
| | TAACTCACGTTGTGCATTGTCAAAGTGTTACATTCGGGCAGCAG |
| | CGGATGATCCTCCAGTATGGTAGCGCGGGTTTCTGTCTCAAAAG |
| | GAGGTAGACGATCCCTACTGTACGGAGTGCGCCGAGACAACCG |
| | AGATCGTGTTGGTCGTAGTGTCATGCCAAATGGAACGCCGGACG |
| | TAGTCATATTTCCTGAAGCAAAACCAGGTGCGGGCGTGACAAA |
| | CAGATCTGCGTCTCCGGTCTCGCCGCTTAGATCGCTCTGTGTAGT |
| | AGTTGTAGTATATCCACTCTCTCAAAGCATCCAGGCGCCCCCTG |
| | GCTTCGGGTTCTATGTAAACTCCTTCATGCGCCGCTGCCCTGATA |
| | ACATCCACCACCGCAGAATAAGCCACACCCAGCCAACCTACAC |
| | ATTCGTTCTGCGAGTCACACACGGGAGGAGCGGGAAGAGCTGG |
| | AAGAACCATGTTTTTTTTTTTATTCCAAAAGATTATCCAAAACCT |
| | CAAAATGAAGATCTATTAAGTGAACGCGCTCCCCTCCGGTGGCG |
| | TGGTCAAACTCTACAGCCAAAGAACAGATAATGGCATTTGTAA |
| | GATGTTGCACAATGGCTTCCAAAAGGCAAACGGCCCTCACGTCC |
| | AAGTGGACGTAAAGGCTAAACCCTTCAGGGTGAATCTCCTCTAT |
| | AAACATTCCAGCACCTTCAACCATGCCCAAATAATTCTCATCTC |
| | GCCACCTTCTCAATATATCTCTAAGCAAATCCCGAATATTAAGT |
| | CCGGCCATTGTAAAAATCTGCTCCAGAGCGCCCTCCACCTTCAG |
| | CCTCAAGCAGCGAATCATGATTGCAAAAATTCAGGTTCCTCACA |
| | GACCTGTATAAGATTCAAAAGCGGAACATTAACAAAAATACCG |
| | CGATCCCGTAGGTCCCTTCGCAGGGCCAGCTGAACATAATCGTG |
| | CAGGTCTGCACGGACCAGCGCGGCCACTTCCCCGCCAGGAACC |
| | ATGACAAAAGAACCCACACTGATTATGACACGCATACTCGGAG |
| | CTATGCTAACCAGCGTAGCCCCGATGTAAGCTTGTTGCATGGGC |
| | GGCGATATAAAATGCAAGGTGCTGCTCAAAAAATCAGGCAAAG |
| | CCTCGCGCAAAAAAGAAAGCACATCGTAGTCATGCTCATGCAG |
| | ATAAAGGCAGGTAAGCTCCGGAACCACCACAGAAAAAGACACC |
| | ATTTTTCTCTCAAACATGTCTGCGGGTTTCTGCATAAACACAAA |
| | ATAAAATAACAAAAAAACATTTAAACATTAGAAGCCTGTCTTAC |
| | AACAGGAAAAACAACCCTTATAAGCATAAGACGGACTACGGCC |
| | ATGCCGGCGTGACCGTAAAAAAACTGGTCACCGTGATTAAAAA |
| | GCACCACCGACAGCTCCTCGGTCATGTCCGGAGTCATAATGTAA |
| | GACTCGGTAAACACATCAGGTTGATTCACATCGGTCAGTGCTAA |
| | AAAGCGACCGAAATAGCCCGGGGGAATACATACCCGCAGGCGT |
| | AGAGACAACATTACAGCCCCCATAGGAGGTATAACAAAATTAA |
| | TAGGAGAGAAAAACACATAAACACCTGAAAAACCCTCCTGCCT |
| | AGGCAAAATAGCACCCTCCCGCTCCAGAACAACATACAGCGCT |
| | TCCACAGCGGCAGCCATAACAGTCAGCCTTACCAGTAAAAAAG |
| | AAAACCTATTAAAAAAACACCACTCGACACGGCACCAGCTCAA |
| | TCAGTCACAGTGTAAAAAAGGGCCAAGTGCAGAGCGAGTATAT |
| | ATAGGACTAAAAAATGACGTAACGGTTAAAGTCCACAAAAAAC |
| | ACCCAGAAAACCGCACGCGAACCTACGCCCAGAAACGAAAGCC |
| | AAAAAACCCACAACTTCCTCAAATCGTCACTTCCGTTTTCCCAC |
| | GTTACGTCACTTCCCATTTTAAGAAAACTACAATTCCCAACACA |
| | TACAAGTTACTCCGCCCTAAAACCTACGTCACCCGCCCCGTTCC |
| | CACGCCCCGCGCCACGTCACAAACTCCACCCCCTCATTATCATA |
| | TTGGCTTCAATCCAAAATAAGGTATATTATTGATGAT |
| SEQ ID NO: 9 | GGAGGACACTTCTCAGAAGGGGTTGTTTTGCTTTTGCTTATTTCC |
| | GTCCATTTCCCTCTCTGCGCGCGGACCTTCCTTTTCCAGATGGTG |
| | AGAGCCGCGGGGACACCCGACGCCGGGGCAGGCTGATCCACGA |
| | TCCTGGGTGTGCGTAACGCCGCCTGGGGCTCCGTGGGCGAGGG |
| | ACGTGTGGGGACAGGTGCACCGGAAACTGCCAGACTGGAGAGT |
| | TGAGGCATCGGAGGCGCGAGAACAGCACTACTACTGCGGCGAG |
| | ACGAGCGCGGCGCATCCCAAAGCCCGGCCAAATGCGCTCGTCC |
| | CTGGGAGGGGAGGGAGGCGCGCCTGGAGCGGGGACAGTCTTGG |
| | TCCGCGCCCTCCTCCCGGGTCTGTGCCGGGACCCGGGACCCGGG |
| | AGCCGTCGCAGGTCTCGGTCCAAGGGGCCCCTTTTCTCGGAAGG |
| | GCGGCGGCCAAGAGCAGGGAAGGTGGATCTCAGGTAGCGAGTC |
| | TGGGCTTCGGGGACGGCGGGGAGGGGAGCCGGACGGGAGGAT |
| | GAGCTCCCCTGGCACCGAGAGCGCGGGAAAGAGCCTGCAGTAC |
| | CGAGTGGACCACCTGCTGAGCGCCGTGGAGAATGAGCTGCAGG |
| | CGGGCAGCGAGAAGGGCGACCCCACAGAGCGCGAACTGCGCGT |
| | GGGCCTGGAGGAGAGCGAGCTGTGGCTGCGCTTCAAGGAGCTC |
| | ACCAATGAGATGATCGTGACCAAGAACGGCAGGAGGATGTTTC |
| | CGGTGCTGAAGGTGAACGTGTCTGGCCTGGACCCCAACGCCATG |
| | TACTCCTTCCTGCTGGACTTCGTGGCGGCGGACAACCACCGCTG |
| | GAAGTACGTGAACGGGGAATGGGTGCCGGGGGGCAAGCCGGA |
| | GCCGCAGGCGCCCAGCTGCGTCTACATCCACCCCGACTCGCCCA |
| | ACTTCGGGGCCCACTGGATGAAGGCTCCCGTCTCCTTCAGCAAA |
| | GTCAAGCTCACCAACAAGCTCAACGGAGGGGGCCAGATCATGC |
| | TGAACTCCTTGCATAAGTATGAGCCTCGAATCCACATAGTGAGA |
| | GTTGGGGGTCCACAGCGCATGATCACCAGCCACTGCTTCCCTGA |
| | GACCCAGTTCATAGCGGTGACTGCTTATCAGAACGAGGAGATC |
| | ACAGCTCTTAAAATTAAGTACAATCCATTTGCAAAAGCTTTCCT |
| | TGATGCAAAGGAAAGAAGTGATCACAAAGAGATGATGGAGGA |
| | ACCCGGAGACAGCCAGCAACCTGGGTACTCCCAATGGGGGTGG |
| | CTTCTTCCTGGAACCAGCACCCTGTGTCCACCTGCAAATCCTCAT |
| | CCTCAGTTTGGAGGTGCCCTCTCCCTCCCCTCCACGCACAGCTGT |
| | GACAGGTACCCAACCCTGAGGAGCCACCGGTCCTCACCCTACCC |
| | CAGCCCCTATGCTCATCGGAACAATTCTCCAACCTATTCTGACA |
| | ACTCACCTGCATGTTTATCCATGCTGCAATCCCATGACAATTGG |
| | TCCAGCCTTGGAATGCCTGCCCATCCCAGCATGCTCCCCGTGAG |
| | CCACAATGCCAGCCCACCTACCAGCTCCAGTCAGTACCCCAGCC |
| | TGTGGTCTGTGAGCAACGGCGCCGTCACCCCGGGCTCCCAGGCA |
| | GCAGCCGTGTCCAACGGGCTGGGGGCCCAGTTCTTCCGGGGCTC |
| | CCCCGCGCACTACACACCCCTCACCCATCCGGTCTCGGCGCCCT |
| | CTTCCTCGGGATCCCCACTGTACGAAGGGGCGGCCGCGGCCACA |
| | GACATCGTGGACAGCCAGTACGACGCCGCAGCCCAAGGCCGCC |
| | TCATAGCCTCATGGACACCTGTGTCGCCACCTTCCATGTGAAGC |
| | AGCAAGGCCCAGGTCCCGAAAGATGCAGTGACTTTTTGTCGTGG |
| | CAGCCAGTGGTGACTGGATTGACCTACTAGGTACCCAGTGGCAG |
| | TCTCAGGTTAAGAAGGAAATGCAGCCTCAGTAACTTCCTTTTCA |
| | AAGCAGTGGAGGAGCACACGGCACCTTTCCCCAGAGCCCCAGC |
| | ATCCCTTGCTCACACCTGCAGTAGCGGTGCTGTCCCAGGTGGCT |
| | TACAGATGAACCCAACTGTGGAGATGATGCAGTTGGCCCAACCT |
| | CACTGACGGTGAAAAAATGTTTGCCAGGGTCCAGAAACTTTTTT |
| | TGGTTTATTTCTCATACAGTGTATTGGCAACTTTGGCACACCAG |
| | AATTTGTAAACTCCACCAGTCCTACTTTAGTGAGATAAAAAGCA |
| | CACTCTTAATCTTCTTCCTTGTTGCTTTCAAGTAGTTAGAGTTGA |
| | GCTGTTAAGGACAGAATAAAATCATAGTTGAGGACAGCAGGTT |
| | TTAGTTGAATTGAAAATTTGACTGCTCTGCCCCCTAGAATGTGT |
| | GTATTTTAAGCATATGTAGCTAATCTCTTGTGTTGTTAAACTATA |
| | ACTGTTTCATATTTTTCTTTTGACAAAGTAGCCAAAGACAATCA |
| | GCAGAAAGCATTTTCTGCAAAATAAACGCAATATGCAAAAAAA |
| | AAAAAAAAAAA |
| SEQ ID NO: 10 | TCTAGAGCCACCATGAGCTCCCCTGGCACCGAGAGCGCGGGAA |
| | AGAGCCTGCAGTACCGAGTGGACCACCTGCTGAGCGCCGTGGA |
| | GAATGAGCTGCAGGCGGGCAGCGAGAAGGGCGACCCCACAGA |
| | GCGCGAACTGCGCGTGGGCCTGGAGGAGAGCGAGCTGTGGCTG |
| | CGCTTCAAGGAGCTCACCAATGAGATGATCGTGACCAAGAACG |
| | GCAGGAGGATGTTTCCGGTGCTGAAGGTGAACGTGTCTGGCCTG |
| | GACCCCAACGCCATGTACTCCTTCCTGCTGGACTTCGTGGCGGC |
| | GGACAACCACCGCTGGAAGTACGTGAACGGGGAATGGGTGCCG |
| | GGGGGCAAGCCGGAGCCGCAGGCGCCCAGCTGCGTCTACATCC |
| | ACCCCGACTCGCCCAACTTCGGGGCCCACTGGATGAAGGCTCCC |
| | GTCTCCTTCAGCAAAGTCAAGCTCACCAACAAGCTCAACGGAG |
| | GGGGCCAGATCATGCTGAACTCCTTGCATAAGTATGAGCCTCGA |
| | ATCCACATAGTGAGAGTTGGGGGTCCACAGCGCATGATCACCA |
| | GCCACTGCTTCCCTGAGACCCAGTTCATAGCGGTGACTGCTAGA |
| | AGTGATCACAAAGAGATGATGGAGGAACCCGGAGACAGCCAGC |
| | AACCTGGGTACTCCCAATGGGGGTGGCTTCTTCCTGGAACCAGC |
| | ACCGTGTGTCCACCTGCAAATCCTCATCCTCAGTTTGGAGGTGC |
| | CCTCTCCCTCCCCTCCACGCACAGCTGTGACAGGTACCCAACCC |
| | TGAGGAGCCACCGGTCCTCACCCTACCCCAGCCCCTATGCTCAT |
| | CGGAACAATTCTCCAACCTATTCTGACAACTCACCTGCATGTTT |
| | ATCCATGCTGCAATCCCATGACAATTGGTCCAGCCTTGGAATGC |
| | CTGCCCATCCCAGCATGCTCCCCGTGAGCCACAATGCCAGCCCA |
| | CCTACCAGCTCCAGTCAGTACCCCAGCCTGTGGTCTGTGAGCAA |
| | CGGCGCCGTCACCCCGGGCTCCCAGGCAGCAGCCGTGTCCAAC |
| | GGGCTGGGGGCCCAGTTCTTCCGGGGCTCCCCCGCGCACTACAC |
| | ACCCCTCACCCATCCGGTCTCGGCGCCCTCTTCCTCGGGATCCCC |
| | ACTGTACGAAGGGGCGGCCGCGGCCACAGACATCGTGGACAGC |
| | CAGTACGACGCCGCAGCCCAAGGCCGCCTCATAGCCTCATGGA |
| | CACCTGTGTCGCCACCTTCCATGTGAGATATC |
| SEQ ID NO: 11 | TCTCTCCNA |
| SEQ ID NO: 12 | MSSPGTESAGKSLQYRVDHLLSAVENELQAGSEKGDPTERELRVG |
| | LEESELWLRFKELTNEMIVTKNGRRMFPVLKVNVSGLDPNAMYSF |
| | LLDFVAADNHRWKYVNGEWVPGGKPEPQAPSCVYIHPDSPNFGA |
| | HWMKAPVSFSKVKLTNKLNGGGQIMLNSLHKYEPRIHIVRVGDPQ |
| | RMITSHCFPETQFIAVTAYQNEEITALKIKYNPFAKAFLDAKERSDH |
| | KEMMEEPGDSQQPGYSQWGWLLPGTSTLCPPANPHPQFGGALSLP |
| | STHSCDRYPTLRSHRSSPYPSPYAHRNNSPTYSDNSPACLSMLQSH |
| | DNWSSLGMPAHPSMLPVSHNASPPTSSSQYPSLWSVSNGAVTPGS |
| | QAAAVTNGLGAQFFRGSPAHYTPLTHPVSAPSSSGSPLYEGAAAAT |
| | NIVDSQYDAAAQGRLIASWTPVSPPSM |
| SEQ ID NO: 13 | CATCATCAATAATATACCTTATTTTGGATTGAAGCCAATATGAT |
| | AATGAGGGGGTGGAGTTTGTGACGTGGCGCGGGGCGTGGGAAC |
| | GGGGCGGGTGACGTAGTAGTGTGGCGGAAGTGTGATGTTGCAA |
| | GTGTGGCGGAACACATGTAAGCGACGGATGTGGCAAAAGTGAC |
| | GTTTTTGGTGTGCGCCGGTGTACACAGGAAGTGACAATTTTCGC |
| | GCGGTTTTAGGCGGATGTTGTAGTAAATTTGGGCGTAACCGAGT |
| | AAGATTTGGCCATTTTCGCGGGAAAACTGAATAAGAGGAAGTG |
| | AAATCTGAATAATTTTGTGTTACTCATAGCGCGTAATACTGTAA |
| | TAGTAATCAATTACGGGGTCATTAGTTCATAGCCCATATATGGA |
| | GTTCCGCGTTACATAACTTACGGTAAATGGCCCGCCTGGCTGAC |
| | CGCCCAACGACCCCCGCCCATTGACGTCAATAATGACGTATGTT |
| | CCCATAGTAACGCCAATAGGGACTTTCCATTGACGTCAATGGGT |
| | GGAGTATTTACGGTAAACTGCCCACTTGGCAGTACATCAAGTGT |
| | ATCATATGCCAAGTACGCCCCCTATTGACGTCAATGACGGTAAA |
| | TGGCCCGCCTGGCATTATGCCCAGTACATGACCTTATGGGACTT |
| | TCCTACTTGGCAGTACATCTACGTATTAGTCATCGCTATTACCAT |
| | GGTGATGCGGTTTTGGCAGTACATCAATGGGCGTGGATAGCGGT |
| | TTGACTCACGGGGATTTCCAAGTCTCCACCCCATTGACGTCAAT |
| | GGGAGTTTGTTTTGGCACCAAAATCAACGGGACTTTCCAAAATG |
| | TCGTAACAACTCCGCCCCATTGACGCAAATGGGCGGTAGGCGTG |
| | TACGGTGGGAGGTCTATATAAGCAGAGCTGGTTTAGTGAACCGT |
| | CAGATCCGCTAGAGATCTGGTACCGTCGACGCGGCCGCTCGAGC |
| | CTAAGCTTCTAGATGCATGCTCGAGCGGCCGCCAGTGTGATGGA |
| | TATCTGCAGAATTCGCCCTTGCTTCTAGAGCCACCATGAGCTCC |
| | CCTGGCACCGAGAGCGCGGGAAAGAGCCTGCAGTACCGAGTGG |
| | ACCACCTGCTGAGCGCCGTGGAGAATGAGCTGCAGGCGGGCAG |
| | CGAGAAGGGCGACCCCACAGAGCGCGAACTGCGCGTGGGCCTG |
| | GAGGAGAGCGAGCTGTGGCTGCGCTTCAAGGAGCTCACCAATG |
| | AGATGATCGTGACCAAGAACGGCAGGAGGATGTTTCCGGTGCT |
| | GAAGGTGAACGTGTCTGGCCTGGACCCCAACGCCATGTACTCCT |
| | TCCTGCTGGACTTCGTGGCGGCGGACAACCACCGCTGGAAGTAC |
| | GTGAACGGGGAATGGGTGCCGGGGGGCAAGCCGGAGCCGCAG |
| | GCGCCCAGCTGCGTCTACATCCACCCCGACTCGCCCAACTTCGG |
| | GGCCCACTGGATGAAGGCTCCCGTCTCCTTCAGCAAAGTCAAGC |
| | TCACCAACAAGCTCAACGGAGGGGGCCAGATCATGCTGAACTC |
| | CTTGCATAAGTATGAGCCTCGAATCCACATAGTGAGAGTTGGGG |
| | GTCCACAGCGCATGATCACCAGCCACTGCTTCCCTGAGACCCAG |
| | TTCATAGCGGTGACTGCTAGAAGTGATCACAAAGAGATGATGG |
| | AGGAACCCGGAGACAGCCAGCAACCTGGGTACTCCCAATGGGG |
| | GTGGCTTCTTCCTGGAACCAGCACCGTGTGTCCACCTGCAAATC |
| | CTCATCCTCAGTTTGGAGGTGCCCTCTCCCTCCCCTCCACGCACA |
| | GCTGTGACAGGTACCCAACCCTGAGGAGCCACCGGTCCTCACCC |
| | TACCCCAGCCCCTATGCTCATCGGAACAATTCTCCAACCTATTCT |
| | GACAACTCACCTGCATGTTTATCCATGCTGCAATCCCATGACAA |
| | TTGGTCCAGCCTTGGAATGCCTGCCCATCCCAGCATGCTCCCCG |
| | TGAGCCACAATGCCAGCCCACCTACCAGCTCCAGTCAGTACCCC |
| | AGCCTGTGGTCTGTGAGCAACGGCGCCGTCACCCCGGGCTCCCA |
| | GGCAGCAGCCGTGTCCAACGGGCTGGGGGCCCAGTTCTTCCGG |
| | GGCTCCCCCGCGCACTACACACCCCTCACCCATCCGGTCTCGGC |
| | GCCCTCTTCCTCGGGATCCCCACTGTACGAAGGGGCGGCCGCGG |
| | CCACAGACATCGTGGACAGCCAGTACGACGCCGCAGCCCAAGG |
| | CCGCCTCATAGCCTCATGGACACCTGTGTCGCCACCTTCCATGT |
| | GAGATATCCGATCCACCGGATCTAGATAACTGATCATAATCAGC |
| | CATACCACATTTGTAGAGGTTTTACTTGCTTTAAAAAACCTCCC |
| | ACACCTCCCCCTGAACCTGAAACATAAAATGAATGCAATTGTTG |
| | TTGTTAACTTGTTTATTGCAGCTTATAATGGTTACAAATAAAGC |
| | AATAGCATCACAAATTTCACAAATAAAGCATTTTTTTCACTGCA |
| | TTCTAGTTGTGGTTTGTCCAAACTCATCAATGTATCTTAACGCGG |
| | ATCTGGAAGGTGCTGAGGTACGATGAGACCCGCACCAGGTGCA |
| | GACCCTGCGAGTGTGGCGGTAAACATATTAGGAACCAGCCTGT |
| | GATGCTGGATGTGACCGAGGAGCTGAGGCCCGATCACTTGGTG |
| | CTGGCCTGCACCCGCGCTGAGTTTGGCTCTAGCGATGAAGATAC |
| | AGATTGAGGTACTGAAATGTGTGGGCGTGGCTTAAGGGTGGGA |
| | AAGAATATATAAGGTGGGGGTCTTATGTAGTTTTGTATCTGTTTT |
| | GCAGCAGCCGCCGCCGCCATGAGCACCAACTCGTTTGATGGAA |
| | GCATTGTGAGCTCATATTTGACAACGCGCATGCCCCCATGGGCC |
| | GGGGTGCGTCAGAATGTGATGGGCTCCAGCATTGATGGTCGCCC |
| | CGTCCTGCCCGCAAACTCTACTACCTTGACCTACGAGACCGTGT |
| | CTGGAACGCCGTTGGAGACTGCAGCCTCCGCCGCCGCTTCAGCC |
| | GCTGCAGCCACCGCCCGCGGGATTGTGACTGACTTTGCTTTCCT |
| | GAGCCCGCTTGCAAGCAGTGCAGCTTCCCGTTCATCCGCCCGCG |
| | ATGACAAGTTGACGGCTCTTTTGGCACAATTGGATTCTTTGACC |
| | CGGGAACTTAATGTCGTTTCTCAGCAGCTGTTGGATCTGCGCCA |
| | GCAGGTTTCTGCCCTGAAGGCTTCCTCCCCTCCCAATGCGGTTTA |
| | AAACATAAATAAAAAACCAGACTCTGTTTGGATTTGGATCAAGC |
| | AAGTGTCTTGCTGTCTTTATTTAGGGGTTTTGCGCGCGCGGTAG |
| | GCCCGGGACCAGCGGTCTCGGTCGTTGAGGGTCCTGTGTATTTT |
| | TTCCAGGACGTGGTAAAGGTGACTCTGGATGTTCAGATACATGG |
| | GCATAAGCCCGTCTCTGGGGTGGAGGTAGCACCACTGCAGAGC |
| | TTCATGCTGCGGGGTGGTGTTGTAGATGATCCAGTCGTAGCAGG |
| | AGCGCTGGGCGTGGTGCCTAAAAATGTCTTTCAGTAGCAAGCTG |
| | ATTGCCAGGGGCAGGCCCTTGGTGTAAGTGTTTACAAAGCGGTT |
| | AAGCTGGGATGGGTGCATACGTGGGGATATGAGATGCATCTTG |
| | GACTGTATTTTTAGGTTGGCTATGTTCCCAGCCATATCCCTCCGG |
| | GGATTCATGTTGTGCAGAACCACCAGCACAGTGTATCCGGTGCA |
| | CTTGGGAAATTTGTCATGTAGCTTAGAAGGAAATGCGTGGAAG |
| | AACTTGGAGACGCCCTTGTGACCTCCAAGATTTTCCATGCATTC |
| | GTCCATAATGATGGCAATGGGCCCACGGGCGGCGGCCTGGGCG |
| | AAGATATTTCTGGGATCACTAACGTCATAGTTGTGTTCCAGGAT |
| | GAGATCGTCATAGGCCATTTTTACAAAGCGCGGGCGGAGGGTG |
| | CCAGACTGCGGTATAATGGTTCCATCCGGCCCAGGGGCGTAGTT |
| | ACCCTCACAGATTTGCATTTCCCACGCTTTGAGTTCAGATGGGG |
| | GGATCATGTCTACCTGCGGGGCGATGAAGAAAACGGTTTCCGG |
| | GGTAGGGGAGATCAGCTGGGAAGAAAGCAGGTTCCTGAGCAGC |
| | TGCGACTTACCGCAGCCGGTGGGCCCGTAAATCACACCTATTAC |
| | CGGCTGCAACTGGTAGTTAAGAGAGCTGCAGCTGCCGTCATCCC |
| | TGAGCAGGGGGGCCACTTCGTTAAGCATGTCCCTGACTCGCATG |
| | TTTTCCCTGACCAAATCCGCCAGAAGGCGCTCGCCGCCCAGCGA |
| | TAGCAGTTCTTGCAAGGAAGCAAAGTTTTTCAACGGTTTGAGAC |
| | CGTCCGCCGTAGGCATGCTTTTGAGCGTTTGACCAAGCAGTTCC |
| | AGGCGGTCCCACAGCTCGGTCACCTGCTCTACGGCATCTCGATC |
| | CAGCATATCTCCTCGTTTCGCGGGTTGGGGCGGCTTTCGCTGTA |
| | CGGCAGTAGTCGGTGCTCGTCCAGACGGGCCAGGGTCATGTCTT |
| | TCCACGGGCGCAGGGTCCTCGTCAGCGTAGTCTGGGTCACGGTG |
| | AAGGGGTGCGCTCCGGGCTGCGCGCTGGCCAGGGTGCGCTTGA |
| | GGCTGGTCCTGCTGGTGCTGAAGCGCTGCCGGTCTTCGCCCTGC |
| | GCGTCGGCCAGGTAGCATTTGACCATGGTGTCATAGTCCAGCCC |
| | CTCCGCGGCGTGGCCCTTGGCGCGCAGCTTGCCCTTGGAGGAGG |
| | CGCCGCACGAGGGGCAGTGCAGACTTTTGAGGGCGTAGAGCTT |
| | GGGCGCGAGAAATACCGATTCCGGGGAGTAGGCATCCGCGCCG |
| | CAGGCCCCGCAGACGGTCTCGCATTCCACGAGCCAGGTGAGCTC |
| | TGGCCGTTCGGGGTCAAAAACCAGGTTTCCCCCATGCTTTTTGA |
| | TGCGTTTCTTACCTCTGGTTTCCATGAGCCGGTGTCCACGCTCGG |
| | TGACGAAAAGGCTGTCCGTGTCCCCGTATACAGACTTGAGAGGC |
| | CTGTCCTCGAGCGGTGTTCCGCGGTCCTCCTCGTATAGAAACTC |
| | GGACCACTCTGAGACAAAGGCTCGCGTCCAGGCCAGCACGAAG |
| | GAGGCTAAGTGGGAGGGGTAGCGGTCGTTGTCCACTAGGGGGT |
| | CCACTCGCTCCAGGGTGTGAAGACACATGTCGCCCTCTTCGGCA |
| | TCAAGGAAGGTGATTGGTTTGTAGGTGTAGGCCACGTGACCGG |
| | GTGTTCCTGAAGGGGGGCTATAAAAGGGGGTGGGGGCGCGTTC |
| | GTCCTCACTCTCTTCCGCATCGCTGTCTGCGAGGGCCAGCTGTTG |
| | GGGTGAGTACTCCCTCTGAAAAGCGGGCATGACTTCTGCGCTAA |
| | GATTGTCAGTTTCCAAAAACGAGGAGGATTTGATATTCACCTGG |
| | CCCGCGGTGATGCCTTTGAGGGTGGCCGCATCCATCTGGTCAGA |
| | AAAGACAATCTTTTTGTTGTCAAGCTTGGTGGCAAACGACCCGT |
| | AGAGGGCGTTGGACAGCAACTTGGCGATGGAGCGCAGGGTTTG |
| | GTTTTTGTCGCGATCGGCGCGCTCCTTGGCCGCGATGTTTAGCTG |
| | CACGTATTCGCGCGCAACGCACCGCCATTCGGGAAAGACGGTG |
| | GTGCGCTCGTCGGGCACCAGGTGCACGCGCCAACCGCGGTTGTG |
| | CAGGGTGACAAGGTCAACGCTGGTGGCTACCTCTCCGCGTAGGC |
| | GCTCGTTGGTCCAGCAGAGGCGGCCGCCCTTGCGCGAGCAGAA |
| | TGGCGGTAGGGGGTCTAGCTGCGTCTCGTCCGGGGGGTCTGCGT |
| | CCACGGTAAAGACCCCGGGCAGCAGGCGCGCGTCGAAGTAGTC |
| | TATCTTGCATCCTTGCAAGTCTAGCGCCTGCTGCCATGCGCGGG |
| | CGGCAAGCGCGCGCTCGTATGGGTTGAGTGGGGGACCCCATGG |
| | CATGGGGTGGGTGAGCGCGGAGGCGTACATGCCGCAAATGTCG |
| | TAAACGTAGAGGGGCTCTCTGAGTATTCCAAGATATGTAGGGTA |
| | GCATCTTCCACCGCGGATGCTGGCGCGCACGTAATCGTATAGTT |
| | CGTGCGAGGGAGCGAGGAGGTCGGGACCGAGGTTGCTACGGGC |
| | GGGCTGCTCTGCTCGGAAGACTATCTGCCTGAAGATGGCATGTG |
| | AGTTGGATGATATGGTTGGACGCTGGAAGACGTTGAAGCTGGC |
| | GTCTGTGAGACCTACCGCGTCACGCACGAAGGAGGCGTAGGAG |
| | TCGCGCAGCTTGTTGACCAGCTCGGCGGTGACCTGCACGTCTAG |
| | GGCGCAGTAGTCCAGGGTTTCCTTGATGATGTCATACTTATCCT |
| | GTCCCTTTTTTTTCCACAGCTCGCGGTTGAGGACAAACTCTTCGC |
| | GGTCTTTCCAGTACTCTTGGATCGGAAACCCGTCGGCCTCCGAA |
| | CGGTAAGAGCCTAGCATGTAGAACTGGTTGACGGCCTGGTAGG |
| | CGCAGCATCCCTTTTCTACGGGTAGCGCGTATGCCTGCGCGGCC |
| | TTCCGGCATGACCAGCATGAAGGGCACGAGCTGCTTCCCAAAG |
| | GCCCCCATCCAAGTATAGGTCTCTACATCGTAGGTGACAAAGAG |
| | ACGCTCGGTGCGAGGATGCGAGCCGATCGGGAAGAACTGGATC |
| | TCCCGCCACCAATTGGAGGAGTGGCTATTGATGTGGTGAAAGTA |
| | GAAGTCCCTGCGACGGGCCGAACACTCGTGCTGGCTTTTGTAAA |
| | AACGTGCGCAGTACTGGCAGCGGTGCACGGGCTGTACATCCTGC |
| | ACGAGGTTGACCTGACGACCGCGCACAAGGAAGCAGAGTGGGA |
| | ATTTGAGCCCCTCGCCTGGCGGGTTTGGCTGGTGGTCTTCTACTT |
| | CGGCTGCTTGTCCTTGACCGTCTGGCTGCTCGAGGGGAGTTACG |
| | GTGGATCGGACCACCACGCCGCGCGAGCCCAAAGTCCAGATGT |
| | CCGCGCGCGGCGGTCGGAGCTTGATGACAACATCGCGCAGATG |
| | GGAGCTGTCCATGGTCTGGAGCTCCCGCGGCGTCAGGTCAGGCG |
| | GGAGCTCCTGCAGGTTTACCTCGCATAGACGGGTCAGGGCGCG |
| | GGCTAGATCCAGGTGATACCTAATTTCCAGGGGCTGGTTGGTGG |
| | CGGCGTCGATGGCTTGCAAGAGGCCGCATCCCCGCGGCGCGAC |
| | TACGGTACCGCGCGGCGGGCGGTGGGCCGCGGGGGTGTCCTTG |
| | GATGATGCATCTAAAAGCGGTGACGCGGGCGAGCCCCCGGAGG |
| | TAGGGGGGGCTCCGGACCCGCCGGGAGAGGGGGCAGGGGCAC |
| | GTCGGCGCCGCGCGCGGGCAGGAGCTGGTGCTGCGCGCGTAGG |
| | TTGCTGGCGAACGCGACGACGCGGCGGTTGATCTCCTGAATCTG |
| | GCGCCTCTGCGTGAAGACGACGGGCCCGGTGAGCTTGAACCTG |
| | AAAGAGAGTTCGACAGAATCAATTTCGGTGTCGTTGACGGCGG |
| | CCTGGCGCAAAATCTCCTGCACGTCTCCTGAGTTGTCTTGATAG |
| | GCGATCTCGGCCATGAACTGCTCGATCTCTTCCTCCTGGAGATC |
| | TCCGCGTCCGGCTCGCTCCACGGTGGCGGCGAGGTCGTTGGAAA |
| | TGCGGGCCATGAGCTGCGAGAAGGCGTTGAGGCCTCCCTCGTTC |
| | CAGACGCGGCTGTAGACCACGCCCCCTTCGGCATCGCGGGCGC |
| | GCATGACCACCTGCGCGAGATTGAGCTCCACGTGCCGGGCGAA |
| | GACGGCGTAGTTTCGCAGGCGCTGAAAGAGGTAGTTGAGGGTG |
| | GTGGCGGTGTGTTCTGCCACGAAGAAGTACATAACCCAGCGTCG |
| | CAACGTGGATTCGTTGATAATTGTTGTGTAGGTACTCCGCCGCC |
| | GAGGGACCTGAGCGAGTCCGCATCGACCGGATCGGAAAACCTC |
| | TCGAGAAAGGCGTCTAACCAGTCACAGTCGCAAGGTAGGCTGA |
| | GCACCGTGGCGGGCGGCAGCGGGCGGCGGTCGGGGTTGTTTCT |
| | GGCGGAGGTGCTGCTGATGATGTAATTAAAGTAGGCGGTCTTGA |
| | GACGGCGGATGGTCGACAGAAGCACCATGTCCTTGGGTCCGGC |
| | CTGCTGAATGCGCAGGCGGTCGGCCATGCCCCAGGCTTCGTTTT |
| | GACATCGGCGCAGGTCTTTGTAGTAGTCTTGCATGAGCCTTTCT |
| | ACCGGCACTTCTTCTTCTCCTTCCTCTTGTCCTGCATCTCTTGCAT |
| | CTATCGCTGCGGCGGCGGCGGAGTTTGGCCGTAGGTGGCGCCCT |
| | CTTCCTCCCATGCGTGTGACCCCGAAGCCCCTCATCGGCTGAAG |
| | CAGGGCTAGGTCGGCGACAACGCGCTCGGCTAATATGGCCTGCT |
| | GCACCTGCGTGAGGGTAGACTGGAAGTCATCCATGTCCACAAA |
| | GCGGTGGTATGCGCCCGTGTTGATGGTGTAAGTGCAGTTGGCCA |
| | TAACGGACCAGTTAACGGTCTGGTGACCCGGCTGCGAGAGCTC |
| | GGTGTACCTGAGACGCGAGTAAGCCCTCGAGTCAAATACGTAG |
| | TCGTTGCAAGTCCGCACCAGGTACTGGTATCCCACCAAAAAGTG |
| | CGGCGGCGGCTGGCGGTAGAGGGGCCAGCGTAGGGTGGCCGGG |
| | GCTCCGGGGGCGAGATCTTCCAACATAAGGCGATGATATCCGTA |
| | GATGTACCTGGACATCCAGGTGATGCCGGCGGCGGTGGTGGAG |
| | GCGCGCGGAAAGTCGCGGACGCGGTTCCAGATGTTGCGCAGCG |
| | GCAAAAAGTGCTCCATGGTCGGGACGCTCTGGCCGGTCAGGCG |
| | CGCGCAATCGTTGACGCTCTAGCGTGCAAAAGGAGAGCCTGTA |
| | AGCGGGCACTCTTCCGTGGTCTGGTGGATAAATTCGCAAGGGTA |
| | TCATGGCGGACGACCGGGGTTCGAGCCCCGTATCCGGCCGTCCG |
| | CCGTGATCCATGCGGTTACCGCCCGCGTGTCGAACCCAGGTGTG |
| | CGACGTCAGACAACGGGGGAGTGCTCCTTTTGGCTTCCTTCCAG |
| | GCGCGGCGGCTGCTGCGCTAGCTTTTTTGGCCACTGGCCGCGCG |
| | CAGCGTAAGCGGTTAGGCTGGAAAGCGAAAGCATTAAGTGGCT |
| | CGCTCCCTGTAGCCGGAGGGTTATTTTCCAAGGGTTGAGTCGCG |
| | GGACCCCCGGTTCGAGTCTCGGACCGGCCGGACTGCGGCGAAC |
| | GGGGGTTTGCCTCCCCGTCATGCAAGACCCCGCTTGCAAATTCC |
| | TCCGGAAACAGGGACGAGCCCCTTTTTTGCTTTTCCCAGATGCA |
| | TCCGGTGCTGCGGCAGATGCGCCCCCCTCCTCAGCAGCGGCAAG |
| | AGCAAGAGCAGCGGCAGACATGCAGGGCACCCTCCCCTCCTCC |
| | TACCGCGTCAGGAGGGGCGACATCCGCGGTTGACGCGGCAGCA |
| | GATGGTGATTACGAACCCCCGCGGCGCCGGGCCCGGCACTACCT |
| | GGACTTGGAGGAGGGCGAGGGCCTGGCGCGGCTAGGAGCGCCC |
| | TCTCCTGAGCGGCACCCAAGGGTGCAGCTGAAGCGTGATACGC |
| | GTGAGGCGTACGTGCCGCGGCAGAACCTGTTTCGCGACCGCGA |
| | GGGAGAGGAGCCCGAGGAGATGCGGGATCGAAAGTTCCACGCA |
| | GGGCGCGAGCTGCGGCATGGCCTGAATCGCGAGCGGTTGCTGC |
| | GCGAGGAGGACTTTGAGCCCGACGCGCGAACCGGGATTAGTCC |
| | CGCGCGCGCACACGTGGCGGCCGCCGACCTGGTAACCGCATAC |
| | GAGCAGACGGTGAACCAGGAGATTAACTTTCAAAAAAGCTTTA |
| | ACAACCACGTGCGTACGCTTGTGGCGCGCGAGGAGGTGGCTAT |
| | AGGACTGATGCATCTGTGGGACTTTGTAAGCGCGCTGGAGCAA |
| | AACCCAAATAGCAAGCCGCTCATGGCGCAGCTGTTCCTTATAGT |
| | GCAGCACAGCAGGGACAACGAGGCATTCAGGGATGCGCTGCTA |
| | AACATAGTAGAGCCCGAGGGCCGCTGGCTGCTCGATTTGATAA |
| | ACATCCTGCAGAGCATAGTGGTGCAGGAGCGCAGCTTGAGCCT |
| | GGCTGACAAGGTGGCCGCCATCAACTATTCCATGCTTAGCCTGG |
| | GCAAGTTTTACGCCCGCAAGATATACCATACCCCTTACGTTCCC |
| | ATAGACAAGGAGGTAAAGATCGAGGGGTTCTACATGCGCATGG |
| | CGCTGAAGGTGCTTACCTTGAGCGACGACCTGGGCGTTTATCGC |
| | AACGAGCGCATCCACAAGGCCGTGAGCGTGAGCCGGCGGCGCG |
| | AGCTCAGCGACCGCGAGCTGATGCACAGCCTGCAAAGGGCCCT |
| | GGCTGGCACGGGCAGCGGCGATAGAGAGGCCGAGTCCTACTTT |
| | GACGCGGGCGCTGACCTGCGCTGGGCCCCAAGCCGACGCGCCC |
| | TGGAGGCAGCTGGGGCCGGACCTGGGCTGGCGGTGGCACCCGC |
| | GCGCGCTGGCAACGTCGGCGGCGTGGAGGAATATGACGAGGAC |
| | GATGAGTACGAGCCAGAGGACGGCGAGTACTAAGCGGTGATGT |
| | TTCTGATCAGATGATGCAAGACGCAACGGACCCGGCGGTGCGG |
| | GCGGCGCTGCAGAGCCAGCCGTCCGGCCTTAACTCCACGGACG |
| | ACTGGCGCCAGGTCATGGACCGCATCATGTCGCTGACTGCGCGC |
| | AATCCTGACGCGTTCCGGCAGCAGCCGCAGGCCAACCGGCTCTC |
| | CGCAATTCTGGAAGCGGTGGTCCCGGCGCGCGCAAACCCCACG |
| | CACGAGAAGGTGCTGGCGATCGTAAACGCGCTGGCCGAAAACA |
| | GGGCCATCCGGCCCGACGAGGCCGGCCTGGTCTACGACGCGCT |
| | GCTTCAGCGCGTGGCTCGTTACAACAGCGGCAACGTGCAGACC |
| | AACCTGGACCGGCTGGTGGGGGATGTGCGCGAGGCCGTGGCGC |
| | AGCGTGAGCGCGCGCAGCAGCAGGGCAACCTGGGCTCCATGGT |
| | TGCACTAAACGCCTTCCTGAGTACACAGCCCGCCAACGTGCCGC |
| | GGGGACAGGAGGACTACACCAACTTTGTGAGCGCACTGCGGCT |
| | AATGGTGACTGAGACACCGCAAAGTGAGGTGTACCAGTCTGGG |
| | CCAGACTATTTTTTCCAGACCAGTAGACAAGGCCTGCAGACCGT |
| | AAACCTGAGCCAGGCTTTCAAAAACTTGCAGGGGCTGTGGGGG |
| | GTGCGGGCTCCCACAGGCGACCGCGCGACCGTGTCTAGCTTGCT |
| | GACGCCCAACTCGCGCCTGTTGCTGCTGCTAATAGCGCCCTTCA |
| | CGGACAGTGGCAGCGTGTCCCGGGACACATACCTAGGTCACTTG |
| | CTGACACTGTACCGCGAGGCCATAGGTCAGGCGCATGTGGACG |
| | AGCATACTTTCCAGGAGATTACAAGTGTCAGCCGCGCGCTGGGG |
| | CAGGAGGACACGGGCAGCCTGGAGGCAACCCTAAACTACCTGC |
| | TGACCAACCGGCGGCAGAAGATCCCCTCGTTGCACAGTTTAAAC |
| | AGCGAGGAGGAGCGCATTTTGCGCTACGTGCAGCAGAGCGTGA |
| | GCCTTAACCTGATGCGCGACGGGGTAACGCCCAGCGTGGCGCT |
| | GGACATGACCGCGCGCAACATGGAACCGGGCATGTATGCCTCA |
| | AACCGGCCGTTTATCAACCGCCTAATGGACTACTTGCATCGCGC |
| | GGCCGCCGTGAACCCCGAGTATTTCACCAATGCCATCTTGAACC |
| | CGCACTGGCTACCGCCCCCTGGTTTCTACACCGGGGGATTCGAG |
| | GTGCCCGAGGGTAACGATGGATTCCTCTGGGACGACATAGACG |
| | ACAGCGTGTTTTCCCCGCAACCGCAGACCCTGCTAGAGTTGCAA |
| | CAGCGCGAGCAGGCAGAGGCGGCGCTGCGAAAGGAAAGCTTCC |
| | GCAGGCCAAGCAGCTTGTCCGATCTAGGCGCTGCGGCCCCGCG |
| | GTCAGATGCTAGTAGCCCATTTCCAAGCTTGATAGGGTCTCTTA |
| | CCAGCACTCGCACCACCCGCCCGCGCCTGCTGGGCGAGGAGGA |
| | GTACCTAAACAACTCGCTGCTGCAGCCGCAGCGCGAAAAAAAC |
| | CTGCCTCCGGCATTTCCCAACAACGGGATAGAGAGCCTAGTGGA |
| | CAAGATGAGTAGATGGAAGACGTACGCGCAGGAGCACAGGGAC |
| | GTGCCAGGCCCGCGCCCGCCCACCCGTCGTCAAAGGCACGACC |
| | GTCAGCGGGGTCTGGTGTGGGAGGACGATGACTCGGCAGACGA |
| | CAGCAGCGTCCTGGATTTGGGAGGGAGTGGCAACCCGTTTGCGC |
| | ACCTTCGCCCCAGGCTGGGGAGAATGTTTTAAAAAAAAAAAAG |
| | CATGATGCAAAATAAAAAACTCACCAAGGCCATGGCACCGAGC |
| | GTTGGTTTTCTTGTATTCCCCTTAGTATGCGGCGCGCGGCGATGT |
| | ATGAGGAAGGTCCTCCTCCCTCCTACGAGAGTGTGGTGAGCGCG |
| | GCGCCAGTGGCGGCGGCGCTGGGTTCTCCCTTCGATGCTCCCCT |
| | GGACCCGCCGTTTGTGCCTCCGCGGTACCTGCGGCCTACCGGGG |
| | GGAGAAACAGCATCCGTTACTCTGAGTTGGCACCCCTATTCGAC |
| | ACCACCCGTGTGTACCTGGTGGACAACAAGTCAACGGATGTGG |
| | CATCCCTGAACTACCAGAACGACCACAGCAACTTTCTGACCACG |
| | GTCATTCAAAACAATGACTACAGCCCGGGGGAGGCAAGCACAC |
| | AGACCATCAATCTTGACGACCGGTCGCACTGGGGCGGCGACCT |
| | GAAAACCATCCTGCATACCAACATGCCAAATGTGAACGAGTTC |
| | ATGTTTACCAATAAGTTTAAGGCGCGGGTGATGGTGTCGCGCTT |
| | GCCTACTAAGGACAATCAGGTGGAGCTGAAATACGAGTGGGTG |
| | GAGTTCACGCTGCCCGAGGGCAACTACTCCGAGACCATGACCAT |
| | AGACCTTATGAACAACGCGATCGTGGAGCACTACTTGAAAGTG |
| | GGCAGACAGAACGGGGTTCTGGAAAGCGACATCGGGGTAAAGT |
| | TTGACACCCGCAACTTCAGACTGGGGTTTGACCCCGTCACTGGT |
| | CTTGTCATGCCTGGGGTATATACAAACGAAGCCTTCCATCCAGA |
| | CATCATTTTGCTGCCAGGATGCGGGGTGGACTTCACCCACAGCC |
| | GCCTGAGCAACTTGTTGGGCATCCGCAAGCGGCAACCCTTCCAG |
| | GAGGGCTTTAGGATCACCTACGATGATCTGGAGGGTGGTAACAT |
| | TCCCGCACTGTTGGATGTGGACGCCTACCAGGCGAGCTTGAAAG |
| | ATGACACCGAACAGGGCGGGGGTGGCGCAGGCGGCAGCAACA |
| | GCAGTGGCAGCGGCGCGGAAGAGAACTCCAACGCGGCAGCCGC |
| | GGCAATGCAGCCGGTGGAGGACATGAACGATCATGCCATTCGC |
| | GGCGACACCTTTGCCACACGGGCTGAGGAGAAGCGCGCTGAGG |
| | CCGAAGCAGCGGCCGAAGCTGCCGCCCCCGCTGCGCAACCCGA |
| | GGTCGAGAAGCCTCAGAAGAAACCGGTGATCAAACCCCTGACA |
| | GAGGACAGCAAGAAACGCAGTTACAACCTAATAAGCAATGACA |
| | GCACCTTCACCCAGTACCGCAGCTGGTACCTTGCATACAACTAC |
| | GGCGACCCTCAGACCGGAATCCGCTCATGGACCCTGCTTTGCAC |
| | TCCTGACGTAACCTGCGGCTCGGAGCAGGTCTACTGGTCGTTGC |
| | CAGACATGATGCAAGACCCCGTGACCTTCCGCTCCACGCGCCAG |
| | ATCAGCAACTTTCCGGTGGTGGGCGCCGAGCTGTTGCCCGTGCA |
| | CTCCAAGAGCTTCTACAACGACCAGGCCGTCTACTCCCAACTCA |
| | TCCGCCAGTTTACCTCTCTGACCCACGTGTTCAATCGCTTTCCCG |
| | AGAACCAGATTTTGGCGCGCCCGCCAGCCCCCACCATCACCACC |
| | GTCAGTGAAAACGTTCCTGCTCTCACAGATCACGGGACGCTACC |
| | GCTGCGCAACAGCATCGGAGGAGTCCAGCGAGTGACCATTACT |
| | GACGCCAGACGCCGCACCTGCCCCTACGTTTACAAGGCCCTGGG |
| | CATAGTCTCGCCGCGCGTCCTATCGAGCCGCACTTTTTGAGCAA |
| | GCATGTCCATCCTTATATCGCCCAGCAATAACACAGGCTGGGGC |
| | CTGCGCTTCCCAAGCAAGATGTTTGGCGGGGCCAAGAAGCGCTC |
| | CGACCAACACCCAGTGCGCGTGCGCGGGCACTACCGCGCGCCC |
| | TGGGGCGCGCACAAACGCGGCCGCACTGGGCGCACCACCGTCG |
| | ATGACGCCATCGACGCGGTGGTGGAGGAGGCGCGCAACTACAC |
| | GCCCACGCCGCCACCAGTGTCCACAGTGGACGCGGCCATTCAG |
| | ACCGTGGTGCGCGGAGCCCGGCGCTATGCTAAAATGAAGAGAC |
| | GGCGGAGGCGCGTAGCACGTCGCCACCGCCGCCGACCCGGCAC |
| | TGCCGCCCAACGCGCGGCGGCGGCCCTGCTTAACCGCGCACGTC |
| | GCACCGGCCGACGGGCGGCCATGCGGGCCGCTCGAAGGCTGGC |
| | CGCGGGTATTGTCACTGTGCCCCCCAGGTCCAGGCGACGAGCGG |
| | CCGCCGCAGCAGCCGCGGCCATTAGTGCTATGACTCAGGGTCGC |
| | AGGGGCAACGTGTATTGGGTGCGCGACTCGGTTAGCGGCCTGC |
| | GCGTGCCCGTGCGCACCCGCCCCCCGCGCAACTAGATTGCAAGA |
| | AAAAACTACTTAGACTCGTACTGTTGTATGTATCCAGCGGCGGC |
| | GGCGCGCAACGAAGCTATGTCCAAGCGCAAAATCAAAGAAGAG |
| | ATGCTCCAGGTCATCGCGCCGGAGATCTATGGCCCCCCGAAGAA |
| | GGAAGAGCAGGATTACAAGCCCCGAAAGCTAAAGCGGGTCAAA |
| | AAGAAAAAGAAAGATGATGATGATGAACTTGACGACGAGGTGG |
| | AACTGCTGCACGCTACCGCGCCCAGGCGACGGGTACAGTGGAA |
| | AGGTCGACGCGTAAAACGTGTTTTGCGACCCGGCACCACCGTAG |
| | TCTTTACGCCCGGTGAGCGCTCCACCCGCACCTACAAGCGCGTG |
| | TATGATGAGGTGTACGGCGACGAGGACCTGCTTGAGCAGGCCA |
| | ACGAGCGCCTCGGGGAGTTTGCCTACGGAAAGCGGCATAAGGA |
| | CATGCTGGCGTTGCCGCTGGACGAGGGCAACCCAACACCTAGC |
| | CTAAAGCCCGTAACACTGCAGCAGGTGCTGCCCGCGCTTGCACC |
| | GTCCGAAGAAAAGCGCGGCCTAAAGCGCGAGTCTGGTGACTTG |
| | GCACCCACCGTGCAGCTGATGGTACCCAAGCGCCAGCGACTGG |
| | AAGATGTCTTGGAAAAAATGACCGTGGAACCTGGGCTGGAGCC |
| | CGAGGTCCGCGTGCGGCCAATCAAGCAGGTGGCGCCGGGACTG |
| | GGCGTGCAGACCGTGGACGTTCAGATACCCACTACCAGTAGCA |
| | CCAGTATTGCCACCGCCACAGAGGGCATGGAGACACAAACGTC |
| | CCCGGTTGCCTCAGCGGTGGCGGATGCCGCGGTGCAGGCGGTC |
| | GCTGCGGCCGCGTCCAAGACCTCTACGGAGGTGCAAACGGACC |
| | CGTGGATGTTTCGCGTTTCAGCCCCCCGGCGCCCGCGCCGTTCG |
| | AGGAAGTACGGCGCCGCCAGCGCGCTACTGCCCGAATATGCCC |
| | TACATCCTTCCATTGCGCCTACCCCCGGCTATCGTGGCTACACCT |
| | ACCGCCCCAGAAGACGAGCAACTACCCGACGCCGAACCACCAC |
| | TGGAACCCGCCGCCGCCGTCGCCGTCGCCAGCCCGTGCTGGCCC |
| | CGATTTCCGTGCGCAGGGTGGCTCGCGAAGGAGGCAGGACCCT |
| | GGTGCTGCCAACAGCGCGCTACCACCCCAGCATCGTTTAAAAGC |
| | CGGTCTTTGTGGTTCTTGCAGATATGGCCCTCACCTGCCGCCTCC |
| | GTTTCCCGGTGCCGGGATTCCGAGGAAGAATGCACCGTAGGAG |
| | GGGCATGGCCGGCCACGGCCTGACGGGCGGCATGCGTCGTGCG |
| | CACCACCGGCGGCGGCGCGCGTCGCACCGTCGCATGCGCGGCG |
| | GTATCCTGCCCCTCCTTATTCCACTGATCGCCGCGGCGATTGGC |
| | GCCGTGCCCGGAATTGCATCCGTGGCCTTGCAGGCGCAGAGAC |
| | ACTGATTAAAAACAAGTTGCATGTGGAAAAATCAAAATAAAAA |
| | GTCTGGACTCTCACGCTCGCTTGGTCCTGTAACTATTTTGTAGAA |
| | TGGAAGACATCAACTTTGCGTCTCTGGCCCCGCGACACGGCTCG |
| | CGCCCGTTCATGGGAAACTGGCAAGATATCGGCACCAGCAATA |
| | TGAGCGGTGGCGCCTTCAGCTGGGGCTCGCTGTGGAGCGGCATT |
| | AAAAATTTCGGTTCCACCGTTAAGAACTATGGCAGCAAGGCCTG |
| | GAACAGCAGCACAGGCCAGATGCTGAGGGATAAGTTGAAAGAG |
| | CAAAATTTCCAACAAAAGGTGGTAGATGGCCTGGCCTCTGGCAT |
| | TAGCGGGGTGGTGGACCTGGCCAACCAGGCAGTGCAAAATAAG |
| | ATTAACAGTAAGCTTGATCCCCGCCCTCCCGTAGAGGAGCCTCC |
| | ACCGGCCGTGGAGACAGTGTCTCCAGAGGGGCGTGGCGAAAAG |
| | CGTCCGCGCCCCGACAGGGAAGAAACTCTGGTGACGCAAATAG |
| | ACGAGCCTCCCTCGTACGAGGAGGCACTAAAGCAAGGCCTGCC |
| | CACCACCCGTCCCATCGCGCCCATGGCTACCGGAGTGCTGGGCC |
| | AGCACACACCCGTAACGCTGGACCTGCCTCCCCCCGCCGACACC |
| | CAGCAGAAACCTGTGCTGCCAGGCCCGACCGCCGTTGTTGTAAC |
| | CCGTCCTAGCCGCGCGTCCCTGCGCCGCGCCGCCAGCGGTCCGC |
| | GATCGTTGCGGCCCGTAGCCAGTGGCAACTGGCAAAGCACACT |
| | GAACAGCATCGTGGGTCTGGGGGTGCAATCCCTGAAGCGCCGA |
| | CGATGCTTCTGATAGCTAACGTGTCGTATGTGTGTCATGTATGC |
| | GTCCATGTCGCCGCCAGAGGAGCTGCTGAGCCGCCGCGCGCCC |
| | GCTTTCCAAGATGGCTACCCCTTCGATGATGCCGCAGTGGTCTT |
| | ACATGCACATCTCGGGCCAGGACGCCTCGGAGTACCTGAGCCCC |
| | GGGCTGGTGCAGTTTGCCCGCGCCACCGAGACGTACTTCAGCCT |
| | GAATAACAAGTTTAGAAACCCCACGGTGGCGCCTACGCACGAC |
| | GTGACCACAGACCGGTCCCAGCGTTTGACGCTGCGGTTCATCCC |
| | TGTGGACCGTGAGGATACTGCGTACTCGTACAAGGCGCGGTTCA |
| | CCCTAGCTGTGGGTGATAACCGTGTGCTGGACATGGCTTCCACG |
| | TACTTTGACATCCGCGGCGTGCTGGACAGGGGCCCTACTTTTAA |
| | GCCCTACTCTGGCACTGCCTACAACGCCCTGGCTCCCAAGGGTG |
| | CCCCAAATCCTTGCGAATGGGATGAAGCTGCTACTGCTCTTGAA |
| | ATAAACCTAGAAGAAGAGGACGATGACAACGAAGACGAAGTA |
| | GACGAGCAAGCTGAGCAGCAAAAAACTCACGTATTTGGGCAGG |
| | CGCCTTATTCTGGTATAAATATTACAAAGGAGGGTATTCAAATA |
| | GGTGTCGAAGGTCAAACACCTAAATATGCCGATAAAACATTTCA |
| | ACCTGAACCTCAAATAGGAGAATCTCAGTGGTACGAAACAGAA |
| | ATTAATCATGCAGCTGGGAGAGTCCTAAAAAAGACTACCCCAA |
| | TGAAACCATGTTACGGTTCATATGCAAAACCCACAAATGAAAAT |
| | GGAGGGCAAGGCATTCTTGTAAAGCAACAAAATGGAAAGCTAG |
| | AAAGTCAAGTGGAAATGCAATTTTTCTCAACTACTGAGGCAGCC |
| | GCAGGCAATGGTGATAACTTGACTCCTAAAGTGGTATTGTACAG |
| | TGAAGATGTAGATATAGAAACCCCAGACACTCATATTTCTTACA |
| | TGCCCACTATTAAGGAAGGTAACTCACGAGAACTAATGGGCCA |
| | ACAATCTATGCCCAACAGGCCTAATTACATTGCTTTTAGGGACA |
| | ATTTTATTGGTCTAATGTATTACAACAGCACGGGTAATATGGGT |
| | GTTCTGGCGGGCCAAGCATCGCAGTTGAATGCTGTTGTAGATTT |
| | GCAAGACAGAAACACAGAGCTTTCATACCAGCTTTTGCTTGATT |
| | CCATTGGTGATAGAACCAGGTACTTTTCTATGTGGAATCAGGCT |
| | GTTGACAGCTATGATCCAGATGTTAGAATTATTGAAAATCATGG |
| | AACTGAAGATGAACTTCCAAATTACTGCTTTCCACTGGGAGGTG |
| | TGATTAATACAGAGACTCTTACCAAGGTAAAACCTAAAACAGG |
| | TCAGGAAAATGGATGGGAAAAAGATGCTACAGAATTTTCAGAT |
| | AAAAATGAAATAAGAGTTGGAAATAATTTTGCCATGGAAATCA |
| | ATCTAAATGCCAACCTGTGGAGAAATTTCCTGTACTCCAACATA |
| | GCGCTGTATTTGCCCGACAAGCTAAAGTACAGTCCTTCCAACGT |
| | AAAAATTTCTGATAACCCAAACACCTACGACTACATGAACAAG |
| | CGAGTGGTGGCTCCCGGGCTAGTGGACTGCTACATTAACCTTGG |
| | AGCACGCTGGTCCCTTGACTATATGGACAACGTCAACCCATTTA |
| | ACCACCACCGCAATGCTGGCCTGCGCTACCGCTCAATGTTGCTG |
| | GGCAATGGTCGCTATGTGCCCTTCCACATCCAGGTGCCTCAGAA |
| | GTTCTTTGCCATTAAAAACCTCCTTCTCCTGCCGGGCTCATACAC |
| | CTACGAGTGGAACTTCAGGAAGGATGTTAACATGGTTCTGCAGA |
| | GCTCCCTAGGAAATGACCTAAGGGTTGACGGAGCCAGCATTAA |
| | GTTTGATAGCATTTGCCTTTACGCCACCTTCTTCCCCATGGCCCA |
| | CAACACCGCCTCCACGCTTGAGGCCATGCTTAGAAACGACACCA |
| | ACGACCAGTCCTTTAACGACTATCTCTCCGCCGCCAACATGCTC |
| | TACCCTATACCCGCCAACGCTACCAACGTGCCCATATCCATCCC |
| | CTCCCGCAACTGGGCGGCTTTCCGCGGCTGGGCCTTCACGCGCC |
| | TTAAGACTAAGGAAACCCCATCACTGGGCTCGGGCTACGACCCT |
| | TATTACACCTACTCTGGCTCTATACCCTACCTAGATGGAACCTTT |
| | TACCTCAACCACACCTTTAAGAAGGTGGCCATTACCTTTGACTC |
| | TTCTGTCAGCTGGCCTGGCAATGACCGCCTGCTTACCCCCAACG |
| | AGTTTGAAATTAAGCGCTCAGTTGACGGGGAGGGTTACAACGTT |
| | GCCCAGTGTAACATGACCAAAGACTGGTTCCTGGTACAAATGCT |
| | AGCTAACTATAACATTGGCTACCAGGGCTTCTATATCCCAGAGA |
| | GCTACAAGGACCGCATGTACTCCTTCTTTAGAAACTTCCAGCCC |
| | ATGAGCCGTCAGGTGGTGGATGATACTAAATACAAGGACTACC |
| | AACAGGTGGGCATCCTACACCAACACAACAACTCTGGATTTGTT |
| | GGCTACCTTGCCCCCACCATGCGCGAAGGACAGGCCTACCCTGC |
| | TAACTTCCCCTATCCGCTTATAGGCAAGACCGCAGTTGACAGCA |
| | TTACCCAGAAAAAGTTTCTTTGCGATCGCACCCTTTGGCGCATC |
| | CCATTCTCCAGTAACTTTATGTCCATGGGCGCACTCACAGACCT |
| | GGGCCAAAACCTTCTCTACGCCAACTCCGCCCACGCGCTAGACA |
| | TGACTTTTGAGGTGGATCCCATGGACGAGCCCACCCTTCTTTAT |
| | GTTTTGTTTGAAGTCTTTGACGTGGTCCGTGTGCACCAGCCGCA |
| | CCGCGGCGTCATCGAAACCGTGTACCTGCGCACGCCCTTCTCGG |
| | CCGGCAACGCCACAACATAAAGAAGCAAGCAACATCAACAACA |
| | GCTGCCGCCATGGGCTCCAGTGAGCAGGAACTGAAAGCCATTG |
| | TCAAAGATCTTGGTTGTGGGCCATATTTTTTGGGCACCTATGAC |
| | AAGCGCTTTCCAGGCTTTGTTTCTCCACACAAGCTCGCCTGCGC |
| | CATAGTCAATACGGCCGGTCGCGAGACTGGGGGCGTACACTGG |
| | ATGGCCTTTGCCTGGAACCCGCACTCAAAAACATGCTACCTCTT |
| | TGAGCCCTTTGGCTTTTCTGACCAGCGACTCAAGCAGGTTTACC |
| | AGTTTGAGTACGAGTCACTCCTGCGCCGTAGCGCCATTGCTTCT |
| | TCCCCCGACCGCTGTATAACGCTGGAAAAGTCCACCCAAAGCGT |
| | ACAGGGGCCCAACTCGGCCGCCTGTGGACTATTCTGCTGCATGT |
| | TTCTCCACGCCTTTGCCAACTGGCCCCAAACTCCCATGGATCAC |
| | AACCCCACCATGAACCTTATTACCGGGGTACCCAACTCCATGCT |
| | CAACAGTCCCCAGGTACAGCCCACCCTGCGTCGCAACCAGGAA |
| | CAGCTCTACAGCTTCCTGGAGCGCCACTCGCCCTACTTCCGCAG |
| | CCACAGTGCGCAGATTAGGAGCGCCACTTCTTTTTGTCACTTGA |
| | AAAACATGTAAAAATAATGTACTAGAGACACTTTCAATAAAGG |
| | CAAATGCTTTTATTTGTACACTCTCGGGTGATTATTTACCCCCAC |
| | CCTTGCCGTCTGCGCCGTTTAAAAATCAAAGGGGTTCTGCCGCG |
| | CATCGCTATGCGCCACTGGCAGGGACACGTTGCGATACTGGTGT |
| | TTAGTGCTCCACTTAAACTCAGGCACAACCATCCGCGGCAGCTC |
| | GGTGAAGTTTTCACTCCACAGGCTGCGCACCATCACCAACGCGT |
| | TTAGCAGGTCGGGCGCCGATATCTTGAAGTCGCAGTTGGGGCCT |
| | CCGCCCTGCGCGCGCGAGTTGCGATACACAGGGTTGCAGCACTG |
| | GAACACTATCAGCGCCGGGTGGTGCACGCTGGCCAGCACGCTCT |
| | TGTCGGAGATCAGATCCGCGTCCAGGTCCTCCGCGTTGCTCAGG |
| | GCGAACGGAGTCAACTTTGGTAGCTGCCTTCCCAAAAAGGGCG |
| | CGTGCCCAGGCTTTGAGTTGCACTCGCACCGTAGTGGCATCAAA |
| | AGGTGACCGTGCCCGGTCTGGGCGTTAGGATACAGCGCCTGCAT |
| | AAAAGCCTTGATCTGCTTAAAAGCCACCTGAGCCTTTGCGCCTT |
| | CAGAGAAGAACATGCCGCAAGACTTGCCGGAAAACTGATTGGC |
| | CGGACAGGCCGCGTCGTGCACGCAGCACCTTGCGTCGGTGTTGG |
| | AGATCTGCACCACATTTCGGCCCCACCGGTTCTTCACGATCTTG |
| | GCCTTGCTAGACTGCTCCTTCAGCGCGCGCTGCCCGTTTTCGCTC |
| | GTCACATCCATTTCAATCACGTGCTCCTTATTTATCATAATGCTT |
| | CCGTGTAGACACTTAAGCTCGCCTTCGATCTCAGCGCAGCGGTG |
| | CAGCCACAACGCGCAGCCCGTGGGCTCGTGATGCTTGTAGGTCA |
| | CCTCTGCAAACGACTGCAGGTACGCCTGCAGGAATCGCCCCATC |
| | ATCGTCACAAAGGTCTTGTTGCTGGTGAAGGTCAGCTGCAACCC |
| | GCGGTGCTCCTCGTTCAGCCAGGTCTTGCATACGGCCGCCAGAG |
| | CTTCCACTTGGTCAGGCAGTAGTTTGAAGTTCGCCTTTAGATCGT |
| | TATCCACGTGGTACTTGTCCATCAGCGCGCGCGCAGCCTCCATG |
| | CCCTTCTCCCACGCAGACACGATCGGCACACTCAGCGGGTTCAT |
| | CACCGTAATTTCACTTTCCGCTTCGCTGGGCTCTTCCTCTTCCTC |
| | TTGCGTCCGCATACCACGCGCCACTGGGTCGTCTTCATTCAGCC |
| | GCCGCACTGTGCGCTTACCTCCTTTGCCATGCTTGATTAGCACCG |
| | GTGGGTTGCTGAAACCCACCATTTGTAGCGCCACATCTTCTCTTT |
| | CTTCCTCGCTGTCCACGATTACCTCTGGTGATGGCGGGCGCTCG |
| | GGCTTGGGAGAAGGGCGCTTCTTTTTCTTCTTGGGCGCAATGGC |
| | CAAATCCGCCGCCGAGGTCGATGGCCGCGGGCTGGGTGTGCGC |
| | GGCACCAGCGCGTCTTGTGATGAGTCTTCCTCGTCCTCGGACTC |
| | GATACGCCGCCTCATCCGCTTTTTTGGGGGCGCCCGGGGAGGCG |
| | GCGGCGACGGGGACGGGGACGACACGTCCTCCATGGTTGGGGG |
| | ACGTCGCGCCGCACCGCGTCCGCGCTCGGGGGTGGTTTCGCGCT |
| | GCTCCTCTTCCCGACTGGCCATTTCCTTCTCCTATAGGCAGAAAA |
| | AGATCATGGAGTCAGTCGAGAAGAAGGACAGCCTAACCGCCCC |
| | CTCTGAGTTCGCCACCACCGCCTCCACCGATGCCGCCAACGCGC |
| | CTACCACCTTCCCCGTCGAGGCACCCCCGCTTGAGGAGGAGGAA |
| | GTGATTATCGAGCAGGACCCAGGTTTTGTAAGCGAAGACGACG |
| | AGGACCGCTCAGTACCAACAGAGGATAAAAAGCAAGACCAGGA |
| | CAACGCAGAGGCAAACGAGGAACAAGTCGGGCGGGGGGACGA |
| | AAGGCATGGCGACTACCTAGATGTGGGAGACGACGTGCTGTTG |
| | AAGCATCTGCAGCGCCAGTGCGCCATTATCTGCGACGCGTTGCA |
| | AGAGCGCAGCGATGTGCCCCTCGCCATAGCGGATGTCAGCCTTG |
| | CCTACGAACGCCACCTATTCTCACCGCGCGTACCCCCCAAACGC |
| | CAAGAAAACGGCACATGCGAGCCCAACCCGCGCCTCAACTTCT |
| | ACCCCGTATTTGCCGTGCCAGAGGTGCTTGCCACCTATCACATC |
| | TTTTTCCAAAACTGCAAGATACCCCTATCCTGCCGTGCCAACCG |
| | CAGCCGAGCGGACAAGCAGCTGGCCTTGCGGCAGGGCGCTGTC |
| | ATACCTGATATCGCCTCGCTCAACGAAGTGCCAAAAATCTTTGA |
| | GGGTCTTGGACGCGACGAGAAGCGCGCGGCAAACGCTCTGCAA |
| | CAGGAAAACAGCGAAAATGAAAGTCACTCTGGAGTGTTGGTGG |
| | AACTCGAGGGTGACAACGCGCGCCTAGCCGTACTAAAACGCAG |
| | CATCGAGGTCACCCACTTTGCCTACCCGGCACTTAACCTACCCC |
| | CCAAGGTCATGAGCACAGTCATGAGTGAGCTGATCGTGCGCCGT |
| | GCGCAGCCCCTGGAGAGGGATGCAAATTTGCAAGAACAAACAG |
| | AGGAGGGCCTACCCGCAGTTGGCGACGAGCAGCTAGCGCGCTG |
| | GCTTCAAACGCGCGAGCCTGCCGACTTGGAGGAGCGACGCAAA |
| | CTAATGATGGCCGCAGTGCTCGTTACCGTGGAGCTTGAGTGCAT |
| | GCAGCGGTTCTTTGCTGACCCGGAGATGCAGCGCAAGCTAGAG |
| | GAAACATTGCACTACACCTTTCGACAGGGCTACGTACGCCAGGC |
| | CTGCAAGATCTCCAACGTGGAGCTCTGCAACCTGGTCTCCTACC |
| | TTGGAATTTTGCACGAAAACCGCCTTGGGCAAAACGTGCTTCAT |
| | TCCACGCTCAAGGGCGAGGCGCGCCGCGACTACGTCCGCGACT |
| | GCGTTTACTTATTTCTATGCTACACCTGGCAGACGGCCATGGGC |
| | GTTTGGCAGCAGTGCTTGGAGGAGTGCAACCTCAAGGAGCTGC |
| | AGAAACTGCTAAAGCAAAACTTGAAGGACCTATGGACGGCCTT |
| | CAACGAGCGCTCCGTGGCCGCGCACCTGGCGGACATCATTTTCC |
| | CCGAACGCCTGCTTAAAACCCTGCAACAGGGTCTGCCAGACTTC |
| | ACCAGTCAAAGCATGTTGCAGAACTTTAGGAACTTTATCCTAGA |
| | GCGCTCAGGAATCTTGCCCGCCACCTGCTGTGCACTTCCTAGCG |
| | ACTTTGTGCCCATTAAGTACCGCGAATGCCCTCCGCCGCTTTGG |
| | GGCCACTGCTACCTTCTGCAGCTAGCCAACTACCTTGCCTACCA |
| | CTCTGACATAATGGAAGACGTGAGCGGTGACGGTCTACTGGAG |
| | TGTCACTGTCGCTGCAACCTATGCACCCCGCACCGCTCCCTGGT |
| | TTGCAATTCGCAGCTGCTTAACGAAAGTCAAATTATCGGTACCT |
| | TTGAGCTGCAGGGTCCCTCGCCTGACGAAAAGTCCGCGGCTCCG |
| | GGGTTGAAACTCACTCCGGGGCTGTGGACGTCGGCTTACCTTCG |
| | CAAATTTGTACCTGAGGACTACCACGCCCACGAGATTAGGTTCT |
| | ACGAAGACCAATCCCGCCCGCCTAATGCGGAGCTTACCGCCTGC |
| | GTCATTACCCAGGGCCACATTCTTGGCCAATTGCAAGCCATCAA |
| | CAAAGCCCGCCAAGAGTTTCTGCTACGAAAGGGACGGGGGGTT |
| | TACTTGGACCCCCAGTCCGGCGAGGAGCTCAACCCAATCCCCCC |
| | GCCGCCGCAGCCCTATCAGCAGCAGCCGCGGGCCCTTGCTTCCC |
| | AGGATGGCACCCAAAAAGAAGCTGCAGCTGCCGCCGCCACCCA |
| | CGGACGAGGAGGAATACTGGGACAGTCAGGCAGAGGAGGTTTT |
| | GGACGAGGAGGAGGAGGACATGATGGAAGACTGGGAGAGCCT |
| | AGACGAGGAAGCTTCCGAGGTCGAAGAGGTGTCAGACGAAACA |
| | CCGTCACCCTCGGTCGCATTCCCCTCGCCGGCGCCCCAGAAATC |
| | GGCAACCGGTTCCAGCATGGCTACAACCTCCGCTCCTCAGGCGC |
| | CGCCGGCACTGCCCGTTCGCCGACCCAACCGTAGATGGGACACC |
| | ACTGGAACCAGGGCCGGTAAGTCCAAGCAGCCGCCGCCGTTAG |
| | CCCAAGAGCAACAACAGCGCCAAGGCTACCGCTCATGGCGCGG |
| | GCACAAGAACGCCATAGTTGCTTGCTTGCAAGACTGTGGGGGC |
| | AACATCTCCTTCGCCCGCCGCTTTCTTCTCTACCATCACGGCGTG |
| | GCCTTCCCCCGTAACATCCTGCATTACTACCGTCATCTCTACAGC |
| | CCATACTGCACCGGCGGCAGCGGCAGCAACAGCAGCGGCCACA |
| | CAGAAGCAAAGGCGACCGGATAGCAAGACTCTGACAAAGCCCA |
| | AGAAATCCACAGCGGCGGCAGCAGCAGGAGGAGGAGCGCTGC |
| | GTCTGGCGCCCAACGAACCCGTATCGACCCGCGAGCTTAGAAA |
| | CAGGATTTTTCCCACTCTGTATGCTATATTTCAACAGAGCAGGG |
| | GCCAAGAACAAGAGCTGAAAATAAAAAACAGGTCTCTGCGATC |
| | CCTCACCCGCAGCTGCCTGTATCACAAAAGCGAAGATCAGCTTC |
| | GGCGCACGCTGGAAGACGCGGAGGCTCTCTTCAGTAAATACTG |
| | CGCGCTGACTCTTAAGGACTAGTTTCGCGCCCTTTCTCAAATTTA |
| | AGCGCGAAAACTACGTCATCTCCAGCGGCCACACCCGGCGCCA |
| | GCACCTGTTGTCAGCGCCATTATGAGCAAGGAAATTCCCACGCC |
| | CTACATGTGGAGTTACCAGCCACAAATGGGACTTGCGGCTGGA |
| | GCTGCCCAAGACTACTCAACCCGAATAAACTACATGAGCGCGG |
| | GACCCCACATGATATCCCGGGTCAACGGAATACGCGCCCACCG |
| | AAACCGAATTCTCCTGGAACAGGCGGCTATTACCACCACACCTC |
| | GTAATAACCTTAATCCCCGTAGTTGGCCCGCTGCCCTGGTGTAC |
| | CAGGAAAGTCCCGCTCCCACCACTGTGGTACTTCCCAGAGACGC |
| | CCAGGCCGAAGTTCAGATGACTAACTCAGGGGCGCAGCTTGCG |
| | GGCGGCTTTCGTCACAGGGTGCGGTCGCCCGGGCAGGGTATAA |
| | CTCACCTGACAATCAGAGGGCGAGGTATTCAGCTCAACGACGA |
| | GTCGGTGAGCTCCTCGCTTGGTCTCCGTCCGGACGGGACATTTC |
| | AGATCGGCGGCGCCGGCCGCTCTTCATTCACGCCTCGTCAGGCA |
| | ATCCTAACTCTGCAGACCTCGTCCTCTGAGCCGCGCTCTGGAGG |
| | CATTGGAACTCTGCAATTTATTGAGGAGTTTGTGCCATCGGTCT |
| | ACTTTAACCCCTTCTCGGGACCTCCCGGCCACTATCCGGATCAA |
| | TTTATTCCTAACTTTGACGCGGTAAAGGACTCGGCGGACGGCTA |
| | CGACTGAATGTTAAGTGGAGAGGCAGAGCAACTGCGCCTGAAA |
| | CACCTGGTCCACTGTCGCCGCCACAAGTGCTTTGCCCGCGACTC |
| | CGGTGAGTTTTGCTACTTTGAATTGCCCGAGGATCATATCGAGG |
| | GCCCGGCGCACGGCGTCCGGCTTACCGCCCAGGGAGAGCTTGC |
| | CCGTAGCCTGATTCGGGAGTTTACCCAGCGCCCCCTGCTAGTTG |
| | AGCGGGACAGGGGACCCTGTGTTCTCACTGTGATTTGCAACTGT |
| | CCTAACCCTGGATTACATCAAGATCCTCTAGTTAATGTCAGGTC |
| | GCCTAAGTCGATTAACTAGAGTACCCGGGGATCTTATTCCCTTT |
| | AACTAATAAAAAAAAATAATAAAGCATCACTTACTTAAAATCA |
| | GTTAGCAAATTTCTGTCCAGTTTATTCAGCAGCACCTCCTTGCCC |
| | TCCTCCCAGCTCTGGTATTGCAGCTTCCTCCTGGCTGCAAACTTT |
| | CTCCACAATCTAAATGGAATGTCAGTTTCCTCCTGTTCCTGTCCA |
| | TCCGCACCCACTATCTTCATGTTGTTGCAGATGAAGCGCGCAAG |
| | ACCGTCTGAAGATACCTTCAACCCCGTGTATCCATATGACACGG |
| | AAACCGGTCCTCCAACTGTGCCTTTTCTTACTCCTCCCTTTGTAT |
| | CCCCCAATGGGTTTCAAGAGAGTCCCCCTGGGGTACTCTCTTTG |
| | CGCCTATCCGAACCTCTAGTTACCTCCAATGGCATGCTTGCGCT |
| | CAAAATGGGCAACGGCCTCTCTCTGGACGAGGCCGGCAACCTT |
| | ACCTCCCAAAATGTAACCACTGTGAGCCCACCTCTCAAAAAAAC |
| | CAAGTCAAACATAAACCTGGAAATATCTGCACCCCTCACAGTTA |
| | CCTCAGAAGCCCTAACTGTGGCTGCCGCCGCACCTCTAATGGTC |
| | GCGGGCAACACACTCACCATGCAATCACAGGCCCCGCTAACCG |
| | TGCACGACTCCAAACTTAGCATTGCCACCCAAGGACCCCTCACA |
| | GTGTCAGAAGGAAAGCTAGCCCTGCAAACATCAGGCCCCCTCA |
| | CCACCACCGATAGCAGTACCCTTACTATCACTGCCTCACCCCCT |
| | CTAACTACTGCCACTGGTAGCTTGGGCATTGACTTGAAAGAGCC |
| | CATTTATACACAAAATGGAAAACTAGGACTAAAGTACGGGGCT |
| | CCTTTGCATGTAACAGACGACCTAAACACTTTGACCGTAGCAAC |
| | TGGTCCAGGTGTGACTATTAATAATACTTCCTTGCAAACTAAAG |
| | TTACTGGAGCCTTGGGTTTTGATTCACAAGGCAATATGCAACTT |
| | AATGTAGCAGGAGGACTAAGGATTGATTCTCAAAACAGACGCC |
| | TTATACTTGATGTTAGTTATCCGTTTGATGCTCAAAACCAACTAA |
| | ATCTAAGACTAGGACAGGGCCCTCTTTTTATAAACTCAGCCCAC |
| | AACTTGGATATTAACTACAACAAAGGCCTTTACTTGTTTACAGC |
| | TTCAAACAATTCCAAAAAGCTTGAGGTTAACCTAAGCACTGCCA |
| | AGGGGTTGATGTTTGACGCTACAGCCATAGCCATTAATGCAGGA |
| | GATGGGCTTGAATTTGGTTCACCTAATGCACCAAACACAAATCC |
| | CCTCAAAACAAAAATTGGCCATGGCCTAGAATTTGATTCAAACA |
| | AGGCTATGGTTCCTAAACTAGGAACTGGCCTTAGTTTTGACAGC |
| | ACAGGTGCCATTACAGTAGGAAACAAAAATAATGATAAGCTAA |
| | CTTTGTGGACCACACCAGCTCCATCTCCTAACTGTAGACTAAAT |
| | GCAGAGAAAGATGCTAAACTCACTTTGGTCTTAACAAAATGTGG |
| | CAGTCAAATACTTGCTACAGTTTCAGTTTTGGCTGTTAAAGGCA |
| | GTTTGGCTCCAATATCTGGAACAGTTCAAAGTGCTCATCTTATT |
| | ATAAGATTTGACGAAAATGGAGTGCTACTAAACAATTCCTTCCT |
| | GGACCCAGAATATTGGAACTTTAGAAATGGAGATCTTACTGAA |
| | GGCACAGCCTATACAAACGCTGTTGGATTTATGCCTAACCTATC |
| | AGCTTATCCAAAATCTCACGGTAAAACTGCCAAAAGTAACATTG |
| | TCAGTCAAGTTTACTTAAACGGAGACAAAACTAAACCTGTAACA |
| | CTAACCATTACACTAAACGGTACACAGGAAACAGGAGACACAA |
| | CTCCAAGTGCATACTCTATGTCATTTTCATGGGACTGGTCTGGCC |
| | ACAACTACATTAATGAAATATTTGCCACATCCTCTTACACTTTTT |
| | CATACATTGCCCAAGAATAAAGAATCGTTTGTGTTATGTTTCAA |
| | CGTGTTTATTTTTCAATTGCAGAAAATTTCAAGTCATTTTTCATT |
| | CAGTAGTATAGCCCCACCACCACATAGCTTATACAGATCACCGT |
| | ACCTTAATCAAACTCACAGAACCCTAGTATTCAACCTGCCACCT |
| | CCCTCCCAACACACAGAGTACACAGTCCTTTCTCCCCGGCTGGC |
| | CTTAAAAAGCATCATATCATGGGTAACAGACATATTCTTAGGTG |
| | TTATATTCCACACGGTTTCCTGTCGAGCCAAACGCTCATCAGTG |
| | ATATTAATAAACTCCCCGGGCAGCTCACTTAAGTTCATGTCGCT |
| | GTCCAGCTGCTGAGCCACAGGCTGCTGTCCAACTTGCGGTTGCT |
| | TAACGGGCGGCGAAGGAGAAGTCCACGCCTACATGGGGGTAGA |
| | GTCATAATCGTGCATCAGGATAGGGCGGTGGTGCTGCAGCAGC |
| | GCGCGAATAAACTGCTGCCGCCGCCGCTCCGTCCTGCAGGAATA |
| | CAACATGGCAGTGGTCTCCTCAGCGATGATTCGCACCGCCCGCA |
| | GCATAAGGCGCCTTGTCCTCCGGGCACAGCAGCGCACCCTGATC |
| | TCACTTAAATCAGCACAGTAACTGCAGCACAGCACCACAATATT |
| | GTTCAAAATCCCACAGTGCAAGGCGCTGTATCCAAAGCTCATGG |
| | CGGGGACCACAGAACCCACGTGGCCATCATACCACAAGCGCAG |
| | GTAGATTAAGTGGCGACCCCTCATAAACACGCTGGACATAAAC |
| | ATTACCTCTTTTGGCATGTTGTAATTCACCACCTCCCGGTACCAT |
| | ATAAACCTCTGATTAAACATGGCGCCATCCACCACCATCCTAAA |
| | CCAGCTGGCCAAAACCTGCCCGCCGGCTATACACTGCAGGGAA |
| | CCGGGACTGGAACAATGACAGTGGAGAGCCCAGGACTCGTAAC |
| | CATGGATCATCATGCTCGTCATGATATCAATGTTGGCACAACAC |
| | AGGCACACGTGCATACACTTCCTCAGGATTACAAGCTCCTCCCG |
| | CGTTAGAACCATATCCCAGGGAACAACCCATTCCTGAATCAGCG |
| | TAAATCCCACACTGCAGGGAAGACCTCGCACGTAACTCACGTTG |
| | TGCATTGTCAAAGTGTTACATTCGGGCAGCAGCGGATGATCCTC |
| | CAGTATGGTAGCGCGGGTTTCTGTCTCAAAAGGAGGTAGACGAT |
| | CCCTACTGTACGGAGTGCGCCGAGACAACCGAGATCGTGTTGGT |
| | CGTAGTGTCATGCCAAATGGAACGCCGGACGTAGTCATATTTCC |
| | TGAAGCAAAACCAGGTGCGGGCGTGACAAACAGATCTGCGTCT |
| | CCGGTCTCGCCGCTTAGATCGCTCTGTGTAGTAGTTGTAGTATAT |
| | CCACTCTCTCAAAGCATCCAGGCGCCCCCTGGCTTCGGGTTCTA |
| | TGTAAACTCCTTCATGCGCCGCTGCCCTGATAACATCCACCACC |
| | GCAGAATAAGCCACACCCAGCCAACCTACACATTCGTTCTGCGA |
| | GTCACACACGGGAGGAGCGGGAAGAGCTGGAAGAACCATGTTT |
| | TTTTTTTTATTCCAAAAGATTATCCAAAACCTCAAAATGAAGAT |
| | CTATTAAGTGAACGCGCTCCCCTCCGGTGGCGTGGTCAAACTCT |
| | ACAGCCAAAGAACAGATAATGGCATTTGTAAGATGTTGCACAA |
| | TGGCTTCCAAAAGGCAAACGGCCCTCACGTCCAAGTGGACGTA |
| | AAGGCTAAACCCTTCAGGGTGAATCTCCTCTATAAACATTCCAG |
| | CACCTTCAACCATGCCCAAATAATTCTCATCTCGCCACCTTCTCA |
| | ATATATCTCTAAGCAAATCCCGAATATTAAGTCCGGCCATTGTA |
| | AAAATCTGCTCCAGAGCGCCCTCCACCTTCAGCCTCAAGCAGCG |
| | AATCATGATTGCAAAAATTCAGGTTCCTCACAGACCTGTATAAG |
| | ATTCAAAAGCGGAACATTAACAAAAATACCGCGATCCCGTAGG |
| | TCCCTTCGCAGGGCCAGCTGAACATAATCGTGCAGGTCTGCACG |
| | GACCAGCGCGGCCACTTCCCCGCCAGGAACCATGACAAAAGAA |
| | CCCACACTGATTATGACACGCATACTCGGAGCTATGCTAACCAG |
| | CGTAGCCCCGATGTAAGCTTGTTGCATGGGCGGCGATATAAAAT |
| | GCAAGGTGCTGCTCAAAAAATCAGGCAAAGCCTCGCGCAAAAA |
| | AGAAAGCACATCGTAGTCATGCTCATGCAGATAAAGGCAGGTA |
| | AGCTCCGGAACCACCACAGAAAAAGACACCATTTTTCTCTCAAA |
| | CATGTCTGCGGGTTTCTGCATAAACACAAAATAAAATAACAAA |
| | AAAACATTTAAACATTAGAAGCCTGTCTTACAACAGGAAAAAC |
| | AACCCTTATAAGCATAAGACGGACTACGGCCATGCCGGCGTGA |
| | CCGTAAAAAAACTGGTCACCGTGATTAAAAAGCACCACCGACA |
| | GCTCCTCGGTCATGTCCGGAGTCATAATGTAAGACTCGGTAAAC |
| | ACATCAGGTTGATTCACATCGGTCAGTGCTAAAAAGCGACCGA |
| | AATAGCCCGGGGGAATACATACCCGCAGGCGTAGAGACAACAT |
| | TACAGCCCCCATAGGAGGTATAACAAAATTAATAGGAGAGAAA |
| | AACACATAAACACCTGAAAAACCCTCCTGCCTAGGCAAAATAG |
| | CACCCTCCCGCTCCAGAACAACATACAGCGCTTCCACAGCGGCA |
| | GCCATAACAGTCAGCCTTACCAGTAAAAAAGAAAACCTATTAA |
| | AAAAACACCACTCGACACGGCACCAGCTCAATCAGTCACAGTG |
| | TAAAAAAGGGCCAAGTGCAGAGCGAGTATATATAGGACTAAAA |
| | AATGACGTAACGGTTAAAGTCCACAAAAAACACCCAGAAAACC |
| | GCACGCGAACCTACGCCCAGAAACGAAAGCCAAAAAACCCACA |
| | ACTTCCTCAAATCGTCACTTCCGTTTTCCCACGTTACGTCACTTC |
| | CCATTTTAAGAAAACTACAATTCCCAACACATACAAGTTACTCC |
| | GCCCTAAAACCTACGTCACCCGCCCCGTTCCCACGCCCCGCGCC |
| | ACGTCACAAACTCCACCCCCTCATTATCATATTGGCTTCAATCC |
| | AAAATAAGGTATATTATTGATGAT |
| SEQ ID NO: 14 | MSSPGTESAGKSLQYRVDHLLSAVENELQAGSEKGDPTERELRVG |
| | LEESELWLRFKELTNEMIVTKNGRRMFPVLKVNVSGLDPNAMYSF |
| | LLDFVAADNHRWKYVNGEWVPGGKPEPQAPSCVYIHPDSPNFGA |
| | HWMKAPVSFSKVKLTNKLNGGGQIMLNSLHKYEPRIHIVRVGGPQ |
| | R |
| | MITSHCFPETQFIAVTARSDHKEMMEEPGDSQQPGYSQWGWLLPG |
| | TSTVCPPANPHPQFGGALSLPSTHSCDRYPTLRSHRSSPYPSPYAHR |
| | NNSPTYSDNSPACLSMLQSHDNWSSLGMPAHPSMLPVSHNASPPT |
| | SSSQYPSLWSVSNGAVTPGSQAAAVSNGLGAQFFRGSPAHYTPLT |
| | HPVSAPSSSGSPLYEGAAAATDIVDSQYDAAAQGRLIASWTPVSPP |
| | SM |
| SEQ ID NO: 15 | WLLPGTSTV |
| SEQ ID NO: 16 | GCGGGGCAGCCTCACACAGAACACACACAGATATGGGTGTACC |
| | CACTCAGCTCCTGTTGCTGTGGCTTACAGTCGTAGTTGTCAGAT |
| | GTGACATCCAGATGACTCAGTCTCCAGCTTCACTGTCTGCATCT |
| | GTGGGAGAAACTGTCACCATCACATGTGGAGCAAGTGAGAATA |
| | TTTACGGTGCTTTAAATTGGTATCAGCGGAAACAGGGAAAATCT |
| | CCTCAGCTCCTGATTTATGGCGCAAGTAATTTGGCAGATGGCAT |
| | GTCATCGAGGTTCAGTGGCAGTGGATCTGGTAGACAGTATTCTC |
| | TCAAGATCAGTAGCCTGCATCCTGACGATTTTGCAACGTATTAC |
| | TGTCAAAATGTATTAAGTAGTCCGTACACGTTCGGAGGGGGGAC |
| | CAAGCTGGAAATAAAACGGGCTGATGCTGCACCAACTGTATCC |
| | ATCTTCCCACCATCCAGTGAGCAGTTAACATCTGGAGGTGCCTC |
| | AGTCGTGTGCTTCTTGAACAACTTCTACCCCAAAGACATCAATG |
| | TCAAGTGGAAGATTGATGGCAGTGAACGACAAAATGGCGTCCT |
| | GAACAGTTGGACTGATCAGGACAGCAAAGACAGCACCTACAGC |
| | ATGAGCAGCACCCTCACGTTGACCAAGGACGAGTATGAACGAC |
| | ATAACAGCTATACCTGTGAGGCCACTCACAAGACACCAACTTCA |
| | CCCATTGTCAAGAGCTTCAACAGGAATGAGTGT [symbol] AGACAAA |
| | GGTCCTGAGACGCCACCACCAGCTCCCCAGCTCCATCCTATCTT |
| | CCCTTCTAAGGTCTTGGAGGCTTCCCCACAAGCGACCTACCACT |
| | GTTGCGGTGCTCCAAACCTCCTCCCCACCTCCTTCTCCTCCTCCT |
| | CCCTTTCCTTGGCTTTTATCATGCTAATATTTGCAGAAAATATTC |
| | AATAAAGTGAGTCTTTGCACAAAAAAAAAAAAAAAAAAAAAA |
| | AAAA |
| SEQ ID NO: 17 | ACGCGGGACACAGTAGTCTCTACAGTCACAGGAGTACACAGGA |
| | CATTGCCATGGGTTGGAGCTGTATCATCTTCTTTCTGGTAGCAA |
| | CAGCTACAGGTGTGCACTCCCAGGTCCAGCTGCAGCAGTCTGGG |
| | CCTGAGGTGGTGAGGCCTGGGGTCTCAGTGAAGATTTCCTGCAA |
| | GGGTTCCGGCTACACATTCACTGATTATGCTATGCACTGGGTGA |
| | AGCAGAGTCATGCAAAGAGTCTCGAGTGGATTGGACTTATTAGT |
| | ACTTACAGTGGTGATACAAAGTACAACCAGAACTTTAAGGGCA |
| | AGGCCACAATGACTGTAGACAAATCCTCCAACACAGCCTATATG |
| | GAACTTGCCAGATTGACATCTGAGGATTCTGCCATCTATTACTG |
| | TGCAAGAGGGGATTATTCCGGTAGTAGGTACTGGTTTGCTTACT |
| | GGGGCCAAGGGACTCTGGTCACTGTCTCTGCAGCCAAAACGAC |
| | ACCCCCATCTGTCTATCCACTGGCCCCTGGATCTGCTGCCCAAA |
| | CTAACTCCATGGTGACCCTGGGATGCCTGGTCAAGGGCTATTTC |
| | CCTGAGCCAGTGACAGTGACCTGGAACTCTGGATCCCTGTCCAG |
| | CGGTGTGCACACCTTCCCAGCTGTCCTGCAGTCTGACCTCTACA |
| | CTCTGAGCAGCTCAGTGACTGTCCCCTCCAGCACCTGGCCCAGC |
| | GAGACCGTCACCTGCAACGTTGCCCACCCGGCCAGCAGCACCA |
| | AGGTGGACAAGAAAATTGTGCCCAGGGATTGTGGTTGTAAGCC |
| | TTGCATATGTACAGTCCCAGAAGTATCATCTGTCTTCATCTTCCC |
| | CCCAAAGCCCAAGGATGTGCTCACCATTACTCTGACTCCTAAGG |
| | TCACGTGTGTTGTGGTAGACATCAGCAAGGATGATCCCGAGGTC |
| | CAGTTCAGCTGGTTTGTAGATGATGTGGAGGTGCACACAGCTCA |
| | GACGCAACCCCGGGAGGAGCAGTTCAACAGCACTTTCCGCTCA |
| | GTCAGTGAACTTCCCATCATGCACCAGGACTGGCTCAATGGCAA |
| | GGAGTTCAAATGCAGGGTCAACAGTGCAGCTTTCCCTGCCCCCA |
| | TCGAGAAAACCATCTCCAAAACCAAAGGCAGACCGAAGGCTCC |
| | ACAGGTGTACACCATTCCACCTCCCAAGGAGCAGATGGCCAAG |
| | GATAAAGTCAGTCTGACCTGCATGATAACAGACTTCTTCCCTGA |
| | AGACATTACTGTGGAGTGGCAGTGGAATGGGCAGCCAGCGGAG |
| | AACTACAAGAACACTCAGCCCATCATGGACACAGATGGCTCTTA |
| | CTTCGTCTACAGCAAGCTCAATGTGCAGAAGAGCAACTGGGAG |
| | GCAGGAAATACTTTCACCTGCTCTGTGTTACATGAGGGCCTGCA |
| | CAACCACCATACTGAGAAGAGCCTCTCCCACTCTCCTGGTAAA [symbol] |
| | [symbol] TCCCAGTGTCCTTGGAGCCCTCTGGCCCTACAGGACTTTGAC |
| | ACCTACCTCCACCCCTCCCTGTATAAATAAAGCACCCAGCACTG |
| | CCTCGGGACCCTGCATAAAAAAAAAAAAAAAAAAAAAAAAAA |
| | AA |
| SEQ ID NO: 18 | LMTQSPASLSASVGETVTITCGASENIYGALNWYQRKQGKSPQLLI |
| | YGASNLADGMSSRFSGSGSGRQYSLKISSLHPDDVATYYCQNVLS |
| | SPYTFGGGTKLEIKKG |
| SEQ ID NO: 19 | MGVPTQLLLLWLTVVVVRC/DIQMTQSPSSLSASVGDRVTITCQAS |
| | ENIYGALNWYQRKPGKSPKLLIYGASNLATGMPSRFSGSGSGTDY |
| | TFTISSLQPEDIATYYCQQVLSSPYTFGGGTKLEIKR/TVAAPSVFIFP |
| | PSDEQLKSGTASVVCLINNFYPREAKVQWKVDNALQSGNSQESVT |
| | EQDSKDSTYSLSSTLTLSKADYEKHKVYACEVTHQGLSSPVTKSFN |
| | RGEC |
| SEQ ID NO: 20 | LEESGPEVVRPGVSVKISCKGSGYTFTDYAMHWVKQSHAKSLEWI |
| | GLISTYSGDTKYNQNFKGKATMTVDKSSNTAYMELARLTSEDSAI |
| | YYCARGDYSGSRYWFAYWGQGTTVTR |
| SEQ ID NO: 21 | GASENIYGALN |
| SEQ ID NO: 22 | GASNLAD |
| SEQ ID NO: 23 | QNVLSSPYT |
| SEQ ID NO: 24 | QASENIYGALN |
| SEQ ID NO: 25 | GASNLAT |
| SEQ ID NO: 26 | QQVLSSPYT |
| SEQ ID NO: 27 | GYTFTDYAMH |
| SEQ ID NO: 28 | LISTYSGDTKYNQNFKG |
| SEQ ID NO: 29 | GDYSGSRYWFAY |
| SEQ ID NO: 30 | LISTYSGDTKYNQKFQG |
| SEQ ID NO: 31 | GDYSGSRYWFAY |
| SEQ ID NO: 99 | MGWSCIIFFLVATATGVHS/QVQLVQSGAEVKKPGASVKVSCKAS |
| | GYTFTDYAMHWVRQAPGQRLEWMGLISTYSGDTKYNQNFQGR |
| | VTMTVDKSASTAYMELSSLRSEDTAVYYCARGDYSGSRYWFAY |
| | WGQGTLVTVSS/ASTKGPSVFPLAPSSKSTSGGTAALGCLVKDYFP |
| | EPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLF |
| | PPKPKDTLMISRTPEVTCVVVDVSHEDPEVKFNWYVDGVEVHNAK |
| | TKPREEQYNSTYRVVSVLTVLHQDWLNGKEYKCKVSNKALPAPIE |
| | KTISKAKGQPREPQVYTLPPSRDELTKNQVSLTCLVKGFYPSDIAVE |
| | WESNGQPENNYKTTPPVLDSDGSFFLYSKLTVDKSRWQQGNVFSC |
| | SVMHEALHNHYTQKSLSLSPGK |
| SEQ ID NO: | ATGGAGTCTCCCTCGGCCCCTCCCCACAGATGGTGCATCCCCTG |
| 100 | GCAGAGGCTCCTGCTCACAGCCTCACTTCTAACCTTCTGGAACC |
| | CGCCCACCACTGCCAAGCTCACTATTGAATCCACGCCGTTCAAT |
| | GTCGCAGAGGGGAAGGAGGTGCTTCTACTTGTCCACAATCTGCC |
| | CCAGCATCTTTTTGGCTACAGCTGGTACAAAGGTGAAAGAGTGG |
| | ATGGCAACCGTCAAATTATAGGATATGTAATAGGAACTCAACA |
| | AGCTACCCCAGGGCCCGCATACAGTGGTCGAGAGATAATATAC |
| | CCCAATGCATCCCTGCTGATCCAGAACATCATCCAGAATGACAC |
| | AGGATTCTACACCCTACACGTCATAAAGTCAGATCTTGTGAATG |
| | AAGAAGCAACTGGCCAGTTCCGGGTATACCCGGAGCTGCCCAA |
| | GCCCTCCATCTCCAGCAACAACTCCAAACCCGTGGAGGACAAG |
| | GATGCTGTGGCCTTCACCTGTGAACCTGAGACTCAGGACGCAAC |
| | CTACCTGTGGTGGGTAAACAATCAGAGCCTCCCGGTCAGTCCCA |
| | GGCTGCAGCTGTCCAATGGCAACAGGACCCTCACTCTATTCAAT |
| | GTCACAAGAAATGACACAGCAAGCTACAAATGTGAAACCCAGA |
| | ACCCAGTGAGTGCCAGGCGCAGTGATTCAGTCATCCTGAATGTC |
| | CTCTATGGCCCGGATGCCCCCACCATTTCCCCTCTAAACACATCT |
| | TACAGATCAGGGGAAAATCTGAACCTCTCCTGCCACGCAGCCTC |
| | TAACCCACCTGCACAGTACTCTTGGTTTGTCAATGGGACTTTCC |
| | AGCAATCCACCCAAGAGCTCTTTATCCCCAACATCACTGTGAAT |
| | AATAGTGGATCCTATACGTGCCAAGCCCATAACTCAGACACTGG |
| | CCTCAATAGGACCACAGTCACGACGATCACAGTCTATGCAGAG |
| | CCACCCAAACCCTTCATCACCAGCAACAACTCCAACCCCGTGGA |
| | GGATGAGGATGCTGTAGCCTTAACCTGTGAACCTGAGATTCAGA |
| | ACACAACCTACCTGTGGTGGGTAAATAATCAGAGCCTCCCGGTC |
| | AGTCCCAGGCTGCAGCTGTCCAATGACAACAGGACCCTCACTCT |
| | ACTCAGTGTCACAAGGAATGATGTAGGACCCTATGAGTGTGGA |
| | ATCCAGAACGAATTAAGTGTTGACCACAGCGACCCAGTCATCCT |
| | GAATGTCCTCTATGGCCCAGACGACCCCACCATTTCCCCCTCAT |
| | ACACCTATTACCGTCCAGGGGTGAACCTCAGCCTCTCCTGCCAT |
| | GCAGCCTCTAACCCACCTGCACAGTATTCTTGGCTGATTGATGG |
| | GAACATCCAGCAACACACACAAGAGCTCTTTATCTCCAACATCA |
| | CTGAGAAGAACAGCGGACTCTATACCTGCCAGGCCAATAACTC |
| | AGCCAGTGGCCACAGCAGGACTACAGTCAAGACAATCACAGTC |
| | TCTGCGGAGCTGCCCAAGCCCTCCATCTCCAGCAACAACTCCAA |
| | ACCCGTGGAGGACAAGGATGCTGTGGCCTTCACCTGTGAACCTG |
| | AGGCTCAGAACACAACCTACCTGTGGTGGGTAAATGGTCAGAG |
| | CCTCCCAGTCAGTCCCAGGCTGCAGCTGTCCAATGGCAACAGGA |
| | CCCTCACTCTATTCAATGTCACAAGAAATGACGCAAGAGCCTAT |
| | GTATGTGGAATCCAGAACTCAGTGAGTGCAAACCGCAGTGACC |
| | CAGTCACCCTGGATGTCCTCTATGGGCCGGACACCCCCATCATT |
| | TCCCCCCCAGACTCGTCTTACCTTTCGGGAGCGGACCTCAACCT |
| | CTCCTGCCACTCGGCCTCTAACCCATCCCCGCAGTATTCTTGGCG |
| | TATCAATGGGATACCGCAGCAACACACACAAGTTCTCTTTATCG |
| | CCAAAATCACGCCAAATAATAACGGGACCTATGCCTGTTTTGTC |
| | TCTAACTTGGCTACTGGCCGCAATAATTCCATAGTCAAGAGCAT |
| | CACAGTCTCTGCATCTGGAACTTCTCCTGGTCTCTCAGCTGGGG |
| | CCACTGTCGGCATCATGATTGGAGTGCTGGTTGGGGTTGCTCTG |
| | ATATAG |
| SEQ ID NO: | ATGACACCGGGCACCCAGTCTCCTTTCTTCCTGCTGCTGCTCCTC |
| 101 | ACAGTGCTTACAGTTGTTACGGGTTCTGGTCATGCAAGCTCTAC |
| | CCCAGGTGGAGAAAAGGAGACTTCGGCTACCCAGAGAAGTTCA |
| | GTGCCCAGCTCTACTGAGAAGAATGCTGTGAGTATGACCAGCA |
| | GCGTACTCTCCAGCCACAGCCCCGGTTCAGGCTCCTCCACCACT |
| | CAGGGACAGGATGTCACTCTGGCCCCGGCCACGGAACCAGCTT |
| | CAGGTTCAGCTGCCCTTTGGGGACAGGATGTCACCTCGGTCCCA |
| | GTCACCAGGCCAGCCCTGGGCTCCACCACCCCGCCAGCCCACGA |
| | TGTCACCTCAGCCCCGGACAACAAGCCAGCCCCGGGCTCCACCG |
| | CCCCCCCAGCCCACGGTGTCACCTCGTATCTTGACACCAGGCCG |
| | GCCCCGGTTTATCTTGCCCCCCCAGCCCATGGTGTCACCTCGGC |
| | CCCGGACAACAGGCCCGCCTTGGGCTCCACCGCCCCTCCAGTCC |
| | ACAATGTCACCTCGGCCTCAGGCTCTGCATCAGGCTCAGCTTCT |
| | ACTCTGGTGCACAACGGCACCTCTGCCAGGGCTACCACAACCCC |
| | AGCCAGCAAGAGCACTCCATTCTCAATTCCCAGCCACCACTCTG |
| | ATACTCCTACCACCCTTGCCAGCCATAGCACCAAGACTGATGCC |
| | AGTAGCACTCACCATAGCACGGTACCTCCTCTCACCTCCTCCAA |
| | TCACAGCACTTCTCCCCAGTTGTCTACTGGGGTCTCTTTCTTTTT |
| | CCTGTCTTTTCACATTTCAAACCTCCAGTTTAATTCCTCTCTGGA |
| | AGATCCCAGCACCGACTACTACCAAGAGCTGCAGAGAGACATT |
| | TCTGAAATGTTTTTGCAGATTTATAAACAAGGGGGTTTTCTGGG |
| | CCTCTCCAATATTAAGTTCAGGCCAGGATCTGTGGTGGTACAAT |
| | TGACTCTGGCCTTCCGAGAAGGTACCATCAATGTCCACGACGTG |
| | GAGACACAGTTCAATCAGTATAAAACGGAAGCAGCCTCTCGAT |
| | ATAACCTGACGATCTCAGACGTCAGCGTGAGTGATGTGCCATTT |
| | CCTTTCTCTGCCCAGTCTGGGGCTGGGGTGCCAGGCTGGGGCAT |
| | CGCGCTGCTGGTGCTGGTCTGTGTTCTGGTTTATCTGGCCATTGT |
| | CTATCTCATTGCCTTGGCTGTCGCTCAGGTTCGCCGAAAGAACT |
| | ACGGGCAGCTGGACATCTTTCCAGCCCGGGATAAATACCATCCT |
| | ATGAGCGAGTACGCTCTTTACCACACCCATGGGCGCTATGTGCC |
| | CCCTAGCAGTCTTTTCCGTAGCCCCTATGAGAAGGTTTCTGCAG |
| | GTAATGGTGGCAGCTATCTCTCTTACACAAACCCAGCAGTGGCA |
| | GCCGCTTCTGCCAACTTGTAG |
| SEQ ID NO: | ATGAGCTCCCCTGGCACCGAGAGCGCGGGAAAGAGCCTGCAGT |
| 102 | ACCGAGTGGACCACCTGCTGAGCGCCGTGGAGAATGAGCTGCA |
| | GGCGGGCAGCGAGAAGGGCGACCCCACAGAGCGCGAACTGCGC |
| | GTGGGCCTGGAGGAGAGCGAGCTGTGGCTGCGCTTCAAGGAGC |
| | TCACCAATGAGATGATCGTGACCAAGAACGGCAGGAGGATGTT |
| | TCCGGTGCTGAAGGTGAACGTGTCTGGCCTGGACCCCAACGCCA |
| | TGTACTCCTTCCTGCTGGACTTCGTGGCGGCGGACAACCACCGC |
| | TGGAAGTACGTGAACGGGGAATGGGTGCCGGGGGGCAAGCCGG |
| | AGCCGCAGGCGCCCAGCTGCGTCTACATCCACCCCGACTCGCCC |
| | AACTTCGGGGCCCACTGGATGAAGGCTCCCGTCTCCTTCAGCAA |
| | AGTCAAGCTCACCAACAAGCTCAACGGAGGGGGCCAGATCATG |
| | CTGAACTCCTTGCATAAGTATGAGCCTCGAATCCACATAGTGAG |
| | AGTTGGGGGTCCACAGCGCATGATCACCAGCCACTGCTTCCCTG |
| | AGACCCAGTTCATAGCGGTGACTGCTAGAAGTGATCACAAAGA |
| | GATGATGGAGGAACCCGGAGACAGCCAGCAACCTGGGTACTCC |
| | CAATGGGGGTGGCTTCTTCCTGGAACCAGCACCGTGTGTCCACC |
| | TGCAAATCCTCATCCTCAGTTTGGAGGTGCCCTCTCCCTCCCCTC |
| | CACGCACAGCTGTGACAGGTACCCAACCCTGAGGAGCCACCGG |
| | TCCTCACCCTACCCCAGCCCCTATGCTCATCGGAACAATTCTCC |
| | AACCTATTCTGACAACTCACCTGCATGTTTATCCATGCTGCAATC |
| | CCATGACAATTGGTCCAGCCTTGGAATGCCTGCCCATCCCAGCA |
| | TGCTCCCCGTGAGCCACAATGCCAGCCCACCTACCAGCTCCAGT |
| | CAGTACCCCAGCCTGTGGTCTGTGAGCAACGGCGCCGTCACCCC |
| | GGGCTCCCAGGCAGCAGCCGTGTCCAACGGGCTGGGGGCCCAG |
| | TTCTTCCGGGGCTCCCCCGCGCACTACACACCCCTCACCCATCC |
| | GGTCTCGGCGCCCTCTTCCTCGGGATCCCCACTGTACGAAGGGG |
| | CGGCCGCGGCCACAGACATCGTGGACAGCCAGTACGACGCCGC |
| | AGCCCAAGGCCGCCTCATAGCCTCATGGACACCTGTGTCGCCAC |
| | CTTCCATGTGA |

1. A composition comprising:
a recombinant replication defective viral vector comprising a nucleic acid sequence encoding an antigen and an E2b deletion; and
a nucleic acid sequence encoding calreticulin.

2. The composition of claim 1, wherein the antigen and calreticulin are expressed together as a fusion protein in a cell.

3-6. (canceled)

7. The composition of claim 1, wherein calreticulin boosts a host immune response to the composition.

8. (canceled)

9. The composition of claim 1, wherein the nucleic acid sequence encoding calreticulin has at least 70%, at least 75%, at least 80%, at least 85%, at least 87%, at least 90%, at least 92%, at least 95%, at least 97%, or at least 99% sequence identity to SEQ ID NO: 107.

10. The composition of claim 1, wherein the antigen is a CEA antigen, a MUC1-C antigen, a Brachyury antigen, a tumor neo-antigen or a tumor-neo-epitope.

11.-16. (canceled)

17. The composition of claim 1, wherein the nucleic acid sequence encoding the antigen or the one or more additional antigens has at least 70%, at least 75%, at least 80%, at least 85%, at least 87%, at least 90%, at least 92%, at least 95%, at least 97%, or at least 99% sequence identity to SEQ ID NO: 1, SEQ ID NO: 3, SEQ ID NO: 4, SEQ ID NO: 100, positions 1057 to 3165 of SEQ ID NO: 2, SEQ ID NO: 5, SEQ ID NO: 6, SEQ ID NO: 7, SEQ ID NO: 101, positions 93, 141-142, 149-151, 392, 404, 406, 422, 430-431, 444-445, or 460 of SEQ ID NO: 7, SEQ ID NO: 9, SEQ ID NO: 10, SEQ ID NO: 14, SEQ ID NO: 102, or positions 1033 to 2283 of SEQ ID NO: 13.

18.-20. (canceled)

21. The composition of claim 1, wherein the replication defective viral vector is an adenovirus subtype 5 (Ad5)-based vector.

22.-24. (canceled)

25. The composition of claim 1, wherein the composition comprises at least 1×10⁹ viral particles, at least 1×10¹⁰ viral particles, at least 1×10¹¹ viral particles, at least 5×10¹¹ viral particles, at least 1×10¹² viral particles, or at least 5×10¹² viral particles in a single dose.

26.-30. (canceled)

31. The composition of claim 1, wherein the composition or the replication-defective virus vector further comprises a nucleic acid sequences encoding a costimulatory molecule.

32.-35. (canceled)

36. The composition of claim 1, wherein the composition further comprises an immune pathway checkpoint modulator, an anti-CEA antibody, a chemotherapeutic agent, a population of engineered natural killer (NK) cells, an IL-15 superagonist complex or combinations thereof.

37.-60. (canceled)

61. A method of treating a subject in need thereof, the method comprising administering to the subject:
a recombinant replication defective viral vector comprising a nucleic acid sequence encoding an antigen; and
a nucleic acid sequence encoding calreticulin.

62. The method of claim 61, wherein the antigen and calreticulin are expressed together as a fusion protein in a cell.

63.-68. (canceled)

69. The method of claim 61, wherein the nucleic acid sequence encoding calreticulin has at least 70%, at least 75%, at least 80%, at least 85%, at least 87%, at least 90%, at least 92%, at least 95%, at least 97%, or at least 99% sequence identity to SEQ ID NO: 107.

70. The method of claim 61, wherein the antigen is a CEA antigen, a MUC1-C antigen, a Brachyury antigen or a tumor neo-antigen or a tumor-neo-epitope.

71.-76. (canceled)

77. The method of claim 61, wherein the nucleic acid sequence encoding the antigen or the one or more additional antigens has at least 70%, at least 75%, at least 80%, at least 85%, at least 87%, at least 90%, at least 92%, at least 95%, at least 97%, or at least 99% sequence identity to SEQ ID NO: 1, SEQ ID NO: 3, SEQ ID NO: 4, SEQ ID NO: 100, or positions 1057 to 3165 of SEQ ID NO: 2, SEQ ID NO: 5, SEQ ID NO: 6, SEQ ID NO: 7, SEQ ID NO: 101, or positions 93, 141-142, 149-151, 392, 404, 406, 422, 430-431, 444-445, or 460 of SEQ ID NO: 7, SEQ ID NO: 9, SEQ ID NO: 10, SEQ ID NO: 14, SEQ ID NO: 102, or positions 1033 to 2283 of SEQ ID NO: 13.

78.-80. (canceled)

81. The method of claim 61, wherein the replication defective viral vector is an adenovirus subtype 5 (Ad5)-based vector.

82.-84. (canceled)

85. The method of claim 61, wherein the method comprises administering at least 1×10⁹ viral particles, at least 1×10¹⁰ viral particles, at least 1×10¹¹ viral particles, at least 5×10¹¹ viral particles, at least 1×10¹² viral particles, or at least 5×10¹² viral particles in a single dose.

86.-90. (canceled)

91. The method of claim 61, wherein the method further comprises administering the replication-defective virus vector, wherein the replication-defective virus vector further comprises a nucleic acid sequences encoding a costimulatory molecule.

92.-95. (canceled)

96. The method of claim 61, wherein the method further comprises administering to the subject an immune pathway checkpoint modulator, an anti-CEA antibody, a chemotherapeutic agent, a population of engineered natural killer (NK) cells, an IL-15 superagonist complex or combinations thereof.

97.-138. (canceled)

139. The method of claim 61, wherein the disease is a cancer.

140.-147. (canceled)


SEQUENCE LISTING (108 sequences)

SEQ ID NO: 1 - DNA, 2109 bp, Artificial Sequence
  Synthetic polynucleotide
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 2 - DNA, 32315 bp, Artificial Sequence
  Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttggtaccgagctcggatccactagtaacggccgccagtgtgctggaattcggcttaaaggtacccagagcagacagccgccaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcggacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 3 - protein, 9 aa, Artificial Sequence
  Synthetic peptide
YLSGANLNL

SEQ ID NO: 4 - protein, 9 aa, Artificial Sequence
  Synthetic peptide
YLSGADLNL

SEQ ID NO: 5 - DNA, 1826 bp, Artificial Sequence
  Synthetic polynucleotide
cgctccacctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaa

SEQ ID NO: 6 - DNA, 1826 bp, Artificial Sequence
  Synthetic polynucleotide
cgctccacctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccctttggggacaggatgtcacctcggtcccagtcaccaggccagccctgggctccaccaccccgccagcccacgatgtcacctcagccccggacaacaagccagccccgggctccaccgcccccccagcccacggtgtcacctcgtatcttgacaccaggccggccccggtttatcttgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggtttatctggccattgtctatctcattgccttggctgtcgctcaggttcgccgaaagaactacgggcagctggacatctttccagcccgggataaataccatcctatgagcgagtacgctctttaccacacccatgggcgctatgtgccccctagcagtcttttccgtagcccctatgagaaggtttctgcaggtaatggtggcagctatctctcttacacaaacccagcagtggcagccgcttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaa

SEQ ID NO: 7 - protein, 475 aa, Artificial Sequence
  Mutated MUC1 protein sequence
MTPGTQSPFFLLLLLTVLTVVTGSGHASSTPGGEKETSATQRSSVPSSTEKNAVSMTSSVLSSHSPGSGSSTTQGQDVTLAPATEPASGSAALWGQDVTSVPVTRPALGSTTPPAHDVTSAPDNKPAPGSTAPPAHGVTSYLDTRPAPVYLAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVYLAIVYLIALAVAQVRRKNYGQLDIFPARDKYHPMSEYALYHTHGRYVPPSSLFRSPYEKVSAGNGGSYLSYTNPAVAAASANL

SEQ ID NO: 8 - DNA, 32040 bp, Artificial Sequence
  Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttctagatgcatgctcgagcggccgccagtgtgatggatatctgcagaattcgcccttgctcgctccacctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccctttggggacaggatgtcacctcggtcccagtcaccaggccagccctgggctccaccaccccgccagcccacgatgtcacctcagccccggacaacaagccagccccgggctccaccgcccccccagcccacggtgtcacctcgtatcttgacaccaggccggccccggtttatcttgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggtttatctggccattgtctatctcattgccttggctgtcgctcaggttcgccgaaagaactacgggcagctggacatctttccagcccgggataaataccatcctatgagcgagtacgctctttaccacacccatgggcgctatgtgccccctagcagtcttttccgtagcccctatgagaaggtttctgcaggtaatggtggcagctatctctcttacacaaacccagcagtggcagccgcttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaacgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 9 - DNA, 2500 bp, Artificial Sequence
  Synthetic polynucleotide
ggaggacacttctcagaaggggttgttttgcttttgcttatttccgtccatttccctctctgcgcgcggaccttccttttccagatggtgagagccgcggggacacccgacgccggggcaggctgatccacgatcctgggtgtgcgtaacgccgcctggggctccgtgggcgagggacgtgtggggacaggtgcaccggaaactgccagactggagagttgaggcatcggaggcgcgagaacagcactactactgcggcgagacgagcgcggcgcatcccaaagcccggccaaatgcgctcgtccctgggaggggagggaggcgcgcctggagcggggacagtcttggtccgcgccctcctcccgggtctgtgccgggacccgggacccgggagccgtcgcaggtctcggtccaaggggccccttttctcggaagggcggcggccaagagcagggaaggtggatctcaggtagcgagtctgggcttcggggacggcggggaggggagccggacgggaggatgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgcttatcagaacgaggagatcacagctcttaaaattaagtacaatccatttgcaaaagctttccttgatgcaaaggaaagaagtgatcacaaagagatgatggaggaacccggagacagccagcaacctgggtactcccaatgggggtggcttcttcctggaaccagcaccctgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtgaagcagcaaggcccaggtcccgaaagatgcagtgactttttgtcgtggcagccagtggtgactggattgacctactaggtacccagtggcagtctcaggttaagaaggaaatgcagcctcagtaacttccttttcaaagcagtggaggagcacacggcacctttccccagagccccagcatcccttgctcacacctgcagtagcggtgctgtcccaggtggcttacagatgaacccaactgtggagatgatgcagttggcccaacctcactgacggtgaaaaaatgtttgccagggtccagaaactttttttggtttatttctcatacagtgtattggcaactttggcacaccagaatttgtaaactccaccagtcctactttagtgagataaaaagcacactcttaatcttcttccttgttgctttcaagtagttagagttgagctgttaaggacagaataaaatcatagttgaggacagcaggttttagttgaattgaaaatttgactgctctgccccctagaatgtgtgtattttaagcatatgtagctaatctcttgtgttgttaaactataactgtttcatatttttcttttgacaaagtagccaaagacaatcagcagaaagcattttctgcaaaataaacgcaatatgcaaaaaaaaaaaaaaaaaa

SEQ ID NO: 10 - DNA, 1251 bp, Artificial Sequence
  Synthetic polynucleotide
tctagagccaccatgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgctagaagtgatcacaaagagatgatggaggaacccggagacagccagcaacctgggtactcccaatgggggtggcttcttcctggaaccagcaccgtgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtgagatatc

SEQ ID NO: 11 - protein, 9 aa, Artificial Sequence
  Synthetic peptide
TCTCTCCNA

SEQ ID NO: 12 - protein, 435 aa, Artificial Sequence
  Synthetic polynucleotide
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGDPQRMITSHCFPETQFIAVTAYQNEEITALKIKYNPFAKAFLDAKERSDHKEMMEEPGDSQQPGYSQWGWLLPGTSTLCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVTNGLGAQFFRGSPAHYTPLTHPVSAPSSSGSPLYEGAAAATNIVDSQYDAAAQGRLIASWTPVSPPSM

SEQ ID NO: 13 - DNA, 31465 bp, Artificial Sequence
  Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttctagatgcatgctcgagcggccgccagtgtgatggatatctgcagaattcgcccttgcttctagagccaccatgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgctagaagtgatcacaaagagatgatggaggaacccggagacagccagcaacctgggtactcccaatgggggtggcttcttcctggaaccagcaccgtgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtgagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 14 - protein, 410 aa, Artificial Sequence
  Synthetic polynucleotide
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTARSDHKEMMEEPGDSQQPGYSQWGWLLPGTSTVCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 15 - protein, 9 aa, Artificial Sequence
  Synthetic peptide
WLLPGTSTV

SEQ ID NO: 16 - DNA, 965 bp, Artificial Sequence
  Synthetic polynucleotide
gcggggcagcctcacacagaacacacacagatatgggtgtacccactcagctcctgttgctgtggcttacagtcgtagttgtcagatgtgacatccagatgactcagtctccagcttcactgtctgcatctgtgggagaaactgtcaccatcacatgtggagcaagtgagaatatttacggtgctttaaattggtatcagcggaaacagggaaaatctcctcagctcctgatttatggcgcaagtaatttggcagatggcatgtcatcgaggttcagtggcagtggatctggtagacagtattctctcaagatcagtagcctgcatcctgacgattttgcaacgtattactgtcaaaatgtattaagtagtccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacaccaacttcacccattgtcaagagcttcaacaggaatgagtgttagagacaaaggtcctgagacgccaccaccagctccccagctccatcctatcttcccttctaaggtcttggaggcttccccacaagcgacctaccactgttgcggtgctccaaacctcctccccacctccttctcctcctcctccctttccttggcttttatcatgctaatatttgcagaaaatattcaataaagtgagtctttgcacaaaaaaaaaaaaaaaaaaaaaaaaaa

SEQ ID NO: 17 - DNA, 1575 bp, Artificial Sequence
  Synthetic polynucleotide
acgcgggacacagtagtctctacagtcacaggagtacacaggacattgccatgggttggagctgtatcatcttctttctggtagcaacagctacaggtgtgcactcccaggtccagctgcagcagtctgggcctgaggtggtgaggcctggggtctcagtgaagatttcctgcaagggttccggctacacattcactgattatgctatgcactgggtgaagcagagtcatgcaaagagtctcgagtggattggacttattagtacttacagtggtgatacaaagtacaaccagaactttaagggcaaggccacaatgactgtagacaaatcctccaacacagcctatatggaacttgccagattgacatctgaggattctgccatctattactgtgcaagaggggattattccggtagtaggtactggtttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatcccagtgtccttggagccctctggccctacaggactttgacacctacctccacccctccctgtataaataaagcacccagcactgcctcgggaccctgcataaaaaaaaaaaaaaaaaaaaaaaaaaaa

SEQ ID NO: 18 - protein, 107 aa, Artificial Sequence
  Synthetic polynucleotide
LMTQSPASLSASVGETVTITCGASENIYGALNWYQRKQGKSPQLLIYGASNLADGMSSRFSGSGSGRQYSLKISSLHPDDVATYYCQNVLSSPYTFGGGTKLEIKKG

SEQ ID NO: 19 - protein, 233 aa, Artificial Sequence
  Synthetic polynucleotide
MGVPTQLLLLWLTVVVVRCDIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKRTVAAPSVFIFPPSDEQLKSGTASVVCLINNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 116 aa, Artificial Sequence
  Synthetic polynucleotide
LEESGPEVVRPGVSVKISCKGSGYTFTDYAMHWVKQSHAKSLEWIGLISTYSGDTKYNQNFKGKATMTVDKSSNTAYMELARLTSEDSAIYYCARGDYSGSRYWFAYWGQGTTVTR

SEQ ID NO: 21 - protein, 11 aa, Artificial Sequence
  Synthetic peptide
GASENIYGALN

SEQ ID NO: 22 - protein, 7 aa, Artificial Sequence
  Synthetic peptide
GASNLAD

SEQ ID NO: 23 - protein, 9 aa, Artificial Sequence
  Synthetic peptide
QNVLSSPYT

SEQ ID NO: 24 - protein, 11 aa, Artificial Sequence
  Synthetic peptide
QASENIYGALN

SEQ ID NO: 25 - protein, 7 aa, Artificial Sequence
  Synthetic peptide
GASNLAT

SEQ ID NO: 26 - protein, 9 aa, Artificial Sequence
  Synthetic peptide
QQVLSSPYT

SEQ ID NO: 27 - protein, 10 aa, Artificial Sequence
  Synthetic peptide
GYTFTDYAMH

SEQ ID NO: 28 - protein, 17 aa, Artificial Sequence
  Synthetic peptide
LISTYSGDTKYNQNFKG

SEQ ID NO: 29 - protein, 12 aa, Artificial Sequence
  Synthetic peptide
GDYSGSRYWFAY

SEQ ID NO: 30 - protein, 17 aa, Artificial Sequence
  Synthetic peptide
LISTYSGDTKYNQKFQG

SEQ ID NO: 31 - protein, 12 aa, Artificial Sequence
  Synthetic peptide
GDYSGSRYWFAY

SEQ ID NO: 32 - protein, 132 aa, Artificial Sequence
  Synthetic polynucleotide
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 33 - protein, 230 aa, Artificial Sequence
  Synthetic polynucleotide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFDDDDKDPPDPHQPDMTKGYCPGGRWGFGDLAVCDGEKYPDGSFWHQWMQTWFTGPQFYFDCVSGGEPLPGPPPPGGCGGAIPSEQPNAP

SEQ ID NO: 34 - protein, 578 aa, Artificial Sequence
  Synthetic polynucleotide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFPLVPRGSPMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYSGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 35 - protein, 220 aa, Artificial Sequence [sequence omitted]

SEQ ID NO: 36 - protein, 729 aa, Artificial Sequence
  Synthetic polynucleotide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAAS

SEQ ID NO: 37 - protein, 30 aa, Artificial Sequence
  Synthetic polynucleotide
TAASDNFQLSQGGQGFAIPIGQAMAIAGQI

SEQ ID NO: 38 - protein, 128 aa, Artificial Sequence
  Synthetic polynucleotide
TAASDNFQLSQGGQGFAIPIGQAMAIAGQIKLPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 39 - protein, 128 aa, Artificial Sequence
  Synthetic polynucleotide
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAE

SEQ ID NO: 40 - protein, 355 aa, Artificial Sequence
  Synthetic polynucleotide
MSNSRRRSLRWSWLLSVLAAVGLGLATAPAQAAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 41 - protein, 364 aa, Artificial Sequence
  Synthetic polynucleotide
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPAFFTDVNQMYDVLLNKSGATGVFTDFPDTGVEFLKGIK

SEQ ID NO: 42 - protein, 313 aa, Artificial Sequence
  Synthetic polynucleotide
MEINVSKLRTDLPQVGVQPYRQVHAHSTGNPHSTVQNEADYHWRKDPELGFFSHIVGNGCIMQVGPVDNGAWDVGGGWNAETYAAVELIESHSTKEEFMTDYRLYIELLRNLADEAGLPKTLDTGSLAGIKTHEYCTNNQPNNHSDHVDPYPYLAKWGISREQFKHDIENGLTIETGWQKNDTGYWYVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPEFRMSQMA

SEQ ID NO: 43 - protein, 166 aa, Artificial Sequence
  IFN-y
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 44 - protein, 233 aa, Artificial Sequence
  TNFa
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 45 - protein, 153 aa, Artificial Sequence
  IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 46 - protein, 99 aa, Artificial Sequence
  IL-8
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 47 - protein, 662 aa, Artificial Sequence
  IL-12
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 48 - protein, 193 aa, Artificial Sequence
  IL-18
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 49 - protein, 177 aa, Artificial Sequence
  IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 50 - protein, 152 aa, Artificial Sequence
  IL-3
MSRLPVLLLLQLLVRPGLQAPMTQTTSLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 51 - protein, 153 aa, Artificial Sequence [sequence omitted]

SEQ ID NO: 52 - protein, 134 aa, Artificial Sequence
  IL-5
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 53 - protein, 212 aa, Artificial Sequence
  IL-6
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 54 - protein, 140 aa, Artificial Sequence
  IL-9
MVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 55 - protein, 178 aa, Artificial Sequence
  IL-10
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 56 - protein, 145 aa, Artificial Sequence [sequence omitted]

SEQ ID NO: 57 - protein, 136 aa, Artificial Sequence [sequence omitted]

SEQ ID NO: 58 - protein, 656 aa, Artificial Sequence
  Synthetic polynucleotide
MEGDGSDPEPPDAGEDSKSENGENAPIYCICRKPDINCFMIGCDNCNEWFHGDCIRITEKMAKAIREWYCRECREKDPKLEIRYRHKKSRERDGNERDSSEPRDEGGGRKRPVPDPNLQRRAGSGTGVGAMLARGSASPHKSSPQPLVATPSQHHQQQQQQIKRSARMCGECEACRRTEDCGHCDFCRDMKKFGGPNKIRQKCRLRQCQLRARESYKYFPSSLSPVTPSESLPRPRRPLPTQQQPQPSQKLGRIREDEGAVASSTVKEPPEATATPEPLSDEDLPLDPDLYQDFCAGAFDDNGLPWMSDTEESPFLDPALRKRAVKVKHVKRREKKSEKKKEERYKRHRQKQKHKDKWKHPERADAKDPASLPQCLGPGCVRPAQPSSKYCSDDCGMKLAANRIYEILPQRIQQWQQSPCIAEEHGKKLLERIRREQQSARTRLQEMERRFHELEAIILRAKQQAVREDEESNEGDSDDTDLQIFCVSCGHPINPRVALRHMERCYAKYESQTSFGSMYPTRIEGATRLFCDVYNPQSKTYCKRLQVLCPEHSRDPKVPADEVCGCPLVRDVFELTGDFCRLPKRQCNRHYCWEKLRRAEVDLERVRVWYKLDELFEQERNVRTAMTNRAGLLALMLHQTIQHDPLTTDLRSSADR

SEQ ID NO: 59 - protein, 124 aa, Artificial Sequence
  Synthetic polynucleotide
MIKLKFGVFFTVLLSSAYAHGTPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

SEQ ID NO: 60 - protein, 258 aa, Artificial Sequence
  Synthetic polynucleotide
MVKIIFVFFIFLSSFSYANDDKLYRADSRPPDEIKQSGGLMPRGQNEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 61 - protein, 124 aa, Artificial Sequence
  Synthetic polynucleotide
MIKLKFGVFFTVLLSSAYAHGTPQNITDLCAEYHNTQIHTLNDKILSYTESLAGNREMAIITFKNGATFQVEVPGSQHIDSQKKAIERMKDTLRIAYLTEAKVEKLCVWNNKTPHAIAAISMAN

SEQ ID NO: 62 - protein, 18 aa, Artificial Sequence
  Hp91 (a non-limiting example sequence)
DPNAPKRPPSAFFLFCSE

SEQ ID NO: 63 - protein, 96 aa, Artificial Sequence
  CCL20 (a non-limiting example)
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 64 - protein, 92 aa, Artificial Sequence
  CCL3 (a non-limiting example)
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 65 - protein, 144 aa, Artificial Sequence
  GM-CSF (a non-limiting example sequence)
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 66 - protein, 204 aa, Artificial Sequence
  CSF (a non-limiting example)
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 67 - protein, 7 aa, Artificial Sequence
  Synthetic peptide
QEINSSY

SEQ ID NO: 68 - protein, 7 aa, Artificial Sequence
  Synthetic peptide
SHPRLSA

SEQ ID NO: 69 - protein, 7 aa, Artificial Sequence
  Synthetic peptide
SMPNPMV

SEQ ID NO: 70 - protein, 7 aa, Artificial Sequence
  Synthetic peptide
GLQQVLL

SEQ ID NO: 71 - protein, 7 aa, Artificial Sequence
  Synthetic peptide
HELSVLL

SEQ ID NO: 72 - protein, 7 aa, Artificial Sequence
  Synthetic peptide
YAPQRLP

SEQ ID NO: 73 - protein, 7 aa, Artificial Sequence
  Synthetic peptide
TPRTLPT

SEQ ID NO: 74 - protein, 7 aa, Artificial Sequence
  Synthetic peptide
APVHSSI

SEQ ID NO: 75 - protein, 7 aa, Artificial Sequence
  Synthetic peptide
APPHALS

SEQ ID NO: 76 - protein, 7 aa, Artificial Sequence
  Synthetic peptide
TFSNRFI

SEQ ID NO: 77 - protein, 7 aa, Artificial Sequence
  Synthetic peptide
VVPTPPY

SEQ ID NO: 78 - protein, 7 aa, Artificial Sequence
  Synthetic peptide
ELAPDSP

SEQ ID NO: 79 - protein, 69 aa, Artificial Sequence
  shiga toxin (a non-limiting example)
TPDCVTGKVEYTKYNDDDTFTVKVGDKELFTNRWNLQSLLLSAQITGMTVTIKQNACHNGGGFSEVIFR

SEQ ID NO: 80 - protein, 560 aa, Artificial Sequence
  diphtheria toxin (a non-limiting example)
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 81 - protein, 114 aa, Artificial Sequence
  Synthetic polynucleotide
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 82 - protein, 297 aa, Artificial Sequence
  Synthetic polynucleotide
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 535 aa, Artificial Sequence
  CRM197 (a non-limiting example)
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 84 - protein, 11 aa, Artificial Sequence
  Synthetic peptide
MAVPMQLSCSR

SEQ ID NO: 85 - protein, 4 aa, Artificial Sequence
  Synthetic peptide
RSTG

SEQ ID NO: 86 - protein, 2 aa, Artificial Sequence
  Synthetic peptide
TR

SEQ ID NO: 87 - protein, 3 aa, Artificial Sequence
  Synthetic peptide
RSQ

SEQ ID NO: 88 - protein, 5 aa, Artificial Sequence
  Synthetic peptide
RSAGE

SEQ ID NO: 89 - protein, 2 aa, Artificial Sequence
  Synthetic peptide
RS

SEQ ID NO: 90 - protein, 2 aa, Artificial Sequence
  Synthetic peptide
GG

SEQ ID NO: 91 - protein, 9 aa, Artificial Sequence
  Synthetic peptide
GSGGSGGSG

SEQ ID NO: 92 - protein, 11 aa, Artificial Sequence
  Synthetic peptide
GGSGGSGGSGG

SEQ ID NO: 93 - protein, 14 aa, Artificial Sequence
  Synthetic peptide
GGSGGSGGSGGSGG

SEQ ID NO: 94 - protein, 17 aa, Artificial Sequence
  Synthetic peptide
GGSGGSGGSGGSGGSGG

SEQ ID NO: 95 - protein, 20 aa, Artificial Sequence
  Synthetic peptide
GGSGGSGGSGGSGGSGGSGG

SEQ ID NO: 96 - protein, 23 aa, Artificial Sequence
  Synthetic peptide
GGSGGSGGSGGSGGSGGSGGSGG

SEQ ID NO: 97 - protein, 16 aa, Artificial Sequence
  Synthetic peptide
GGSGGSGGSGGSGGSG

SEQ ID NO: 98 - protein, 16 aa, Artificial Sequence
  Synthetic peptide
GSGGSGGSGGSGGSGG

SEQ ID NO: 99 - protein, 470 aa, Artificial Sequence
  Synthetic polynucleotide
MGWSCIIFFLVATATGVHSQVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - DNA, 2109 bp, Artificial Sequence
  Synthetic polynucleotide
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcggacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 101 - DNA, 1428 bp, Artificial Sequence
  Synthetic polynucleotide
atgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccctttggggacaggatgtcacctcggtcccagtcaccaggccagccctgggctccaccaccccgccagcccacgatgtcacctcagccccggacaacaagccagccccgggctccaccgcccccccagcccacggtgtcacctcgtatcttgacaccaggccggccccggtttatcttgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggtttatctggccattgtctatctcattgccttggctgtcgctcaggttcgccgaaagaactacgggcagctggacatctttccagcccgggataaataccatcctatgagcgagtacgctctttaccacacccatgggcgctatgtgccccctagcagtcttttccgtagcccctatgagaaggtttctgcaggtaatggtggcagctatctctcttacacaaacccagcagtggcagccgcttctgccaacttgtag

SEQ ID NO: 102 - DNA, 1233 bp, Artificial Sequence
  Synthetic polynucleotide
atgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgctagaagtgatcacaaagagatgatggaggaacccggagacagccagcaacctgggtactcccaatgggggtggcttcttcctggaaccagcaccgtgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtga

SEQ ID NO: 103 - protein, 1331 aa, Homo Sapiens [sequence omitted]

SEQ ID NO: 104 - protein, 155 aa, Homo Sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 105 - protein, 476 aa, Homo Sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSPIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 106 - protein, 234 aa, Homo sapiens
MCFPKVLSDDMKKLKARMVMLLPTSAQGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 107 - DNA, 1929 bp, Artificial Sequence
  Synthetic polynucleotide
gcggcgtccgtccgtactgcagagccgctgccggagggtcgttttaaagggcccgcgcgttgccgccccctcggcccgccatgctgctatccgtgccgctgctgctcggcctcctcggcctggccgtcgccgagcctgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctccaaaaaaaaaaaaaaaaaa

SEQ ID NO: 108 - protein, 8 aa, Artificial Sequence
  Synthetic peptide
TCACACCT